(19) United States
(12) Patent Application Publication
(10) Pub. No.: US 2024/0132464 A1
(43) Pub. Date: Apr. 25, 2024

(54) HETEROCYCLIC COMPOUNDS AS E3 LIGASE INHIBITORS

(71) Applicant: Accutar Biotechnology Inc., Cranbury, NJ (US)

(72) Inventors: Ji Liu, Seattle, WA (US); Yimin Qian, Plainsboro, NJ (US); Pin Huang, Shanghai (CN); Xiangyan Sun, Shanghai (CN); Ke Liu, Shanghai (CN); Jie Fan, New York, NY (US)

(73) Assignee: Accutar Biotechnology Inc., Cranbury, NJ (US)

(21) Appl. No.: 18/476,063

(22) Filed: Sep. 27, 2023

Related U.S. Application Data
(60) Provisional application No. 63/377,479, filed on Sep. 28, 2022.

(51) Int. Cl. C07D 401/04; C07D 239/48; C07D 239/50; C07D 241/42; C07D 401/10; C07D 401/14; C07D 403/12; C07D 409/04; C07D 471/04; C07D 519/00

(52) U.S. Cl. CPC C07D 401/04; C07D 239/48; C07D 239/50; C07D 241/42; C07D 401/10; C07D 401/14; C07D 403/12; C07D 409/04; C07D 471/04; C07D 519/00

(57) ABSTRACT

The present disclosure relates to novel compounds that inhibit glucose-induced degradation-deficient (GID) E3 ligase, pharmaceutical compositions containing such compounds, and their use in prevention and treatment of cancer and related diseases and conditions.

This application claims priority from U.S. Provisional Patent Application No. 63/377,479, filed Sep. 28, 2022, which is hereby incorporated by reference in its entirety.

### FIELD OF THE DISCLOSURE

The present invention relates to compounds that inhibit glucose-induced degradation-deficient (GID) E3 ligase and pharmaceutical compositions comprising the compounds and methods of use therefor.

### BACKGROUND OF THE DISCLOSURE

Glucose-induced degradation-deficient (GID) E3 ligase is an enzyme involved in the degradation of superfluous gluconeogenic enzymes. See, e.g., Lampert, Fabienne, et al. “The multi-subunit GID/CTLH E3 ubiquitin ligase promotes cell proliferation and targets the transcription factor Hbp1 for degradation.” Elife 7 (2018): e35528. Glucose-induced degradation protein-4 homolog (GID4) is one of the gastric inhibitory polypeptide (GIP) family and part of a GID family that composes the CTLH E3 ligase complex. Among the E3 ligases for which 3D crystal structures are reported with bound ligands or partner substrate proteins are, for example, GID4 (Class CTHL). GID4 expression is regulated by the GID E3 ligase. GID4 recognizes N-terminal proline residues selectively in substrates to mark their ubiquitination and proteasomal degradation, but the mechanism of molecular recognition remained elusive. Dong et al. (Molecular Basis of GD4-Mediated Recognition of Degrons for the Pro/N-End Rule Pathway. Nat. Chem. Biol. 2018, 14, 466-473) reported the cocrystal structures of the globular domain of GID4 with N-end proline fragments and observed that the terminal proline forms key interactions with the residues at the bottom of a deep substrate binding pocket. In addition, the binding of degrons induces dynamic conformational change around the binding pocket to form hydrophobic interaction with the residues in the pocket.

Moreover, the GID4 subunit is a recognition component of the proteolytic pathway termed the Pro/N-end rule pathway, which conditionally destroys gluconeogenic enzymes. See, e.g., Chen, Shun-Jia, et al. “An N-end rule pathway that recognizes proline and destroys gluconeogenic enzymes.” Science 355.6323 (2017): eaa13655. For example, gluconeogenic enzymes Fbp1, Icl1, Mdh2, and Pck1 are conditionally short-lived substrates of the Gid4-dependent Pro/N-degron pathway. See, e.g., Varshavsky, Alexander. “N-degron and C-degron pathways of protein degradation.” Proceedings of the National Academy of Sciences 116.2 (2019): 358-366. Misregulation of gluconeogenic enzymes can promote cancer, diabetes, and other human diseases. See, e.g., A. Dunkler, J. Muller, N. Johnsson, Detecting protein-protein interactions with the splitubiquitin sensor. Methods Mol. Biol. 786, 115-130 (2012); D. G. Fraenkel, Yeast Intermediary Metabolism. (Cold Spring Harbor Laboratory Press, Cold Spring Harbor, NY, 2011). Accordingly, there is a need for novel E3 ligase inhibitors for treating diseases, such as cancer. One way to design such inhibitors is to inhibit the binding between GID4, which is a substrate-recognition subunit of the CTLH E3 ubiquitin-ligase complex, and its degron peptide Pro-Gly-Leu-Trp (i.e., PGLW).

### SUMMARY OF THE DISCLOSURE

In some embodiments, the present disclosure is directed to a compound of Formula (1) or a tautomer, stereoisomer or a mixture of stereoisomers, or a pharmaceutically acceptable salt, or hydrate, or deuterated derivative thereof:

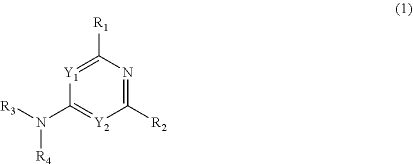

wherein:
Y₁ is either:
   chosen from CH, N, and CX₁, wherein X₁ is a halogen, or
   Y₁ and R₁ together form an optionally substituted 5 to 7 membered ring G;
Y₂ is chosen from CH, N, and CX₁, wherein X₁ is a halogen;
R₁ is either:
chosen from NHR₅, H, and C₁-C₄ alkyl, wherein R₅ is chosen from H and optionally substituted C₁-C₃ alkyl, or
   Y₁ and R₁ together form a 3- to 7-membered saturated or unsaturated ring, wherein the ring has 0 to 3 heteroatoms;
R₂ is chosen from NHR₆, H, and C₁-C₄ alkyl, wherein R₆ is chosen from H and optionally substituted C₁-C₃ alkyl; and
R₃ and R₄ are each independently chosen from hydrogen, an optionally substituted 5 to 7 membered ring, and optionally substituted C₁-C₃ alkyl; or
   R₃ and R₄ together form a 5 to 9 membered ring system D, wherein the ring is chosen from monocyclic rings and bicyclic rings, and wherein the monocyclic rings and bicyclic rings are optionally substituted with a halogen, a hydroxyl, an optionally substituted phenyl, an acetyl group, a 5 membered heteroaryl, an optionally substituted 9 membered heterobicyclic aryl, an optionally substituted benzimidazolone, and/or an optionally substituted C₁-C₃ alkyl.

In some embodiments, the compound of Formula (1) is a compound of Formula (1a):

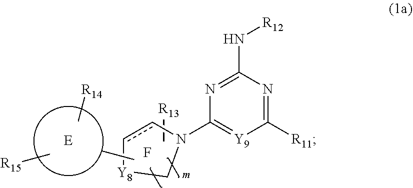

wherein:
m is 1, 2, or 3;
the dashed bond in ring F is either a single bond or a double bond; R₁₁ is chosen from a C₁-C₃ alkyl and NHR₂₁, wherein R₂₁ is chosen from H and a C₁-C₃ alkyl;
R₁₂ is chosen from H and a C₁-C₄ alkyl optionally substituted with a hydroxyl;
R₁₃ is either:
   absent,
   chosen from a halogen, a hydroxyl, and a C₁-C₃ alkyl optionally substituted with a hydroxyl, or
   R₁₃ and Y₈ together form a 5 membered heteroaryl ring;
Y₈ is either:
   chosen from O, CH₂, an acetyl group, and NR₁₄, wherein R₁₄ is chosen from H and a C₁-C₃ alkyl optionally substituted with a hydroxyl, or
   R₁₃ and Y₈ together form a 5 membered heteroaryl ring;
ring system E is chosen from 6 membered aryls and 9 membered bicyclic heteroaryls;
Y₉ is chosen from N and CX₂, wherein X₂ is chosen from H and a halogen;
R₁₄ is absent or a halogen; and
R₁₅ is absent or chosen from a halogen, a hydroxyl, a 5 membered heteroaryl having 1 to 3 heteroatoms and optionally substituted with up to two methyl groups,

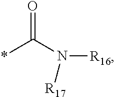

a trifluoromethyl group, a C₁-C₃ alkoxy group,

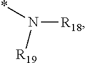

and

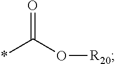

wherein:
R₁₆ is either:
   chosen from H and a C₁-C₆ alkyl optionally substituted with a tertiary amine, or
   R₁₆ and R₁₇ together form a 5 to 6 membered non-aromatic ring optionally substituted with up to two C₁-C₃ alkyls each independently optionally substituted with a hydroxyl;
R₁₇ is either:
   chosen from H and a C₁-C₃ alkyl, or
   R₁₆ and R₁₇ together form a 5 to 6 membered non-aromatic ring optionally substituted with up to two C₁-C₃ alkyls each independently optionally substituted with a hydroxyl;
R₁₈ is either:
   chosen from H and a C₁-C₆ alkyl optionally substituted with a tertiary amine or a hydroxyl, or
   R₁₈ and R₁₉ together form a 4 to 6 membered non-aromatic ring optionally substituted with a C₁-C₃ alkyl optionally substituted with a tertiary amine or a hydroxyl;
R₁₉ is either:
   chosen from H and a C₁-C₃ alkyl, or
   R₁₈ and R₁₉ together form a 4 to 6 membered non-aromatic ring optionally substituted with a C₁-C₃ alkyl optionally substituted with a tertiary amine or a hydroxyl; and
R₂₀ is chosen from C₁-C₃ alkyls;
   and wherein Formula (1a) encompasses all tautomers, stereoisomers, deuterated derivatives, pharmaceutically acceptable salts, and mixtures thereof.

In some embodiments, the ring system E is chosen from:

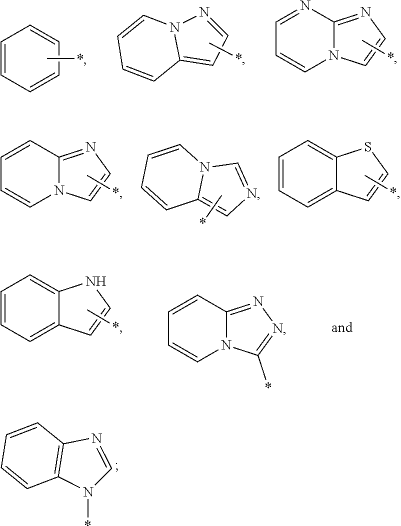

wherein each asterisk (*) denotes a point of attachment within Formula (1a).

In some embodiments, the compound of Formula (1a) is chosen from Formula (1aa) and Formula (1ab):

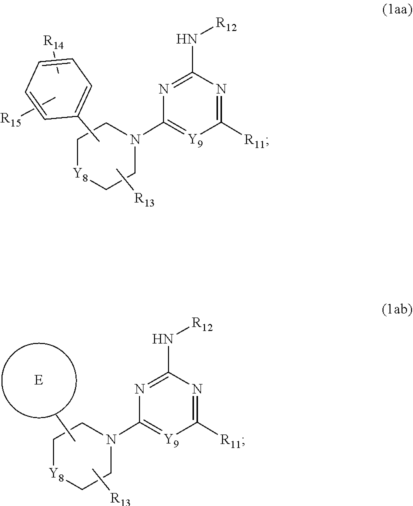

wherein R₁₄ and R₁₅ are not both absent, and wherein each of Formula (1aa) and Formula (1ab) encompass all tautomers, stereoisomers, pharmaceutically acceptable salts, and mixtures thereof.

In some embodiments, the compound of Formula (1) the compound is a compound of Formula (1 b):

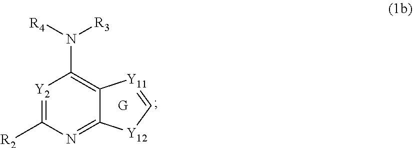

wherein:
each of Y₁₁ and Y₁₂ are chosen from N and CH;
at least one of Y₁₁ and Y₁₂ is N;
   and wherein Formula (1 b) encompasses all tautomers, stereoisomers, deuterated derivatives, pharmaceutically acceptable salts, and mixtures thereof.

In some embodiments, the compound of Formula (1 b) is a compound of Formula (1ba):

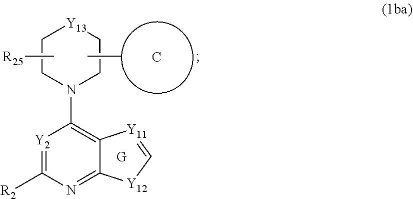

wherein:
   ring C is chosen from 9 membered bicyclic heteroaryls and 6 membered aryls optionally substituted with a halogen and/or a hydroxyl;
   Y₁₃ is chosen from CH₂, O, and NR₂₄ wherein R₂₄ is chosen from H and a C₁-C₃ alkyl optionally substituted with hydroxyl;
   R₂₅ is absent or chosen from a halogen, a hydroxyl, and a C₁-C₃ alkyl optionally substituted with a tertiary amine;
      and wherein Formula (1ba) encompasses all tautomers, stereoisomers, deuterated derivatives, pharmaceutically acceptable salts, and mixtures thereof.

In some embodiments, the compound of Formula (1) is a compound of Formula (1c):

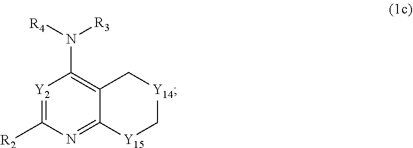

wherein:
   Y₁₅ is chosen from NH and CH₂;
   Y₁₄ is chosen from O, S, and CH₂;
   at least one of Y₁₅ and Y₁₄ is not CH₂;
      and wherein Formula (1c) encompasses all tautomers, stereoisomers, deuterated derivatives, pharmaceutically acceptable salts, and mixtures thereof.

In some embodiments, the compound of Formula (1c) is a compound of Formula (1ca):

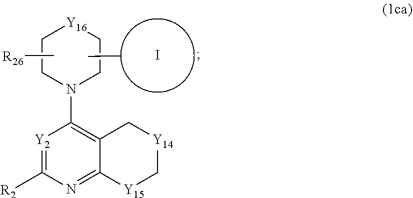

wherein:
   ring I is chosen from a 9 membered bicyclic heteroaryl and a 5 or 6 membered aryl or heteroaryl ring optionally substituted with a halogen and/or a hydroxyl;
   Y₁₆ is chosen from CH₂, O, and NR₂₇ wherein R₂₇ is chosen from H and a C₁-C₃ alkyl optionally substituted with hydroxyl;
   R₂₆ is absent or chosen from a halogen, a hydroxyl, and a C₁-C₃ alkyl optionally substituted with a tertiary amine;
      and wherein Formula (1 ca) encompasses all tautomers, stereoisomers, deuterated derivatives, pharmaceutically acceptable salts, and mixtures thereof.

In some embodiments, the compound is chosen from Formula (1), Formula (1b), and Formula (1c); and R₃ is a hydrogen or an optionally substituted C₁-C₃ alkyl.

In some embodiments, R₄ is chosen from C₁-C₃ alkyl optionally substituted with a 9 membered bicyclic heteroaryl,

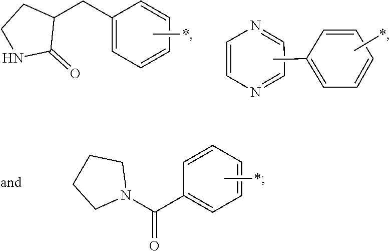

wherein the asterisks (*) denotes a point of attachment in Formula (1).

Also disclosed are compounds of Formula (2) or a tautomer, stereoisomer or a mixture of stereoisomers, or a pharmaceutically acceptable salt, or hydrate, or deuterated derivative thereof:

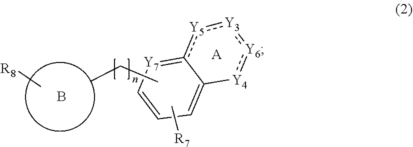

wherein:
Y₃ is chosen from CH, CH₂, N, CO, and NH;
Y₄ is chosen from CH, N, and CH₂;
Y₅ is chosen from CH, CH₂, CNHR₁₀, CO, N, 5 membered heteroaryl rings comprising two nitrogen atoms and three carbon atoms wherein the heteroaryl ring is optionally substituted with a methyl, and NH, wherein R₁₀ is chosen from H and C₁-C₄ alkyl;
Y₆ is chosen from CH and CH₂;
Y₇ is chosen from CH, N, and C—OH;
R₇ is absent or chosen from OH, halogen, 5 membered heteroaryl rings comprising two nitrogen atoms and three carbon atoms wherein the heteroaryl ring is optionally substituted with a methyl, and methyl;
ring A is an aromatic or non-aromatic 6-membered heterocyclic ring;
n is 0 or 1;
ring system B is chosen from:
   5 to 6 membered rings optionally comprising up to three nitrogen atoms, a sulfur atom, and/or an oxygen atom,
   bicyclic 9 or 10 membered rings comprising at least one aromatic ring, optionally comprising up to three nitrogen atoms, a sulfur atom, and/or a carbonyl, and bicyclic 7 or 8 membered aliphatic rings comprising an amine, and
wherein ring system B is optionally substituted with up to two halogens, a methyl, and/or NH₂;
R₈ is absent or chosen from:
   aliphatic amines comprising up to two nitrogen atoms, up to 7 carbon atoms, and optionally an alkoxy,
   N-substituted amides wherein at least one N-substituent comprises up to 7 carbon atoms and, optionally, an amine, a hydroxyl, and/or an alkoxy, and
   C-substituted carbonyls wherein the C-substituent comprises up to 6 carbon atoms and a tertiary amine;
      and wherein Formula (2) encompasses all tautomers, stereoisomers, isotopes, pharmaceutically acceptable salts, and mixtures thereof.

In some embodiments, the compound of Formula (2) is a compound of Formula (2a):

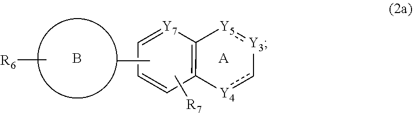

wherein:
Y₃ is chosen from N, NH, CH, CO, and C—NH₂;
Y₄ is chosen from N and CH;
Y₅ is chosen from N, NH, CH, CO, and C—NH₂;
at least one of Y₃, Y₄, and Y₅ is N;
each dashed bond in ring A independently represents a single or double bond, and
R₇ is absent or chosen from OH, halogen, and methyl;
   and wherein Formula (2a) encompasses all tautomers, stereoisomers, deuterated derivatives, pharmaceutically acceptable salts, and mixtures thereof.

In some embodiments, the compound of Formula (2a) is a compound of Formula (2aa):

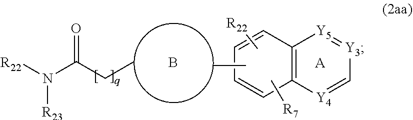

wherein:
q is 0 or 1;
R₂₂ is chosen from H and a C₁-C₃ alkyl;
R₂₃ consists of H, C, optionally N, and optionally O; and
R₂₃ comprises up to 7 carbon atoms and, optionally, an amine, a hydroxyl, and/or an alkoxy;
ring A is an aromatic 6-membered heterocyclic ring;
   and wherein Formula (2aa) encompasses all tautomers, stereoisomers, deuterated derivatives, pharmaceutically acceptable salts, and mixtures thereof.

In some embodiments, ring system B consists of C, H, optionally N, optionally S, optionally F, and optionally Cl.

In some embodiments, ring system B is chosen from

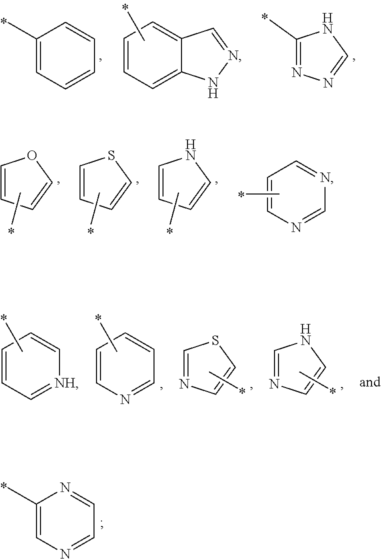

wherein each asterisk (*) denotes a point of attachment within Formula (2), Formula (2a), or Formula (2aa).

In some embodiments, the compound of Formula (2) is a compound of Formula (2b):

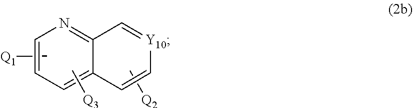

wherein:
Y₁₀ is N or CH;
Q₁ is chosen from a 5 to 6 membered rings optionally comprising up to two nitrogen atoms and a bicyclic 7 or 8 membered aliphatic rings comprising an amine; and
Q₂ and Q₃ are each independently a 5 membered heteroaryl ring comprising two nitrogen atoms and three carbon atoms wherein each heteroaryl ring is optionally substituted with a methyl;
   and wherein Formula (2b) encompasses all tautomers, stereoisomers, deuterated derivatives, pharmaceutically acceptable salts, and mixtures thereof.

Disclosed are pharmaceutical compositions comprising the compound of Formula (1) or Formula (2) and at least one additional component chosen from pharmaceutically acceptable carriers, pharmaceutically acceptable vehicles, and pharmaceutically acceptable excipients.

In some embodiments, the compound of Formula (1) or Formula (2) is present in a therapeutically effective amount.

Disclosed are methods of treating cancer in a subject in need thereof, comprising administering to the subject an effective amount of a compound of Formula (1) or Formula (2) or of the pharmaceutical composition comprising Formula (1) or Formula (2).

In some embodiments, the cancer is breast cancer. In some embodiments, the cancer is pancreatic cancer. In some embodiments, the cancer is colorectal cancer.

Disclosed are uses of a compound of Formula (1) or Formula (2) or a pharmaceutical composition comprising Formula (1) or Formula (2), in the preparation of a medicament.

Disclosed are methods of inhibiting E3 ligase in a cell, comprising contacting the cell Formula (1) or Formula (2) or a pharmaceutical composition comprising Formula (1) or Formula (2).

In some embodiments the cancer is selected from breast cancer, lung cancer, pancreatic cancer, colorectal cancer, gall bladder cancer, thyroid cancer, bile duct cancer, ovarian cancer, endometrial cancer, prostate cancer, and esophageal cancer.

In some embodiments, the method further comprises administering to the subject in combination with an anti-cancer agent.

### DETAILED DESCRIPTION OF THE DISCLOSURE

### Definitions

A dash (“-”) that is not between two letters or symbols is used to indicate a point of attachment for a substituent. For example, —CN is attached through the carbon atom.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅-6 alkyl.

The term “acyl” as used herein refers to R—C(O)— groups such as, but not limited to, (alkyl)-C(O)—, (alkenyl)-C(O)—, (alkynyl)-C(O)—, (aryl)-C(O)—, (cycloalkyl)-C(O)—, (heteroaryl)-C(O)—, and (heterocyclyl)-C(O)—, wherein the group is attached to the parent molecular structure through the carbonyl functionality. In some embodiments, it is a C₁₋₁₀ acyl radical which refers to the total number of chain or ring atoms of the, for example, alkyl, alkenyl, alkynyl, aryl, cycloalkyl, or heteroaryl, portion plus the carbonyl carbon of acyl. For example, a C₄-acyl has three other ring or chain atoms plus carbonyl.

The term “alkenyl” as used herein refers to an unsaturated straight or branched hydrocarbon having at least one carbon-carbon double bond, such as a straight or branched group of 2-8 carbon atoms, referred to herein as (C₂-C₈)alkenyl. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkyl” as used herein refers to a saturated straight or branched hydrocarbon, such as a straight or branched group of 1-8 carbon atoms, referred to herein as C₁-8 alkyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl. In some embodiments, “alkyl” is a straight-chain hydrocarbon. In some embodiments, “alkyl” is a branched hydrocarbon.

The term “alkoxy” means a straight or branched chain saturated hydrocarbon containing 1-12 carbon atoms containing a terminal “O” in the chain, e.g., —O(alkyl). Examples of alkoxy groups include, without limitation, methoxy, ethoxy, propoxy, butoxy, t-butoxy, or pentoxy groups.

The term “alkylene” as used herein referes to a divalent alkyl radical. Representative examples of C₁₋₁₀ alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene and n-decylene.

The term “alkynyl” as used herein refers to an unsaturated straight or branched hydrocarbon having at least one carbon-carbon triple bond, such as a straight or branched group of 2-8 carbon atoms, referred to herein as (C₂-C₈)alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “alkylamino” as used herein refers to an alkyl group which is substituted with or contains an amino group, wherein the amino group is optionally substituted with 1 or 2 alkyl groups. An exemplary alkylamino group includes, but is not limited to, —(CH₂)₂—N(CH₃)₂. An example of a C₄ alkylamino group includes but is not limited to —(CH₂)₂—N(CH₃)₂.

The term “aryl” as used herein refers to a mono-, bi-, or other multi-carbocyclic, aromatic ring system with 5 to 14 ring atoms. The aryl group can optionally be fused to one or more rings selected from aryls, cycloalkyls, heteroaryls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include but are not limited to a monocyclic aromatic ring system, wherein the ring comprises 6 carbon atoms.

The term “cyano” as used herein refers to —CN.

The term “cycloalkyl” as used herein refers to a saturated or unsaturated cyclic, bicyclic, or bridged bicyclic hydrocarbon group of 3-16 carbons, or 3-8 carbons, referred to herein as “(C₃-C₅)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone.

Cycloalkyl groups can be fused to other cycloalkyl (saturated or partially unsaturated), aryl, or heterocyclyl groups, to form a bicycle, tetracycle, etc. The term “cycloalkyl” also includes bridged and spiro-fused cyclic structures which may or may not contain heteroatoms.

The terms “halo” or “halogen” as used herein refer to —F, —Cl, —Br, and/or —I.

“Haloalkyl” means an alkyl group substituted with one or more halogens. Examples of haloalkyl groups include, but are not limited to, trifluoromethyl, difluoromethyl, pentafluoroethyl, trichloromethyl, etc.

The term “heteroaryl” as used herein refers to a mono-, bi-, or multi-cyclic, aromatic ring system containing one or more heteroatoms, for example 1-3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Exemplary heteroaryl groups include, but are not limited to, a monocyclic aromatic ring, wherein the ring comprises 2-5 carbon atoms and 1-3 heteroatoms, referred to herein as “(C₂-C₅)heteroaryl.” Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups also include, but are not limited to, a bicyclic aromatic ring, wherein the ring comprises 5-14 carbon atoms and 1-3 heteroatoms, referred to herein as “(C₅-C₁₄)heteroaryl.” Representative examples of heteroaryl include, but not limited to, indazolyl, indolyl, azaindolyl, indolinyl, benzotriazolyl, benzoxadiazolyl, imidazolyl, cinnolinyl, imidazopyridyl, pyrazolopyridyl, pyrrolopyridyl, quinolinyl, isoquinolinyl, quinazolinyl, quinazolinonyl, indolinonyl, isoindolinonyl, tetrahydronaphthyl, tetrahydroquinolinyl, tetrahydroisoquinolinyl, and the like.

The terms “heterocycle,” “heterocyclyl,” or “heterocyclic” as used herein each refer to a saturated or unsaturated 3- to 18-membered ring containing one, two, three, or four heteroatoms independently selected from nitrogen, oxygen, phosphorus, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

The term “hydroxyalkyl” as used herein refer to an alkyl group which is substituted with or contains a hydroxy group. An exemplary hydroxyalkyl group includes, but is not limited to, —CH₂—OH. An exemplary C₁ hydroxyalkyl group is —CH₂—OH.

“Spirocycloalkyl” or “spirocyclyl” means carbogenic bicyclic ring systems with both rings connected through a single atom. The rings can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. A (C₃₋₁₂)spirocycloalkyl is a spirocycle containing between 3 and 12 carbon atoms.

“Spiroheterocycloalkyl” or “spiroheterocyclyl” means a spirocycle wherein at least one of the rings is a heterocycle one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

“Isomers” means compounds having the same number and kind of atoms, and hence the same molecular weight, but differing with respect to the arrangement or configuration of the atoms in space.

“Stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

It is well-known in the art that the biological and pharmacological activity of a compound is sensitive to the stereochemistry of the compound. For example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the disclosure from this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. In some embodiments, an enantiomer or stereoisomer may be provided substantially free of the corresponding enantiomer.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Thus, if one enantiomer is pharmacologically more active, less toxic, or has a preferred disposition in the body than the other enantiomer, it would be therapeutically more beneficial to administer that enantiomer preferentially. In this way, the patient undergoing treatment would be exposed to a lower total dose of the drug and to a lower dose of an enantiomer that is possibly toxic or an inhibitor of the other enantiomer.

The compounds disclosed herein may exist as tautomers and both tautomeric forms are intended to be encompassed by the scope of the present disclosure, even if only one tautomeric structure is depicted.

Additionally, unless otherwise stated, structures described herein are also meant to include compounds that differ only in the presence of one or more isotopically enriched atoms. For example, compounds having the present structures except for the replacement of hydrogen by deuterium (²H) or tritium (³H), or the replacement of a carbon by a ¹³C- or ¹⁴C-carbon atom are within the scope of this disclosure. Such compounds may be useful as, for example, analytical tools, probes in biological assays, or therapeutic agents.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present disclosure that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the present disclosure. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

As used herein, “a pharmaceutically acceptable salt” and/or “deuterated derivative thereof” is intended to encompass pharmaceutically acceptable salts of any one of the referenced compounds, deuterated derivatives of any one of the referenced compounds, and pharmaceutically acceptable salts of those deuterated derivatives.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.). Chemical names were generated using PerkinElmer ChemDraw© Professional, version 17.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. In some embodiments, an enantiomer or stereoisomer may be provided substantially free of the corresponding enantiomer.

As used herein, “cancer” refers to diseases, disorders, and conditions that involve abnormal cell growth with the potential to invade or spread to other parts of the body. Exemplary cancers include, but are not limited to, breast cancer, lung cancer, ovarian cancer, endometrial cancer, prostate cancer, and esophageal cancer.

“Subject” refers to an animal, such as a mammal, that has been or will be the object of treatment, observation, or experiment. The methods described herein may be useful for both human therapy and veterinary applications. In one embodiment, the subject refers to, for example, primates (e.g., humans, male or female), cows, sheep, goats, horses, dogs, cats, rabbits, rats, mice, fish, birds and the like. In some embodiments, the subject is a primate. In some embodiments, the subject is a human.

As used herein, the term “inhibit,” “inhibition,” or “inhibiting” refers to the reduction or suppression of a given condition, symptom, or disorder, or disease, or a significant decrease in the baseline activity of a biological activity or process.

As used herein, the term “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically (e.g., through stabilization of a discernible symptom), physiologically, (e.g., through stabilization of a physical parameter), or both. In yet another embodiment, “treat,” “treating,” or “treatment” refers to preventing or delaying the onset or development or progression of the disease or disorder.

As used herein, a subject is “in need of” a treatment if such subject would benefit biologically, medically or in quality of life from such treatment.

### Compounds

Disclosed are compounds of Formula (1) or a tautomer, stereoisomer or a mixture of stereoisomers, or a pharmaceutically acceptable salt, or hydrate, or deuterated derivative thereof:

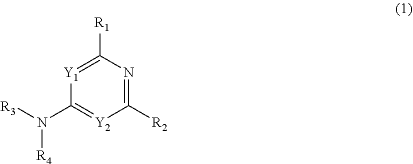

wherein:
Y₁ is either:
   chosen from CH, N, and CX₁, wherein X₁ is a halogen, or
   Y₁ and R₁ together form an optionally substituted 3 to 7 membered saturated or unsaturated ring G, wherein the ring has 0 to 3 heteroatoms;
Y₂ is chosen from CH, N, and CX₁, wherein X₁ is a halogen;
R₁ is either:
chosen from NHR₅, H, and C₁-C₄ alkyl, wherein R₅ is chosen from H and optionally substituted C₁-C₃ alkyl, or
   Y₁ and R₁ together form a 3 to 7 membered saturated or unsaturated ring G, wherein the ring has 0 to 3 heteroatoms;
   R₂ is chosen from NHR₆, H, and C₁-C₄ alkyl, wherein R₆ is chosen from H and optionally substituted C₁-C₃ alkyl; and
   R₃ and R₄ are each independently chosen from hydrogen, an optionally substituted 5 to 7 membered ring, and optionally substituted C₁-C₃ alkyl; or

R₃ and R₄ together form a 5 to 9 membered ring system D, wherein the ring is chosen from monocyclic rings and bicyclic rings, and wherein the monocyclic rings and bicyclic rings are optionally substituted with a halogen, a hydroxyl, an optionally substituted phenyl, an acetyl group, a 5 membered heteroaryl, an optionally substituted 9 membered heterobicyclic aryl, an optionally substituted benzimidazolone, and/or an optionally substituted C₁-C₃ alkyl. In some embodiments, ring G of the compound of Formula (1) is a 5 to 7 membered ring. In some embodiments, the compound of Formula (1) is a compound of Formula (1a):

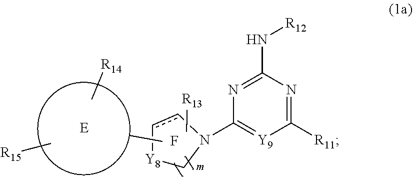

wherein:
m is 1, 2, or 3;
the dashed bond in ring F is either a single bond or a double bond; R₁₁ is chosen from a C₁-C₃ alkyl and NHR₂₁, wherein R₂₁ is chosen from H and a C₁-C₃ alkyl;
R₁₂ is chosen from H and a C₁-C₄ alkyl optionally substituted with a hydroxyl;
R₁₃ is either:
   absent,
   chosen from a halogen, a hydroxyl, and a C₁-C₃ alkyl optionally substituted with a hydroxyl, or
   R₁₃ and Y₈ together form a 5 membered heteroaryl ring;
Y₈ is either:
   chosen from O, CH₂, an acetyl group, and NR₁₄, wherein R₁₄ is chosen from H and a C₁-C₃ alkyl optionally substituted with a hydroxyl, or R₁₃ and Y₈ together form a 5 membered heteroaryl ring;
ring system E is chosen from 6 membered aryls and 9 membered bicyclic heteroaryls;
Y₉ is chosen from N and CX₂, wherein X₂ is chosen from H and a halogen;
R₁₄ is absent or a halogen; and
R₁₅ is absent or chosen from a halogen, a hydroxyl, a 5 membered heteroaryl having 1 to 3 heteroatoms and optionally substituted with up to two methyl groups,

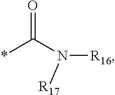

a trifluoromethyl group, a C₁-C₃ alkoxy group,

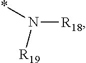

and

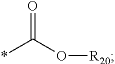

wherein:
R₁₆ is either:
   chosen from H and a C₁-C₆ alkyl optionally substituted with a tertiary amine, or
   R₁₆ and R₁₇ together form a 5 to 6 membered non-aromatic ring optionally substituted with up to two C₁-C₃ alkyls each independently optionally substituted with a hydroxyl;
R₁₇ is either:
   chosen from H and a C₁-C₃ alkyl, or
   R₁₆ and R₁₇ together form a 5 to 6 membered non-aromatic ring optionally substituted with up to two C₁-C₃ alkyls each independently optionally substituted with a hydroxyl;
R₁₈ is either:
   chosen from H and a C₁-C₆ alkyl optionally substituted with a tertiary amine or a hydroxyl, or
   R₁₈ and R₁₉ together form a 4 to 6 membered non-aromatic ring optionally substituted with a C₁-C₃ alkyl optionally substituted with a tertiary amine or a hydroxyl;
R₁₉ is either:
   chosen from H and a C₁-C₃ alkyl, or
   R₁₈ and R₁₉ together form a 4 to 6 membered non-aromatic ring optionally substituted with a C₁-C₃ alkyl optionally substituted with a tertiary amine or a hydroxyl; and
R₂₀ is chosen from C₁-C₃ alkyls;
   and wherein Formula (1a) encompasses all tautomers, stereoisomers, deuterated derivatives, pharmaceutically acceptable salts, and mixtures thereof.

In some embodiments, ring system E is chosen from:

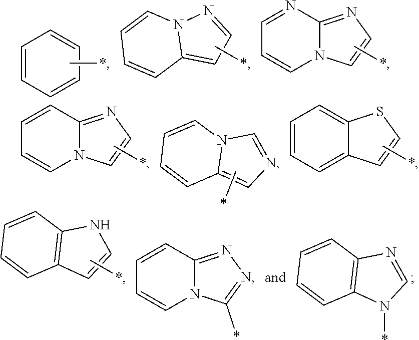

wherein each asterisk (*) denotes a point of attachment within Formula (1a).

In some embodiments, the compound of Formula (1a) is chosen from Formula (1aa) and Formula (1ab):

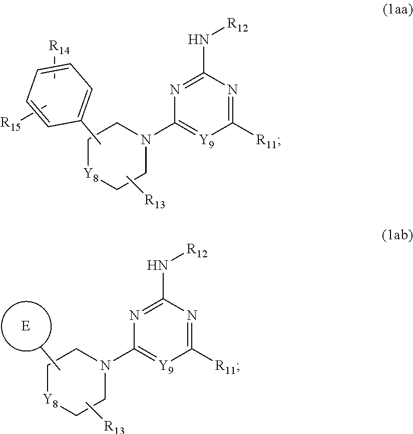

wherein R₁₄ and R₁₅ are not both absent, and wherein each of Formula (1aa) and Formula (1ab) encompass all tautomers, stereoisomers, pharmaceutically acceptable salts, and mixtures thereof.

In some embodiments, the compound of Formula (1) the compound is a compound of Formula (1b):

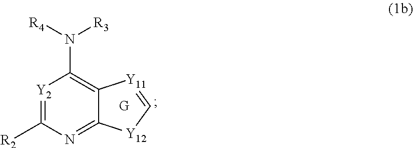

wherein:
   each of Y₁₁ and Y₁₂ are chosen from N and CH;
   at least one of Y₁₁ and Y₁₂ is N;
      and wherein Formula (1 b) encompasses all tautomers, stereoisomers, deuterated derivatives, pharmaceutically acceptable salts, and mixtures thereof.

In some embodiments, the compound of Formula (1 b) is a compound of Formula (1ba):

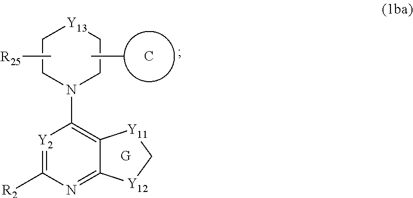

wherein:
   ring C is chosen from 9 membered bicyclic heteroaryls and 6 membered aryls optionally substituted with a halogen and/or a hydroxyl;
   Y₁₃ is chosen from CH₂, O, and NR₂₄ wherein R₂₄ is chosen from H and a C₁-C₃ alkyl optionally substituted with hydroxyl;
   R₂₅ is absent or chosen from a halogen, a hydroxyl, and a C₁-C₃ alkyl optionally substituted with a tertiary amine;
      and wherein Formula (1ba) encompasses all tautomers, stereoisomers, deuterated derivatives, pharmaceutically acceptable salts, and mixtures thereof.

In some embodiments, the compound of Formula (1) is a compound of Formula (1c):

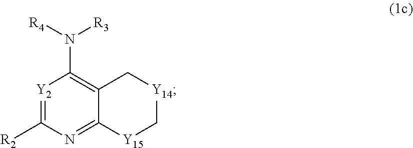

wherein:
   Y₁₅ is chosen from NH and CH₂;
   Y₁₄ is chosen from O, S, and CH₂;
   at least one of Y₁₅ and Y₁₄ is not CH₂;
      and wherein Formula (1c) encompasses all tautomers, stereoisomers, deuterated derivatives, pharmaceutically acceptable salts, and mixtures thereof.

In some embodiments, the compound of Formula (1c) is a compound of Formula (1ca):

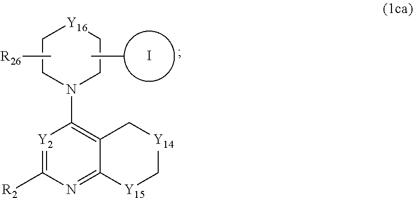

wherein:
ring I is chosen from a 9 membered bicyclic heteroaryl and a 5 or 6 membered aryl or heteroaryl ring optionally substituted with a halogen and/or a hydroxyl;
Y₁₆ is chosen from CH₂, O, and NR₂₇ wherein R₂₇ is chosen from H and a C₁-C₃ alkyl optionally substituted with hydroxyl;
R₂₆ is absent or chosen from a halogen, a hydroxyl, and a C₁-C₃ alkyl optionally substituted with a tertiary amine;
   and wherein Formula (1 ca) encompasses all tautomers, stereoisomers, deuterated derivatives, pharmaceutically acceptable salts, and mixtures thereof.

In some embodiments, the compound is chosen from Formula (1), Formula (1b), and Formula (1c); and R₃ is a hydrogen or an optionally substituted C₁-C₃ alkyl.

In some embodiments, R₄ is chosen from C₁-C₃ alkyl optionally substituted with a 9 membered bicyclic heteroaryl,

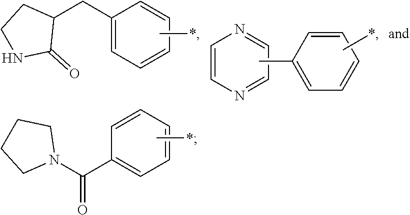

wherein the asterisks (*) denotes a point of attachment in Formula (1).

Also disclosed are compounds of Formula (2) or a tautomer, stereoisomer or a mixture of stereoisomers, or a pharmaceutically acceptable salt, or hydrate, or deuterated derivative thereof:

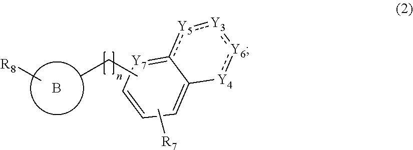

wherein:
Y₃ is chosen from CH, CH₂, N, CO, and NH;
Y₄ is chosen from CH, N, and CH₂;
Y₅ is chosen from CH, CH₂, CNHR₁₀, CO, N, 5 membered heteroaryl rings comprising two nitrogen atoms and three carbon atoms wherein the heteroaryl ring is optionally substituted with a methyl, and NH, wherein R₁₀ is chosen from H and C₁-C₄ alkyl;
Y₆ is chosen from CH and CH₂;
Y₇ is chosen from CH, N, and C—OH;
R₇ is absent or chosen from OH, halogen, 5 membered heteroaryl rings comprising two nitrogen atoms and three carbon atoms wherein the heteroaryl ring is optionally substituted with a methyl, and methyl;
ring A is an aromatic or non-aromatic 6-membered heterocyclic ring;
n is 0 or 1;
ring system B is chosen from:
   5 to 6 membered rings optionally comprising up to three nitrogen atoms, a sulfur atom, and/or an oxygen atom,
   bicyclic 9 or 10 membered rings comprising at least one aromatic ring, optionally comprising up to three nitrogen atoms, a sulfur atom, and/or a carbonyl, and bicyclic 7 or 8 membered aliphatic rings comprising an amine, and
wherein ring system B is optionally substituted with up to two halogens, a methyl, and/or NH₂;
R₆ is absent or chosen from:
   aliphatic amines comprising up to two nitrogen atoms, up to 7 carbon atoms, and optionally an alkoxy,
   N-substituted amides wherein at least one N-substituent comprises up to 7 carbon atoms and, optionally, an amine, a hydroxyl, and/or an alkoxy, and
   C-substituted carbonyls wherein the C-substituent comprises up to 6 carbon atoms and a tertiary amine;
      and wherein Formula (2) encompasses all tautomers, stereoisomers, isotopes, pharmaceutically acceptable salts, and mixtures thereof.

In some embodiments, the compound of Formula (2) is a compound of Formula (2a):

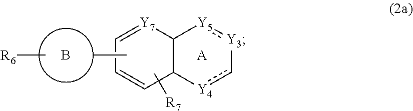

wherein:
Y₃ is chosen from N, NH, CH, CO, and C—NH₂;
Y₄ is chosen from N and CH;
Y₅ is chosen from N, NH, CH, CO, and C—NH₂;
at least one of Y₃, Y₄, and Y₅ is N;
each dashed bond in ring A independently represents a single or double bond, and
R₇ is absent or chosen from OH, halogen, and methyl;
   and wherein Formula (2a) encompasses all tautomers, stereoisomers, deuterated derivatives, pharmaceutically acceptable salts, and mixtures thereof.

In some embodiments, the compound of Formula (2a) is a compound of Formula (2aa):

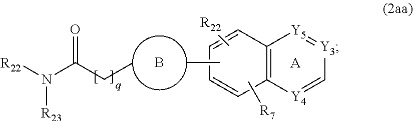

wherein:
q is 0 or 1;
R₂₂ is chosen from H and a C₁-C₃ alkyl;
R₂₃ consists of H, C, optionally N, and optionally O; and
R₂₃ comprises up to 7 carbon atoms and, optionally, an amine, a hydroxyl, and/or an alkoxy;
ring A is an aromatic 6-membered heterocyclic ring;
   and wherein Formula (2aa) encompasses all tautomers, stereoisomers, deuterated derivatives, pharmaceutically acceptable salts, and mixtures thereof.

In some embodiments, ring system B consists of C, H, optionally N, optionally S, optionally F, and optionally Cl.

In some embodiments, ring system B is chosen from

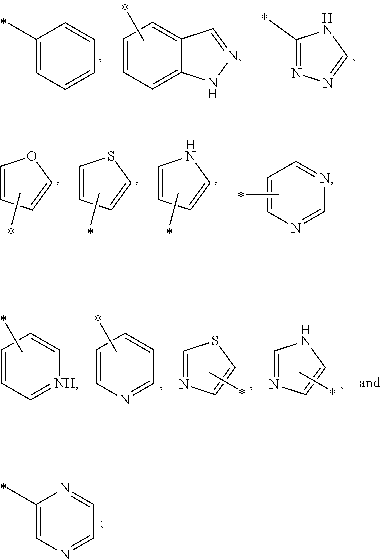

wherein each asterisk (*) denotes a point of attachment within Formula (2), Formula (2a), or Formula (2aa).

In some embodiments, the compound of Formula (2) is a compound of Formula (2b):

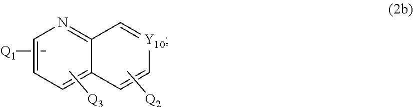

wherein:
Y₁₀ is N or CH;
Q₁ is chosen from a 5 to 6 membered rings optionally comprising up to two nitrogen atoms and a bicyclic 7 or 8 membered aliphatic rings comprising an amine; and
Q₂ and Q₃ are each independently a 5 membered heteroaryl ring comprising two nitrogen atoms and three carbon atoms wherein each heteroaryl ring is optionally substituted with a methyl;
   and wherein Formula (2b) encompasses all tautomers, stereoisomers, deuterated derivatives, pharmaceutically acceptable salts, and mixtures thereof.

In some embodiments, the compound of Formula (2) is chosen from

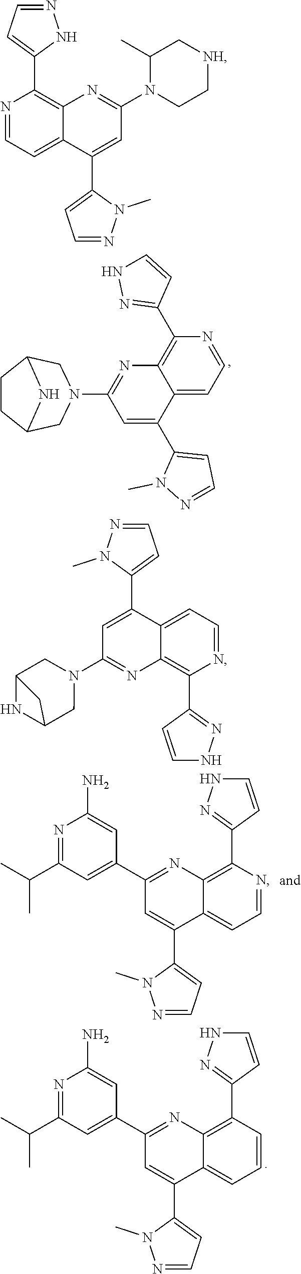

In some embodiments, provided herein is a compound chosen from the compounds listed in Table 1 or a tautomer, stereoisomer or a mixture of stereoisomers, or a pharmaceutically acceptable salt, or hydrate, or deuterated derivative of any of the foregoing.

TABLE 1
Exemplary Compounds of the Present Disclosure

| Compound | | |
|---|---|---|
| No. | Chemical Structure | IUPAC Name |
| 1 | 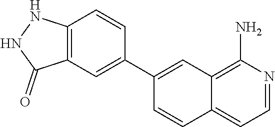 [SMILES: Nc1nccc2ccc(-c3ccc4[nH][nH]c(=O)c4c3)cc12] | 5-(1-aminoisoquinolin-7-yl)-1,2-dihydro-3H- indazol-3-one |
| 2 | 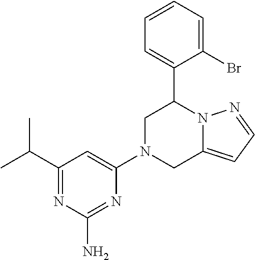 [SMILES: CC(C)c1cc(N2Cc3ccnn3C(c3ccccc3Br)C2)nc(N)n1] | 4-(7-(2-bromophenyl)-6,7- dihydropyrazolo[1,5-a]pyrazin-5(4H)-yl)-6- isopropylpyrimidin-2-amine |
| 3 | 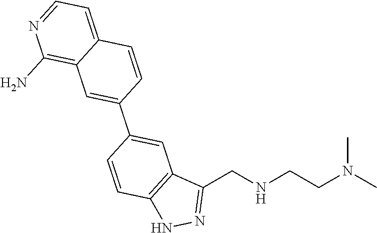 [SMILES: CN(C)CCNCc1n[nH]c2ccc(-c3ccc4ccnc(N)c4c3)cc12] | N1-((5-(1-aminoisoquinolin-7-yl)-1H-indazol- 3-yl)methyl)-N2,N2-dimethylethane-1,2- diamine |
| 4 | 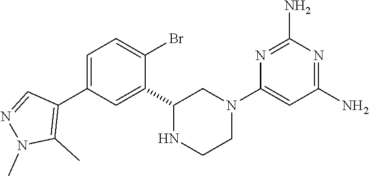 [SMILES: Cc1c(-c2ccc(Br)c([C@@H]3CN(c4cc(N)nc(N)n4)CCN3)c2)cnn1C] | (R/S)-6-(3-(2-bromo-5-(1,5-dimethyl-1H- pyrazol-4-yl)phenyl)piperazin-1- yl)pyrimidine-2,4-diamine |
| 5 | 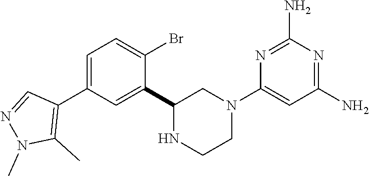 [SMILES: Cc1c(-c2ccc(Br)c([C@H]3CN(c4cc(N)nc(N)n4)CCN3)c2)cnn1C] | (S/R)-6-(3-(2-bromo-5-(1,5-dimethyl-1H- pyrazol-4-yl)phenyl)piperazin-1- yl)pyrimidine-2,4-diamine |
| 6 | 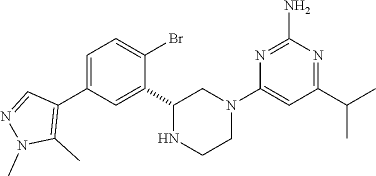 [SMILES: Cc1c(-c2ccc(Br)c([C@@H]3CN(c4cc(C(C)C)nc(N)n4)CCN3)c2)cnn1C] | (R/S)-4-(3-(2-bromo-5-(1,5-dimethyl-1H- pyrazol-4-yl)phenyl)piperazin-1-yl)-6- isopropylpyrimidin-2-amine |
| 7 | 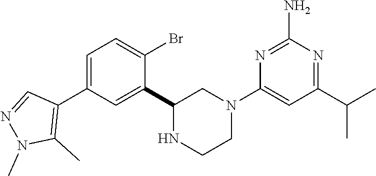 [SMILES: Cc1c(-c2ccc(Br)c([C@H]3CN(c4cc(C(C)C)nc(N)n4)CCN3)c2)cnn1C] | (S/R)-4-(3-(2-bromo-5-(1,5-dimethyl-1H- pyrazol-4-yl)phenyl)piperazin-1-yl)-6- isopropylpyrimidin-2-amine |
| 8 | 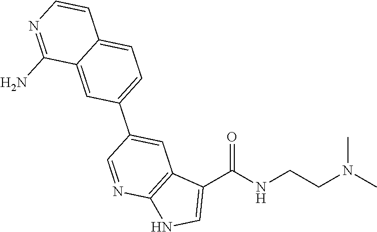 [SMILES: CN(C)CCNC(=O)c1c[nH]c2ncc(-c3ccc4ccnc(N)c4c3)cc12] | 5-(1-aminoisoquinolin-7-yl)-N-(2- (dimethylamino)ethyl)-1H-pyrrolo[2,3- b]pyridine-3-carboxamide |
| 9 | 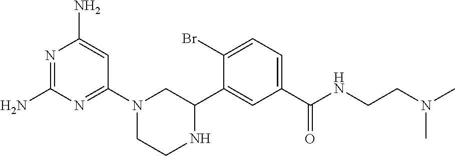 [SMILES: CN(C)CCNC(=O)c1ccc(Br)c(C2CN(c3cc(N)nc(N)n3)CCN2)c1] | 4-bromo-3-(4-(2,6-diaminopyrimidin-4- yl)piperazin-2-yl)-N-(2- (dimethylamino)ethyl)benzamide |
| 10 | 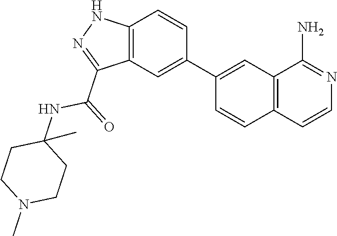 [SMILES: CN1CCC(C)(NC(=O)c2n[nH]c3ccc(-c4ccc5ccnc(N)c5c4)cc23)CC1] | 5-(1-aminoisoquinolin-7-yl)-N-(1,4- dimethylpiperidin-4-yl)-1H-indazole-3- carboxamide |
| 11 | 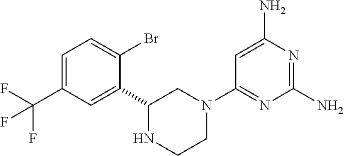 [SMILES: Nc1cc(N2CCN[C@H](c3cc(C(F)(F)F)ccc3Br)C2)nc(N)n1] | (R/S)-6-(3-(2-bromo-5- (trifluoromethyl)phenyl)piperazin-1- yl)pyrimidine-2,4-diamine |
| 12 | 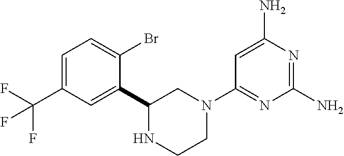 [SMILES: Nc1cc(N2CCN[C@@H](c3cc(C(F)(F)F)ccc3Br)C2)nc(N)n1] | (S/R)-6-(3-(2-bromo-5- (trifluoromethyl)phenyl)piperazin-1- yl)pyrimidine-2,4-diamine |
| 13 | 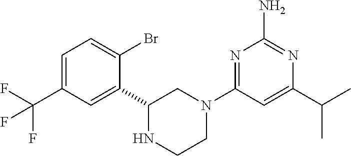 [SMILES: CC(C)c1cc(N2CCN[C@H](c3cc(C(F)(F)F)ccc3Br)C2)nc(N)n1] | (R/S)-4-(3-(2-bromo-5- (trifluoromethyl)phenyl)piperazin-1-yl)-6- isopropylpyrimidin-2-amine |
| 14 | 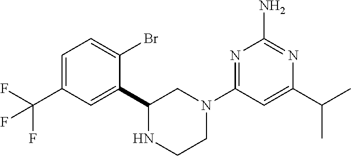 [SMILES: CC(C)c1cc(N2CCN[C@@H](c3cc(C(F)(F)F)ccc3Br)C2)nc(N)n1] | (S/R)-4-(3-(2-bromo-5- (trifluoromethyl)phenyl)piperazin-1-yl)-6- isopropylpyrimidin-2-amine |
| 15 | 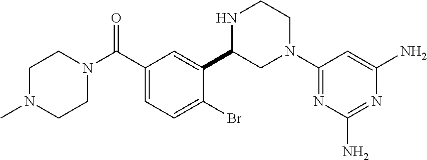 [SMILES: CN1CCN(C(=O)c2ccc(Br)c([C@@H]3CN(c4cc(N)nc(N)n4)CCN3)c2)CC1] | (R/S)-(4-bromo-3-(4-(2,6-diaminopyrimidin- 4-yl)piperazin-2-yl)phenyl)(4- methylpiperazin-1-yl)methanone |
| 16 | 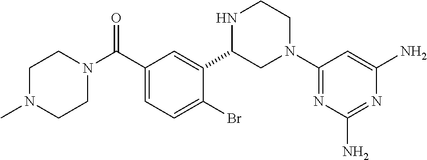 [SMILES: CN1CCN(C(=O)c2ccc(Br)c([C@H]3CN(c4cc(N)nc(N)n4)CCN3)c2)CC1] | (S/R)-(4-bromo-3-(4-(2,6-diaminopyrimidin- 4-yl)piperazin-2-yl)phenyl)(4- methylpiperazin-1-yl)methanone |
| 17 | 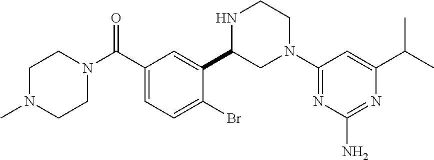 [SMILES: CC(C)c1cc(N2CCN[C@H](c3cc(C(=O)N4CCN(C)CC4)ccc3Br)C2)nc(N)n1] | (R/S)-(3-(4-(2-amino-6-isopropylpyrimidin-4- yl)piperazin-2-yl)-4-bromophenyl)(4- methylpiperazin-1-yl)methanone |
| 18 | 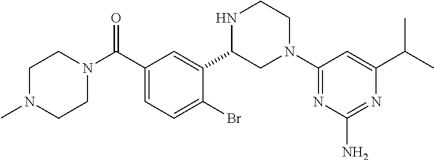 | (S/R)-(3-(4-(2-amino-6-isopropylpyrimidin-4- yl)piperazin-2-yl)-4-bromophenyl)(4- methylpiperazin-1-yl)methanone |
| 19 | 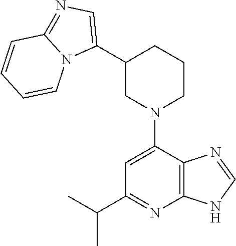 [SMILES: CC(C)c1cc(N2CCCC(c3cnc4ccccn34)C2)c2nc[nH]c2n1] | 7-(3-(imidazo[1,2-a]pyridin-3-yl)piperidin-1- yl)-5-isopropyl-3H-imidazo[4,5-b]pyridine |
| 20 | 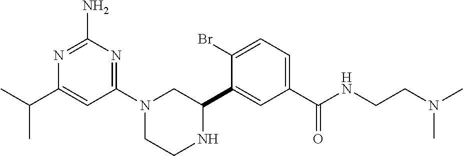 | (R/S)-3-(4-(2-amino-6-isopropylpyrimidin-4- yl)piperazin-2-yl)-4-bromo-N-(2- (dimethylamino)ethyl)benzamide |
| 21 | 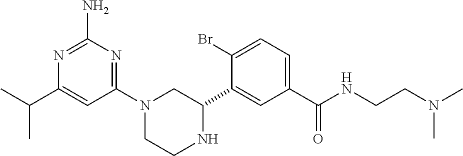 | (S/R)-3-(4-(2-amino-6-isopropylpyrimidin-4- yl)piperazin-2-yl)-4-bromo-N-(2- (dimethylamino)ethyl)benzamide |
| 22 | 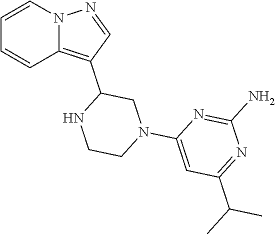 [SMILES: CC(C)c1cc(N2CCNC(c3cnn4ccccc34)C2)nc(N)n1] | 4-isopropyl-6-(3-(pyrazolo[1,5-a]pyridin-3- yl)piperazin-1-yl)pyrimidin-2-amine |
| 23 | 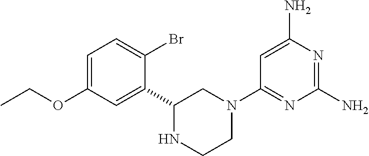 | (R/S)-6-(3-(2-bromo-5- ethoxyphenyl)piperazin-1-yl)pyrimidine-2,4- diamine |
| 24 | 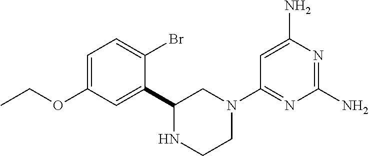 | (S/R)-6-(3-(2-bromo-5- ethoxyphenyl)piperazin-1-yl)pyrimidine-2,4- diamine |
| 25 | 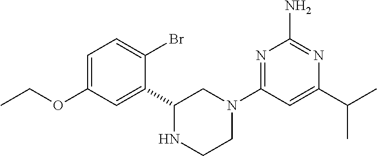 | (R/S)-4-(3-(2-bromo-5- ethoxyphenyl)piperazin-1-yl)-6- isopropylpyrimidin-2-amine |
| 26 | 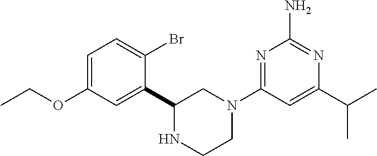 | (S/R)-4-(3-(2-bromo-5- ethoxyphenyl)piperazin-1-yl)-6- isopropylpyrimidin-2-amine |
| 27 | 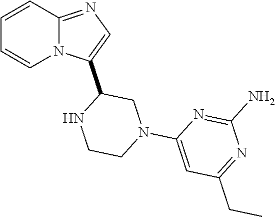 | (R/S)-4-ethyl-6-(3-(imidazo[1,2-a]pyridin-3- yl)piperazin-1-yl)pyrimidin-2-amine |
| 28 | 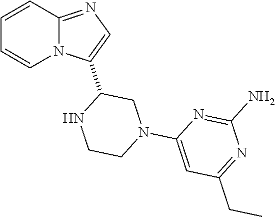 | (S/R)-4-ethyl-6-(3-(imidazo[1,2-a]pyridin-3- yl)piperazin-1-yl)pyrimidin-2-amine |
| 29 | 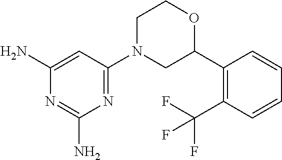 [SMILES: Nc1cc(N2CCOC(c3ccccc3C(F)(F)F)C2)nc(N)n1] | 6-(2-(2- (trifluoromethyl)phenyl)morpholino)pyrimidine- 2,4-diamine |
| 30 | 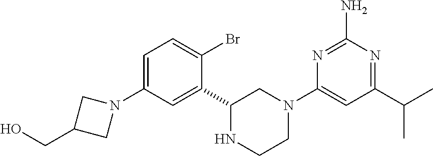 | (R/S)-(1-(3-(4-(2-amino-6- isopropylpyrimidin-4-yl)piperazin-2-yl)-4- bromophenyl)azetidin-3-yl)methanol |
| 31 | 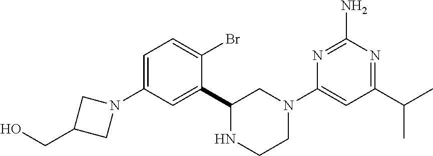 | (S/R)-(1-(3-(4-(2-amino-6- isopropylpyrimidin-4-yl)piperazin-2-yl)-4- bromophenyl)azetidin-3-yl)methanol |
| 32 | 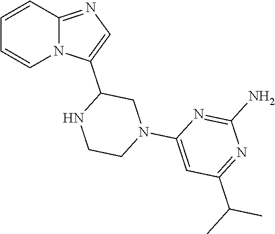 [SMILES: CC(C)c1cc(N2CCNC(c3cnc4ccccn34)C2)nc(N)n1] | 4-(3-(imidazo[1,2-a]pyridin-3-yl)piperazin-1- yl)-6-isopropylpyrimidin-2-amine |
| 33 | 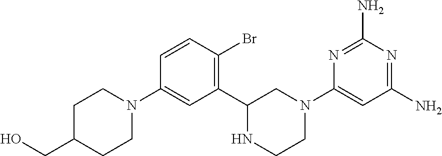 [SMILES: Nc1cc(N2CCNC(c3cc(N4CCC(CO)CC4)ccc3Br)C2)nc(N)n1] | (1-(4-bromo-3-(4-(2,6-diaminopyrimidin-4- yl)piperazin-2-yl)phenyl)piperidin-4- yl)methanol |
| 34 | 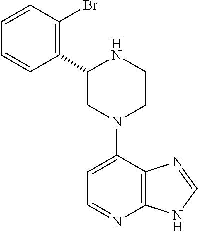 [SMILES: Brc1ccccc1[C@H]1CN(c2ccnc3[nH]cnc23)CCN1] | (S/R)-7-(3-(2-bromophenyl)piperazin-1-yl)- 3H-imidazo[4,5-b]pyridine |
| 35 | 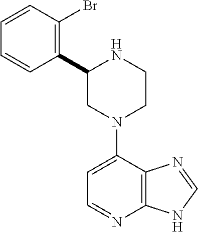 [SMILES: Brc1ccccc1[C@@H]1CN(c2ccnc3[nH]cnc23)CCN1] | (R/S)-7-(3-(2-bromophenyl)piperazin-1-yl)- 3H-imidazo[4,5-b]pyridine |
| 36 | 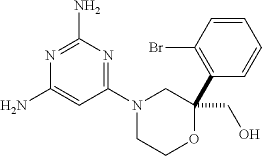 [SMILES: Nc1cc(N2CCO[C@](CO)(c3ccccc3Br)C2)nc(N)n1] | (R/S)-(2-(2-bromophenyl)-4-(2,6- diaminopyrimidin-4-yl)morpholin-2- yl)methanol |
| 37 | 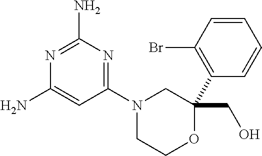 [SMILES: Nc1cc(N2CCO[C@@](CO)(c3ccccc3Br)C2)nc(N)n1] | (S/R)-(2-(2-bromophenyl)-4-(2,6- diaminopyrimidin-4-yl)morpholin-2- yl)methanol |
| 38 | 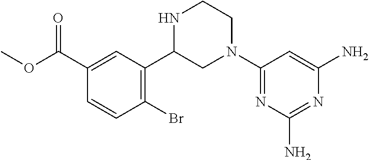 [SMILES: COC(=O)c1ccc(Br)c(C2CN(c3cc(N)nc(N)n3)CCN2)c1] | methyl 4-bromo-3-(4-(2,6-diaminopyrimidin- 4-yl)piperazin-2-yl)benzoate |
| 39 | 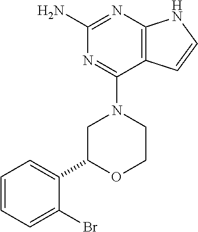 [SMILES: Nc1nc(N2CCO[C@H](c3ccccc3Br)C2)c2cc[nH]c2n1] | (R/S)-4-(2-(2-bromophenyl)morpholino)-7H- pyrrolo[2,3-d]pyrimidin-2-amine |
| 40 | 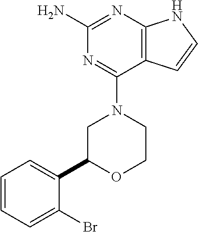 [SMILES: Nc1nc(N2CCO[C@@H](c3ccccc3Br)C2)c2cc[nH]c2n1] | (S/R)-4-(2-(2-bromophenyl)morpholino)-7H- pyrrolo[2,3-d]pyrimidin-2-amine |
| 41 | 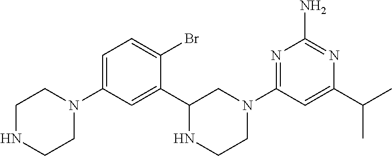 [SMILES: CC(C)c1cc(N2CCNC(c3cc(N4CCNCC4)ccc3Br)C2)nc(N)n1] | 4-(3-(2-bromo-5-(piperazin-1- yl)phenyl)piperazin-1-yl)-6- isopropylpyrimidin-2-amine |
| 42 | 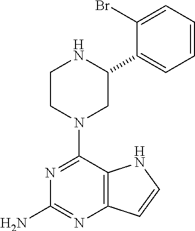 [SMILES: Nc1nc(N2CCN[C@H](c3ccccc3Br)C2)c2[nH]ccc2n1] | (R/S)-4-(3-(2-bromophenyl)piperazin-1-yl)- 5H-pyrrolo[3,2-d]pyrimidin-2-amine |
| 43 | 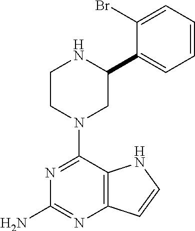 [SMILES: Nc1nc(N2CCN[C@@H](c3ccccc3Br)C2)c2[nH]ccc2n1] | (S/R)-4-(3-(2-bromophenyl)piperazin-1-yl)- 5H-pyrrolo[3,2-d]pyrimidin-2-amine |
| 44 | 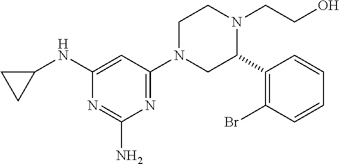 [SMILES: Nc1nc(NC2CC2)cc(N2CCN(CCO)[C@H](c3ccccc3Br)C2)n1] | (R/S)-2-(4-(2-amino-6- (cyclopropylamino)pyrimidin-4-yl)-2-(2- bromophenyl)piperazin-1-yl)ethan-1-ol |
| 45 | 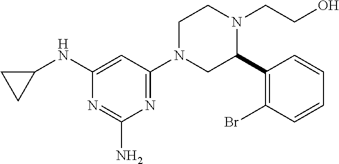 [SMILES: Nc1nc(NC2CC2)cc(N2CCN(CCO)[C@@H](c3ccccc3Br)C2)n1] | (S/R)-2-(4-(2-amino-6- (cyclopropylamino)pyrimidin-4-yl)-2-(2- bromophenyl)piperazin-1-yl)ethan-1-ol |
| 46 | 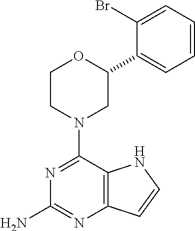 [SMILES: Nc1nc(N2CCO[C@H](c3ccccc3Br)C2)c2[nH]ccc2n1] | (R/S)-4-(2-(2-bromophenyl)morpholino)-5H- pyrrolo[3,2-d]pyrimidin-2-amine |
| 47 | 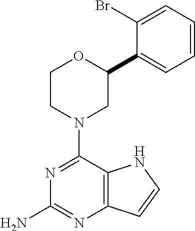 [SMILES: Nc1nc(N2CCO[C@@H](c3ccccc3Br)C2)c2[nH]ccc2n1] | (S/R)-4-(2-(2-bromophenyl)morpholino)-5H- pyrrolo[3,2-d]pyrimidin-2-amine |
| 48 | 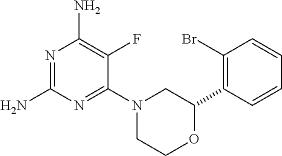 | (S/R)-6-(2-(2-bromophenyl)morpholino)-5- fluoropyrimidine-2,4-diamine |
| 49 | 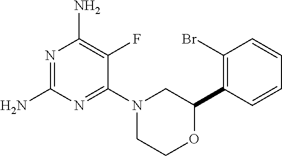 | (R/S)-6-(2-(2-bromophenyl)morpholino)-5- fluoropyrimidine-2,4-diamine |
| 50 | 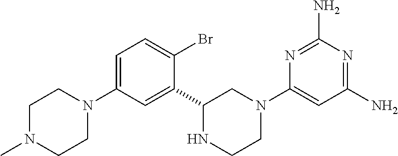 | (R/S)-6-(3-(2-bromo-5-(4-methylpiperazin-1- yl)phenyl)piperazin-1-yl)pyrimidine-2,4- diamine |
| 51 | 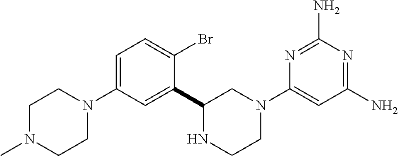 | (S/R)-6-(3-(2-bromo-5-(4-methylpiperazin-1- yl)phenyl)piperazin-1-yl)pyrimidine-2,4- diamine |
| 52 | 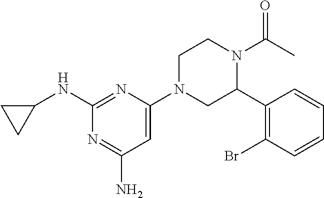 [SMILES: CC(=O)N1CCN(c2cc(N)nc(NC3CC3)n2)CC1c1ccccc1Br] | 1-(4-(6-amino-2- (cyclopropylamino)pyrimidin-4-yl)-2-(2- bromophenyl)piperazin-1-yl)ethan-1-one |
| 53 | 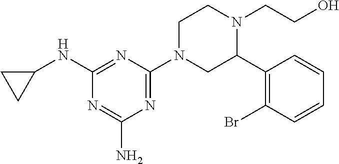 [SMILES: Nc1nc(NC2CC2)nc(N2CCN(CCO)C(c3ccccc3Br)C2)n1] | 2-(4-(4-amino-6-(cyclopropylamino)-1,3,5- triazin-2-yl)-2-(2-bromophenyl)piperazin-1- yl)ethan-1-ol |
| 54 | 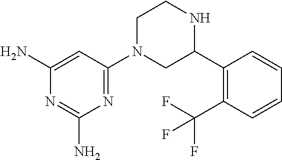 [SMILES: Nc1cc(N2CCNC(c3ccccc3C(F)(F)F)C2)nc(N)n1] | 6-(3-(2-(trifluoromethyl)phenyl)piperazin-1- yl)pyrimidine-2,4-diamine |
| 55 | 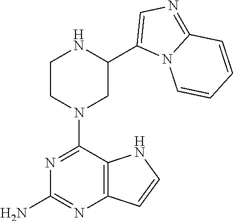 [SMILES: Nc1nc(N2CCNC(c3cnc4ccccn34)C2)c2[nH]ccc2n1] | 4-(3-(imidazo[1,2-a]pyridin-3-yl)piperazin-1- yl)-5H-pyrrolo[3,2-d]pyrimidin-2-amine |
| 56 | 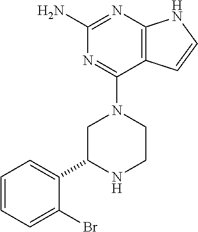 [SMILES: Nc1nc(N2CCN[C@H](c3ccccc3Br)C2)c2cc[nH]c2n1] | (R/S)-4-(3-(2-bromophenyl)piperazin-1-yl)- 7H-pyrrolo[2,3-d]pyrimidin-2-amine |
| 57 | 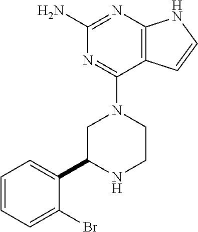 [SMILES: Nc1nc(N2CCN[C@@H](c3ccccc3Br)C2)c2cc[nH]c2n1] | (S/R)-4-(3-(2-bromophenyl)piperazin-1-yl)- 7H-pyrrolo[2,3-d]pyrimidin-2-amine |
| 58 | 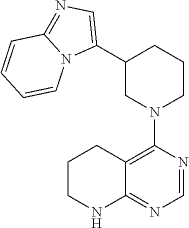 [SMILES: c1ccn2c(C3CCCN(c4ncnc5c4CCCN5)C3)cnc2c1] | 4-(3-(imidazo[1,2-a]pyridin-3-yl)piperidin-1- yl)-5,6,7,8-tetrahydropyrido[2,3-d]pyrimidine |
| 59 | 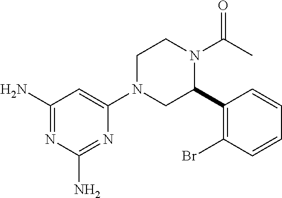 [SMILES: CC(=O)N1CCN(c2cc(N)nc(N)n2)C[C@@H]1c1ccccc1Br] | (S/R)-1-(2-(2-bromophenyl)-4-(2,6- diaminopyrimidin-4-yl)piperazin-1-yl)ethan- 1-one |
| 60 | 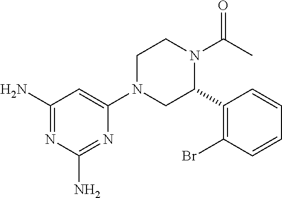 [SMILES: CC(=O)N1CCN(c2cc(N)nc(N)n2)C[C@H]1c1ccccc1Br] | (R/S)-1-(2-(2-bromophenyl)-4-(2,6- diaminopyrimidin-4-yl)piperazin-1-yl)ethan- 1-one |
| 61 | 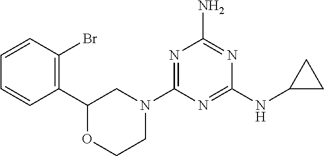 [SMILES: Nc1nc(NC2CC2)nc(N2CCOC(c3ccccc3Br)C2)n1] | 6-(2-(2-bromophenyl)morpholino)-N2- cyclopropyl-1,3,5-triazine-2,4-diamine |
| 62 | 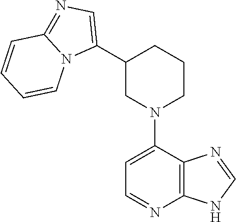 [SMILES: c1ccn2c(C3CCCN(c4ccnc5[nH]cnc45)C3)cnc2c1] | 7-(3-(imidazo[1,2-a]pyridin-3-yl)piperidin-1- yl)-3H-imidazo[4,5-b]pyridine |
| 63 | 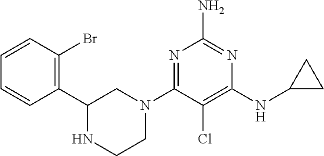 [SMILES: Nc1nc(NC2CC2)c(Cl)c(N2CCNC(c3ccccc3Br)C2)n1] | 6-(3-(2-bromophenyl)piperazin-1-yl)-5- chloro-N4-cyclopropylpyrimidine-2,4-diamine |
| 64 | 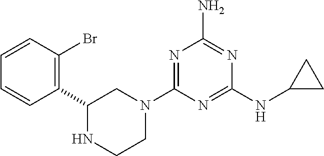 [SMILES: Nc1nc(NC2CC2)nc(N2CCN[C@H](c3ccccc3Br)C2)n1] | (R/S)-6-(3-(2-bromophenyl)piperazin-1-yl)- N2-cyclopropyl-1,3,5-triazine-2,4-diamine |
| 65 | 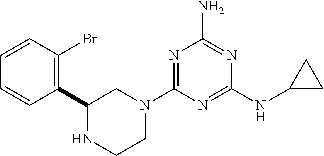 [SMILES: Nc1nc(NC2CC2)nc(N2CCN[C@@H](c3ccccc3Br)C2)n1] | (S/R)-6-(3-(2-bromophenyl)piperazin-1-yl)- N2-cyclopropyl-1,3,5-triazine-2,4-diamine |
| 66 | 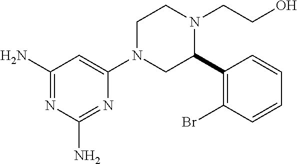 [SMILES: Nc1cc(N2CCN(CCO)[C@@H](c3ccccc3Br)C2)nc(N)n1] | (S/R)-2-(2-(2-bromophenyl)-4-(2,6- diaminopyrimidin-4-yl)piperazin-1-yl)ethan- 1-ol |
| 67 | 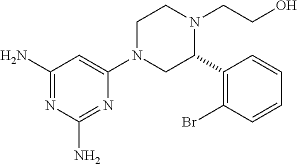 [SMILES: Nc1cc(N2CCN(CCO)[C@H](c3ccccc3Br)C2)nc(N)n1] | (R/S)-2-(2-(2-bromophenyl)-4-(2,6- diaminopyrimidin-4-yl)piperazin-1-yl)ethan- 1-ol |
| 68 | 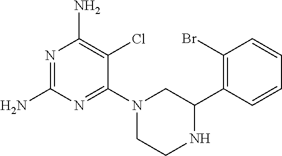 [SMILES: Nc1nc(N)c(Cl)c(N2CCNC(c3ccccc3Br)C2)n1] | 6-(3-(2-bromophenyl)piperazin-1-yl)-5- chloropyrimidine-2,4-diamine |
| 69 | 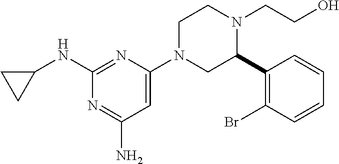 [SMILES: Nc1cc(N2CCN(CCO)[C@@H](c3ccccc3Br)C2)nc(NC2CC2)n1] | (S/R)-2-(4-(6-amino-2- (cyclopropylamino)pyrimidin-4-yl)-2-(2- bromophenyl)piperazin-1-yl)ethan-1-ol |
| 70 | 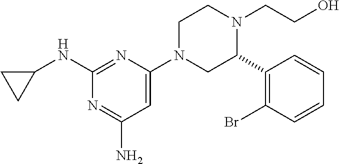 [SMILES: Nc1cc(N2CCN(CCO)[C@H](c3ccccc3Br)C2)nc(NC2CC2)n1] | (R/S)-2-(4-(6-amino-2- (cyclopropylamino)pyrimidin-4-yl)-2-(2- bromophenyl)piperazin-1-yl)ethan-1-ol |
| 71 | 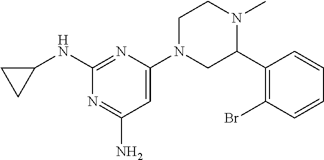 [SMILES: CN1CCN(c2cc(N)nc(NC3CC3)n2)CC1c1ccccc1Br] | 6-(3-(2-bromophenyl)-4-methylpiperazin-1- yl)-N2-cyclopropylpyrimidine-2,4-diamine |
| 72 | 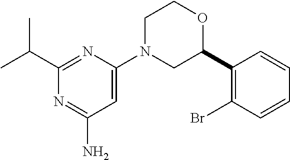 [SMILES: CC(C)c1nc(N)cc(N2CCO[C@@H](c3ccccc3Br)C2)n1] | (S/R)-6-(2-(2-bromophenyl)morpholino)-2- isopropylpyrimidin-4-amine |
| 73 | 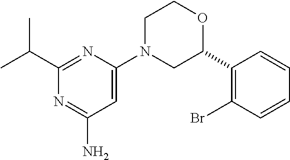 [SMILES: CC(C)c1nc(N)cc(N2CCO[C@H](c3ccccc3Br)C2)n1] | (R/S)-6-(2-(2-bromophenyl)morpholino)-2- isopropylpyrimidin-4-amine |
| 74 | 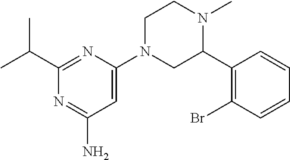 [SMILES: CC(C)c1nc(N)cc(N2CCN(C)C(c3ccccc3Br)C2)n1] | 6-(3-(2-bromophenyl)-4-methylpiperazin-1- yl)-2-isopropylpyrimidin-4-amine |
| 75 | 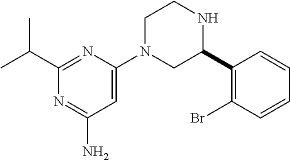 [SMILES: CC(C)c1nc(N)cc(N2CCN[C@@H](c3ccccc3Br)C2)n1] | (S/R)-6-(3-(2-bromophenyl)piperazin-1-yl)-2- isopropylpyrimidin-4-amine |
| 76 | 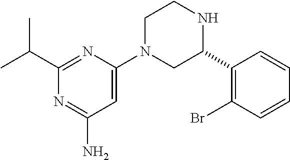 [SMILES: CC(C)c1nc(N)cc(N2CCN[C@H](c3ccccc3Br)C2)n1] | (R/S)-6-(3-(2-bromophenyl)piperazin-1-yl)-2- isopropylpyrimidin-4-amine |
| 77 | 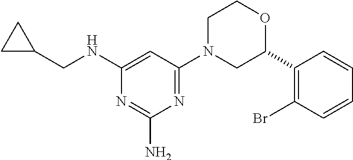 [SMILES: Nc1nc(NCC2CC2)cc(N2CCO[C@H](c3ccccc3Br)C2)n1] | (R/S)-6-(2-(2-bromophenyl)morpholino)-N4- (cyclopropylmethyl)pyrimidine-2,4-diamine |
| 78 | 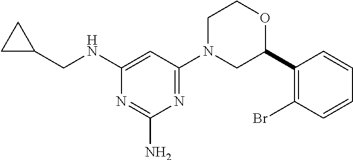 [SMILES: Nc1nc(NCC2CC2)cc(N2CCO[C@@H](c3ccccc3Br)C2)n1] | (S/R)-6-(2-(2-bromophenyl)morpholino)-N4- (cyclopropylmethyl)pyrimidine-2,4-diamine |
| 79 | 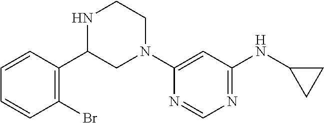 [SMILES: Brc1ccccc1C1CN(c2cc(NC3CC3)ncn2)CCN1] | 6-(3-(2-bromophenyl)piperazin-1-yl)-N- cyclopropylpyrimidin-4-amine |
| 80 | 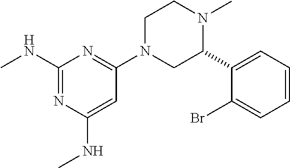 [SMILES: CNc1cc(N2CCN(C)[C@H](c3ccccc3Br)C2)nc(NC)n1] | (R/S)-6-(3-(2-bromophenyl)-4- methylpiperazin-1-yl)-N2,N4- dimethylpyrimidine-2,4-diamine |
| 81 | 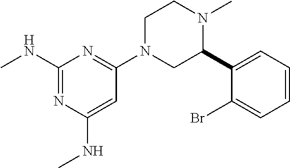 [SMILES: CNc1cc(N2CCN(C)[C@@H](c3ccccc3Br)C2)nc(NC)n1] | (S/R)-6-(3-(2-bromophenyl)-4- methylpiperazin-1-yl)-N2,N4- dimethylpyrimidine-2,4-diamine |
| 82 | 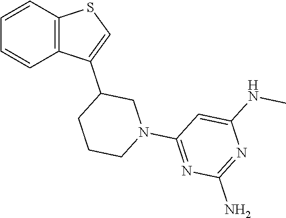 [SMILES: CNc1cc(N2CCCC(c3csc4ccccc34)C2)nc(N)n1] | 6-(3-(benzo[b]thiophen-3-yl)piperidin-1-yl)- N4-methylpyrimidine-2,4-diamine |
| 83 | 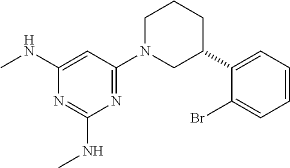 [SMILES: CNc1cc(N2CCC[C@H](c3ccccc3Br)C2)nc(NC)n1] | (R/S)-6-(3-(2-bromophenyl)piperidin-1-yl)- N2,N4-dimethylpyrimidine-2,4-diamine |
| 84 | 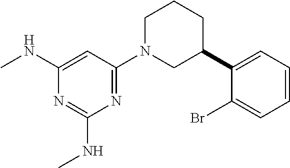 [SMILES: CNc1cc(N2CCC[C@@H](c3ccccc3Br)C2)nc(NC)n1] | (S/R)-6-(3-(2-bromophenyl)piperidin-1-yl)- N2,N4-dimethylpyrimidine-2,4-diamine |
| 85 | 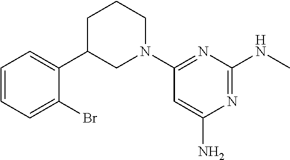 [SMILES: CNc1nc(N)cc(N2CCCC(c3ccccc3Br)C2)n1] | 6-(3-(2-bromophenyl)piperidin-1-yl)-N2- methylpyrimidine-2,4-diamine |
| 86 | 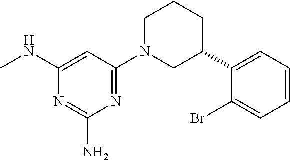 [SMILES: CNc1cc(N2CCC[C@H](c3ccccc3Br)C2)nc(N)n1] | (R/S)-6-(3-(2-bromophenyl)piperidin-1-yl)- N4-methylpyrimidine-2,4-diamine |
| 87 | 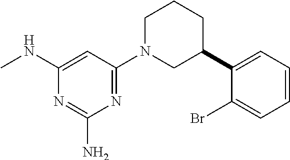 [SMILES: CNc1cc(N2CCC[C@@H](c3ccccc3Br)C2)nc(N)n1] | (S/R)-6-(3-(2-bromophenyl)piperidin-1-yl)- N4-methylpyrimidine-2,4-diamine |
| 88 | 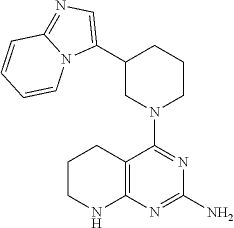 [SMILES: Nc1nc2c(c(N3CCCC(c4cnc5ccccn45)C3)n1)CCCN2] | 4-(3-(imidazo[1,2-a]pyridin-3-yl)piperidin-1- yl)-5,6,7,8-tetrahydropyrido[2,3-d]pyrimidin- 2-amine |
| 89 | 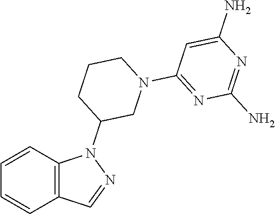 [SMILES: Nc1cc(N2CCCC(n3ncc4ccccc43)C2)nc(N)n1] | 6-(3-(1H-indazol-1-yl)piperidin-1- yl)pyrimidine-2,4-diamine |
| 90 | 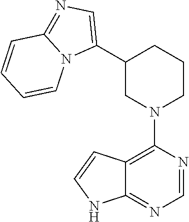 [SMILES: c1ccn2c(C3CCCN(c4ncnc5[nH]ccc45)C3)cnc2c1] | 4-(3-(imidazo[1,2-a]pyridin-3-yl)piperidin-1- yl)-7H-pyrrolo[2,3-d]pyrimidine |
| 91 | 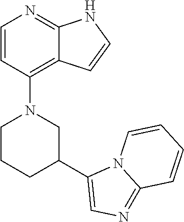 [SMILES: c1ccn2c(C3CCCN(c4ccnc5[nH]ccc45)C3)cnc2c1] | 3-(1-(1H-pyrrolo[2,3-b]pyridin-4-yl)piperidin- 3-yl)imidazo[1,2-a]pyridine |
| 92 | 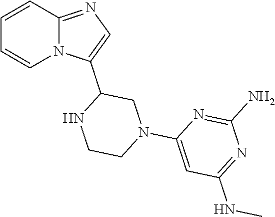 [SMILES: CNc1cc(N2CCNC(c3cnc4ccccn34)C2)nc(N)n1] | 6-(3-(imidazo[1,2-a]pyridin-3-yl)piperazin-1- yl)-N4-methylpyrimidine-2,4-diamine |
| 93 | 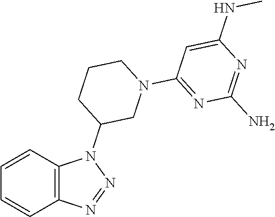 [SMILES: CNc1cc(N2CCCC(n3nnc4ccccc43)C2)nc(N)n1] | 6-(3-(1H-benzo[d][1,2,3]triazol-1-yl)piperidin- 1-yl)-N4-methylpyrimidine-2,4-diamine |
| 94 | 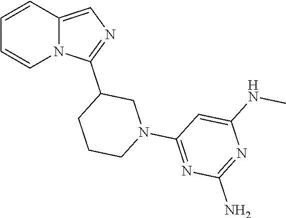 [SMILES: CNc1cc(N2CCCC(c3ncc4ccccn34)C2)nc(N)n1] | 6-(3-(imidazo[1,5-a]pyridin-3-yl)piperidin-1- yl)-N4-methylpyrimidine-2,4-diamine |
| 95 | 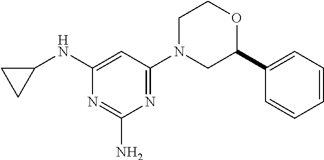 [SMILES: Nc1nc(NC2CC2)cc(N2CCO[C@@H](c3ccccc3)C2)n1] | (S/R)-N4-cyclopropyl-6-(2- phenylmorpholino)pyrimidine-2,4-diamine |
| 96 | 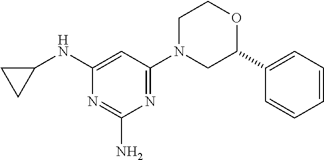 [SMILES: Nc1nc(NC2CC2)cc(N2CCO[C@H](c3ccccc3)C2)n1] | (R/S)-N4-cyclopropyl-6-(2- phenylmorpholino)pyrimidine-2,4-diamine |
| 97 | 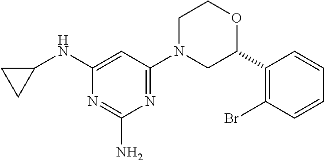 [SMILES: Nc1nc(NC2CC2)cc(N2CCO[C@H](c3ccccc3Br)C2)n1] | (R/S)-6-(2-(2-bromophenyl)morpholino)-N4- cyclopropylpyrimidine-2,4-diamine |
| 98 | 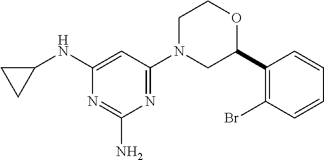 [SMILES: Nc1nc(NC2CC2)cc(N2CCO[C@@H](c3ccccc3Br)C2)n1] | (S/R)-6-(2-(2-bromophenyl)morpholino)-N4- cyclopropylpyrimidine-2,4-diamine |
| 99 | 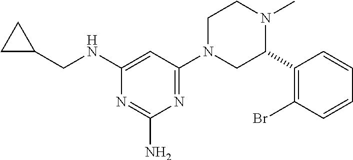 [SMILES: CN1CCN(c2cc(NCC3CC3)nc(N)n2)C[C@H]1c1ccccc1Br] | (R/S)-6-(3-(2-bromophenyl)-4- methylpiperazin-1-yl)-N4- (cyclopropylmethyl)pyrimidine-2,4-diamine |
| 100 | 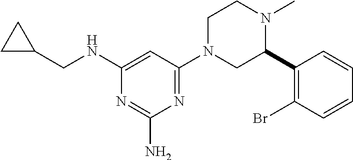 [SMILES: CN1CCN(c2cc(NCC3CC3)nc(N)n2)C[C@@H]1c1ccccc1Br] | (S/R)-6-(3-(2-bromophenyl)-4- methylpiperazin-1-yl)-N4- (cyclopropylmethyl)pyrimidine-2,4-diamine |
| 101 | 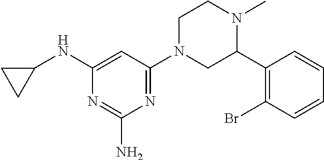 [SMILES: CN1CCN(c2cc(NC3CC3)nc(N)n2)CC1c1ccccc1Br] | 6-(3-(2-bromophenyl)-4-methylpiperazin-1- yl)-N4-cyclopropylpyrimidine-2,4-diamine |
| 102 | 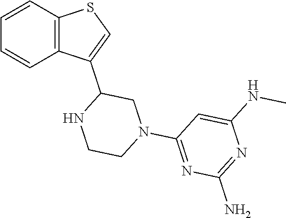 [SMILES: CNc1cc(N2CCNC(c3csc4ccccc34)C2)nc(N)n1] | 6-(3-(benzo[b]thiophen-3-yl)piperazin-1-yl)- N4-methylpyrimidine-2,4-diamine |
| 103 | 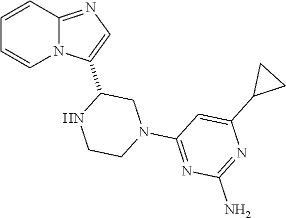 | (S/R)-4-cyclopropyl-6-(3-(imidazo[1,2- a]pyridin-3-yl)piperazin-1-yl)pyrimidin-2- amine |
| 104 | 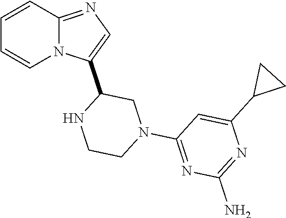 | (R/S)-4-cyclopropyl-6-(3-(imidazo[1,2- a]pyridin-3-yl)piperazin-1-yl)pyrimidin-2- amine |
| 105 | 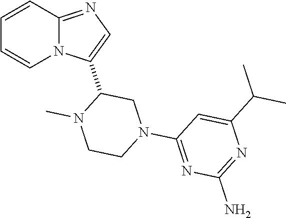 | (S/R)-4-(3-(imidazo[1,2-a]pyridin-3-yl)-4- methylpiperazin-1-yl)-6-isopropylpyrimidin-2- amine |
| 106 | 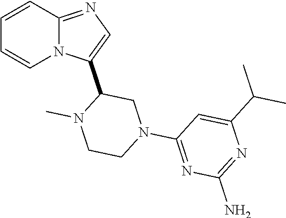 | (R/S)-4-(3-(imidazo[1,2-a]pyridin-3-yl)-4- methylpiperazin-1-yl)-6-isopropylpyrimidin-2- amine |
| 107 | 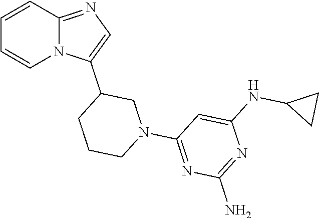 [SMILES: Nc1nc(NC2CC2)cc(N2CCCC(c3cnc4ccccn34)C2)n1] | N4-cyclopropyl-6-(3-(imidazo[1,2-a]pyridin- 3-yl)piperidin-1-yl)pyrimidine-2,4-diamine |
| 108 | 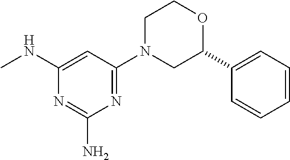 [SMILES: CNc1cc(N2CCO[C@H](c3ccccc3)C2)nc(N)n1] | (R/S)-N4-methyl-6-(2- phenylmorpholino)pyrimidine-2,4-diamine |
| 109 | 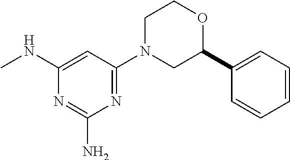 [SMILES: CNc1cc(N2CCO[C@@H](c3ccccc3)C2)nc(N)n1] | (S/R)-N4-methyl-6-(2- phenylmorpholino)pyrimidine-2,4-diamine |
| 110 | 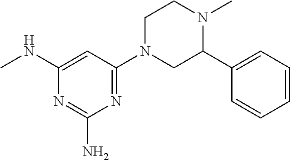 [SMILES: CNc1cc(N2CCN(C)C(c3ccccc3)C2)nc(N)n1] | N4-methyl-6-(4-methyl-3-phenylpiperazin-1- yl)pyrimidine-2,4-diamine |
| 111 | 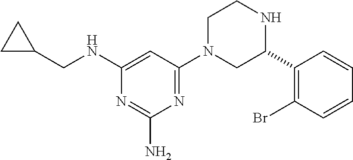 [SMILES: Nc1nc(NCC2CC2)cc(N2CCN[C@H](c3ccccc3Br)C2)n1] | (R/S)-6-(3-(2-bromophenyl)piperazin-1-yl)- N4-(cyclopropylmethyl)pyrimidine-2,4- diamine |
| 112 | 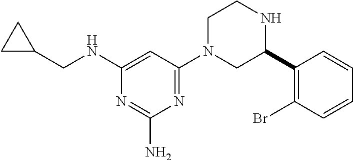 [SMILES: Nc1nc(NCC2CC2)cc(N2CCN[C@@H](c3ccccc3Br)C2)n1] | (S/R)-6-(3-(2-bromophenyl)piperazin-1-yl)- N4-(cyclopropylmethyl)pyrimidine-2,4- diamine |
| 113 | 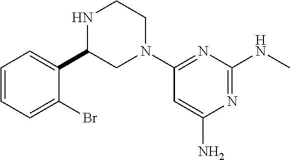 | (R/S)-6-(3-(2-bromophenyl)piperazin-1-yl)- N2-methylpyrimidine-2,4-diamine |
| 114 | 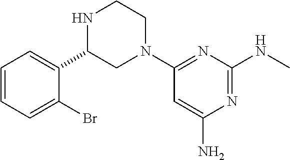 | (S/R)-6-(3-(2-bromophenyl)piperazin-1-yl)- N2-methylpyrimidine-2,4-diamine |
| 115 | 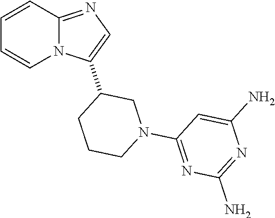 | (S/R)-6-(3-(imidazo[1,2-a]pyridin-3- yl)piperidin-1-yl)pyrimidine-2,4-diamine |
| 116 | 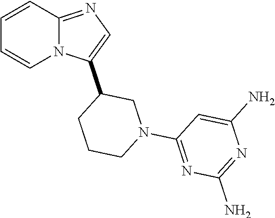 | (R/S)-6-(3-(imidazo[1,2-a]pyridin-3- yl)piperidin-1-yl)pyrimidine-2,4-diamine |
| 117 | 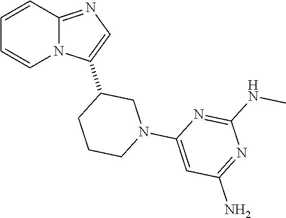 | (S/R)-6-(3-(imidazo[1,2-a]pyridin-3- yl)piperidin-1-yl)-N2-methylpyrimidine-2,4- diamine |
| 118 | 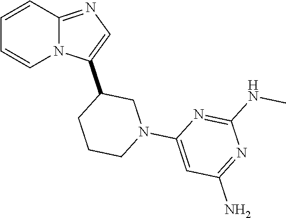 | (R/S)-6-(3-(imidazo[1,2-a]pyridin-3- yl)piperidin-1-yl)-N2-methylpyrimidine-2,4- diamine |
| 119 | 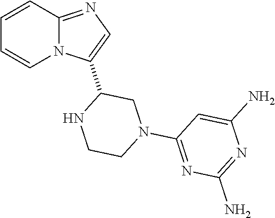 | (S/R)-6-(3-(imidazo[1,2-a]pyridin-3- yl)piperazin-1-yl)pyrimidine-2,4-diamine |
| 120 | 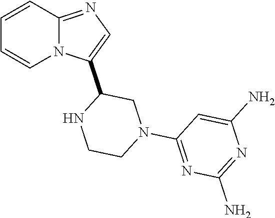 | (R/S)-6-(3-(imidazo[1,2-a]pyridin-3- yl)piperazin-1-yl)pyrimidine-2,4-diamine |
| 121 | 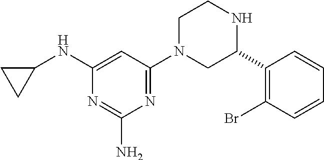 [SMILES: Nc1nc(NC2CC2)cc(N2CCN[C@H](c3ccccc3Br)C2)n1] | (R/S)-6-(3-(2-bromophenyl)piperazin-1-yl)- N4-cyclopropylpyrimidine-2,4-diamine |
| 122 | 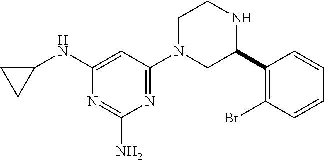 [SMILES: Nc1nc(NC2CC2)cc(N2CCN[C@@H](c3ccccc3Br)C2)n1] | (S/R)-6-(3-(2-bromophenyl)piperazin-1-yl)- N4-cyclopropylpyrimidine-2,4-diamine |
| 123 | 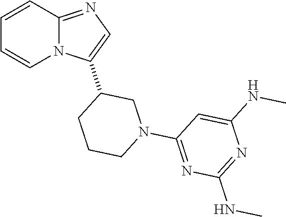 | (S/R)-6-(3-(imidazo[1,2-a]pyridin-3- yl)piperidin-1-yl)-N2,N4-dimethylpyrimidine- 2,4-diamine |
| 124 | 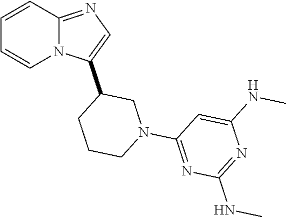 | (R/S)-6-(3-(imidazo[1,2-a]pyridin-3- yl)piperidin-1-yl)-N2,N4-dimethylpyrimidine- 2,4-diamine |
| 125 | 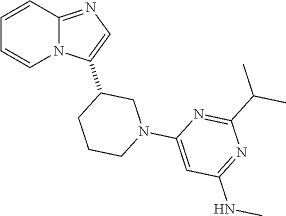 | (S/R)-6-(3-(imidazo[1,2-a]pyridin-3- yl)piperidin-1-yl)-2-isopropyl-N- methylpyrimidin-4-amine |
| 126 | 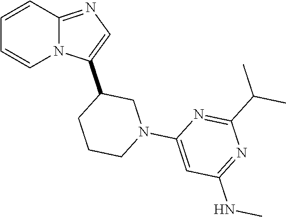 | (R/S)-6-(3-(imidazo[1,2-a]pyridin-3- yl)piperidin-1-yl)-2-isopropyl-N- methylpyrimidin-4-amine |
| 127 | 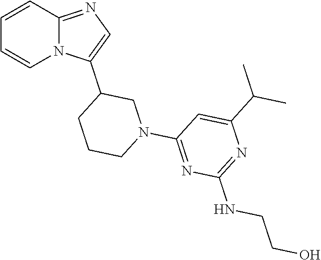 [SMILES: CC(C)c1cc(N2CCCC(c3cnc4ccccn34)C2)nc(NCCO)n1] | 2-((4-(3-(imidazo[1,2-a]pyridin-3-yl)piperidin- 1-yl)-6-isopropylpyrimidin-2-yl)amino)ethan- 1-ol |
| 128 | 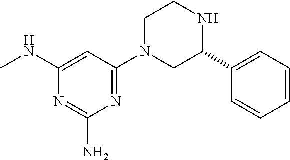 [SMILES: CNc1cc(N2CCN[C@H](c3ccccc3)C2)nc(N)n1] | (R/S)-N4-methyl-6-(3-phenylpiperazin-1- yl)pyrimidine-2,4-diamine |
| 129 | 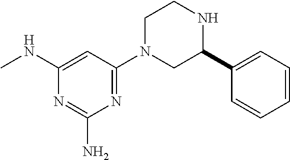 [SMILES: CNc1cc(N2CCN[C@@H](c3ccccc3)C2)nc(N)n1] | (S/R)-N4-methyl-6-(3-phenylpiperazin-1- yl)pyrimidine-2,4-diamine |
| 130 | 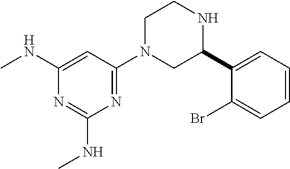 [SMILES: CNc1cc(N2CCN[C@@H](c3ccccc3Br)C2)nc(NC)n1] | (S/R)-6-(3-(2-bromophenyl)piperazin-1-yl)- N2,N4-dimethylpyrimidine-2,4-diamine |
| 131 | 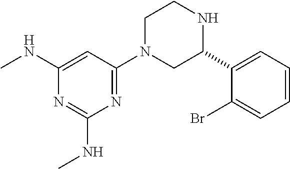 [SMILES: CNc1cc(N2CCN[C@H](c3ccccc3Br)C2)nc(NC)n1] | (R/S)-6-(3-(2-bromophenyl)piperazin-1-yl)- N2,N4-dimethylpyrimidine-2,4-diamine |
| 132 | 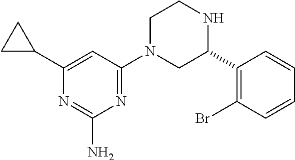 [SMILES: Nc1nc(C2CC2)cc(N2CCN[C@H](c3ccccc3Br)C2)n1] | (R/S)-4-(3-(2-bromophenyl)piperazin-1-yl)-6- cyclopropylpyrimidin-2-amine |
| 133 | 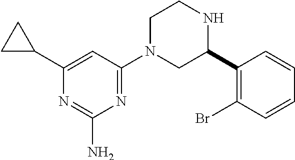 [SMILES: Nc1nc(C2CC2)cc(N2CCN[C@@H](c3ccccc3Br)C2)n1] | (S/R)-4-(3-(2-bromophenyl)piperazin-1-yl)-6- cyclopropylpyrimidin-2-amine |
| 134 | 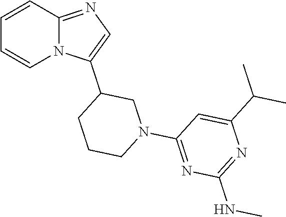 [SMILES: CNc1nc(C(C)C)cc(N2CCCC(c3cnc4ccccn34)C2)n1] | 4-(3-(imidazo[1,2-a]pyridin-3-yl)piperidin-1- yl)-6-isopropyl-N-methylpyrimidin-2-amine |
| 135 | 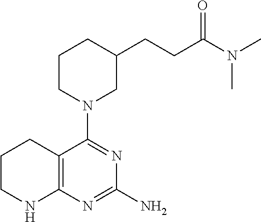 [SMILES: CN(C)C(=O)CCC1CCCN(c2nc(N)nc3c2CCCN3)C1] | 3-(1-(2-amino-5,6,7,8-tetrahydropyrido[2,3- d]pyrimidin-4-yl)piperidin-3-yl)-N,N- dimethylpropanamide |
| 136 | 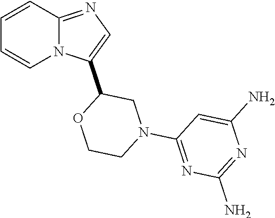 | (R/S)-6-(2-(imidazo[1,2-a]pyridin-3- yl)morpholino)pyrimidine-2,4-diamine |
| 137 | 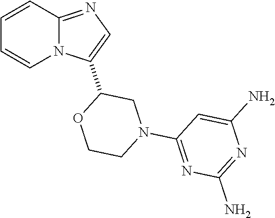 | (S/R)-6-(2-(imidazo[1,2-a]pyridin-3- yl)morpholino)pyrimidine-2,4-diamine |
| 138 | 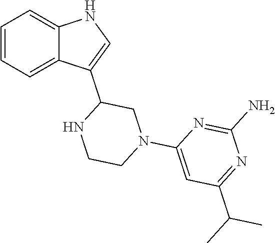 [SMILES: CC(C)c1cc(N2CCNC(c3c[nH]c4ccccc34)C2)nc(N)n1] | 4-(3-(1H-indol-3-yl)piperazin-1-yl)-6- isopropylpyrimidin-2-amine |
| 139 | 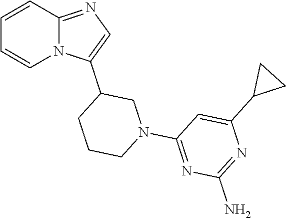 [SMILES: Nc1nc(C2CC2)cc(N2CCCC(c3cnc4ccccn34)C2)n1] | 4-cyclopropyl-6-(3-(imidazo[1,2-a]pyridin-3- yl)piperidin-1-yl)pyrimidin-2-amine |
| 140 | 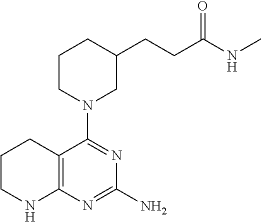 [SMILES: CNC(=O)CCC1CCCN(c2nc(N)nc3c2CCCN3)C1] | 3-(1-(2-amino-5,6,7,8-tetrahydropyrido[2,3- d]pyrimidin-4-yl)piperidin-3-yl)-N- methylpropanamide |
| 141 | 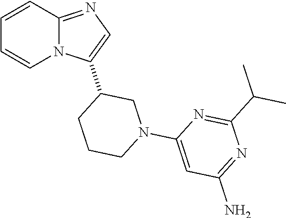 | (S/R)-6-(3-(imidazo[1,2-a]pyridin-3- yl)piperidin-1-yl)-2-isopropylpyrimidin-4- amine |
| 142 | 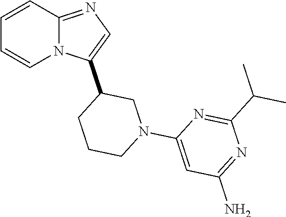 | (R/S)-6-(3-(imidazo[1,2-a]pyridin-3- yl)piperidin-1-yl)-2-isopropylpyrimidin-4- amine |
| 143 | 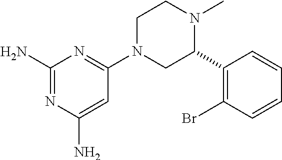 [SMILES: CN1CCN(c2cc(N)nc(N)n2)C[C@H]1c1ccccc1Br] | (R/S)-6-(3-(2-bromophenyl)-4- methylpiperazin-1-yl)pyrimidine-2,4-diamine |
| 144 | 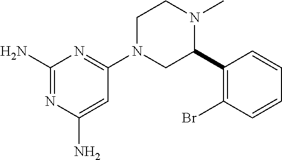 [SMILES: CN1CCN(c2cc(N)nc(N)n2)C[C@@H]1c1ccccc1Br] | (S/R)-6-(3-(2-bromophenyl)-4- methylpiperazin-1-yl)pyrimidine-2,4-diamine |
| 145 | 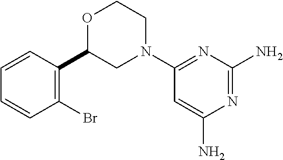 | (R/S)-6-(2-(2- bromophenyl)morpholino)pyrimidine-2,4- diamine |
| 146 | 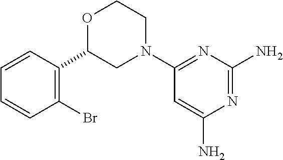 | (S/R)-6-(2-(2- bromophenyl)morpholino)pyrimidine-2,4- diamine |
| 147 | 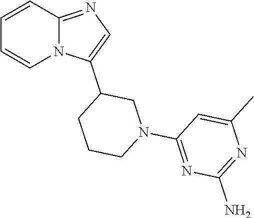 [SMILES: Cc1cc(N2CCCC(c3cnc4ccccn34)C2)nc(N)n1] | 4-(3-(imidazo[1,2-a]pyridin-3-yl)piperidin-1- yl)-6-methylpyrimidin-2-amine |
| 148 | 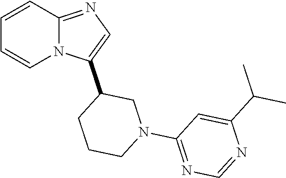 | (R/S)-3-(1-(6-isopropylpyrimidin-4- yl)piperidin-3-yl)imidazo[1,2-a]pyridine |
| 149 | 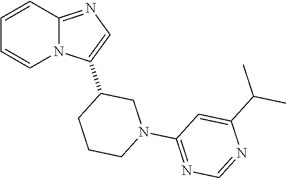 | (S/R)-3-(1-(6-isopropylpyrimidin-4- yl)piperidin-3-yl)imidazo[1,2-a]pyridine |
| 150 | 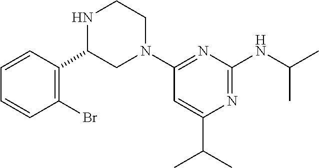 | (S/R)-4-(3-(2-bromophenyl)piperazin-1-yl)- N,6-diisopropylpyrimidin-2-amine |
| 151 | 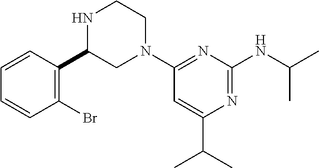 | (R/S)-4-(3-(2-bromophenyl)piperazin-1-yl)- N,6-diisopropylpyrimidin-2-amine |
| 152 | 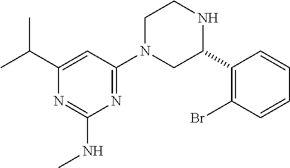 [SMILES: CNc1nc(C(C)C)cc(N2CCN[C@H](c3ccccc3Br)C2)n1] | (R/S)-4-(3-(2-bromophenyl)piperazin-1-yl)-6- isopropyl-N-methylpyrimidin-2-amine |
| 153 | 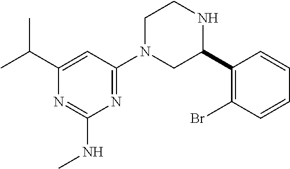 [SMILES: CNc1nc(C(C)C)cc(N2CCN[C@@H](c3ccccc3Br)C2)n1] | (S/R)-4-(3-(2-bromophenyl)piperazin-1-yl)-6- isopropyl-N-methylpyrimidin-2-amine |
| 154 | 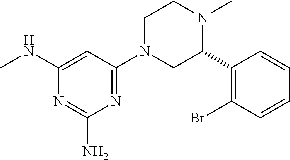 [SMILES: CNc1cc(N2CCN(C)[C@H](c3ccccc3Br)C2)nc(N)n1] | (R/S)-6-(3-(2-bromophenyl)-4- methylpiperazin-1-yl)-N4-methylpyrimidine- 2,4-diamine |
| 155 | 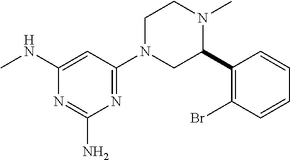 [SMILES: CNc1cc(N2CCN(C)[C@@H](c3ccccc3Br)C2)nc(N)n1] | (S/R)-6-(3-(2-bromophenyl)-4- methylpiperazin-1-yl)-N4-methylpyrimidine- 2,4-diamine |
| 156 | 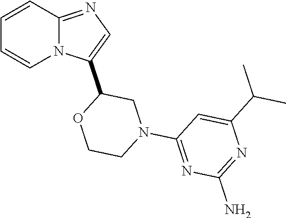 | (R/S)-4-(2-(imidazo[1,2-a]pyridin-3- yl)morpholino)-6-isopropylpyrimidin-2-amine |
| 157 | 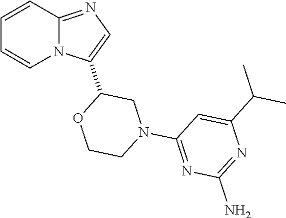 | (S/R)-4-(2-(imidazo[1,2-a]pyridin-3- yl)morpholino)-6-isopropylpyrimidin-2-amine |
| 158 | 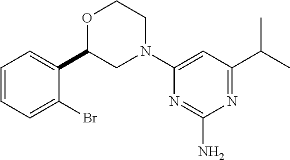 | (R/S)-4-(2-(2-bromophenyl)morpholino)-6- isopropylpyrimidin-2-amine |
| 159 | 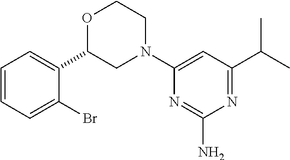 | (S/R)-4-(2-(2-bromophenyl)morpholino)-6- isopropylpyrimidin-2-amine |
| 160 | 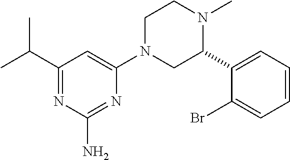 [SMILES: CC(C)c1cc(N2CCN(C)[C@H](c3ccccc3Br)C2)nc(N)n1] | (R/S)-4-(3-(2-bromophenyl)-4- methylpiperazin-1-yl)-6-isopropylpyrimidin-2- amine |
| 161 | 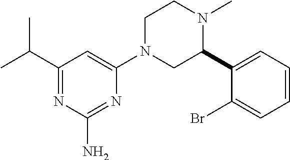 [SMILES: CC(C)c1cc(N2CCN(C)[C@@H](c3ccccc3Br)C2)nc(N)n1] | (S/R)-4-(3-(2-bromophenyl)-4- methylpiperazin-1-yl)-6-isopropylpyrimidin-2- amine |
| 162 | 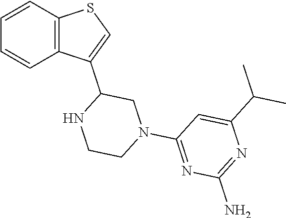 [SMILES: CC(C)c1cc(N2CCNC(c3csc4ccccc34)C2)nc(N)n1] | 4-(3-(benzo[b]thiophen-3-yl)piperazin-1-yl)- 6-isopropylpyrimidin-2-amine |
| 163 | 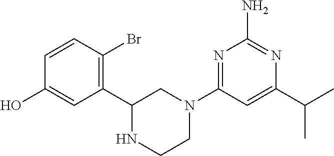 [SMILES: CC(C)c1cc(N2CCNC(c3cc(O)ccc3Br)C2)nc(N)n1] | 3-(4-(2-amino-6-isopropylpyrimidin-4- yl)piperazin-2-yl)-4-bromophenol |
| 164 | 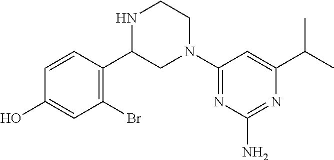 [SMILES: CC(C)c1cc(N2CCNC(c3ccc(O)cc3Br)C2)nc(N)n1] | 4-(4-(2-amino-6-isopropylpyrimidin-4- yl)piperazin-2-yl)-3-bromophenol |
| 165 | 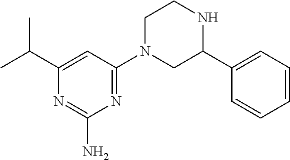 [SMILES: CC(C)c1cc(N2CCNC(c3ccccc3)C2)nc(N)n1] | 4-isopropyl-6-(3-phenylpiperazin-1- yl)pyrimidin-2-amine |
| 166 | 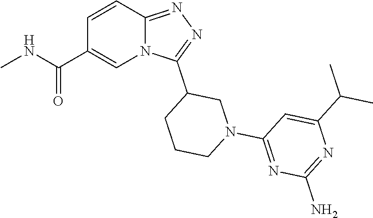 [SMILES: CNC(=O)c1ccc2nnc(C3CCCN(c4cc(C(C)C)nc(N)n4)C3)n2c1] | 3-(1-(2-amino-6-isopropylpyrimidin-4- yl)piperidin-3-yl)-N-methyl-[1,2,4]triazolo[4,3- a]pyridine-6-carboxamide |
| 167 | 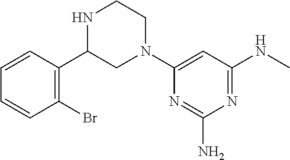 [SMILES: CNc1cc(N2CCNC(c3ccccc3Br)C2)nc(N)n1] | 3-(1-(2-amino-6-isopropylpyrimidin-4- yl)piperidin-3-yl)-N-methyl-[1,2,4]triazolo[4,3- a]pyridine-6-carboxamide |
| 168 | 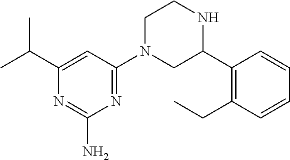 [SMILES: CCc1ccccc1C1CN(c2cc(C(C)C)nc(N)n2)CCN1] | 4-(3-(2-ethylphenyl)piperazin-1-yl)-6- isopropylpyrimidin-2-amine |
| 169 | 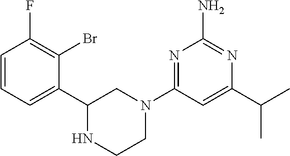 [SMILES: CC(C)c1cc(N2CCNC(c3cccc(F)c3Br)C2)nc(N)n1] | 4-(3-(2-bromo-3-fluorophenyl)piperazin-1- yl)-6-isopropylpyrimidin-2-amine |
| 170 | 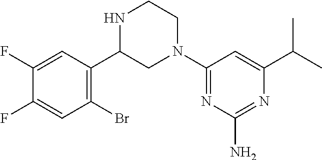 [SMILES: CC(C)c1cc(N2CCNC(c3cc(F)c(F)cc3Br)C2)nc(N)n1] | 4-(3-(2-bromo-4,5-difluorophenyl)piperazin- 1-yl)-6-isopropylpyrimidin-2-amine |
| 171 | 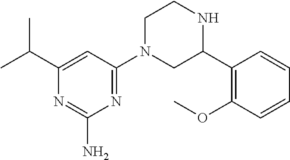 [SMILES: COc1ccccc1C1CN(c2cc(C(C)C)nc(N)n2)CCN1] | 4-isopropyl-6-(3-(2- methoxyphenyl)piperazin-1-yl)pyrimidin-2- amine |
| 172 | 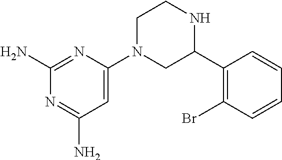 [SMILES: Nc1cc(N2CCNC(c3ccccc3Br)C2)nc(N)n1] | 6-(3-(2-bromophenyl)piperazin-1- yl)pyrimidine-2,4-diamine |
| 173 | 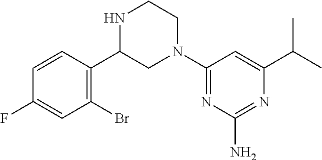 [SMILES: CC(C)c1cc(N2CCNC(c3ccc(F)cc3Br)C2)nc(N)n1] | 4-(3-(2-bromo-4-fluorophenyl)piperazin-1- yl)-6-isopropylpyrimidin-2-amine |
| 174 | 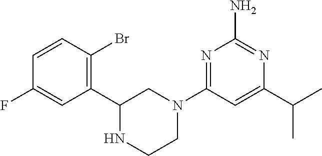 [SMILES: CC(C)c1cc(N2CCNC(c3cc(F)ccc3Br)C2)nc(N)n1] | 4-(3-(2-bromo-5-fluorophenyl)piperazin-1- yl)-6-isopropylpyrimidin-2-amine |
| 175 | 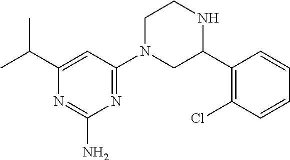 [SMILES: CC(C)c1cc(N2CCNC(c3ccccc3Cl)C2)nc(N)n1] | 4-(3-(2-chlorophenyl)piperazin-1-yl)-6- isopropylpyrimidin-2-amine |
| 176 | 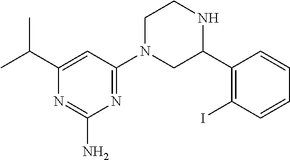 [SMILES: CC(C)c1cc(N2CCNC(c3ccccc3I)C2)nc(N)n1] | 4-(3-(2-iodophenyl)piperazin-1-yl)-6- isopropylpyrimidin-2-amine |
| 177 | 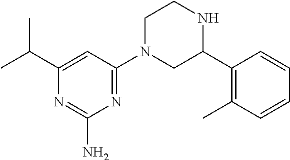 [SMILES: Cc1ccccc1C1CN(c2cc(C(C)C)nc(N)n2)CCN1] | 4-isopropyl-6-(3-(o-tolyl)piperazin-1- yl)pyrimidin-2-amine |
| 178 | 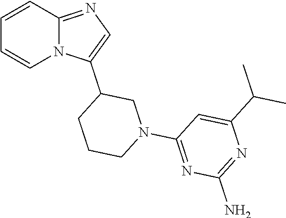 [SMILES: CC(C)c1cc(N2CCCC(c3cnc4ccccn34)C2)nc(N)n1] | 4-(3-(imidazo[1,2-a]pyridin-3-yl)piperidin-1- yl)-6-isopropylpyrimidin-2-amine |
| 179 | 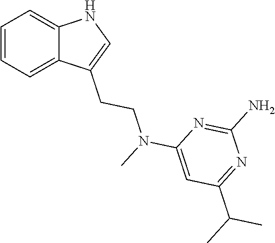 [SMILES: CC(C)c1cc(N(C)CCc2c[nH]c3ccccc23)nc(N)n1] | N4-(2-(1H-indol-3-yl)ethyl)-6-isopropyl-N4- methylpyrimidine-2,4-diamine |
| 180 | 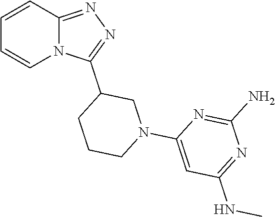 [SMILES: CNc1cc(N2CCCC(c3nnc4ccccn34)C2)nc(N)n1] | 6-(3-([1,2,4]triazolo[4,3-a]pyridin-3- yl)piperidin-1-yl)-N4-methylpyrimidine-2,4- diamine |
| 181 | 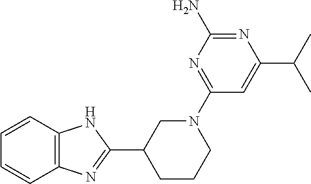 [SMILES: CC(C)c1cc(N2CCCC(c3nc4ccccc4[nH]3)C2)nc(N)n1] | 4-(3-(1H-benzo[d]imidazol-2-yl)piperidin-1- yl)-6-isopropylpyrimidin-2-amine |
| 182 | 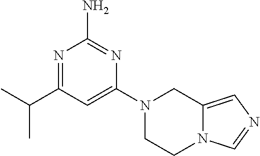 [SMILES: CC(C)c1cc(N2CCn3cncc3C2)nc(N)n1] | 4-(5,6-dihydroimidazo[1,5-a]pyrazin-7(8H)- yl)-6-isopropylpyrimidin-2-amine |
| 183 | 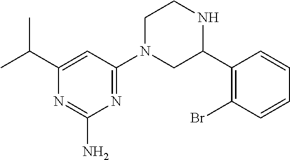 [SMILES: CC(C)c1cc(N2CCNC(c3ccccc3Br)C2)nc(N)n1] | 4-(3-(2-bromophenyl)piperazin-1-yl)-6- isopropylpyrimidin-2-amine |
| 184 | 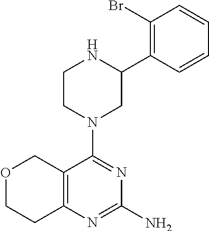 [SMILES: Nc1nc2c(c(N3CCNC(c4ccccc4Br)C3)n1)COCC2] | 4-(3-(2-bromophenyl)piperazin-1-yl)-7,8- dihydro-5H-pyrano[4,3-d]pyrimidin-2-amine |
| 185 | 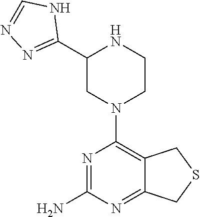 [SMILES: Nc1nc2c(c(N3CCNC(c4nnc[nH]4)C3)n1)CSC2] | 4-(3-(4H-1,2,4-triazol-3-yl)piperazin-1-yl)- 5,7-dihydrothieno[3,4-d]pyrimidin-2-amine |
| 186 | 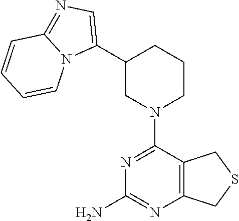 [SMILES: Nc1nc2c(c(N3CCCC(c4cnc5ccccn45)C3)n1)CSC2] | 4-(3-(imidazo[1,2-a]pyridin-3-yl)piperidin-1- yl)-5,7-dihydrothieno[3,4-d]pyrimidin-2- amine |
| 187 | 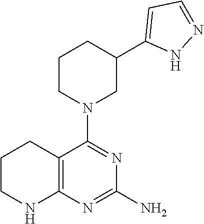 [SMILES: Nc1nc2c(c(N3CCCC(c4ccn[nH]4)C3)n1)CCCN2] | 4-(3-(1H-pyrazol-5-yl)piperidin-1-yl)-5,6,7,8- tetrahydropyrido[2,3-d]pyrimidin-2-amine |
| 188 | 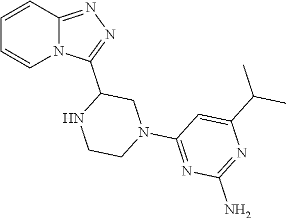 [SMILES: CC(C)c1cc(N2CCNC(c3nnc4ccccn34)C2)nc(N)n1] | 4-(3-([1,2,4]triazolo[4,3-a]pyridin-3- yl)piperazin-1-yl)-6-isopropylpyrimidin-2- amine |
| 189 | 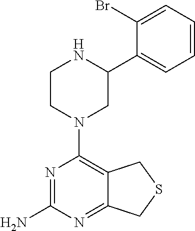 [SMILES: Nc1nc2c(c(N3CCNC(c4ccccc4Br)C3)n1)CSC2] | 4-(3-(2-bromophenyl)piperazin-1-yl)-5,7- dihydrothieno[3,4-d]pyrimidin-2-amine |
| 190 | 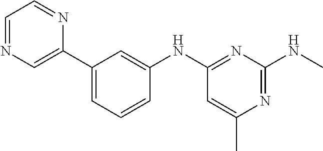 [SMILES: CNc1nc(C)cc(Nc2cccc(-c3cnccn3)c2)n1] | N2,6-dimethyl-N4-(3-(pyrazin-2- yl)phenyl)pyrimidine-2,4-diamine |
| 191 | 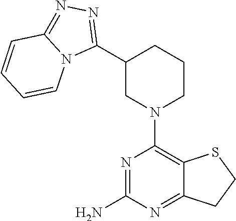 [SMILES: Nc1nc2c(c(N3CCCC(c4nnc5ccccn45)C3)n1)SCC2] | 4-(3-([1,2,4]triazolo[4,3-a]pyridin-3- yl)piperidin-1-yl)-6,7-dihydrothieno[3,2- d]pyrimidin-2-amine |
| 192 | 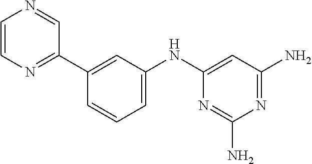 [SMILES: Nc1cc(Nc2cccc(-c3cnccn3)c2)nc(N)n1] | N4-(3-(pyrazin-2-yl)phenyl)pyrimidine-2,4,6- triamine |
| 193 | 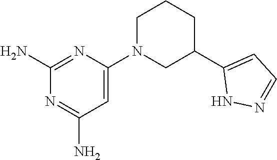 [SMILES: Nc1cc(N2CCCC(c3ccn[nH]3)C2)nc(N)n1] | 6-(3-(1H-pyrazol-5-yl)piperidin-1- yl)pyrimidine-2,4-diamine |
| 194 | 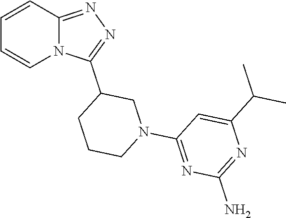 [SMILES: CC(C)c1cc(N2CCCC(c3nnc4ccccn34)C2)nc(N)n1] | 4-(3-([1,2,4]triazolo[4,3-a]pyridin-3- yl)piperidin-1-yl)-6-isopropylpyrimidin-2- amine |
| 195 | 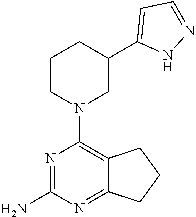 [SMILES: Nc1nc2c(c(N3CCCC(c4ccn[nH]4)C3)n1)CCC2] | 4-(3-(1H-pyrazol-5-yl)piperidin-1-yl)-6,7- dihydro-5H-cyclopenta[d]pyrimidin-2-amine |
| 196 | 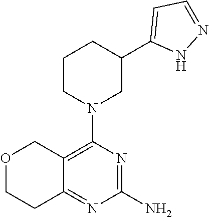 [SMILES: Nc1nc2c(c(N3CCCC(c4ccn[nH]4)C3)n1)COCC2] | 4-(3-(1H-pyrazol-5-yl)piperidin-1-yl)-7,8- dihydro-5H-pyrano[4,3-d]pyrimidin-2-amine |
| 197 | 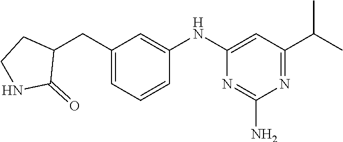 [SMILES: CC(C)c1cc(Nc2cccc(CC3CCNC3=O)c2)nc(N)n1] | 3-(3-((2-amino-6-isopropylpyrimidin-4- yl)amino)benzyl)pyrrolidin-2-one |
| 198 | 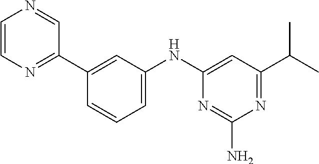 [SMILES: CC(C)c1cc(Nc2cccc(-c3cnccn3)c2)nc(N)n1] | 6-isopropyl-N4-(3-(pyrazin-2- yl)phenyl)pyrimidine-2,4-diamine |
| 199 | 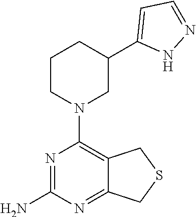 [SMILES: Nc1nc2c(c(N3CCCC(c4ccn[nH]4)C3)n1)CSC2] | 4-(3-(1H-pyrazol-5-yl)piperidin-1-yl)-5,7- dihydrothieno[3,4-d]pyrimidin-2-amine |
| 200 | 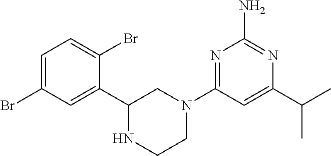 [SMILES: CC(C)c1cc(N2CCNC(c3cc(Br)ccc3Br)C2)nc(N)n1] | 4-(3-(2,5-dibromophenyl)piperazin-1-yl)-6- isopropylpyrimidin-2-amine |
| 201 | 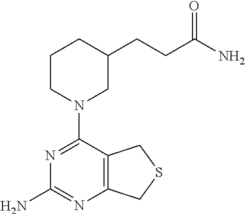 [SMILES: NC(=O)CCC1CCCN(c2nc(N)nc3c2CSC3)C1] | 3-(1-(2-amino-5,7-dihydrothieno[3,4- d]pyrimidin-4-yl)piperidin-3-yl)propanamide |
| 202 | 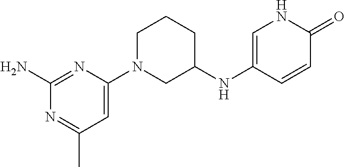 [SMILES: Cc1cc(N2CCCC(Nc3ccc(=O)[nH]c3)C2)nc(N)n1] | 5-((1-(2-amino-6-methylpyrimidin-4- yl)piperidin-3-yl)amino)pyridin-2(1H)-one |
| 203 | 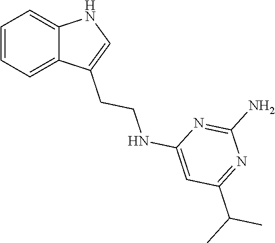 [SMILES: CC(C)c1cc(NCCc2c[nH]c3ccccc23)nc(N)n1] | N4-(2-(1H-indol-3-yl)ethyl)-6- isopropylpyrimidine-2,4-diamine |
| 204 | 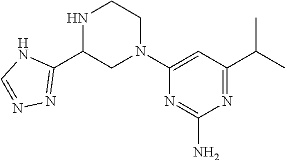 [SMILES: CC(C)c1cc(N2CCNC(c3nnc[nH]3)C2)nc(N)n1] | 4-(3-(4H-1,2,4-triazol-3-yl)piperazin-1-yl)-6- isopropylpyrimidin-2-amine |
| 205 | 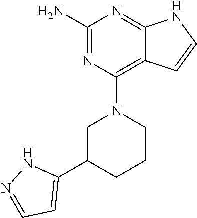 [SMILES: Nc1nc(N2CCCC(c3ccn[nH]3)C2)c2cc[nH]c2n1] | 4-(3-(1H-pyrazol-5-yl)piperidin-1-yl)-7H- pyrrolo[2,3-d]pyrimidin-2-amine |
| 206 | 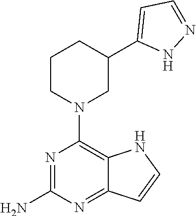 [SMILES: Nc1nc(N2CCCC(c3ccn[nH]3)C2)c2[nH]ccc2n1] | 4-(3-(1H-pyrazol-5-yl)piperidin-1-yl)-5H- pyrrolo[3,2-d]pyrimidin-2-amine |
| 207 | 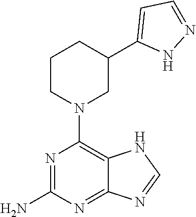 [SMILES: Nc1nc(N2CCCC(c3ccn[nH]3)C2)c2[nH]cnc2n1] | 6-(3-(1H-pyrazol-5-yl)piperidin-1-yl)-7H- purin-2-amine |
| 208 | 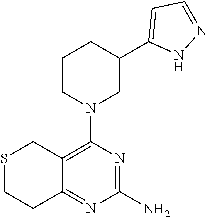 [SMILES: Nc1nc2c(c(N3CCCC(c4ccn[nH]4)C3)n1)CSCC2] | 4-(3-(1H-pyrazol-5-yl)piperidin-1-yl)-7,8- dihydro-5H-thiopyrano[4,3-d]pyrimidin-2- amine |
| 209 | 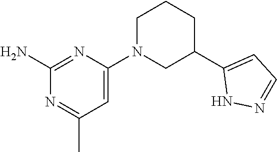 [SMILES: Cc1cc(N2CCCC(c3ccn[nH]3)C2)nc(N)n1] | 4-(3-(1H-pyrazol-5-yl)piperidin-1-yl)-6- methylpyrimidin-2-amine |
| 210 | 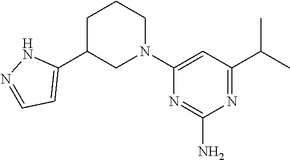 [SMILES: CC(C)c1cc(N2CCCC(c3ccn[nH]3)C2)nc(N)n1] | 4-(3-(1H-pyrazol-5-yl)piperidin-1-yl)-6- isopropylpyrimidin-2-amine |
| 211 | 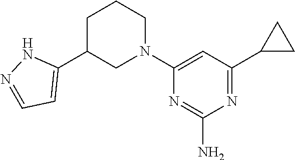 [SMILES: Nc1nc(C2CC2)cc(N2CCCC(c3ccn[nH]3)C2)n1] | 4-(3-(1H-pyrazol-5-yl)piperidin-1-yl)-6- cyclopropylpyrimidin-2-amine |
| 212 | 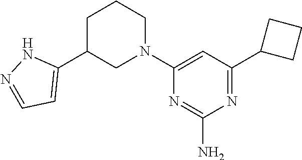 [SMILES: Nc1nc(C2CCC2)cc(N2CCCC(c3ccn[nH]3)C2)n1] | 4-(3-(1H-pyrazol-5-yl)piperidin-1-yl)-6- cyclobutylpyrimidin-2-amine |
| 213 | 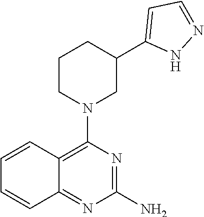 [SMILES: Nc1nc(N2CCCC(c3ccn[nH]3)C2)c2ccccc2n1] | 4-(3-(1H-pyrazol-5-yl)piperidin-1- yl)quinazolin-2-amine |
| 214 | 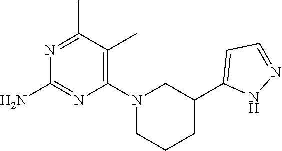 [SMILES: Cc1nc(N)nc(N2CCCC(c3ccn[nH]3)C2)c1C] | 4-(3-(1H-pyrazol-5-yl)piperidin-1-yl)-5,6- dimethylpyrimidin-2-amine |
| 215 | 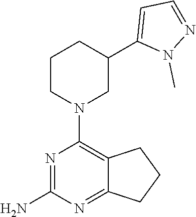 [SMILES: Cn1nccc1C1CCCN(c2nc(N)nc3c2CCC3)C1] | 4-(3-(1 methyl-1H-pyrazol-5-yl)piperidin-1- yl)-6,7-dihydro-5H-cyclopenta[d]pyrimidin-2- amine |
| 216 | 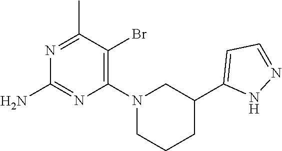 [SMILES: Cc1nc(N)nc(N2CCCC(c3ccn[nH]3)C2)c1Br] | 4-(3-(1H-pyrazol-5-yl)piperidin-1-yl)-5- bromo-6-methylpyrimidin-2-amine |
| 217 | 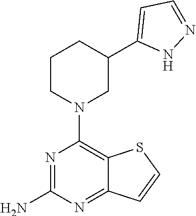 [SMILES: Nc1nc(N2CCCC(c3ccn[nH]3)C2)c2sccc2n1] | 4-(3-(1H-pyrazol-5-yl)piperidin-1- yl)thieno[3,2-d]pyrimidin-2-amine |
| 218 | 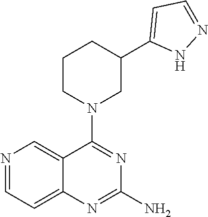 [SMILES: Nc1nc(N2CCCC(c3ccn[nH]3)C2)c2cnccc2n1] | 4-(3-(1H-pyrazol-5-yl)piperidin-1- yl)pyrido[4,3-d]pyrimidin-2-amine |
| 219 | 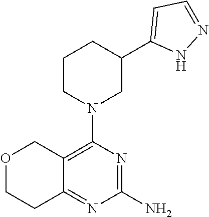 [SMILES: Nc1nc2c(c(N3CCCC(c4ccn[nH]4)C3)n1)COCC2] | 4-(3-(1H-pyrazol-5-yl)piperidin-1-yl)-5,8- dihydro-6H-pyrano[3,4-d]pyrimidin-2-amine |
| 220 | 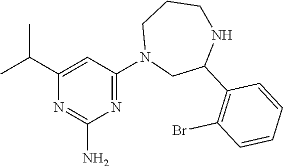 [SMILES: CC(C)c1cc(N2CCCNC(c3ccccc3Br)C2)nc(N)n1] | 4-(3-(2-bromophenyl)-1,4-diazepan-1-yl)-6- isopropylpyrimidin-2-amine |
| 221 | 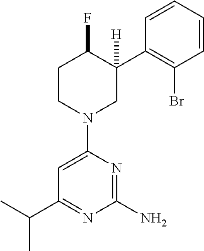 [SMILES: CC(C)c1cc(N2CC[C@@H](F)[C@@H](c3ccccc3Br)C2)nc(N)n1] | 4-((3S,4R)-3-(2-bromophenyl)-4- fluoropiperidin-1-yl)-6-isopropylpyrimidin-2- amine |
| 222 | 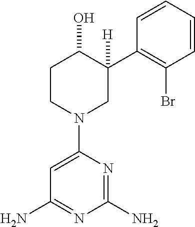 [SMILES: Nc1cc(N2CC[C@H](O)[C@@H](c3ccccc3Br)C2)nc(N)n1] | (3,4-trans)-3-(2-bromophenyl)-1-(2,6- diaminopyrimidin-4-yl)piperidin-4-ol |
| 223 | 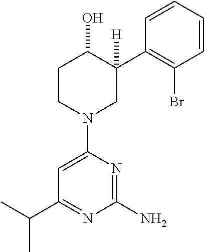 [SMILES: CC(C)c1cc(N2CC[C@H](O)[C@@H](c3ccccc3Br)C2)nc(N)n1] | (3,4-trans)-1-(2-amino-6-isopropylpyrimidin- 4-yl)-3-(2-bromophenyl)piperidin-4-ol |
| 224 | 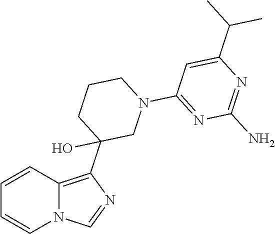 [SMILES: CC(C)c1cc(N2CCCC(O)(c3ncn4ccccc34)C2)nc(N)n1] | 1-(2-amino-6-isopropylpyrimidin-4-yl)-3- (imidazo[1,5-a]pyridin-1-yl)piperidin-3-ol |
| 225 | 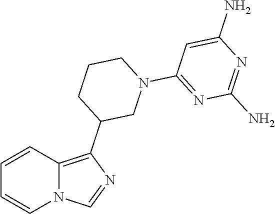 [SMILES: Nc1cc(N2CCCC(c3ncn4ccccc34)C2)nc(N)n1] | 6-(3-(imidazo[1,5-a]pyridin-1-yl)piperidin-1- yl)pyrimidine-2,4-diamine |
| 226 | 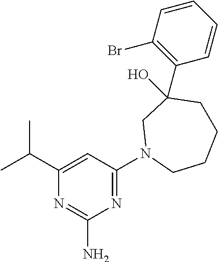 [SMILES: CC(C)c1cc(N2CCCCC(O)(c3ccccc3Br)C2)nc(N)n1] | 1-(2-amino-6-isopropylpyrimidin-4-yl)-3-(2- bromophenyl)azepan-3-ol |
| 227 | 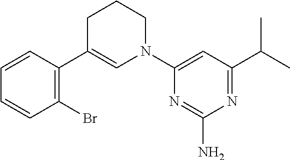 [SMILES: CC(C)c1cc(N2C=C(c3ccccc3Br)CCC2)nc(N)n1] | 4-(5-(2-bromophenyl)-3,4-dihydropyridin- 1(2H)-yl)-6-isopropylpyrimidin-2-amine |
| 228 | 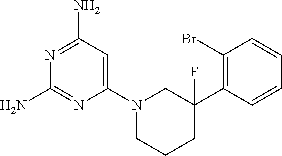 [SMILES: Nc1cc(N2CCCC(F)(c3ccccc3Br)C2)nc(N)n1] | 6-(3-(2-bromophenyl)-3-fluoropiperidin-1- yl)pyrimidine-2,4-diamine |
| 229 | 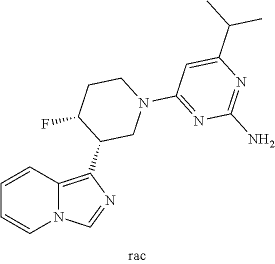 [SMILES: CC(C)c1cc(N2CC[C@@H](F)[C@@H](c3ncn4ccccc34)C2)nc(N)n1] | 4-((3R,4R)-4-fluoro-3-(imidazo[1,5-a]pyridin- 1-yl)piperidin-1-yl)-6-isopropylpyrimidin-2- amine |
| 230 | 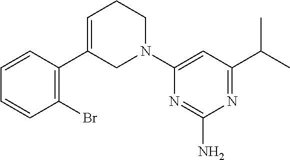 [SMILES: CC(C)c1cc(N2CCC=C(c3ccccc3Br)C2)nc(N)n1] | 4-(5-(2-bromophenyl)-3,6-dihydropyridin- 1(2H)-yl)-6-isopropylpyrimidin-2-amine |
| 231 | 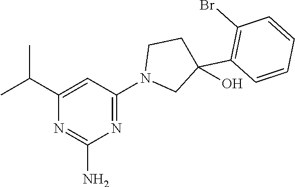 [SMILES: CC(C)c1cc(N2CCC(O)(c3ccccc3Br)C2)nc(N)n1] | 1-(2-amino-6-isopropylpyrimidin-4-yl)-3-(2- bromophenyl)pyrrolidin-3-ol |
| 232 | 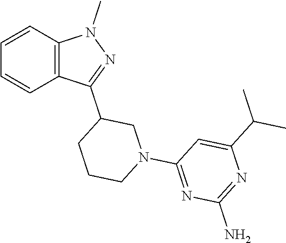 [SMILES: CC(C)c1cc(N2CCCC(c3nn(C)c4ccccc34)C2)nc(N)n1] | 4-isopropyl-6-(3-(1-methyl-1H-indazol-3- yl)piperidin-1-yl)pyrimidin-2-amine |
| 233 | 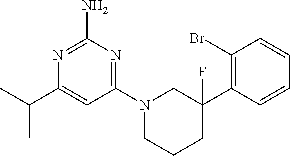 [SMILES: CC(C)c1cc(N2CCCC(F)(c3ccccc3Br)C2)nc(N)n1] | 4-(3-(2-bromophenyl)-3-fluoropiperidin-1-yl)- 6-isopropylpyrimidin-2-amine |
| 234 | 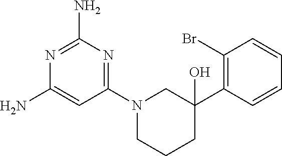 [SMILES: Nc1cc(N2CCCC(O)(c3ccccc3Br)C2)nc(N)n1] | 3-(2-bromophenyl)-1-(2,6-diaminopyrimidin- 4-yl)piperidin-3-ol |
| 235 | 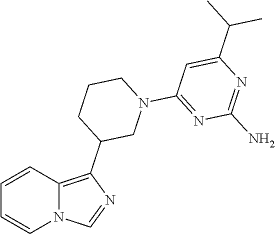 [SMILES: CC(C)c1cc(N2CCCC(c3ncn4ccccc34)C2)nc(N)n1] | 4-(3-(imidazo[1,5-a]pyridin-1-yl)piperidin-1- yl)-6-isopropylpyrimidin-2-amine |
| 236 | 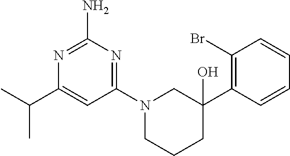 [SMILES: CC(C)c1cc(N2CCCC(O)(c3ccccc3Br)C2)nc(N)n1] | 1-(2-amino-6-isopropylpyrimidin-4-yl)-3-(2- bromophenyl)piperidin-3-ol |
| 237 | 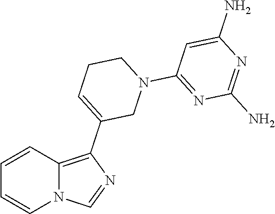 [SMILES: Nc1cc(N2CCC=C(c3ncn4ccccc34)C2)nc(N)n1] | 6-(5-(imidazo[1,5-a]pyridin-1-yl)-3,6- dihydropyridin-1(2H)-yl)pyrimidine-2,4- diamine |
| 238 | 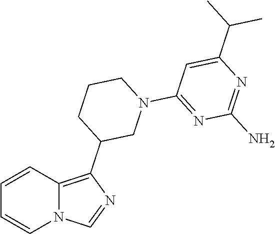 [SMILES: CC(C)c1cc(N2CCCC(c3ncn4ccccc34)C2)nc(N)n1] | 4-(5-(imidazo[1,5-a]pyridin-1-yl)-3,6- dihydropyridin-1(2H)-yl)-6- isopropylpyrimidin-2-amine |
| 239 | 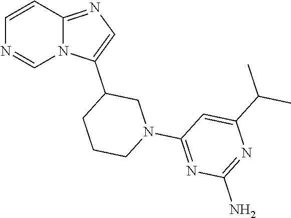 [SMILES: CC(C)c1cc(N2CCCC(c3cnc4ccncn34)C2)nc(N)n1] | 4-(3-(imidazo[1,2-c]pyrimidin-3-yl)piperidin- 1-yl)-6-isopropylpyrimidin-2-amine |
| 240 | 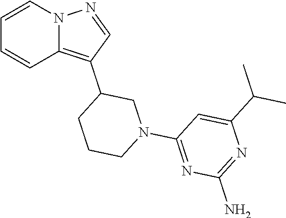 [SMILES: CC(C)c1cc(N2CCCC(c3cnn4ccccc34)C2)nc(N)n1] | 4-isopropyl-6-(3-(pyrazolo[1,5-a]pyridin-3- yl)piperidin-1-yl)pyrimidin-2-amine |
| 241 | 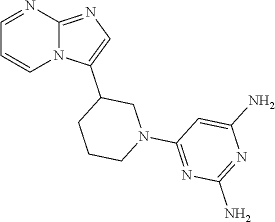 [SMILES: Nc1cc(N2CCCC(c3cnc4ncccn34)C2)nc(N)n1] | 6-(3-(imidazo[1,2-a]pyrimidin-3-yl)piperidin- 1-yl)pyrimidine-2,4-diamine |
| 242 | 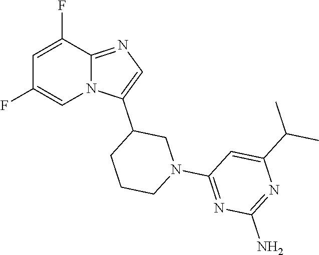 [SMILES: CC(C)c1cc(N2CCCC(c3cnc4c(F)cc(F)cn34)C2)nc(N)n1] | 4-(3-(6,8-difluoroimidazo[1,2-a]pyridin-3- yl)piperidin-1-yl)-6-isopropylpyrimidin-2- amine |
| 243 | 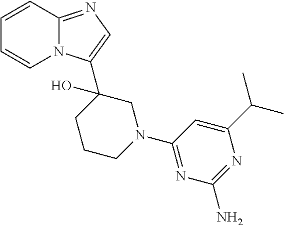 [SMILES: CC(C)c1cc(N2CCCC(O)(c3cnc4ccccn34)C2)nc(N)n1] | 1-(2-amino-6-isopropylpyrimidin-4-yl)-3- (imidazo[1,2-a]pyridin-3-yl)piperidin-3-ol |
| 244 | 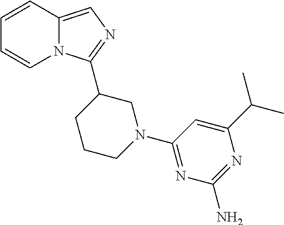 [SMILES: CC(C)c1cc(N2CCCC(c3ncc4ccccn34)C2)nc(N)n1] | 4-(3-(imidazo[1,5-a]pyridin-3-yl)piperidin-1- yl)-6-isopropylpyrimidin-2-amine |
| 245 | 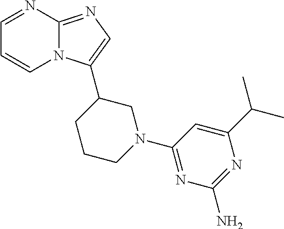 [SMILES: CC(C)c1cc(N2CCCC(c3cnc4ncccn34)C2)nc(N)n1] | 4-(3-(imidazo[1,2-a]pyrimidin-3-yl)piperidin- 1-yl)-6-isopropylpyrimidin-2-amine |
| 246 | 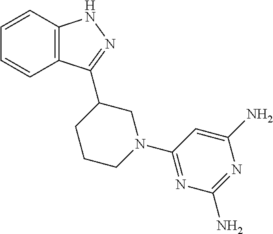 [SMILES: Nc1cc(N2CCCC(c3n[nH]c4ccccc34)C2)nc(N)n1] | 6-(3-(1H-indazol-3-yl)piperidin-1- yl)pyrimidine-2,4-diamine |
| 251 | 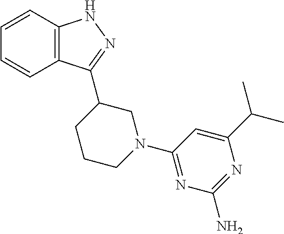 [SMILES: CC(C)c1cc(N2CCCC(c3n[nH]c4ccccc34)C2)nc(N)n1] | 4-(3-(1H-indazol-3-yl)piperidin-1-yl)-6- isopropylpyrimidin-2-amine |
| 252 | 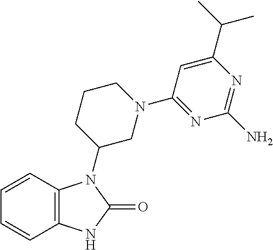 [SMILES: CC(C)c1cc(N2CCCC(n3c(=O)[nH]c4ccccc43)C2)nc(N)n1] | 1-(1-(2-amino-6-isopropylpyrimidin-4- yl)piperidin-3-yl)-1,3-dihydro-2H- benzo[d]imidazol-2-one |
| 253 | 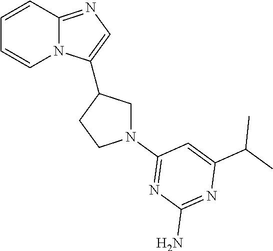 [SMILES: CC(C)c1cc(N2CCC(c3cnc4ccccn34)C2)nc(N)n1] | 4-(3-(imidazo[1,2-a]pyridin-3-yl)pyrrolidin-1- yl)-6-isopropylpyrimidin-2-amine |
| 254 | 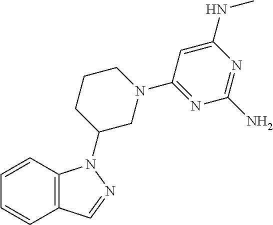 [SMILES: CNc1cc(N2CCCC(n3ncc4ccccc43)C2)nc(N)n1] | 6-(3-(1H-indazol-1-yl)piperidin-1-yl)-N4- methylpyrimidine-2,4-diamine |
| 255 | 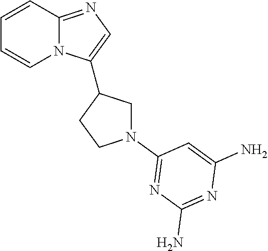 [SMILES: Nc1cc(N2CCC(c3cnc4ccccn34)C2)nc(N)n1] | 6-(3-(imidazo[1,2-a]pyridin-3-yl)pyrrolidin-1- yl)pyrimidine-2,4-diamine |
| 256 | 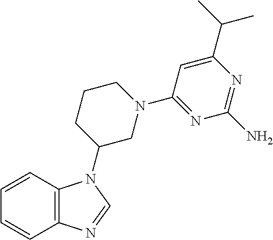 [SMILES: CC(C)c1cc(N2CCCC(n3cnc4ccccc43)C2)nc(N)n1] | 4-(3-(1H-benzo[d]imidazol-1-yl)piperidin-1- yl)-6-isopropylpyrimidin-2-amine |
| 257 | 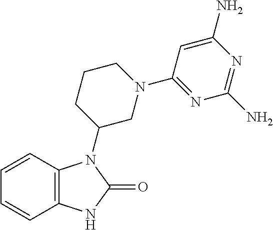 [SMILES: Nc1cc(N2CCCC(n3c(=O)[nH]c4ccccc43)C2)nc(N)n1] | 1-(1-(2,6-diaminopyrimidin-4-yl)piperidin-3- yl)-1,3-dihydro-2H-benzo[d]imidazol-2-one |
| 258 | 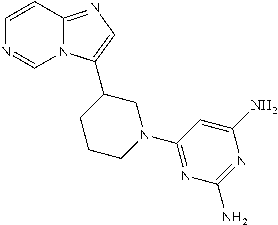 [SMILES: Nc1cc(N2CCCC(c3cnc4ccncn34)C2)nc(N)n1] | 6-(3-(imidazo[1,2-c]pyrimidin-3-yl)piperidin- 1-yl)pyrimidine-2,4-diamine |
| 259 | 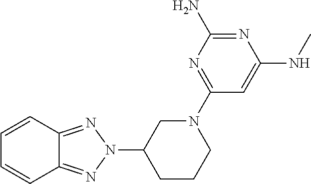 [SMILES: CNc1cc(N2CCCC(n3nc4ccccc4n3)C2)nc(N)n1] | 6-(3-(2H-benzo[d][1,2,3]triazol-2-yl)piperidin- 1-yl)-N4-methylpyrimidine-2,4-diamine |
| 260 | 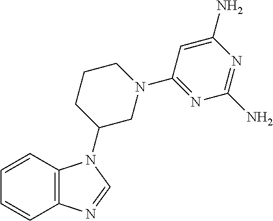 [SMILES: Nc1cc(N2CCCC(n3cnc4ccccc43)C2)nc(N)n1] | 6-(3-(1H-benzo[d]imidazol-1-yl)piperidin-1- yl)pyrimidine-2,4-diamine |
| 261 | 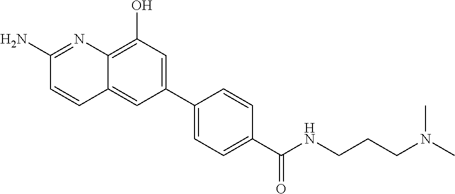 [SMILES: CN(C)CCCNC(=O)c1ccc(-c2cc(O)c3nc(N)ccc3c2)cc1] | 4-(2-amino-8-hydroxyquinolin-6-yl)-N-(3- (dimethylamino)propyl)benzamide |
| 262 | 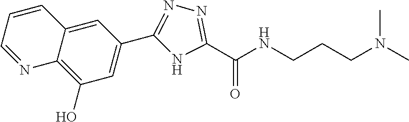 [SMILES: CN(C)CCCNC(=O)c1nnc(-c2cc(O)c3ncccc3c2)[nH]1] | N-(3-(dimethylamino)propyl)-5-(8- hydroxyquinolin-6-yl)-4H-1,2,4-triazole-3- carboxamide |
| 263 | 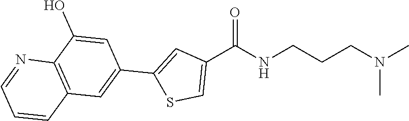 [SMILES: CN(C)CCCNC(=O)c1csc(-c2cc(O)c3ncccc3c2)c1] | N-(3-(dimethylamino)propyl)-5-(8- hydroxyquinolin-6-yl)-4H-1,2,4-triazole-3- carboxamide |
| 264 | 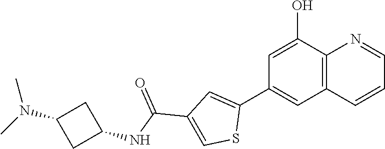 [SMILES: CN(C)[C@H]1C[C@@H](NC(=O)c2csc(-c3cc(O)c4ncccc4c3)c2)C1] | N-(cis-3-(dimethylamino)cyclobutyl)-5-(8- hydroxyquinolin-6-yl)thiophene-3- carboxamide |
| 265 | 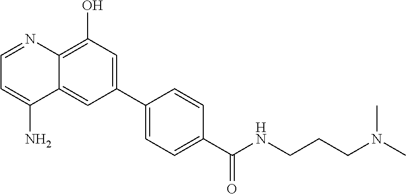 [SMILES: CN(C)CCCNC(=O)c1ccc(-c2cc(O)c3nccc(N)c3c2)cc1] | 4-(4-amino-8-hydroxyquinolin-6-yl)-N-(3- (dimethylamino)propyl)benzamide |
| 266 | 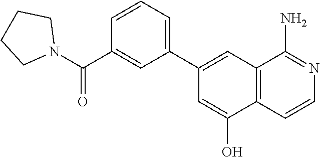 [SMILES: Nc1nccc2c(O)cc(-c3cccc(C(=O)N4CCCC4)c3)cc12] | (3-(1-amino-5-hydroxyisoquinolin-7- yl)phenyl)(pyrrolidin-1-yl)methanone |
| 267 | 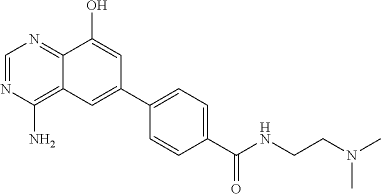 [SMILES: CN(C)CCNC(=O)c1ccc(-c2cc(O)c3ncnc(N)c3c2)cc1] | 4-(4-amino-8-hydroxyquinazolin-6-yl)-N-(2- (dimethylamino)ethyl)benzamide |
| 268 | 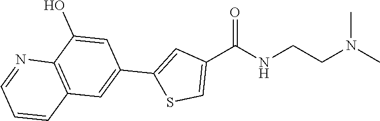 [SMILES: CN(C)CCNC(=O)c1csc(-c2cc(O)c3ncccc3c2)c1] | N-(2-(dimethylamino)ethyl)-5-(8- hydroxyquinolin-6-yl)thiophene-3- carboxamide |
| 269 | 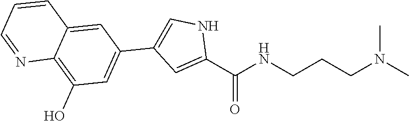 [SMILES: CN(C)CCCNC(=O)c1cc(-c2cc(O)c3ncccc3c2)c[nH]1] | N-(3-(dimethylamino)propyl)-4-(8- hydroxyquinolin-6-yl)-1H-pyrrole-2- carboxamide |
| 270 | 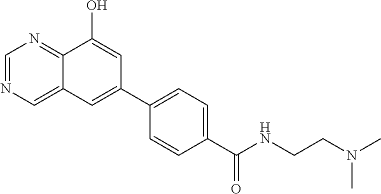 [SMILES: CN(C)CCNC(=O)c1ccc(-c2cc(O)c3ncncc3c2)cc1] | N-(2-(dimethylamino)ethyl)-4-(8- hydroxyquinazolin-6-yl)benzamide |
| 271 | 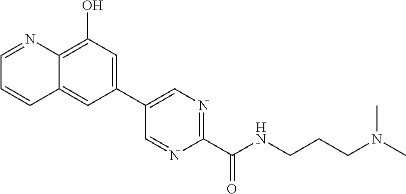 [SMILES: CN(C)CCCNC(=O)c1ncc(-c2cc(O)c3ncccc3c2)cn1] | N-(3-(dimethylamino)propyl)-5-(8- hydroxyquinolin-6-yl)pyrimidine-2- carboxamide |
| 272 | 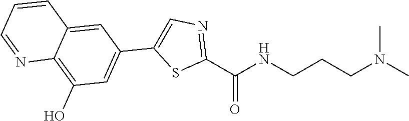 [SMILES: CN(C)CCCNC(=O)c1ncc(-c2cc(O)c3ncccc3c2)s1] | N-(3-(dimethylamino)propyl)-5-(8- hydroxyquinolin-6-yl)thiazole-2-carboxamide |
| 273 | 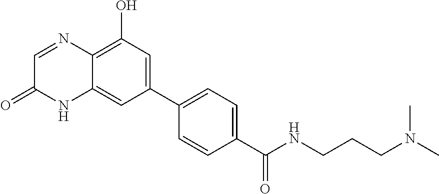 [SMILES: CN(C)CCCNC(=O)c1ccc(-c2cc(O)c3ncc(=O)[nH]c3c2)cc1] | N-(3-(dimethylamino)propyl)-4-(8-hydroxy-3- oxo-3,4-dihydroquinoxalin-6-yl)benzamide |
| 274 | 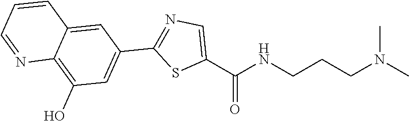 [SMILES: CN(C)CCCNC(=O)c1cnc(-c2cc(O)c3ncccc3c2)s1] | N-(3-(dimethylamino)propyl)-2-(8- hydroxyquinolin-6-yl)thiazole-5-carboxamide |
| 275 | 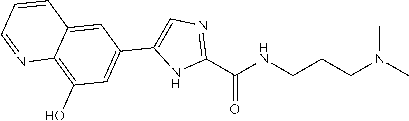 [SMILES: CN(C)CCCNC(=O)c1ncc(-c2cc(O)c3ncccc3c2)[nH]1] | N-(3-(dimethylamino)propyl)-5-(8- hydroxyquinolin-6-yl)-1H-imidazole-2- carboxamide |
| 276 | 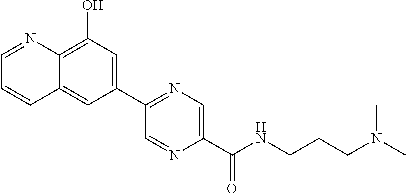 [SMILES: CN(C)CCCNC(=O)c1cnc(-c2cc(O)c3ncccc3c2)cn1] | N-(3-(dimethylamino)propyl)-5-(8- hydroxyquinolin-6-yl)pyrazine-2- carboxamide |
| 277 | 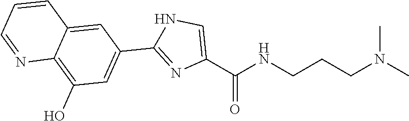 [SMILES: CN(C)CCCNC(=O)c1c[nH]c(-c2cc(O)c3ncccc3c2)n1] | N-(3-(dimethylamino)propyl)-2-(8- hydroxyquinolin-6-yl)-1H-imidazole-4- carboxamide |
| 278 | 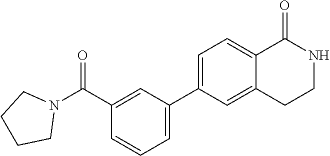 [SMILES: O=C1NCCc2cc(-c3cccc(C(=O)N4CCCC4)c3)ccc21] | 6-(3-(pyrrolidine-1-carbonyl)phenyl)-3,4- dihydroisoquinolin-1(2H)-one |
| 279 | 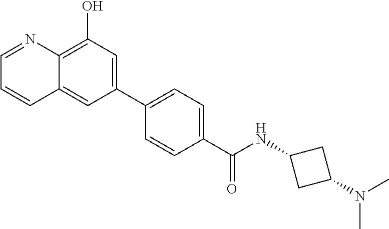 [SMILES: CN(C)[C@H]1C[C@@H](NC(=O)c2ccc(-c3cc(O)c4ncccc4c3)cc2)C1] | N-(cis-3-(dimethylamino)cyclobutyl)-4-(8- hydroxyquinolin-6-yl)benzamide |
| 280 | 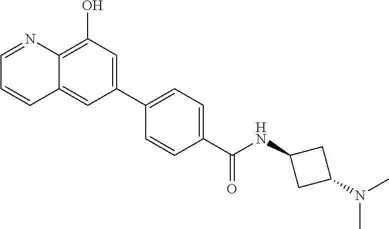 [SMILES: CN(C)[C@H]1C[C@H](NC(=O)c2ccc(-c3cc(O)c4ncccc4c3)cc2)C1] | N-(trans-3-(dimethylamino)cyclobutyl)-4-(8- hydroxyquinolin-6-yl)benzamide |
| 281 | 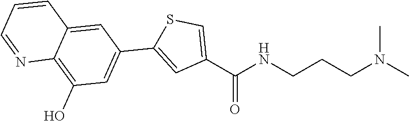 [SMILES: CN(C)CCCNC(=O)c1csc(-c2cc(O)c3ncccc3c2)c1] | N-(3-(dimethylamino)propyl)-5-(8- hydroxyquinolin-6-yl)thiophene-3- carboxamide |
| 282 | 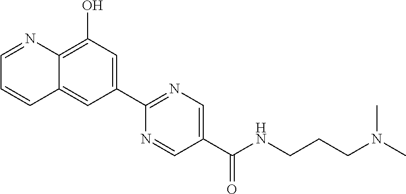 [SMILES: CN(C)CCCNC(=O)c1cnc(-c2cc(O)c3ncccc3c2)nc1] | N-(3-(dimethylamino)propyl)-2-(8- hydroxyquinolin-6-yl)pyrimidine-5- carboxamide |
| 283 | 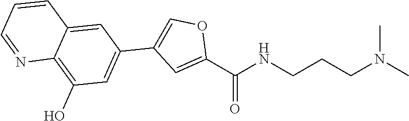 [SMILES: CN(C)CCCNC(=O)c1cc(-c2cc(O)c3ncccc3c2)co1] | N-(3-(dimethylamino)propyl)-4-(8- hydroxyquinolin-6-yl)furan-2-carboxamide |
| 284 | 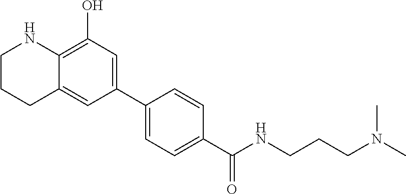 [SMILES: CN(C)CCCNC(=O)c1ccc(-c2cc(O)c3c(c2)CCCN3)cc1] | N-(3-(dimethylamino)propyl)-4-(8-hydroxy- 1,2,3,4-tetrahydroquinolin-6-yl)benzamide |
| 285 | 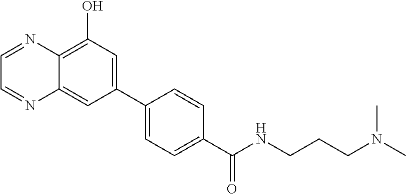 [SMILES: CN(C)CCCNC(=O)c1ccc(-c2cc(O)c3nccnc3c2)cc1] | N-(3-(dimethylamino)propyl)-4-(8- hydroxyquinoxalin-6-yl)benzamide |
| 286 | 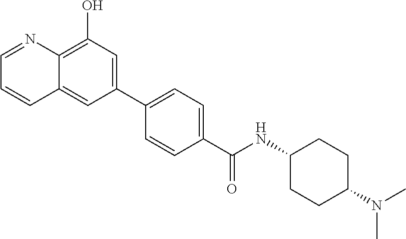 [SMILES: CN(C)[C@H]1CC[C@@H](NC(=O)c2ccc(-c3cc(O)c4ncccc4c3)cc2)CC1] | N-(cis-4-(dimethylamino)cyclohexyl)-4-(8- hydroxyquinolin-6-yl)benzamide |
| 287 | 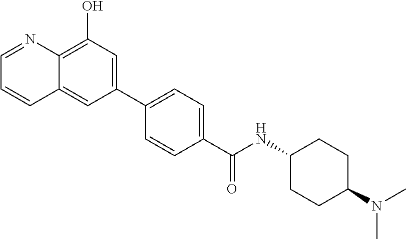 [SMILES: CN(C)[C@H]1CC[C@H](NC(=O)c2ccc(-c3cc(O)c4ncccc4c3)cc2)CC1] | N-(trans-4-(dimethylamino)cyclohexyl)-4-(8- hydroxyquinolin-6-yl)benzamide |
| 288 | 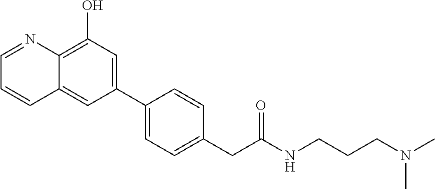 [SMILES: CN(C)CCCNC(=O)Cc1ccc(-c2cc(O)c3ncccc3c2)cc1] | N-(3-(dimethylamino)propyl)-2-(4-(8- hydroxyquinolin-6-yl)phenyl)acetamide |
| 289 | 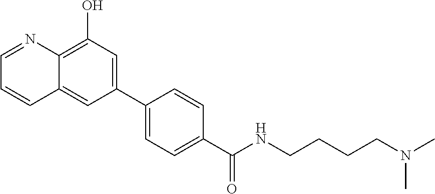 [SMILES: CN(C)CCCCNC(=O)c1ccc(-c2cc(O)c3ncccc3c2)cc1] | N-(4-(dimethylamino)butyl)-4-(8- hydroxyquinolin-6-yl)benzamide |
| 290 | 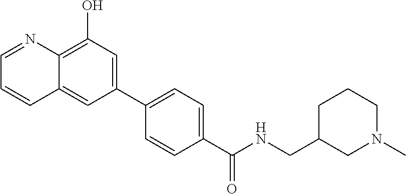 [SMILES: CN1CCCC(CNC(=O)c2ccc(-c3cc(O)c4ncccc4c3)cc2)C1] | 4-(8-hydroxyquinolin-6-yl)-N-((1- methylpiperidin-3-yl)methyl)benzamide |
| 291 | 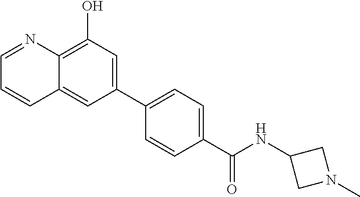 [SMILES: CN1CC(NC(=O)c2ccc(-c3cc(O)c4ncccc4c3)cc2)C1] | 4-(8-hydroxyquinolin-6-yl)-N-(1- methylazetidin-3-yl)benzamide |
| 292 | 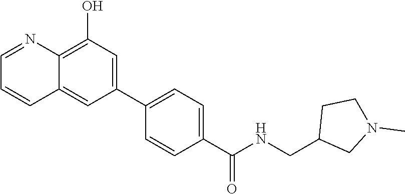 [SMILES: CN1CCC(CNC(=O)c2ccc(-c3cc(O)c4ncccc4c3)cc2)C1] | 4-(8-hydroxyquinolin-6-yl)-N-((1- methylpyrrolidin-3-yl)methyl)benzamide |
| 293 | 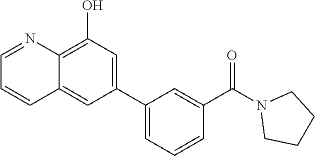 [SMILES: O=C(c1cccc(-c2cc(O)c3ncccc3c2)c1)N1CCCC1] | (3-(8-hydroxyquinolin-6-yl)phenyl)(pyrrolidin- 1-yl)methanone |
| 294 | 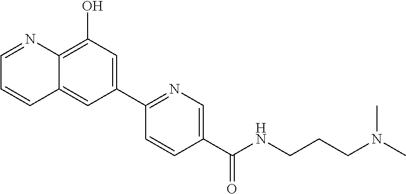 [SMILES: CN(C)CCCNC(=O)c1ccc(-c2cc(O)c3ncccc3c2)nc1] | N-(3-(dimethylamino)propyl)-6-(8- hydroxyquinolin-6-yl)nicotinamide |
| 295 | 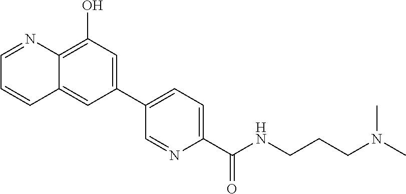 [SMILES: CN(C)CCCNC(=O)c1ccc(-c2cc(O)c3ncccc3c2)cn1] | N-(3-(dimethylamino)propyl)-5-(8- hydroxyquinolin-6-yl)picolinamide |
| 296 | 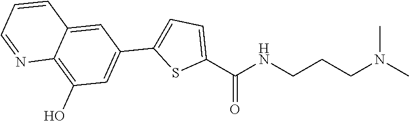 [SMILES: CN(C)CCCNC(=O)c1ccc(-c2cc(O)c3ncccc3c2)s1] | N-(3-(dimethylamino)propyl)-5-(8- hydroxyquinolin-6-yl)thiophene-2- carboxamide |
| 297 | 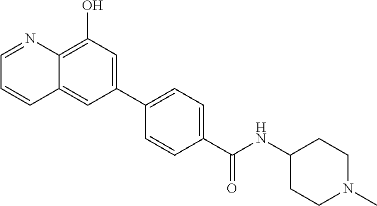 [SMILES: CN1CCC(NC(=O)c2ccc(-c3cc(O)c4ncccc4c3)cc2)CC1] | 4-(8-hydroxyquinolin-6-yl)-N-(1- methylpiperidin-4-yl)benzamide |
| 298 | 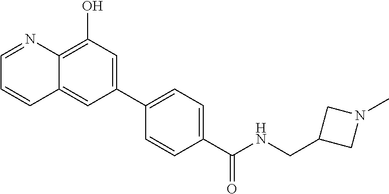 [SMILES: CN1CC(CNC(=O)c2ccc(-c3cc(O)c4ncccc4c3)cc2)C1] | 4-(8-hydroxyquinolin-6-yl)-N-((1- methylazetidin-3-yl)methyl)benzamide |
| 299 | 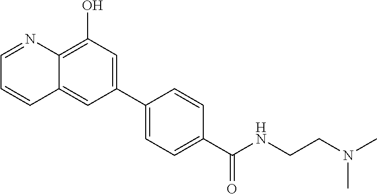 [SMILES: CN(C)CCNC(=O)c1ccc(-c2cc(O)c3ncccc3c2)cc1] | N-(2-(dimethylamino)ethyl)-4-(8- hydroxyquinolin-6-yl)benzamide |
| 300 | 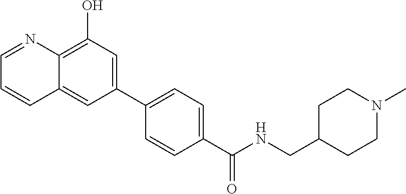 [SMILES: CN1CCC(CNC(=O)c2ccc(-c3cc(O)c4ncccc4c3)cc2)CC1] | 4-(8-hydroxyquinolin-6-yl)-N-((1- methylpiperidin-4-yl)methyl)benzamide |
| 301 | 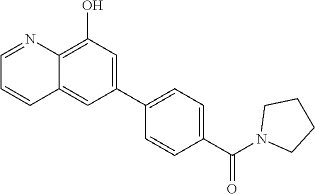 [SMILES: O=C(c1ccc(-c2cc(O)c3ncccc3c2)cc1)N1CCCC1] | (4-(8-hydroxyquinolin-6-yl)phenyl)(pyrrolidin- 1-yl)methanone |
| 302 | 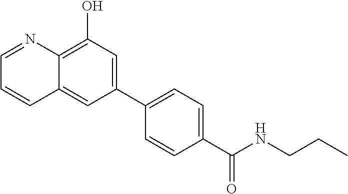 [SMILES: CCCNC(=O)c1ccc(-c2cc(O)c3ncccc3c2)cc1] | 4-(8-hydroxyquinolin-6-yl)-N- propylbenzamide |
| 303 | 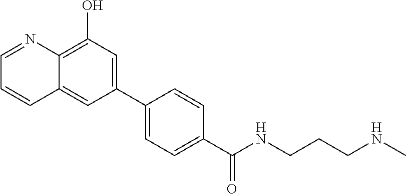 [SMILES: CNCCCNC(=O)c1ccc(-c2cc(O)c3ncccc3c2)cc1] | 4-(8-hydroxyquinolin-6-yl)-N-(3- (methylamino)propyl)benzamide |
| 304 | 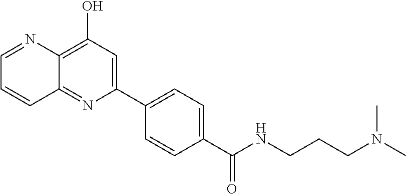 [SMILES: CN(C)CCCNC(=O)c1ccc(-c2cc(O)c3ncccc3n2)cc1] | N-(3-(dimethylamino)propyl)-4-(4-hydroxy- 1,5-naphthyridin-2-yl)benzamide |
| 305 | 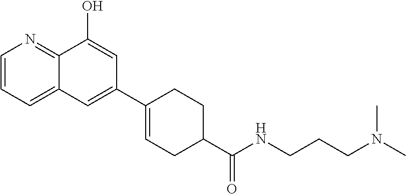 [SMILES: CN(C)CCCNC(=O)C1CC=C(c2cc(O)c3ncccc3c2)CC1] | N-(3-(dimethylamino)propyl)-4-(8- hydroxyquinolin-6-yl)cyclohex-3-ene-1- carboxamide |
| 306 | 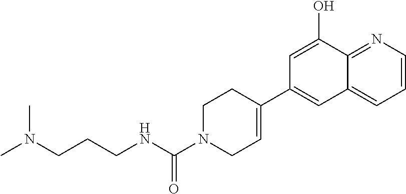 [SMILES: CN(C)CCCNC(=O)N1CC=C(c2cc(O)c3ncccc3c2)CC1] | N-(3-(dimethylamino)propyl)-4-(8- hydroxyquinolin-6-yl)-3,6-dihydropyridine- 1(2H)-carboxamide |
| 307 | 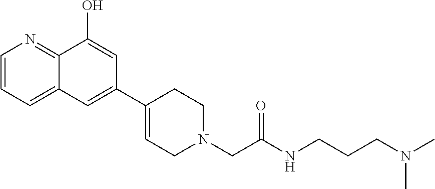 [SMILES: CN(C)CCCNC(=O)CN1CC=C(c2cc(O)c3ncccc3c2)CC1] | N-(3-(dimethylamino)propyl)-2-(4-(8- hydroxyquinolin-6-yl)-3,6-dihydropyridin- 1(2H)-yl)acetamide |
| 308 | 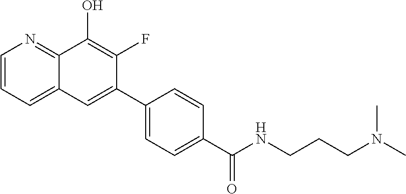 [SMILES: CN(C)CCCNC(=O)c1ccc(-c2cc3cccnc3c(O)c2F)cc1] | N-(3-(dimethylamino)propyl)-4-(7-fluoro-8- hydroxyquinolin-6-yl)benzamide |
| 309 | 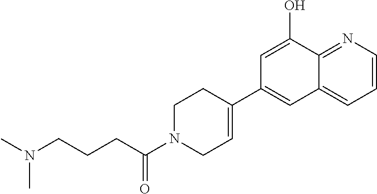 [SMILES: CN(C)CCCC(=O)N1CC=C(c2cc(O)c3ncccc3c2)CC1] | 4-(dimethylamino)-1-(4-(8-hydroxyquinolin- 6-yl)-3,6-dihydropyridin-1(2H)-yl)butan-1- one |
| 310 | 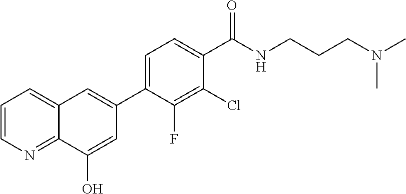 [SMILES: CN(C)CCCNC(=O)c1ccc(-c2cc(O)c3ncccc3c2)c(F)c1Cl] | 2-chloro-N-(3-(dimethylamino)propyl)-3- fluoro-4-(8-hydroxyquinolin-6-yl)benzamide |
| 311 | 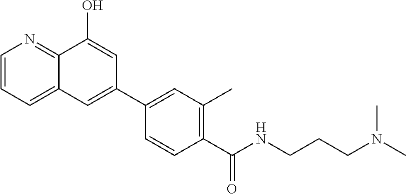 [SMILES: Cc1cc(-c2cc(O)c3ncccc3c2)ccc1C(=O)NCCCN(C)C] | N-(3-(dimethylamino)propyl)-4-(8- hydroxyquinolin-6-yl)-2-methylbenzamide |
| 312 | 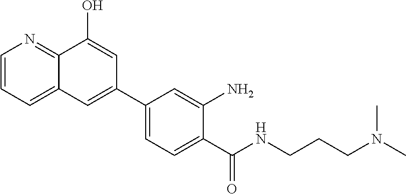 [SMILES: CN(C)CCCNC(=O)c1ccc(-c2cc(O)c3ncccc3c2)cc1N] | 2-amino-N-(3-(dimethylamino)propyl)-4-(8- hydroxyquinolin-6-yl)benzamide |
| 313 | 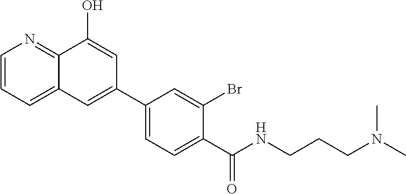 [SMILES: CN(C)CCCNC(=O)c1ccc(-c2cc(O)c3ncccc3c2)cc1Br] | 2-bromo-N-(3-(dimethylamino)propyl)-4-(8- hydroxyquinolin-6-yl)benzamide |
| 314 | 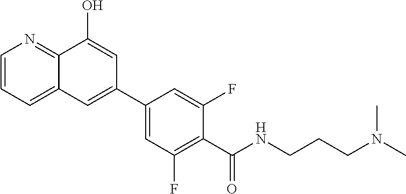 [SMILES: CN(C)CCCNC(=O)c1c(F)cc(-c2cc(O)c3ncccc3c2)cc1F] | N-(3-(dimethylamino)propyl)-2,6-difluoro-4- (8-hydroxyquinolin-6-yl)benzamide |
| 315 | 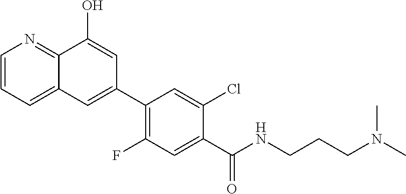 [SMILES: CN(C)CCCNC(=O)c1cc(F)c(-c2cc(O)c3ncccc3c2)cc1Cl] | 2-chloro-N-(3-(dimethylamino)propyl)-5- fluoro-4-(8-hydroxyquinolin-6-yl)benzamide |
| 316 | 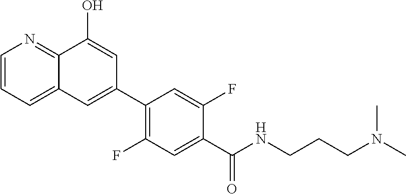 [SMILES: CN(C)CCCNC(=O)c1cc(F)c(-c2cc(O)c3ncccc3c2)cc1F] | N-(3-(dimethylamino)propyl)-2,5-difluoro-4- (8-hydroxyquinolin-6-yl)benzamide |
| 317 | 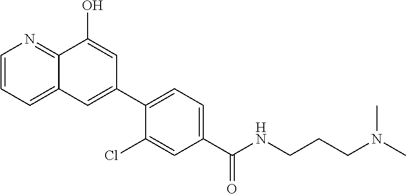 [SMILES: CN(C)CCCNC(=O)c1ccc(-c2cc(O)c3ncccc3c2)c(Cl)c1] | 3-chloro-N-(3-(dimethylamino)propyl)-4-(8- hydroxyquinolin-6-yl)benzamide |
| 318 | 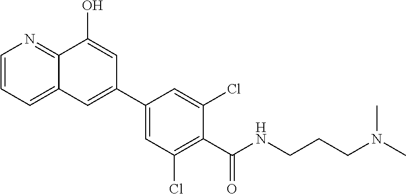 [SMILES: CN(C)CCCNC(=O)c1c(Cl)cc(-c2cc(O)c3ncccc3c2)cc1Cl] | 2,6-dichloro-N-(3-(dimethylamino)propyl)-4- (8-hydroxyquinolin-6-yl)benzamide |
| 319 | 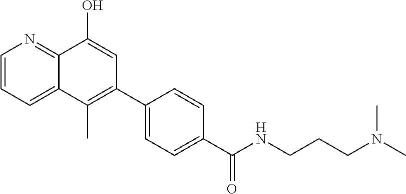 [SMILES: Cc1c(-c2ccc(C(=O)NCCCN(C)C)cc2)cc(O)c2ncccc12] | N-(3-(dimethylamino)propyl)-4-(8-hydroxy-5- methylquinolin-6-yl)benzamide |
| 320 | 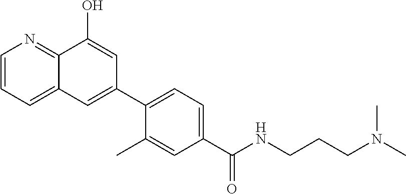 [SMILES: Cc1cc(C(=O)NCCCN(C)C)ccc1-c1cc(O)c2ncccc2c1] | N-(3-(dimethylamino)propyl)-4-(8- hydroxyquinolin-6-yl)-3-methylbenzamide |
| 321 | 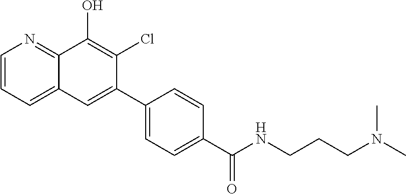 [SMILES: CN(C)CCCNC(=O)c1ccc(-c2cc3cccnc3c(O)c2Cl)cc1] | 4-(7-chloro-8-hydroxyquinolin-6-yl)-N-(3- (dimethylamino)propyl)benzamide |
| 322 | 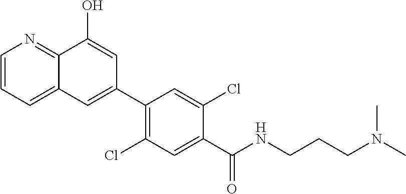 [SMILES: CN(C)CCCNC(=O)c1cc(Cl)c(-c2cc(O)c3ncccc3c2)cc1Cl] | 2,5-dichloro-N-(3-(dimethylamino)propyl)-4- (8-hydroxyquinolin-6-yl)benzamide |
| 323 | 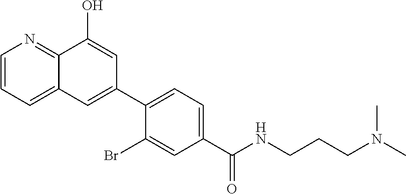 [SMILES: CN(C)CCCNC(=O)c1ccc(-c2cc(O)c3ncccc3c2)c(Br)c1] | 3-bromo-N-(3-(dimethylamino)propyl)-4-(8- hydroxyquinolin-6-yl)benzamide |
| 324 | 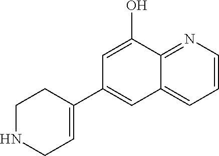 [SMILES: Oc1cc(C2=CCNCC2)cc2cccnc12] | 6-(1,2,3,6-tetrahydropyridin-4-yl)quinolin-8-ol |
| 325 | 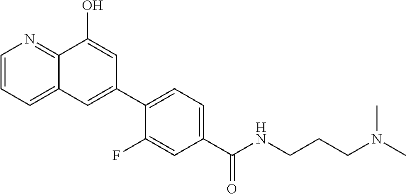 [SMILES: CN(C)CCCNC(=O)c1ccc(-c2cc(O)c3ncccc3c2)c(F)c1] | N-(3-(dimethylamino)propyl)-3-fluoro-4-(8- hydroxyquinolin-6-yl)benzamide |
| 326 | 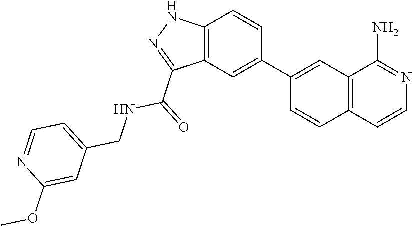 [SMILES: COc1cc(CNC(=O)c2n[nH]c3ccc(-c4ccc5ccnc(N)c5c4)cc23)ccn1] | 5-(1-aminoisoquinolin-7-yl)-N-((2- methoxypyridin-4-yl)methyl)-1H-indazole-3- carboxamide |
| 327 | 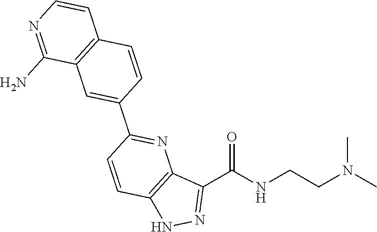 [SMILES: CN(C)CCNC(=O)c1n[nH]c2ccc(-c3ccc4ccnc(N)c4c3)nc12] | 5-(1-aminoisoquinolin-7-yl)-N-(2- (dimethylamino)ethyl)-1H-pyrazolo[4,3- b]pyridine-3-carboxamide |
| 328 | 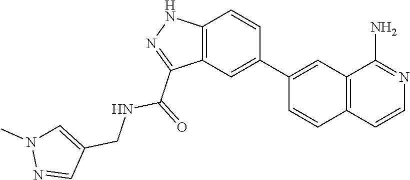 [SMILES: Cn1cc(CNC(=O)c2n[nH]c3ccc(-c4ccc5ccnc(N)c5c4)cc23)cn1] | 5-(1-aminoisoquinolin-7-yl)-N-((1-methyl-1H- pyrazol-4-yl)methyl)-1H-indazole-3- carboxamide |
| 329 | 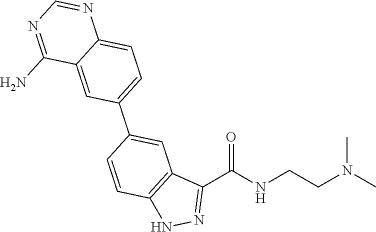 [SMILES: CN(C)CCNC(=O)c1n[nH]c2ccc(-c3ccc4ncnc(N)c4c3)cc12] | 5-(4-aminoquinazolin-6-yl)-N-(2- (dimethylamino)ethyl)-1H-indazole-3- carboxamide |
| 330 | 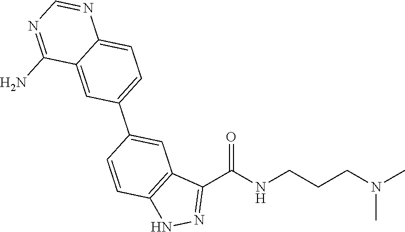 [SMILES: CN(C)CCCNC(=O)c1n[nH]c2ccc(-c3ccc4ncnc(N)c4c3)cc12] | 5-(4-aminoquinazolin-6-yl)-N-(3- (dimethylamino)propyl)-1H-indazole-3- carboxamide |
| 331 | 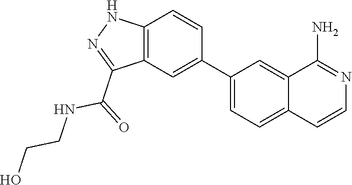 [SMILES: Nc1nccc2ccc(-c3ccc4[nH]nc(C(=O)NCCO)c4c3)cc12] | 5-(1-aminoisoquinolin-7-yl)-N-(2- hydroxyethyl)-1H-indazole-3-carboxamide |
| 332 | 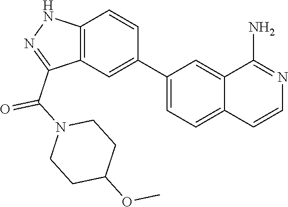 [SMILES: COC1CCN(C(=O)c2n[nH]c3ccc(-c4ccc5ccnc(N)c5c4)cc23)CC1] | (5-(1-aminoisoquinolin-7-yl)-1H-indazol-3- yl)(4-methoxypiperidin-1-yl)methanone |
| 333 | 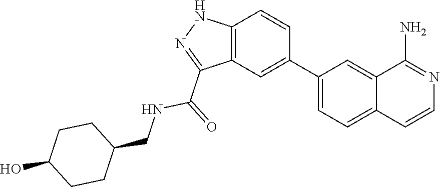 [SMILES: Nc1nccc2ccc(-c3ccc4[nH]nc(C(=O)NC[C@H]5CC[C@@H](O)CC5)c4c3)cc12] | 5-(1-aminoisoquinolin-7-yl)-N-((cis-4- hydroxycyclohexyl)methyl)-1H-indazole-3- carboxamide |
| 334 | 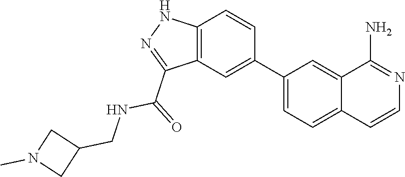 [SMILES: CN1CC(CNC(=O)c2n[nH]c3ccc(-c4ccc5ccnc(N)c5c4)cc23)C1] | 5-(1-aminoisoquinolin-7-yl)-N-((1- methylazetidin-3-yl)methyl)-1H-indazole-3- carboxamide |
| 335 | 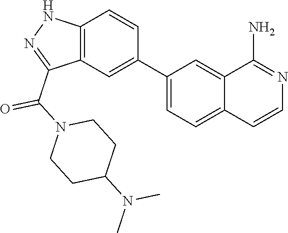 [SMILES: CN(C)C1CCN(C(=O)c2n[nH]c3ccc(-c4ccc5ccnc(N)c5c4)cc23)CC1] | (5-(1-aminoisoquinolin-7-yl)-1H-indazol-3- yl)(4-(dimethylamino)piperidin-1- yl)methanone |
| 336 | 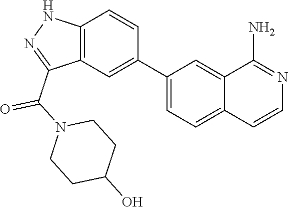 [SMILES: Nc1nccc2ccc(-c3ccc4[nH]nc(C(=O)N5CCC(O)CC5)c4c3)cc12] | (5-(1-aminoisoquinolin-7-yl)-1H-indazol-3- yl)(4-hydroxypiperidin-1-yl)methanone |
| 337 | 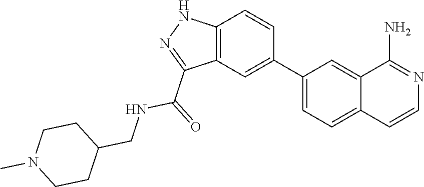 [SMILES: CN1CCC(CNC(=O)c2n[nH]c3ccc(-c4ccc5ccnc(N)c5c4)cc23)CC1] | 5-(1-aminoisoquinolin-7-yl)-N-((1- methylpiperidin-4-yl)methyl)-1H-indazole-3- carboxamide |
| 338 | 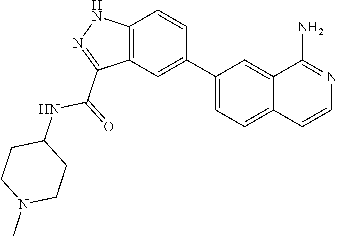 [SMILES: CN1CCC(NC(=O)c2n[nH]c3ccc(-c4ccc5ccnc(N)c5c4)cc23)CC1] | 5-(1-aminoisoquinolin-7-yl)-N-(1- methylpiperidin-4-yl)-1H-indazole-3- carboxamide |
| 339 | 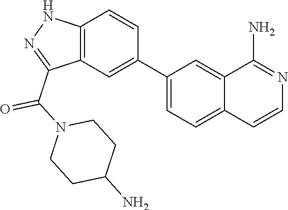 [SMILES: Nc1nccc2ccc(-c3ccc4[nH]nc(C(=O)N5CCC(N)CC5)c4c3)cc12] | (5-(1-aminoisoquinolin-7-yl)-1H-indazol-3- yl)(4-aminopiperidin-1-yl)methanone |
| 340 | 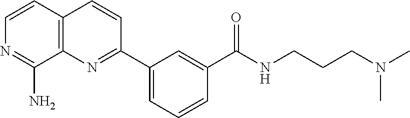 [SMILES: CN(C)CCCNC(=O)c1cccc(-c2ccc3ccnc(N)c3n2)c1] | 3-(8-amino-1,7-naphthyridin-2-yl)-N-(3- (dimethylamino)propyl)benzamide |
| 341 | 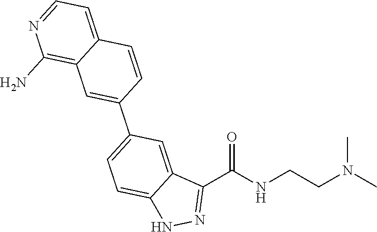 [SMILES: CN(C)CCNC(=O)c1n[nH]c2ccc(-c3ccc4ccnc(N)c4c3)cc12] | 5-(1-aminoisoquinolin-7-yl)-N-(2- (dimethylamino)ethyl)-1H-indazole-3- carboxamide |
| 342 | 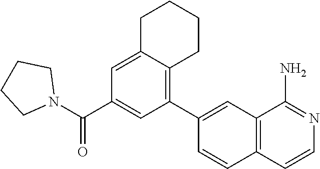 [SMILES: Nc1nccc2ccc(-c3cc(C(=O)N4CCCC4)cc4c3CCCC4)cc12] | (4-(1-aminoisoquinolin-7-yl)-5,6,7,8- tetrahydronaphthalen-2-yl)(pyrrolidin-1- yl)methanone |
| 343 | 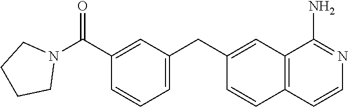 [SMILES: Nc1nccc2ccc(Cc3cccc(C(=O)N4CCCC4)c3)cc12] | (3-((1-aminoisoquinolin-7- yl)methyl)phenyl)(pyrrolidin-1-yl)methanone |
| 344 | 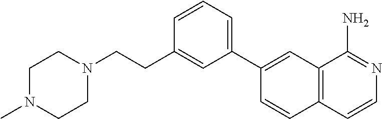 [SMILES: CN1CCN(CCc2cccc(-c3ccc4ccnc(N)c4c3)c2)CC1] | 7-(3-(2-(4-methylpiperazin-1- yl)ethyl)phenyl)isoquinolin-1-amine |
| 345 | 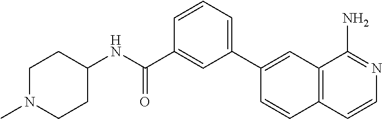 [SMILES: CN1CCC(NC(=O)c2cccc(-c3ccc4ccnc(N)c4c3)c2)CC1] | 3-(1-aminoisoquinolin-7-yl)-N-(1- methylpiperidin-4-yl)benzamide |
| 346 | 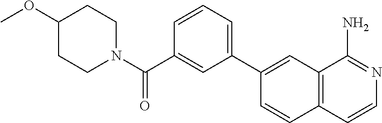 [SMILES: COC1CCN(C(=O)c2cccc(-c3ccc4ccnc(N)c4c3)c2)CC1] | (3-(1-aminoisoquinolin-7-yl)phenyl)(4- methoxypiperidin-1-yl)methanone |
| 347 | 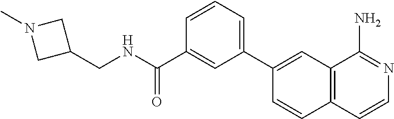 [SMILES: CN1CC(CNC(=O)c2cccc(-c3ccc4ccnc(N)c4c3)c2)C1] | 3-(1-aminoisoquinolin-7-yl)-N-((1- methylazetidin-3-yl)methyl)benzamide |
| 348 | 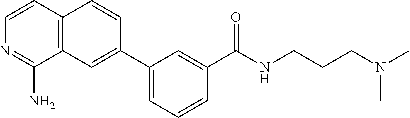 [SMILES: CN(C)CCCNC(=O)c1cccc(-c2ccc3ccnc(N)c3c2)c1] | 3-(1-aminoisoquinolin-7-yl)-N-(3- (dimethylamino)propyl)benzamide |
| 349 | 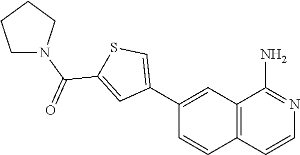 [SMILES: Nc1nccc2ccc(-c3csc(C(=O)N4CCCC4)c3)cc12] | (4-(1-aminoisoquinolin-7-yl)thiophen-2- yl)(pyrrolidin-1-yl)methanone |
| 350 | 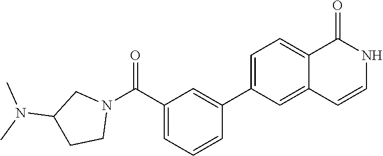 [SMILES: CN(C)C1CCN(C(=O)c2cccc(-c3ccc4c(=O)[nH]ccc4c3)c2)C1] | 6-(3-(3-(dimethylamino)pyrrolidine-1- carbonyl)phenyl)isoquinolin-1(2H)-one |
| 351 | 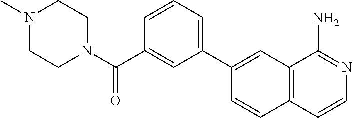 [SMILES: CN1CCN(C(=O)c2cccc(-c3ccc4ccnc(N)c4c3)c2)CC1] | (3-(1-aminoisoquinolin-7-yl)phenyl)(4- methylpiperazin-1-yl)methanone |
| 352 | 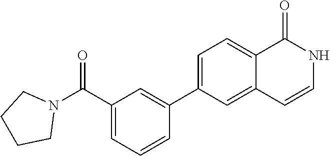 [SMILES: O=C(c1cccc(-c2ccc3c(=O)[nH]ccc3c2)c1)N1CCCC1] | 6-(3-(pyrrolidine-1- carbonyl)phenyl)isoquinolin-1(2H)-one |
| 353 | 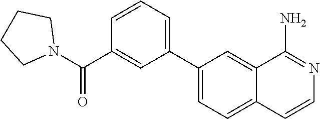 [SMILES: Nc1nccc2ccc(-c3cccc(C(=O)N4CCCC4)c3)cc12] | (3-(1-aminoisoquinolin-7- yl)phenyl)(pyrrolidin-1-yl)methanone |
| 354 | 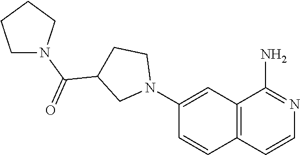 [SMILES: Nc1nccc2ccc(N3CCC(C(=O)N4CCCC4)C3)cc12] | (1-(1-aminoisoquinolin-7-yl)pyrrolidin-3- yl)(pyrrolidin-1-yl)methanone |
| 355 | 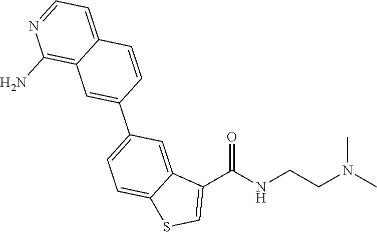 [SMILES: CN(C)CCNC(=O)c1csc2ccc(-c3ccc4ccnc(N)c4c3)cc12] | 5-(1-aminoisoquinolin-7-yl)-N-(2- (dimethylamino)ethyl)benzo[b]thiophene-3- carboxamide |
| 356 | 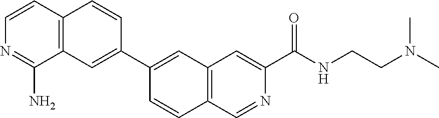 [SMILES: CN(C)CCNC(=O)c1cc2cc(-c3ccc4ccnc(N)c4c3)ccc2cn1] | 1′-amino-N-(2-(dimethylamino)ethyl)-[6,7′- biisoquinoline]-3-carboxamide |
| 357 | 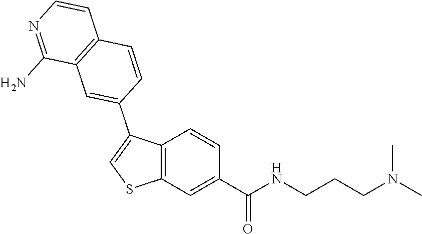 [SMILES: CN(C)CCCNC(=O)c1ccc2c(-c3ccc4ccnc(N)c4c3)csc2c1] | 3-(1-aminoisoquinolin-7-yl)-N-(3- (dimethylamino)propyl)benzo[b]thiophene-6- carboxamide |
| 358 | 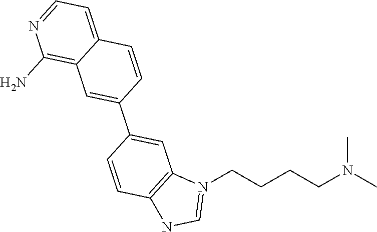 | 7-(1-(4-(dimethylamino)butyl)-1H- benzo[d]imidazol-6-yl)isoquinolin-1-amine |
| 359 | 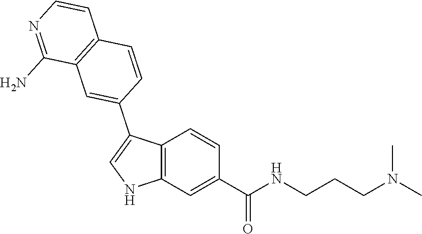 [SMILES: CN(C)CCCNC(=O)c1ccc2c(-c3ccc4ccnc(N)c4c3)c[nH]c2c1] | 3-(1-aminoisoquinolin-7-yl)-N-(3- (dimethylamino)propyl)-1H-indole-6- carboxamide |
| 360 | 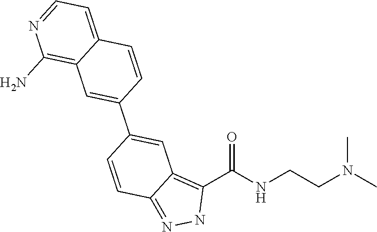 [SMILES: CN(C)CCNC(=O)c1[nH]nc2ccc(-c3ccc4ccnc(N)c4c3)cc12] | 5-(1-aminoisoquinolin-7-yl)-N-(2- (dimethylamino)ethyl)-2-methyl-2H-indazole- 3-carboxamide |
| 361 | 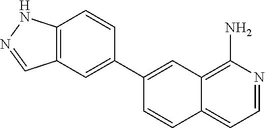 [SMILES: Nc1nccc2ccc(-c3ccc4[nH]ncc4c3)cc12] | 7-(1H-indazol-5-yl)isoquinolin-1-amine |
| 362 | 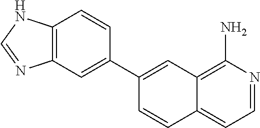 [SMILES: Nc1nccc2ccc(-c3ccc4[nH]cnc4c3)cc12] | 7-(1H-benzo[d]imidazol-5-yl)isoquinolin-1- amine |
| 363 | 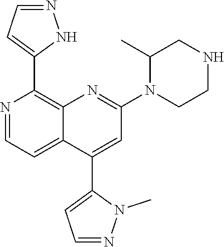 [SMILES: CC1CNCCN1c1cc(-c2ccnn2C)c2ccnc(-c3ccn[nH]3)c2n1] | 4-(1-methyl-1H-pyrazol-5-yl)-2-(2- methylpiperazin-1-yl)-8-(1H-pyrazol-5-yl)- 1,7-naphthyridine |
| 364 | 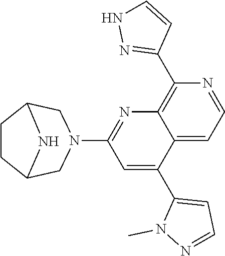 [SMILES: Cn1nccc1-c1cc(N2CC3CCC(C2)N3)nc2c(-c3cc[nH]n3)nccc12] | 2-(3,8-diazabicyclo[3.2.1]octan-3-yl)-4-(1- methyl-1H-pyrazol-5-yl)-8-(1H-pyrazol-3-yl)- 1,7-naphthyridine |
| 365 | 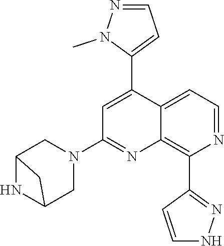 [SMILES: Cn1nccc1-c1cc(N2CC3CC(C2)N3)nc2c(-c3cc[nH]n3)nccc12] | 3-(4-(1-methyl-1H-pyrazol-5-yl)-8-(1H- pyrazol-3-yl)-1,7-naphthyridin-2-yl)-3,6- diazabicyclo[3.1.1]heptane |
| 366 | 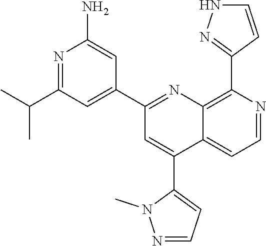 [SMILES: CC(C)c1cc(-c2cc(-c3ccnn3C)c3ccnc(-c4cc[nH]n4)c3n2)cc(N)n1] | 6-isopropyl-4-(4-(1-methyl-1H-pyrazol-5-yl)- 8-(1H-pyrazol-3-yl)-1,7-naphthyridin-2- yl)pyridin-2-amine |
| 367 | 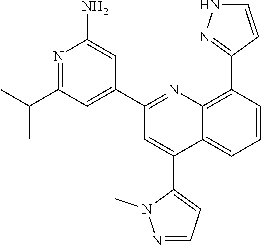 [SMILES: CC(C)c1cc(-c2cc(-c3ccnn3C)c3cccc(-c4cc[nH]n4)c3n2)cc(N)n1] | 6-isopropyl-4-(4-(1-methyl-1H-pyrazol-5-yl)- 8-(1H-pyrazol-3-yl)quinolin-2-yl)pyridin-2- amine |
| 368 | 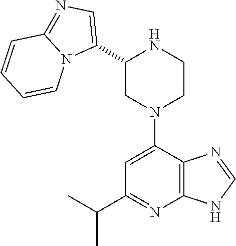 [SMILES: CC(C)c1cc(N2CCN[C@@H](c3cnc4ccccn34)C2)c2nc[nH]c2n1] | (R/S)-7-(3-(imidazo[1,2-a]pyridin-3- yl)piperazin-1-yl)-5-isopropyl-3H- imidazo[4,5-b]pyridine |
| 369 | 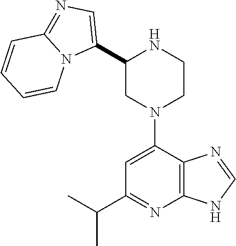 [SMILES: CC(C)c1cc(N2CCN[C@H](c3cnc4ccccn34)C2)c2nc[nH]c2n1] | (S/R)-7-(3-(imidazo[1,2-a]pyridin-3- yl)piperazin-1-yl)-5-isopropyl-3H- imidazo[4,5-b]pyridine |
| 370 | 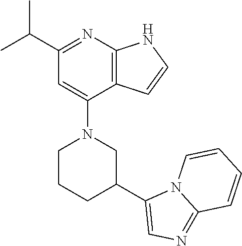 [SMILES: CC(C)c1cc(N2CCCC(c3cnc4ccccn34)C2)c2cc[nH]c2n1] | 3-(1-(6-isopropyl-1H-pyrrolo[2,3-b]pyridin-4- yl)piperidin-3-yl)imidazo[1,2-a]pyridine |
| 371 | 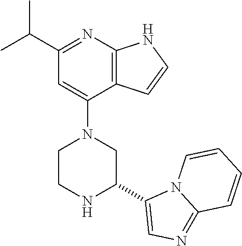 | (R/S)-3-(4-(6-isopropyl-1H-pyrrolo[2,3- b]pyridin-4-yl)piperazin-2-yl)imidazo[1,2- a]pyridine |
| 372 | 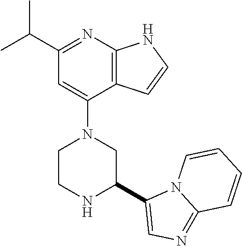 | (S/R)-3-(4-(6-isopropyl-1H-pyrrolo[2,3- b]pyridin-4-yl)piperazin-2-yl)imidazo[1,2- a]pyridine |
| 373 | 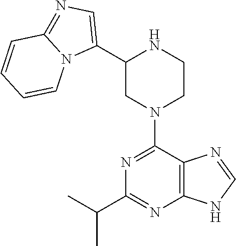 [SMILES: CC(C)c1nc(N2CCNC(c3cnc4ccccn34)C2)c2nc[nH]c2n1] | 6-(3-(imidazo[1,2-a]pyridin-3-yl)piperazin-1- yl)-2-isopropyl-9H-purine |
| 374 | 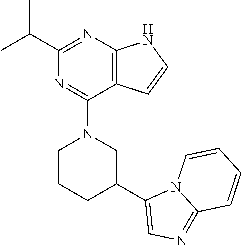 [SMILES: CC(C)c1nc(N2CCCC(c3cnc4ccccn34)C2)c2cc[nH]c2n1] | 4-(3-(imidazo[1,2-a]pyridin-3-yl)piperidin-1- yl)-2-isopropyl-7H-pyrrolo[2,3-d]pyrimidine |
| 375 | 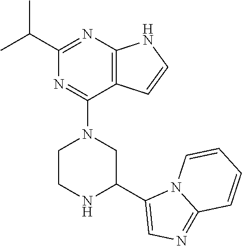 [SMILES: CC(C)c1nc(N2CCNC(c3cnc4ccccn34)C2)c2cc[nH]c2n1] | 4-(3-(imidazo[1,2-a]pyridin-3-yl)piperazin-1- yl)-2-isopropyl-7H-pyrrolo[2,3-d]pyrimidine |
| 376 | 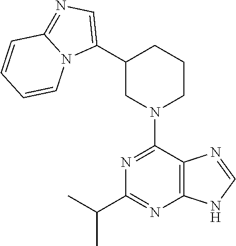 [SMILES: CC(C)c1nc(N2CCCC(c3cnc4ccccn34)C2)c2nc[nH]c2n1] | 6-(3-(imidazo[1,2-a]pyridin-3-yl)piperidin-1- yl)-2-isopropyl-9H-purine |
| 377 | 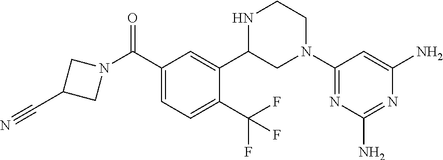 [SMILES: N#CC1CN(C(=O)c2ccc(C(F)(F)F)c(C3CN(c4cc(N)nc(N)n4)CCN3)c2)C1] | 1-(3-(4-(2,6-diaminopyrimidin-4-yl)piperazin- 2-yl)-4-(trifluoromethyl)benzoyl)azetidine-3- carbonitrile |
| 378 | 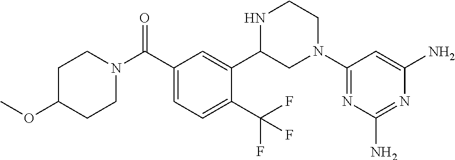 [SMILES: COC1CCN(C(=O)c2ccc(C(F)(F)F)c(C3CN(c4cc(N)nc(N)n4)CCN3)c2)CC1] | (3-(4-(2,6-diaminopyrimidin-4-yl)piperazin-2- yl)-4-(trifluoromethyl)phenyl)(4- methoxypiperidin-1-yl)methanone |
| 379 | 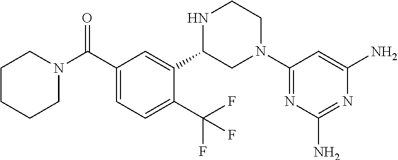 [SMILES: Nc1cc(N2CCN[C@@H](c3cc(C(=O)N4CCCCC4)ccc3C(F)(F)F)C2)nc(N)n1] | (S)-(3-(4-(2,6-diaminopyrimidin-4- yl)piperazin-2-yl)-4- (trifluoromethyl)phenyl)(piperidin-1- yl)methanone |
| 380 | 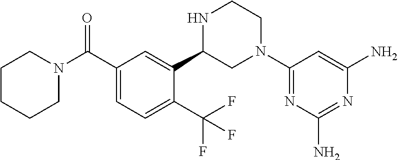 [SMILES: Nc1cc(N2CCN[C@H](c3cc(C(=O)N4CCCCC4)ccc3C(F)(F)F)C2)nc(N)n1] | (R)-(3-(4-(2,6-diaminopyrimidin-4- yl)piperazin-2-yl)-4- (trifluoromethyl)phenyl)(piperidin-1- yl)methanone |
| 381 | 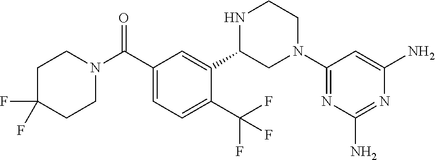 [SMILES: Nc1cc(N2CCN[C@@H](c3cc(C(=O)N4CCC(F)(F)CC4)ccc3C(F)(F)F)C2)nc(N)n1] | (S)-(3-(4-(2,6-diaminopyrimidin-4- yl)piperazin-2-yl)-4- (trifluoromethyl)phenyl)(4,4-difluoropiperidin- 1-yl)methanone |
| 382 | 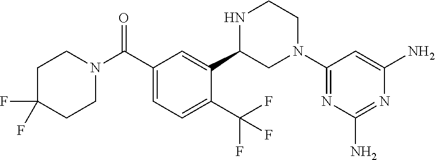 [SMILES: Nc1cc(N2CCN[C@H](c3cc(C(=O)N4CCC(F)(F)CC4)ccc3C(F)(F)F)C2)nc(N)n1] | (R)-(3-(4-(2,6-diaminopyrimidin-4- yl)piperazin-2-yl)-4- (trifluoromethyl)phenyl)(4,4-difluoropiperidin- 1-yl)methanone |
| 383 | 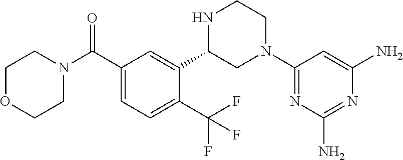 [SMILES: Nc1cc(N2CCN[C@@H](c3cc(C(=O)N4CCOCC4)ccc3C(F)(F)F)C2)nc(N)n1] | (S)-(3-(4-(2,6-diaminopyrimidin-4- yl)piperazin-2-yl)-4- (trifluoromethyl)phenyl)(morpholino) methanone |
| 384 | 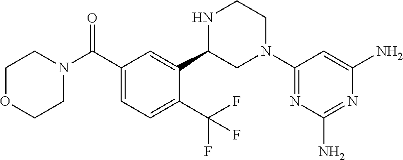 [SMILES: Nc1cc(N2CCN[C@H](c3cc(C(=O)N4CCOCC4)ccc3C(F)(F)F)C2)nc(N)n1] | (R)-(3-(4-(2,6-diaminopyrimidin-4- yl)piperazin-2-yl)-4- (trifluoromethyl)phenyl)(morpholino) methanone |

### Pharmaceutical Compositions

Pharmaceutical compositions of the present disclosure comprise at least one compound of Formula (1) (e.g., Formula (1a), Formula (1aa), Formula (1ab), Formula (1b), Formula (1ba), Formula (1c), Formula (1ca)), Formula (2) (e.g., Formula (2a), Formula (2aa), Formula (2b)), or a tautomer, stereoisomer or a mixture of stereoisomers, or a pharmaceutically acceptable salt, or hydrate, or deuterated derivative thereof formulated together with one or more pharmaceutically acceptable carriers. These formulations include those suitable for oral, rectal, topical, buccal and parenteral (e.g., subcutaneous, intramuscular, intradermal, or intravenous) administration. The most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one compound of the present disclosure as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the present disclosure may be prepared by any of the well-known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of the present disclosure with a liquid or finely divided solid carrier, or both, and then, if necessary, shaping the product. For example, a tablet may be prepared by compressing or molding a powder or granules of at least one compound of the present disclosure, which may be optionally combined with one or more accessory ingredients. Compressed tablets may be prepared by compressing, in a suitable machine, at least one compound of the present disclosure in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the present disclosure is moistened with an inert liquid diluent.

Formulations suitable for buccal (sub-lingual) administration include lozenges comprising at least one compound of the present disclosure in a flavored base, usually sucrose and acacia or tragacanth, and pastilles comprising the at least one compound in an inert base such as gelatin and glycerin or sucrose and acacia.

Formulations of the present disclosure suitable for parenteral administration comprise sterile aqueous preparations of at least one compound of Formula (1) (e.g., Formula (1a), Formula (1aa), Formula (1ab), Formula (1b), Formula (1ba), Formula (1c), Formula (1 ca)), Formula (2) (e.g., Formula (2a), Formula (2aa), Formula (2b)), or a tautomer, stereoisomer or a mixture of stereoisomers, or a pharmaceutically acceptable salt, or hydrate, or deuterated derivative thereof, which are approximately isotonic with the blood of the intended recipient. These preparations are administered intravenously, although administration may also be effected by means of subcutaneous, intramuscular, or intradermal injection. Such preparations may conveniently be prepared by admixing at least one compound described herein with water and rendering the resulting solution sterile and isotonic with the blood. Injectable compositions according to the present disclosure may contain from about 0.1 to about 5% w/w of the active compound.

Formulations suitable for rectal administration are presented as unit-dose suppositories. These may be prepared by admixing at least one compound as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one compound of Formula (1) (e.g., Formula (1a), Formula (1aa), Formula (1ab), Formula (1 b), Formula (1ba), Formula (1c), Formula (1ca)), Formula (2) (e.g., Formula (2a), Formula (2aa), Formula (2b)), or a tautomer, stereoisomer or a mixture of stereoisomers, or a pharmaceutically acceptable salt, or hydrate, or deuterated derivative thereof) is generally present at a concentration of from about 0.1% to about 15% w/w of the composition, for example, from about 0.5 to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 1 μg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50(4):219-244 (1966) and the following Table for Equivalent Surface Area Dosage Factors).

TABLE 2
Equivalent Surface Area Dosage Factors.

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

### Methods of Treatment

In some embodiments, a compound of Formula (1) (e.g., Formula (1a), Formula (1aa), Formula (1ab), Formula (1b), Formula (1ba), Formula (1c), Formula (1ca)), Formula (2) (e.g., Formula (2a), Formula (2aa), Formula (2b)), or a tautomer, stereoisomer or a mixture of stereoisomers, or a pharmaceutically acceptable salt, or hydrate, or deuterated derivative thereof, is administered to treat cancer in a subject in need thereof. In some embodiments, the cancer is chosen from breast cancer, lung cancer, pancreatic cancer, colorectal cancer, gall bladder cancer, thyroid cancer, bile duct cancer, ovarian cancer, endometrial cancer, prostate cancer, and esophageal cancer. In some embodiments the cancer is selected from breast cancer, lung cancer, pancreatic cancer, colorectal cancer, gall bladder cancer, thyroid cancer, bile duct cancer, ovarian cancer, endometrial cancer, prostate cancer, and esophageal cancer. In some embodiments, the method further comprises administering to the subject in combination with an anti-cancer agent. In some embodiments, the cancer is breast cancer. In some embodiments, the cancer is lung cancer. In some embodiments, the cancer is pancreatic cancer. In some embodiments, the cancer is colorectal cancer. In some embodiments, the cancer is gall bladder cancer. In some embodiments, the cancer is thyroid cancer. In some embodiments, the cancer is bile duct cancer. In some embodiments, the cancer is ovarian cancer. In some embodiments, the cancer is endometrial cancer. In some embodiments, the cancer is prostate cancer. In some embodiments, the cancer is esophageal cancer. In some embodiments, the therapeutic treatment is for the treatment of glucose-induced degradation-deficient (GID) E3 ligase associated diseases and conditions.

In some embodiments, a compound of Formula (1) (e.g., Formula (1a), Formula (1aa), Formula (1ab), Formula (1b), Formula (1ba), Formula (1c), Formula (1ca)), Formula (2) (e.g., Formula (2a), Formula (2aa), Formula (2b)), or a tautomer, stereoisomer or a mixture of stereoisomers, or a pharmaceutically acceptable salt, or hydrate, or deuterated derivative thereof, is administered as a pharmaceutical composition.

In some embodiments, the invention provides for methods for inhibiting glucose-induced degradation-deficient E3 ligase activity in a cell, comprising contacting the cell in which inhibition of glucose-induced degradation-deficient (GID) E3 ligase activity is desired with an effective amount of a compound of Formula (1) (e.g., Formula (1a), Formula (1aa), Formula (1ab), Formula (1b), Formula (1ba), Formula (1c), Formula (1ca)), Formula (2) (e.g., Formula (2a), Formula (2aa), Formula (2b)), pharmaceutically acceptable salts thereof or pharmaceutical compositions containing the compound or pharmaceutically acceptable salt thereof. In one embodiment, the contacting is in vitro. In one embodiment, the contacting is in vivo.

As used herein, the term “contacting” refers to the bringing together of indicated moieties in an in vitro system or an in vivo system. For example, “contacting” a glucose-induced degradation-deficient E3 ligase with a compound provided herein includes the administration of a compound provided herein to an individual or patient, such as a human, having glucose-induced degradation-deficient E3 ligase, as well as, for example, introducing a compound provided herein into a sample containing a cellular or purified preparation containing the glucose-induced degradation-deficient E3 ligase.

In one embodiment, a cell in which inhibition of glucose-induced degradation-deficient E3 ligase activity is desired is contacted with an effective amount of a compound of Formula (1) (e.g., Formula (1a), Formula (1aa), Formula (1ab), Formula (1b), Formula (1ba), Formula (1c), Formula (1ca)), and/or Formula (2) (e.g., Formula (2a), Formula (2aa), Formula (2b)) to negatively modulate the activity of glucose-induced degradation-deficient E3 ligase. In some embodiments, a therapeutically effective amount of pharmaceutically acceptable salt or pharmaceutical compositions containing the compound of Formula (1) (e.g., Formula (1a), Formula (1aa), Formula (1ab), Formula (1b), Formula (1ba), Formula (1c), Formula (1ca)), and/or Formula (2) (e.g., Formula (2a), Formula (2aa), Formula (2b)) may be used.

By negatively modulating the activity of glucose-induced degradation-deficient E3 ligase, the methods described herein are designed to inhibit undesired cellular proliferation resulting from enhanced glucose-induced degradation-deficient E3 ligase activity within the cell. The cells may be contacted in a single dose or multiple doses in accordance with a particular treatment regimen to effect the desired negative modulation of glucose-induced degradation-deficient E3 ligase.

The concentration and route of administration to the patient will vary depending on the cancer to be treated.

In one embodiment, a compound of Formula (1) (e.g., Formula (1a), Formula (1aa), Formula (1ab), Formula (1b), Formula (1ba), Formula (1c), Formula (1ca)), Formula (2) (e.g., Formula (2a), Formula (2aa), Formula (2b)), or a tautomer, stereoisomer or a mixture of stereoisomers, or a pharmaceutically acceptable salt, or hydrate, or deuterated derivative thereof, is administered in combination with another therapeutic agent, e.g., chemotherapy, or used in combination with other treatments, such as radiation or surgical intervention, either as an adjuvant prior to surgery or post-operatively.

In some embodiments, the subject has been previously treated with an anti-cancer agent. In some embodiments, a compound of Formula (1) (e.g., Formula (1a), Formula (1 aa), Formula (1ab), Formula (1 b), Formula (1ba), Formula (1c), Formula (1ca)), Formula (2) (e.g., Formula (2a), Formula (2aa), Formula (2b)), or a pharmaceutically acceptable salt or solvate thereof, as defined herein, may be administered to a subject in combination with an anti-cancer agent. In some embodiments, the anti-cancer agent is BRAF inhibitor. In some embodiments, the anti-cancer agent is MEK inhibitor. In some embodiments, the anti-cancer agent is ERK inhibitor. In some embodiments, the anti-cancer agent is SHP2 inhibitor. In some embodiments, the anti-cancer agent is SOS1 inhibitor. In some embodiments, the anti-cancer agent is PI3K inhibitor. In some embodiments, the anti-cancer agent is AKT inhibitor. In some embodiments, the anti-cancer agent is PD1/PDL1 inhibitor. In some embodiments, the anti-cancer agent is NRF2 inhibitor. In some embodiments, the anti-cancer agent is AMPK activator. In some embodiments, the anti-cancer agent is WNT inhibitor. In some embodiments, the anti-cancer agent is an mTOR inhibitor. In some embodiments, the anti-cancer agent is an Insulin-like Growth Factor 1 receptor (IGF-1R) inhibitor. In some embodiments, the anti-cancer agent is an epidermal growth factor receptor (EGFR) inhibitor. In some embodiments, the EGFR inhibitor is cetuximab. In some embodiments, the EGFR inhibitor is afatinib.

Also provided herein is a compound of Formula (1) (e.g., Formula (1a), Formula (1aa), Formula (1ab), Formula (1b), Formula (1ba), Formula (1c), Formula (1ca)), Formula (2) (e.g., Formula (2a), Formula (2aa), Formula (2b)), or a pharmaceutically acceptable salt or solvate thereof, or a pharmaceutical composition thereof as defined herein for use in therapy.

Also provided herein is a compound of Formula (1) (e.g., Formula (1a), Formula (1aa), Formula (1ab), Formula (1b), Formula (1ba), Formula (1c), Formula (1ca)), Formula (2) (e.g., Formula (2a), Formula (2aa), Formula (2b)), or a pharmaceutically acceptable salt or solvate thereof or a pharmaceutical composition thereof as defined herein for use in the treatment of cancer.

Also provided herein is a compound of Formula (1) (e.g., Formula (1a), Formula (1aa), Formula (1ab), Formula (1b), Formula (1ba), Formula (1c), Formula (1ca)), Formula (2) (e.g., Formula (2a), Formula (2aa), Formula (2b)), or a pharmaceutically acceptable salt or solvate thereof for use in the inhibition of glucose-induced degradation-deficient E3 ligase.

Also provided herein is a compound of Formula (1) (e.g., Formula (1a), Formula (1aa), Formula (1ab), Formula (1b), Formula (1ba), Formula (1c), Formula (1ca)), Formula (2) (e.g., Formula (2a), Formula (2aa), Formula (2b)), or a pharmaceutically acceptable salt or solvate thereof or a pharmaceutical composition thereof as defined herein, for use in the treatment of a glucose-induced degradation-deficient E3 ligase associated disease or disorder.

Also provided herein is the use of a compound of Formula (1) (e.g., Formula (1a), Formula (1aa), Formula (1ab), Formula (1b), Formula (1ba), Formula (1c), Formula (1ca)), Formula (2) (e.g., Formula (2a), Formula (2aa), Formula (2b)), or a pharmaceutically acceptable salt or solvate thereof, as defined herein in the manufacture of a medicament for the treatment of cancer.

Also provided herein is a use of a compound of Formula (1) (e.g., Formula (1a), Formula (1aa), Formula (1ab), Formula (1b), Formula (1ba), Formula (1c), Formula (1ca)), Formula (2) (e.g., Formula (2a), Formula (2aa), Formula (2b)), or a pharmaceutically acceptable salt or solvate thereof, as defined herein in the manufacture of a medicament for the inhibition of activity of glucose-induced degradation-deficient E3 ligase.

Also provided herein is the use of a compound of Formula (1) (e.g., Formula (1a), Formula (1aa), Formula (1ab), Formula (1b), Formula (1ba), Formula (1c), Formula (1ca)), Formula (2) (e.g., Formula (2a), Formula (2aa), Formula (2b)), or a pharmaceutically acceptable salt or solvate thereof, as defined herein, in the manufacture of a medicament for the treatment of a glucose-induced degradation-deficient E3 ligase associated disease or disorder.

Non-limiting embodiments of the present disclosure include:
1. A compound of Formula (1) or a tautomer, stereoisomer or a mixture of stereoisomers, or a pharmaceutically acceptable salt, or hydrate, or deuterated derivative thereof:

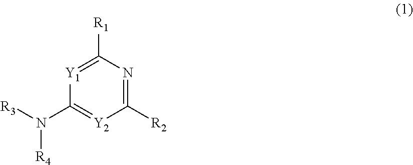

wherein:
Y₁ is either:
   chosen from CH, N, and CX₁, wherein X₁ is a halogen, or
   Y₁ and R₁ together form an optionally substituted 3 to 7 membered saturated or unsaturated ring G, wherein the ring has 0 to 3 heteroatoms;
Y₂ is chosen from CH, N, and CX₁, wherein X₁ is a halogen;
R₁ is either:
   chosen from NHR₅, H, and C₁-C₄ alkyl, wherein R₅ is chosen from H and optionally substituted C₁-C₃ alkyl, or
   Y₁ and R₁ together form a 3 to 7 membered saturated or unsaturated ring G, wherein the ring has 0 to 3 heteroatoms;
R₂ is chosen from NHR₆, H, and C₁-C₄ alkyl, wherein R₆ is chosen from H and optionally substituted C₁-C₃ alkyl; and
R₃ and R₄ are each independently chosen from hydrogen, an optionally substituted 5 to 7 membered ring, and optionally substituted C₁-C₃ alkyl; or
   R₃ and R₄ together form a 5 to 9 membered ring system D, wherein the ring is chosen from monocyclic rings and bicyclic rings, and wherein the monocyclic rings and bicyclic rings are optionally substituted with a halogen, a hydroxyl, an optionally substituted phenyl, an acetyl group, a 5 membered heteroaryl, an optionally substituted 9 membered heterobicyclic aryl, an optionally substituted benzimidazolone, and/or an optionally substituted C₁-C₃ alkyl.
2. The compound according to embodiment 1, wherein the compound is a compound of Formula (1a):

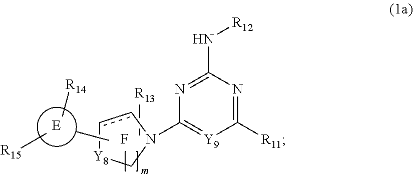

wherein:
m is 1, 2, or 3;
the dashed bond in ring F is either a single bond or a double bond;
R₁₁ is chosen from a C₁-C₃ alkyl and NHR₂₁, wherein R₂₁ is chosen from H and a C₁-C₃ alkyl;
R₁₂ is chosen from H and a C₁-C₄ alkyl optionally substituted with a hydroxyl;
R₁₃ is either:
   absent,
   chosen from a halogen, a hydroxyl, and a C₁-C₃ alkyl optionally substituted with a hydroxyl, or
   R₁₃ and Y₈ together form a 5 membered heteroaryl ring;
Y₈ is either:
   chosen from O, CH₂, an acetyl group, and NR₁₄, wherein R₁₄ is chosen from H and a C₁-C₃ alkyl optionally substituted with a hydroxyl, or
   R₁₃ and Y₈ together form a 5 membered heteroaryl ring; ring system E is chosen from 6 membered aryls and 9 membered bicyclic heteroaryls;
Y₉ is chosen from N and CX₂, wherein X₂ is chosen from H and a halogen;
R₁₄ is absent or a halogen; and
R₁₅ is absent or chosen from a halogen, a hydroxyl, a 5 membered heteroaryl having 1 to 3 heteroatoms and optionally substituted with up to two methyl groups,

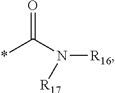

a trifluoromethyl group, a C₁-C₃ alkoxy group,

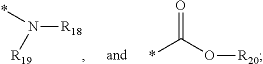

wherein:
   R₁₆ is either:
      chosen from H and a C₁-C₆ alkyl optionally substituted with a tertiary amine, or
      R₁₆ and R₁₇ together form a 5 to 6 membered non-aromatic ring optionally substituted with up to two C₁-C₃ alkyls each independently optionally substituted with a hydroxyl;
   R₁₇ is either:
      chosen from H and a C₁-C₃ alkyl, or
      R₁₆ and R₁₇ together form a 5 to 6 membered non-aromatic ring optionally substituted with up to two C₁-C₃ alkyls each independently optionally substituted with a hydroxyl;
   R₁₈ is either:
      chosen from H and a C₁-C₆ alkyl optionally substituted with a tertiary amine or a hydroxyl, or
      R₁₈ and R₁₉ together form a 4 to 6 membered non-aromatic ring optionally substituted with a C₁-C₃ alkyl optionally substituted with a tertiary amine or a hydroxyl;
   R₁₉ is either:
      chosen from H and a C₁-C₃ alkyl, or
      R₁₈ and R₁₉ together form a 4 to 6 membered non-aromatic ring optionally substituted with a C₁-C₃ alkyl optionally substituted with a tertiary amine or a hydroxyl; and
   R₂₀ is chosen from C₁-C₃ alkyls;
and wherein Formula (1a) encompasses all tautomers, stereoisomers, deuterated derivatives, pharmaceutically acceptable salts, and mixtures thereof.
3. The compound according to embodiment 2, wherein ring system E is chosen from:

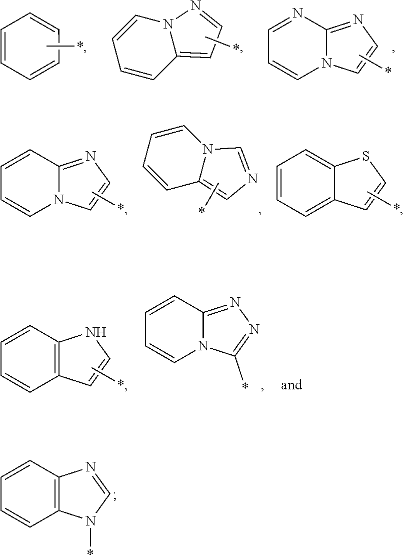

wherein each asterisk (*) denotes a point of attachment within Formula (1a).
4. The compound according to embodiment 3, wherein the compound is chosen from Formula (1aa) and Formula (1ab):

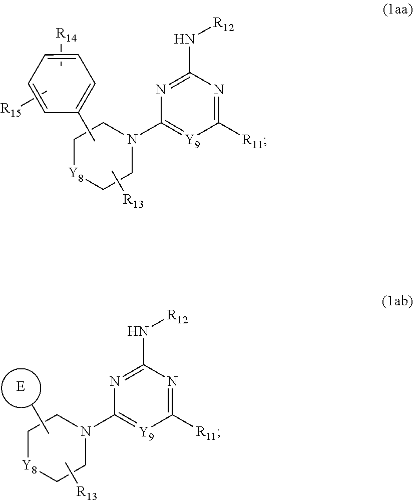

wherein R₁₄ and R₁₅ are not both absent, and wherein each of Formula (1aa) and Formula (1ab) encompass all tautomers, stereoisomers, pharmaceutically acceptable salts, and mixtures thereof.
5. The compound according to embodiment 1, wherein the compound is a compound of Formula (1 b):

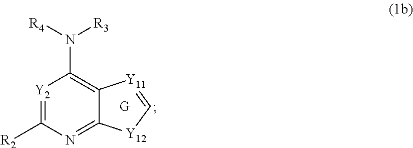

wherein:
each of Y₁₁ and Y₁₂ are chosen from N and CH;
at least one of Y₁₁ and Y₁₂ is N;
and wherein Formula (1 b) encompasses all tautomers, stereoisomers, deuterated derivatives, pharmaceutically acceptable salts, and mixtures thereof.
6. The compound according to embodiment 5, wherein the compound is a compound of Formula (1ba):

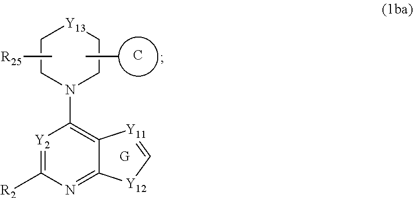

wherein:
ring C is chosen from 9 membered bicyclic heteroaryls and 6 membered aryls optionally substituted with a halogen and/or a hydroxyl;
Y₁₃ is chosen from CH₂, O, and NR₂₄ wherein R₂₄ is chosen from H and a C₁-C₃ alkyl optionally substituted with hydroxyl;
R₂₅ is absent or chosen from a halogen, a hydroxyl, and a C₁-C₃ alkyl optionally substituted with a tertiary amine;
and wherein Formula (1ba) encompasses all tautomers, stereoisomers, deuterated derivatives, pharmaceutically acceptable salts, and mixtures thereof.
7. The compound according to embodiment 1, wherein the compound is a compound of Formula (1c):

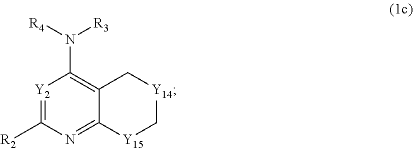

wherein:
   Y₁₅ is chosen from NH and CH₂;
   Y₁₄ is chosen from O, S, and CH₂;
   at least one of Y₁₅ and Y₁₄ is not CH₂;
and wherein Formula (1c) encompasses all tautomers, stereoisomers, deuterated derivatives, pharmaceutically acceptable salts, and mixtures thereof.
8. The compound according to embodiment 7, wherein the compound is a compound of Formula (1ca):

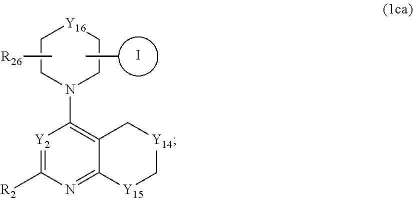

wherein:
ring I is chosen from a 9 membered bicyclic heteroaryl and a 5 or 6 membered aryl or heteroaryl ring optionally substituted with a halogen and/or a hydroxyl;
Y₁₆ is chosen from CH₂, O, and NR₂₇ wherein R₂₇ is chosen from H and a C₁-C₃ alkyl optionally substituted with hydroxyl;
R₂₆ is absent or chosen from a halogen, a hydroxyl, and a C₁-C₃ alkyl optionally substituted with a tertiary amine;
and wherein Formula (1 ca) encompasses all tautomers, stereoisomers, deuterated derivatives, pharmaceutically acceptable salts, and mixtures thereof.
9. The compound according to any one of embodiments 1, 5, and 7, wherein the compound is chosen from Formula (1), Formula (1b), and Formula (1c); and R₃ is a hydrogen or an optionally substituted C₁-C₃ alkyl.
10. The compound according to embodiment 9, wherein R₄ is chosen from:
   C₁-C₃ alkyl optionally substituted with a 9 membered bicyclic heteroaryl,

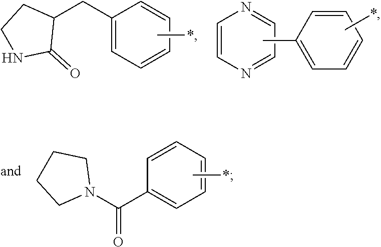

wherein the asterisks (*) denotes a point of attachment in Formula (1).
11. The compound according to embodiment 1, wherein R₃ and R₄ together form a 5 to 9 membered ring system D.
12. The compound according to embodiment 11, wherein the 5 to 9 membered ring system D is chosen from monocyclic rings optionally substituted with a halogen, a hydroxyl, an optionally substituted phenyl, an acetyl group, a 5 membered heteroaryl, an optionally substituted 9 membered heterobicyclic aryl, an optionally substituted benzimidazolone, and/or an optionally substituted C₁-C₃ alkyl.
13. The compound according to embodiment 1, wherein R₃ and R₄ together form a 5 to 9 membered monocyclic ring substituted with a substituted phenyl.
14. The compound according to embodiment 13, wherein the 5 to 9 membered monocyclic ring is a 6-membered heterocyclyl.
15. The compound according to embodiment 13, wherein the substituted phenyl is substituted with 1 or 2 groups chosen from halogen, haloalkyl, 4 to 10-membered heteroaryl, 4 to 10-membered heterocyclyl, —C(O)Rₐ, —C(O)NR_{b}R_{c}, —C(O)OR_{b}, C₁-C₄ alkyl, C₃-C₆ cycloalkyl, C₃-C₆ cycloalkoxy, and C₁-C₄ alkoxy, wherein Rₐ, R_{b}, and R_{c} are independently chosen from hydrogen, C₁-C₄ alkyl, C₃-C₆ cycloalkyl, C₁-C₄ alkylamino, 4 to 10-membered heteroaryl, and 4 to 10-membered heterocyclyl,
   or R_{b} and R_{c} together form a 4 to 6-membered heterocyclyl optionally substituted with 1 or 2 groups chosen from cyano, halogen, C₁-C₄ alkyl, C₁-C₄ alkoxy, and C₁-C₄ hydroxyalkyl, and
   wherein the 4 to 10-membered heteroaryl and 4 to 10-membered heterocyclyl are each optionally substituted with 1, 2, or 3 groups chosen from cyano, halogen, C₁-C₄ alkyl, C₁-C₄ alkoxy, C₁-C₄ haloalkyl, C₁-C₄ hydroxyalkyl, and C₃-C₆ cycloalkyl.
16. The compound according to embodiment 13, wherein the substituted phenyl is substituted with 1 or 2 substituents chose from halogen, C₁-C₄ haloalkyl, 5 to 10-membered heteroaryl, 4 to 6-membered heterocyclyl, —C(O)NR_{b}R_{c}, and C₁-C₂ alkoxy,
   wherein the 5 to 10-membered heteroaryl is optionally substituted with 1 or 2 methyl; and
   wherein R_{b} and R_{c} are independently chosen from hydrogen and C₄ alkylamino; or
   R_{b} and R_{c} together form a 4 to 6-membered heterocyclyl which is optionally substituted with 1 or 2 groups chosen from cyano, halogen, methyl, —OMe, and —CH₂OH.
17. The compound according to embodiment 13, wherein the substituted phenyl is optionally substituted with 1 or 2 substituents chosen from bromo, —CF₃, —OEt,

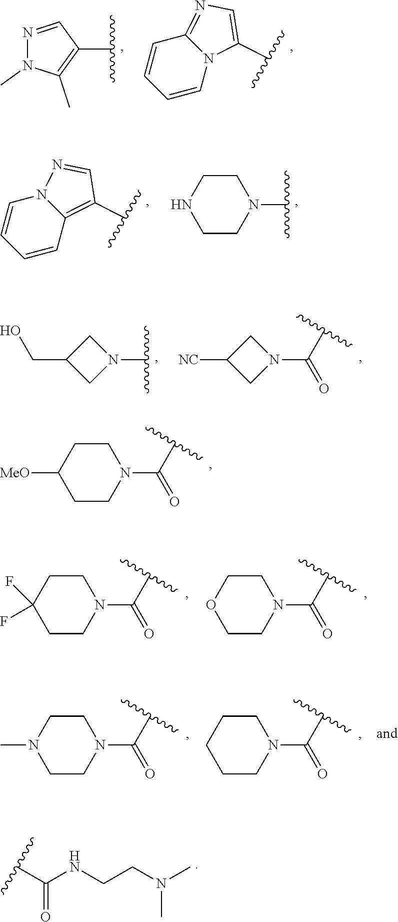

18. The compound according to embodiment 13, wherein the substituted phenyl is substituted with a substituent chosen from halogen and C₁-C₄ haloalkyl and a substituent chosen from —CF₃, —OEt,

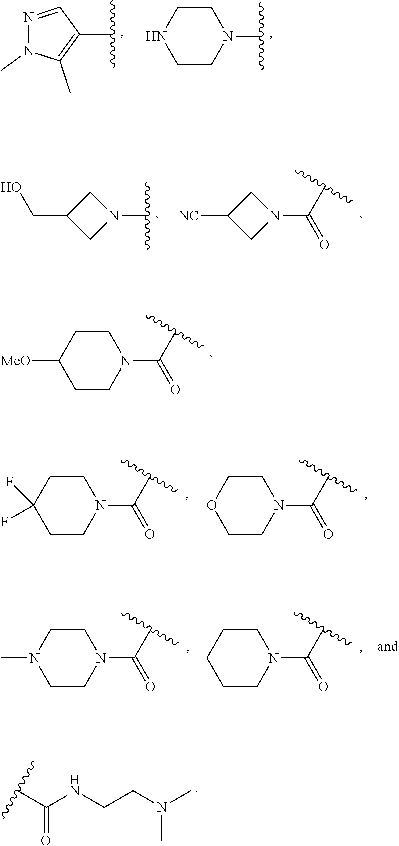

19. The compound according to embodiment 1, wherein the compound is chosen from:
4-(7-(2-bromophenyl)-6,7-dihydropyrazolo[1,5-a]pyrazin-5(4H)-yl)-6-isopropylpyrimidin-2-amine;
(R/S)-6-(3-(2-bromo-5-(1,5-dimethyl-1H-pyrazol-4-yl)phenyl)piperazin-1-yl)pyrimidine-2,4-diamine;
(S/R)-6-(3-(2-bromo-5-(1,5-dimethyl-1H-pyrazol-4-yl)phenyl)piperazin-1-yl)pyrimidine-2,4-diamine;
(R/S)-4-(3-(2-bromo-5-(1,5-dimethyl-1H-pyrazol-4-yl)phenyl)piperazin-1-yl)-6-isopropylpyrimidin-2-amine;
(S/R)-4-(3-(2-bromo-5-(1,5-dimethyl-1H-pyrazol-4-yl)phenyl)piperazin-1-yl)-6-isopropylpyrimidin-2-amine;
4-bromo-3-(4-(2,6-diaminopyrimidin-4-yl)piperazin-2-yl)-N-(2-(dimethylamino)ethyl)benzamide;
(R/S)-6-(3-(2-bromo-5-(trifluoromethyl)phenyl)piperazin-1-yl)pyrimidine-2,4-diamine;
(S/R)-6-(3-(2-bromo-5-(trifluoromethyl)phenyl)piperazin-1-yl)pyrimidine-2,4-diamine;
(R/S)-4-(3-(2-bromo-5-(trifluoromethyl)phenyl)piperazin-1-yl)-6-isopropylpyrimidin-2-amine;
(S/R)-4-(3-(2-bromo-5-(trifluoromethyl)phenyl)piperazin-1-yl)-6-isopropylpyrimidin-2-amine;
(R/S)-(4-bromo-3-(4-(2,6-diaminopyrimidin-4-yl)piperazin-2-yl)phenyl)(4-methylpiperazin-1-yl)methanone;
(S/R)-(4-bromo-3-(4-(2,6-diaminopyrimidin-4-yl)piperazin-2-yl)phenyl)(4-methylpiperazin-1-yl)methanone;
(R/S)-(3-(4-(2-amino-6-isopropylpyrimidin-4-yl)piperazin-2-yl)-4-bromophenyl)(4-methylpiperazin-1-yl)methanone;
(S/R)-(3-(4-(2-amino-6-isopropylpyrimidin-4-yl)piperazin-2-yl)-4-bromophenyl)(4-methylpiperazin-1-yl)methanone;
7-(3-(imidazo[1,2-a]pyridin-3-yl)piperidin-1-yl)-5-isopropyl-3H-imidazo[4,5-b]pyridine;
(R/S)-3-(4-(2-amino-6-isopropylpyrimidin-4-yl)piperazin-2-yl)-4-bromo-N-(2-(dimethylamino)ethyl)benzamide;
(S/R)-3-(4-(2-amino-6-isopropylpyrimidin-4-yl)piperazin-2-yl)-4-bromo-N-(2-(dimethylamino)ethyl)benzamide;
4-isopropyl-6-(3-(pyrazolo[1,5-a]pyridin-3-yl)piperazin-1-yl)pyrimidin-2-amine;
(R/S)-6-(3-(2-bromo-5-ethoxyphenyl)piperazin-1-yl)pyrimidine-2,4-diamine;
(S/R)-6-(3-(2-bromo-5-ethoxyphenyl)piperazin-1-yl)pyrimidine-2,4-diamine;
(R/S)-4-(3-(2-bromo-5-ethoxyphenyl)piperazin-1-yl)-6-isopropylpyrimidin-2-amine;
(S/R)-4-(3-(2-bromo-5-ethoxyphenyl)piperazin-1-yl)-6-isopropylpyrimidin-2-amine;
(R/S)-4-ethyl-6-(3-(imidazo[1,2-a]pyridin-3-yl)piperazin-1-yl)pyrimidin-2-amine;
(S/R)-4-ethyl-6-(3-(imidazo[1,2-a]pyridin-3-yl)piperazin-1-yl)pyrimidin-2-amine;
6-(2-(2-(trifluoromethyl)phenyl)morpholino)pyrimidine-2,4-diamine;
(R/S)-(1-(3-(4-(2-amino-6-isopropylpyrimidin-4-yl)piperazin-2-yl)-4-bromophenyl)azetidin-3-yl)methanol;
(S/R)-(1-(3-(4-(2-amino-6-isopropylpyrimidin-4-yl)piperazin-2-yl)-4-bromophenyl)azetidin-3-yl)methanol;
4-(3-(imidazo[1,2-a]pyridin-3-yl)piperazin-1-yl)-6-isopropylpyrimidin-2-amine;
(1-(4-bromo-3-(4-(2,6-diaminopyrimidin-4-yl)piperazin-2-yl)phenyl)piperidin-4-yl)methanol;
(S/R)-7-(3-(2-bromophenyl)piperazin-1-yl)-3H-imidazo[4,5-b]pyridine;
(R/S)-7-(3-(2-bromophenyl)piperazin-1-yl)-3H-imidazo[4,5-b]pyridine;
(R/S)-(2-(2-bromophenyl)-4-(2,6-diaminopyrimidin-4-yl)morpholin-2-yl)methanol;
(S/R)-(2-(2-bromophenyl)-4-(2,6-diaminopyrimidin-4-yl)morpholin-2-yl)methanol;
methyl 4-bromo-3-(4-(2,6-diaminopyrimidin-4-yl)piperazin-2-yl)benzoate;
(R/S)-4-(2-(2-bromophenyl)morpholino)-7H-pyrrolo[2,3-d]pyrimidin-2-amine;
(S/R)-4-(2-(2-bromophenyl)morpholino)-7H-pyrrolo[2,3-d]pyrimidin-2-amine;
4-(3-(2-bromo-5-(piperazin-1-yl)phenyl)piperazin-1-yl)-6-isopropylpyrimidin-2-amine;
(R/S)-4-(3-(2-bromophenyl)piperazin-1-yl)-5H-pyrrolo[3,2-d]pyrimidin-2-amine;
(S/R)-4-(3-(2-bromophenyl)piperazin-1-yl)-5H-pyrrolo[3,2-d]pyrimidin-2-amine;
(R/S)-2-(4-(2-amino-6-(cyclopropylamino)pyrimidin-4-yl)-2-(2-bromophenyl)piperazin-1-yl)ethan-1-ol;
(S/R)-2-(4-(2-amino-6-(cyclopropylamino)pyrimidin-4-yl)-2-(2-bromophenyl)piperazin-1-yl)ethan-1-ol;
(R/S)-4-(2-(2-bromophenyl)morpholino)-5H-pyrrolo[3,2-d]pyrimidin-2-amine;
(S/R)-4-(2-(2-bromophenyl)morpholino)-5H-pyrrolo[3,2-d]pyrimidin-2-amine;
(S/R)-6-(2-(2-bromophenyl)morpholino)-5-fluoropyrimidine-2,4-diamine;
(R/S)-6-(2-(2-bromophenyl)morpholino)-5-fluoropyrimidine-2,4-diamine;
(R/S)-6-(3-(2-bromo-5-(4-methylpiperazin-1-yl)phenyl)piperazin-1-yl)pyrimidine-2,4-diamine;
(S/R)-6-(3-(2-bromo-5-(4-methylpiperazin-1-yl)phenyl)piperazin-1-yl)pyrimidine-2,4-diamine;
1-(4-(6-amino-2-(cyclopropylamino)pyrimidin-4-yl)-2-(2-bromophenyl)piperazin-1-yl)ethan-1-one;
2-(4-(4-amino-6-(cyclopropylamino)-1,3,5-triazin-2-yl)-2-(2-bromophenyl)piperazin-1-yl)ethan-1-ol;
6-(3-(2-(trifluoromethyl)phenyl)piperazin-1-yl)pyrimidine-2,4-diamine;
4-(3-(imidazo[1,2-a]pyridin-3-yl)piperazin-1-yl)-5H-pyrrolo[3,2-d]pyrimidin-2-amine;
(R/S)-4-(3-(2-bromophenyl)piperazin-1-yl)-7H-pyrrolo[2,3-d]pyrimidin-2-amine;
(S/R)-4-(3-(2-bromophenyl)piperazin-1-yl)-7H-pyrrolo[2,3-d]pyrimidin-2-amine;
4-(3-(imidazo[1,2-a]pyridin-3-yl)piperidin-1-yl)-5,6,7,8-tetrahydropyrido[2,3-d]pyrimidine;
(S/R)-1-(2-(2-bromophenyl)-4-(2,6-diaminopyrimidin-4-yl)piperazin-1-yl)ethan-1-one;
(R/S)-1-(2-(2-bromophenyl)-4-(2,6-diaminopyrimidin-4-yl)piperazin-1-yl)ethan-1-one;
6-(2-(2-bromophenyl)morpholino)-N2-cyclopropyl-1,3,5-triazine-2,4-diamine;
7-(3-(imidazo[1,2-a]pyridin-3-yl)piperidin-1-yl)-3H-imidazo[4,5-b]pyridine;
6-(3-(2-bromophenyl)piperazin-1-yl)-5-chloro-N4-cyclopropylpyrimidine-2,4-diamine;
(R/S)-6-(3-(2-bromophenyl)piperazin-1-yl)-N2-cyclopropyl-1,3,5-triazine-2,4-diamine;
(S/R)-6-(3-(2-bromophenyl)piperazin-1-yl)-N2-cyclopropyl-1,3,5-triazine-2,4-diamine;
(S/R)-2-(2-(2-bromophenyl)-4-(2,6-diaminopyrimidin-4-yl)piperazin-1-yl)ethan-1-ol;
(R/S)-2-(2-(2-bromophenyl)-4-(2,6-diaminopyrimidin-4-yl)piperazin-1-yl)ethan-1-ol;
6-(3-(2-bromophenyl)piperazin-1-yl)-5-chloropyrimidine-2,4-diamine;
(S/R)-2-(4-(6-amino-2-(cyclopropylamino)pyrimidin-4-yl)-2-(2-bromophenyl)piperazin-1-yl)ethan-1-ol;
(R/S)-2-(4-(6-amino-2-(cyclopropylamino)pyrimidin-4-yl)-2-(2-bromophenyl)piperazin-1-yl)ethan-1-ol;
6-(3-(2-bromophenyl)-4-methylpiperazin-1-yl)-N2-cyclopropylpyrimidine-2,4-diamine;
(S/R)-6-(2-(2-bromophenyl)morpholino)-2-isopropylpyrimidin-4-amine;
(R/S)-6-(2-(2-bromophenyl)morpholino)-2-isopropylpyrimidin-4-amine;
6-(3-(2-bromophenyl)-4-methylpiperazin-1-yl)-2-isopropylpyrimidin-4-amine;
(S/R)-6-(3-(2-bromophenyl)piperazin-1-yl)-2-isopropylpyrimidin-4-amine;
(R/S)-6-(3-(2-bromophenyl)piperazin-1-yl)-2-isopropylpyrimidin-4-amine;
(R/S)-6-(2-(2-bromophenyl)morpholino)-N4-(cyclopropylmethyl)pyrimidine-2,4-diamine;
(S/R)-6-(2-(2-bromophenyl)morpholino)-N4-(cyclopropylmethyl)pyrimidine-2,4-diamine;
6-(3-(2-bromophenyl)piperazin-1-yl)-N-cyclopropylpyrimidin-4-amine;
(R/S)-6-(3-(2-bromophenyl)-4-methylpiperazin-1-yl)-N2,N4-dimethylpyrimidine-2,4-diamine;
(S/R)-6-(3-(2-bromophenyl)-4-methylpiperazin-1-yl)-N2,N4-dimethylpyrimidine-2,4-diamine;
6-(3-(benzo[b]thiophen-3-yl)piperidin-1-yl)-N4-methylpyrimidine-2,4-diamine;
(R/S)-6-(3-(2-bromophenyl)piperidin-1-yl)-N2,N4-dimethylpyrimidine-2,4-diamine;
(S/R)-6-(3-(2-bromophenyl)piperidin-1-yl)-N2,N4-dimethylpyrimidine-2,4-diamine;
6-(3-(2-bromophenyl)piperidin-1-yl)-N2-methylpyrimidine-2,4-diamine;
(R/S)-6-(3-(2-bromophenyl)piperidin-1-yl)-N4-methylpyrimidine-2,4-diamine;
(S/R)-6-(3-(2-bromophenyl)piperidin-1-yl)-N4-methylpyrimidine-2,4-diamine;
4-(3-(imidazo[1,2-a]pyridin-3-yl)piperidin-1-yl)-5,6,7,8-tetrahydropyrido[2,3-d]pyrimidin-2-amine;
6-(3-(1H-indazol-1-yl)piperidin-1-yl)pyrimidine-2,4-diamine;
4-(3-(imidazo[1,2-a]pyridin-3-yl)piperidin-1-yl)-7H-pyrrolo[2,3-d]pyrimidine;
3-(1-(1H-pyrrolo[2,3-b]pyridin-4-yl)piperidin-3-yl)imidazo[1,2-a]pyridine;
6-(3-(imidazo[1,2-a]pyridin-3-yl)piperazin-1-yl)-N4-methylpyrimidine-2,4-diamine;
6-(3-(1H-benzo[d][1,2,3]triazol-1-yl)piperidin-1-yl)-N4-methylpyrimidine-2,4-diamine;
6-(3-(imidazo[1,5-a]pyridin-3-yl)piperidin-1-yl)-N4-methylpyrimidine-2,4-diamine;
(S/R)-N4-cyclopropyl-6-(2-phenylmorpholino)pyrimidine-2,4-diamine;
(R/S)-N4-cyclopropyl-6-(2-phenylmorpholino)pyrimidine-2,4-diamine;
(R/S)-6-(2-(2-bromophenyl)morpholino)-N4-cyclopropylpyrimidine-2,4-diamine;
(S/R)-6-(2-(2-bromophenyl)morpholino)-N4-cyclopropylpyrimidine-2,4-diamine;
(R/S)-6-(3-(2-bromophenyl)-4-methylpiperazin-1-yl)-N4-(cyclopropylmethyl)pyrimidine-2,4-diamine;
(S/R)-6-(3-(2-bromophenyl)-4-methylpiperazin-1-yl)-N4-(cyclopropylmethyl)pyrimidine-2,4-diamine;
6-(3-(2-bromophenyl)-4-methylpiperazin-1-yl)-N4-cyclopropylpyrimidine-2,4-diamine;
6-(3-(benzo[b]thiophen-3-yl)piperazin-1-yl)-N4-methylpyrimidine-2,4-diamine;
(S/R)-4-cyclopropyl-6-(3-(imidazo[1,2-a]pyridin-3-yl)piperazin-1-yl)pyrimidin-2-amine;
(R/S)-4-cyclopropyl-6-(3-(imidazo[1,2-a]pyridin-3-yl)piperazin-1-yl)pyrimidin-2-amine;
(S/R)-4-(3-(imidazo[1,2-a]pyridin-3-yl)-4-methylpiperazin-1-yl)-6-isopropylpyrimidin-2-amine;
(R/S)-4-(3-(imidazo[1,2-a]pyridin-3-yl)-4-methylpiperazin-1-yl)-6-isopropylpyrimidin-2-amine;
N4-cyclopropyl-6-(3-(imidazo[1,2-a]pyridin-3-yl)piperidin-1-yl)pyrimidine-2,4-diamine;
(R/S)-N4-methyl-6-(2-phenylmorpholino)pyrimidine-2,4-diamine;
(S/R)-N4-methyl-6-(2-phenylmorpholino)pyrimidine-2,4-diamine;
N4-methyl-6-(4-methyl-3-phenylpiperazin-1-yl)pyrimidine-2,4-diamine;
(R/S)-6-(3-(2-bromophenyl)piperazin-1-yl)-N4-(cyclopropylmethyl)pyrimidine-2,4-diamine;
(S/R)-6-(3-(2-bromophenyl)piperazin-1-yl)-N4-(cyclopropylmethyl)pyrimidine-2,4-diamine;
(R/S)-6-(3-(2-bromophenyl)piperazin-1-yl)-N2-methylpyrimidine-2,4-diamine;
(S/R)-6-(3-(2-bromophenyl)piperazin-1-yl)-N2-methylpyrimidine-2,4-diamine;
(S/R)-6-(3-(imidazo[1,2-a]pyridin-3-yl)piperidin-1-yl)pyrimidine-2,4-diamine;
(R/S)-6-(3-(imidazo[1,2-a]pyridin-3-yl)piperidin-1-yl)pyrimidine-2,4-diamine;
(S/R)-6-(3-(imidazo[1,2-a]pyridin-3-yl)piperidin-1-yl)-N2-methylpyrimidine-2,4-diamine;
(R/S)-6-(3-(imidazo[1,2-a]pyridin-3-yl)piperidin-1-yl)-N2-methylpyrimidine-2,4-diamine;
(S/R)-6-(3-(imidazo[1,2-a]pyridin-3-yl)piperazin-1-yl)pyrimidine-2,4-diamine;
(R/S)-6-(3-(imidazo[1,2-a]pyridin-3-yl)piperazin-1-yl)pyrimidine-2,4-diamine;
(R/S)-6-(3-(2-bromophenyl)piperazin-1-yl)-N4-cyclopropylpyrimidine-2,4-diamine;
(S/R)-6-(3-(2-bromophenyl)piperazin-1-yl)-N4-cyclopropylpyrimidine-2,4-diamine;
(S/R)-6-(3-(imidazo[1,2-a]pyridin-3-yl)piperidin-1-yl)-N2,N4-dimethylpyrimidine-2,4-diamine;
(R/S)-6-(3-(imidazo[1,2-a]pyridin-3-yl)piperidin-1-yl)-N2,N4-dimethylpyrimidine-2,4-diamine;
(S/R)-6-(3-(imidazo[1,2-a]pyridin-3-yl)piperidin-1-yl)-2-isopropyl-N-methylpyrimidin-4-amine;
(R/S)-6-(3-(imidazo[1,2-a]pyridin-3-yl)piperidin-1-yl)-2-isopropyl-N-methylpyrimidin-4-amine;
2-((4-(3-(imidazo[1,2-a]pyridin-3-yl)piperidin-1-yl)-6-isopropylpyrimidin-2-yl)amino)ethan-1-ol;
(R/S)-N4-methyl-6-(3-phenylpiperazin-1-yl)pyrimidine-2,4-diamine;
(S/R)-N4-methyl-6-(3-phenylpiperazin-1-yl)pyrimidine-2,4-diamine;
(S/R)-6-(3-(2-bromophenyl)piperazin-1-yl)-N2,N4-dimethylpyrimidine-2,4-diamine;
(R/S)-6-(3-(2-bromophenyl)piperazin-1-yl)-N2,N4-dimethylpyrimidine-2,4-diamine;
(R/S)-4-(3-(2-bromophenyl)piperazin-1-yl)-6-cyclopropylpyrimidin-2-amine;
(S/R)-4-(3-(2-bromophenyl)piperazin-1-yl)-6-cyclopropylpyrimidin-2-amine;
4-(3-(imidazo[1,2-a]pyridin-3-yl)piperidin-1-yl)-6-isopropyl-N-methylpyrimidin-2-amine;
3-(1-(2-amino-5,6,7,8-tetrahydropyrido[2,3-d]pyrimidin-4-yl)piperidin-3-yl)-N,N-dimethylpropanamide;
(R/S)-6-(2-(imidazo[1,2-a]pyridin-3-yl)morpholino)pyrimidine-2,4-diamine;
(S/R)-6-(2-(imidazo[1,2-a]pyridin-3-yl)morpholino)pyrimidine-2,4-diamine;
4-(3-(1H-indol-3-yl)piperazin-1-yl)-6-isopropylpyrimidin-2-amine;
4-cyclopropyl-6-(3-(imidazo[1,2-a]pyridin-3-yl)piperidin-1-yl)pyrimidin-2-amine;
3-(1-(2-amino-5,6,7,8-tetrahydropyrido[2,3-d]pyrimidin-4-yl)piperidin-3-yl)-N-methylpropanamide;
(S/R)-6-(3-(imidazo[1,2-a]pyridin-3-yl)piperidin-1-yl)-2-isopropylpyrimidin-4-amine;
(R/S)-6-(3-(imidazo[1,2-a]pyridin-3-yl)piperidin-1-yl)-2-isopropylpyrimidin-4-amine;
(R/S)-6-(3-(2-bromophenyl)-4-methylpiperazin-1-yl)pyrimidine-2,4-diamine;
(S/R)-6-(3-(2-bromophenyl)-4-methylpiperazin-1-yl)pyrimidine-2,4-diamine;
(R/S)-6-(2-(2-bromophenyl)morpholino)pyrimidine-2,4-diamine;
(S/R)-6-(2-(2-bromophenyl)morpholino)pyrimidine-2,4-diamine;
4-(3-(imidazo[1,2-a]pyridin-3-yl)piperidin-1-yl)-6-methylpyrimidin-2-amine;
(R/S)-3-(1-(6-isopropylpyrimidin-4-yl)piperidin-3-yl)imidazo[1,2-a]pyridine;
(S/R)-3-(1-(6-isopropylpyrimidin-4-yl)piperidin-3-yl)imidazo[1,2-a]pyridine;
(S/R)-4-(3-(2-bromophenyl)piperazin-1-yl)-N,6-diisopropylpyrimidin-2-amine;
(R/S)-4-(3-(2-bromophenyl)piperazin-1-yl)-N,6-diisopropylpyrimidin-2-amine;
(R/S)-4-(3-(2-bromophenyl)piperazin-1-yl)-6-isopropyl-N-methylpyrimidin-2-amine;
(S/R)-4-(3-(2-bromophenyl)piperazin-1-yl)-6-isopropyl-N-methylpyrimidin-2-amine;
(R/S)-6-(3-(2-bromophenyl)-4-methylpiperazin-1-yl)-N4-methylpyrimidine-2,4-diamine;
(S/R)-6-(3-(2-bromophenyl)-4-methylpiperazin-1-yl)-N4-methylpyrimidine-2,4-diamine;
(R/S)-4-(2-(imidazo[1,2-a]pyridin-3-yl)morpholino)-6-isopropylpyrimidin-2-amine;
(S/R)-4-(2-(imidazo[1,2-a]pyridin-3-yl)morpholino)-6-isopropylpyrimidin-2-amine;
(R/S)-4-(2-(2-bromophenyl)morpholino)-6-isopropylpyrimidin-2-amine;
(S/R)-4-(2-(2-bromophenyl)morpholino)-6-isopropylpyrimidin-2-amine;
(R/S)-4-(3-(2-bromophenyl)-4-methylpiperazin-1-yl)-6-isopropylpyrimidin-2-amine;
(S/R)-4-(3-(2-bromophenyl)-4-methylpiperazin-1-yl)-6-isopropylpyrimidin-2-amine;
4-(3-(benzo[b]thiophen-3-yl)piperazin-1-yl)-6-isopropylpyrimidin-2-amine;
3-(4-(2-amino-6-isopropylpyrimidin-4-yl)piperazin-2-yl)-4-bromophenol;
4-(4-(2-amino-6-isopropylpyrimidin-4-yl)piperazin-2-yl)-3-bromophenol;
4-isopropyl-6-(3-phenylpiperazin-1-yl)pyrimidin-2-amine;
3-(1-(2-amino-6-isopropylpyrimidin-4-yl)piperidin-3-yl)-N-methyl-[1,2,4]triazolo[4,3-a]pyridine-6-carboxamide;
3-(1-(2-amino-6-isopropylpyrimidin-4-yl)piperidin-3-yl)-N-methyl-[1,2,4]triazolo[4,3-a]pyridine-6-carboxamide;
4-(3-(2-ethylphenyl)piperazin-1-yl)-6-isopropylpyrimidin-2-amine;
4-(3-(2-bromo-3-fluorophenyl)piperazin-1-yl)-6-isopropylpyrimidin-2-amine;
4-(3-(2-bromo-4,5-difluorophenyl)piperazin-1-yl)-6-isopropylpyrimidin-2-amine;
4-isopropyl-6-(3-(2-methoxyphenyl)piperazin-1-yl)pyrimidin-2-amine;
6-(3-(2-bromophenyl)piperazin-1-yl)pyrimidine-2,4-diamine;
4-(3-(2-bromo-4-fluorophenyl)piperazin-1-yl)-6-isopropylpyrimidin-2-amine;
4-(3-(2-bromo-5-fluorophenyl)piperazin-1-yl)-6-isopropylpyrimidin-2-amine;
4-(3-(2-chlorophenyl)piperazin-1-yl)-6-isopropylpyrimidin-2-amine;
4-(3-(2-iodophenyl)piperazin-1-yl)-6-isopropylpyrimidin-2-amine;
4-isopropyl-6-(3-(o-tolyl)piperazin-1-yl)pyrimidin-2-amine;
4-(3-(imidazo[1,2-a]pyridin-3-yl)piperidin-1-yl)-6-isopropylpyrimidin-2-amine;
N4-(2-(1H-indol-3-yl)ethyl)-6-isopropyl-N4-methylpyrimidine-2,4-diamine;
6-(3-([1,2,4]triazolo[4,3-a]pyridin-3-yl)piperidin-1-yl)-N4-methylpyrimidine-2,4-diamine;
4-(3-(1H-benzo[d]imidazol-2-yl)piperidin-1-yl)-6-isopropylpyrimidin-2-amine;
4-(5,6-dihydroimidazo[1,5-a]pyrazin-7(8H)-yl)-6-isopropylpyrimidin-2-amine;
4-(3-(2-bromophenyl)piperazin-1-yl)-6-isopropylpyrimidin-2-amine;
4-(3-(2-bromophenyl)piperazin-1-yl)-7,8-dihydro-5H-pyrano[4,3-d]pyrimidin-2-amine;
4-(3-(4H-1,2,4-triazol-3-yl)piperazin-1-yl)-5,7-dihydrothieno[3,4-d]pyrimidin-2-amine;
4-(3-(imidazo[1,2-a]pyridin-3-yl)piperidin-1-yl)-5,7-dihydrothieno[3,4-d]pyrimidin-2-amine;
4-(3-(1H-pyrazol-5-yl)piperidin-1-yl)-5,6,7,8-tetrahydropyrido[2,3-d]pyrimidin-2-amine;
4-(3-([1,2,4]triazolo[4,3-a]pyridin-3-yl)piperazin-1-yl)-6-isopropylpyrimidin-2-amine;
4-(3-(2-bromophenyl)piperazin-1-yl)-5,7-dihydrothieno[3,4-d]pyrimidin-2-amine;
N2,6-dimethyl-N4-(3-(pyrazin-2-yl)phenyl)pyrimidine-2,4-diamine;
4-(3-([1,2,4]triazolo[4,3-a]pyridin-3-yl)piperidin-1-yl)-6,7-dihydrothieno[3,2-d]pyrimidin-2-amine;
N4-(3-(pyrazin-2-yl)phenyl)pyrimidine-2,4,6-triamine;
6-(3-(1H-pyrazol-5-yl)piperidin-1-yl)pyrimidine-2,4-diamine;
4-(3-([1,2,4]triazolo[4,3-a]pyridin-3-yl)piperidin-1-yl)-6-isopropylpyrimidin-2-amine;
4-(3-(1H-pyrazol-5-yl)piperidin-1-yl)-6,7-dihydro-5H-cyclopenta[d]pyrimidin-2-amine;
4-(3-(1H-pyrazol-5-yl)piperidin-1-yl)-7,8-dihydro-5H-pyrano[4,3-d]pyrimidin-2-amine;
3-(3-((2-amino-6-isopropylpyrimidin-4-yl)amino)benzyl)pyrrolidin-2-one;
6-isopropyl-N4-(3-(pyrazin-2-yl)phenyl)pyrimidine-2,4-diamine;
4-(3-(1H-pyrazol-5-yl)piperidin-1-yl)-5,7-dihydrothieno[3,4-d]pyrimidin-2-amine;
4-(3-(2,5-dibromophenyl)piperazin-1-yl)-6-isopropylpyrimidin-2-amine;
3-(1-(2-amino-5,7-dihydrothieno[3,4-d]pyrimidin-4-yl)piperidin-3-yl)propanamide;
5-((1-(2-amino-6-methylpyrimidin-4-yl)piperidin-3-yl)amino)pyridin-2(1H)-one;
N4-(2-(1H-indol-3-yl)ethyl)-6-isopropylpyrimidine-2,4-diamine;
4-(3-(4H-1,2,4-triazol-3-yl)piperazin-1-yl)-6-isopropylpyrimidin-2-amine;
4-(3-(1H-pyrazol-5-yl)piperidin-1-yl)-7H-pyrrolo[2,3-d]pyrimidin-2-amine;
4-(3-(1H-pyrazol-5-yl)piperidin-1-yl)-5H-pyrrolo[3,2-d]pyrimidin-2-amine;
6-(3-(1H-pyrazol-5-yl)piperidin-1-yl)-7H-purin-2-amine;
4-(3-(1H-pyrazol-5-yl)piperidin-1-yl)-7,8-dihydro-5H-thiopyrano[4,3-d]pyrimidin-2-amine;
4-(3-(1H-pyrazol-5-yl)piperidin-1-yl)-6-methylpyrimidin-2-amine;
4-(3-(1H-pyrazol-5-yl)piperidin-1-yl)-6-isopropylpyrimidin-2-amine;
4-(3-(1H-pyrazol-5-yl)piperidin-1-yl)-6-cyclopropylpyrimidin-2-amine;
4-(3-(1H-pyrazol-5-yl)piperidin-1-yl)-6-cyclobutylpyrimidin-2-amine;
4-(3-(1H-pyrazol-5-yl)piperidin-1-yl)quinazolin-2-amine;
4-(3-(1H-pyrazol-5-yl)piperidin-1-yl)-5,6-dimethylpyrimidin-2-amine;
4-(3-(1-Methyl-1H-pyrazol-5-yl)piperidin-1-yl)-6,7-dihydro-5H-cyclopenta[d]pyrimidin-2-amine;
4-(3-(1H-pyrazol-5-yl)piperidin-1-yl)-5-bromo-6-methylpyrimidin-2-amine;
4-(3-(1H-pyrazol-5-yl)piperidin-1-yl)thieno[3,2-d]pyrimidin-2-amine;
4-(3-(1H-pyrazol-5-yl)piperidin-1-yl)pyrido[4,3-d]pyrimidin-2-amine;
4-(3-(1H-pyrazol-5-yl)piperidin-1-yl)-5,8-dihydro-6H-pyrano[3,4-d]pyrimidin-2-amine;
4-(3-(2-bromophenyl)-1,4-diazepan-1-yl)-6-isopropylpyrimidin-2-amine;
4-(3-(2-bromophenyl)-3-fluoropiperidin-1-yl)-6-isopropylpyrimidin-2-amine;
3-(2-bromophenyl)-1-(2,6-diaminopyrimidin-4-yl)piperidin-3-ol;
1-(2-amino-6-isopropylpyrimidin-4-yl)-3-(2-bromophenyl)piperidin-3-ol;
1-(2-amino-6-isopropylpyrimidin-4-yl)-3-(imidazo[1,5-a]pyridin-1-yl)piperidin-3-ol;
6-(3-(imidazo[1,5-a]pyridin-1-yl)piperidin-1-yl)pyrimidine-2,4-diamine;
1-(2-amino-6-isopropylpyrimidin-4-yl)-3-(2-bromophenyl)azepan-3-ol;
4-(5-(2-bromophenyl)-3,4-dihydropyridin-1(2H)-yl)-6-isopropylpyrimidin-2-amine;
6-(3-(2-bromophenyl)-3-fluoropiperidin-1-yl)pyrimidine-2,4-diamine;
rac-4-((3R,4R)-4-fluoro-3-(imidazo[1,5-a]pyridin-1-yl)piperidin-1-yl)-6-isopropylpyrimidin-2-amine;
4-(5-(2-bromophenyl)-3,6-dihydropyridin-1(2H)-yl)-6-isopropylpyrimidin-2-amine;
1-(2-amino-6-isopropylpyrimidin-4-yl)-3-(2-bromophenyl)pyrrolidin-3-ol;
4-isopropyl-6-(3-(1-methyl-1H-indazol-3-yl)piperidin-1-yl)pyrimidin-2-amine;
4-(3-(2-bromophenyl)-3-fluoropiperidin-1-yl)-6-isopropylpyrimidin-2-amine;
3-(2-bromophenyl)-1-(2,6-diaminopyrimidin-4-yl)piperidin-3-ol;
4-(3-(imidazo[1,5-a]pyridin-1-yl)piperidin-1-yl)-6-isopropylpyrimidin-2-amine;
1-(2-amino-6-isopropylpyrimidin-4-yl)-3-(2-bromophenyl)piperidin-3-ol;
6-(5-(imidazo[1,5-a]pyridin-1-yl)-3,6-dihydropyridin-1(2H)-yl)pyrimidine-2,4-diamine;
4-(5-(imidazo[1,5-a]pyridin-1-yl)-3,6-dihydropyridin-1(2H)-yl)-6-isopropylpyrimidin-2-amine;
4-(3-(imidazo[1,2-c]pyrimidin-3-yl)piperidin-1-yl)-6-isopropylpyrimidin-2-amine;
4-isopropyl-6-(3-(pyrazolo[1,5-a]pyridin-3-yl)piperidin-1-yl)pyrimidin-2-amine;
6-(3-(imidazo[1,2-a]pyrimidin-3-yl)piperidin-1-yl)pyrimidine-2,4-diamine;
4-(3-(6,8-difluoroimidazo[1,2-a]pyridin-3-yl)piperidin-1-yl)-6-isopropylpyrimidin-2-amine;
1-(2-amino-6-isopropylpyrimidin-4-yl)-3-(imidazo[1,2-a]pyridin-3-yl)piperidin-3-ol;
4-(3-(imidazo[1,5-a]pyridin-3-yl)piperidin-1-yl)-6-isopropylpyrimidin-2-amine;
4-(3-(imidazo[1,2-a]pyrimidin-3-yl)piperidin-1-yl)-6-isopropylpyrimidin-2-amine;
6-(3-(1H-indazol-3-yl)piperidin-1-yl)pyrimidine-2,4-diamine;
4-isopropyl-6-(3-(1-methyl-1H-indol-3-yl)piperidin-1-yl)pyrimidin-2-amine;
6-(3-(1-methyl-1H-indol-3-yl)piperidin-1-yl)pyrimidine-2,4-diamine;
4-(3-(1H-indol-3-yl)piperidin-1-yl)-6-isopropylpyrimidin-2-amine;
6-(3-(1H-indol-3-yl)piperidin-1-yl)pyrimidine-2,4-diamine;
4-(3-(1H-indazol-3-yl)piperidin-1-yl)-6-isopropylpyrimidin-2-amine;
1-(1-(2-amino-6-isopropylpyrimidin-4-yl)piperidin-3-yl)-1,3-dihydro-2H-benzo[d]imidazol-2-one;
4-(3-(imidazo[1,2-a]pyridin-3-yl)pyrrolidin-1-yl)-6-isopropylpyrimidin-2-amine;
6-(3-(1H-indazol-1-yl)piperidin-1-yl)-N4-methylpyrimidine-2,4-diamine;
6-(3-(imidazo[1,2-a]pyridin-3-yl)pyrrolidin-1-yl)pyrimidine-2,4-diamine;
4-(3-(1H-benzo[d]imidazol-1-yl)piperidin-1-yl)-6-isopropylpyrimidin-2-amine;
1-(1-(2,6-diaminopyrimidin-4-yl)piperidin-3-yl)-1,3-dihydro-2H-benzo[d]imidazol-2-one;
6-(3-(imidazo[1,2-c]pyrimidin-3-yl)piperidin-1-yl)pyrimidine-2,4-diamine;
6-(3-(2H-benzo[d][1,2,3]triazol-2-yl)piperidin-1-yl)-N4-methylpyrimidine-2,4-diamine;
6-(3-(1H-benzo[d]imidazol-1-yl)piperidin-1-yl)pyrimidine-2,4-diamine;
1-(3-(4-(2,6-diaminopyrimidin-4-yl)piperazin-2-yl)-4-(trifluoromethyl)benzoyl)azetidine-3-carbonitrile;
(3-(4-(2,6-diaminopyrimidin-4-yl)piperazin-2-yl)-4-(trifluoromethyl)phenyl)(4-methoxypiperidin-1-yl)methanone;
(S)-(3-(4-(2,6-diaminopyrimidin-4-yl)piperazin-2-yl)-4-(trifluoromethyl)phenyl)(piperidin-1-yl)methanone;
(R)-(3-(4-(2,6-diaminopyrimidin-4-yl)piperazin-2-yl)-4-(trifluoromethyl)phenyl)(piperidin-1-yl)methanone;
(S)-(3-(4-(2,6-diaminopyrimidin-4-yl)piperazin-2-yl)-4-(trifluoromethyl)phenyl)(4,4-difluoropiperidin-1-yl)methanone;
(R)-(3-(4-(2,6-diaminopyrimidin-4-yl)piperazin-2-yl)-4-(trifluoromethyl)phenyl)(4,4-difluoropiperidin-1-yl)methanone;
(S)-(3-(4-(2,6-diaminopyrimidin-4-yl)piperazin-2-yl)-4-(trifluoromethyl)phenyl)(morpholino)methanone; and
(R)-(3-(4-(2,6-diaminopyrimidin-4-yl)piperazin-2-yl)-4-(trifluoromethyl)phenyl)(morpholino)methanone.
   20. The compound according to embodiment 1, wherein the compound is chosen from:
(R/S)-6-(3-(2-bromo-5-(1,5-dimethyl-1H-pyrazol-4-yl)phenyl)piperazin-1-yl)pyrimidine-2,4-diamine;
(S/R)-6-(3-(2-bromo-5-(1,5-dimethyl-1H-pyrazol-4-yl)phenyl)piperazin-1-yl)pyrimidine-2,4-diamine;
(R/S)-4-(3-(2-bromo-5-(1,5-dimethyl-1H-pyrazol-4-yl)phenyl)piperazin-1-yl)-6-isopropylpyrimidin-2-amine;
(S/R)-4-(3-(2-bromo-5-(1,5-dimethyl-1H-pyrazol-4-yl)phenyl)piperazin-1-yl)-6-isopropylpyrimidin-2-amine;
4-bromo-3-(4-(2,6-diaminopyrimidin-4-yl)piperazin-2-yl)-N-(2-(dimethylamino)ethyl)benzamide;
(R/S)-6-(3-(2-bromo-5-(trifluoromethyl)phenyl)piperazin-1-yl)pyrimidine-2,4-diamine;
(S/R)-6-(3-(2-bromo-5-(trifluoromethyl)phenyl)piperazin-1-yl)pyrimidine-2,4-diamine;
(R/S)-4-(3-(2-bromo-5-(trifluoromethyl)phenyl)piperazin-1-yl)-6-isopropylpyrimidin-2-amine;
(S/R)-4-(3-(2-bromo-5-(trifluoromethyl)phenyl)piperazin-1-yl)-6-isopropylpyrimidin-2-amine; (R/S)-(4-bromo-3-(4-(2,6-diaminopyrimidin-4-yl)piperazin-2-yl)phenyl)(4-methylpiperazin-1-yl)methanone;
(S/R)-(4-bromo-3-(4-(2,6-diaminopyrimidin-4-yl)piperazin-2-yl)phenyl)(4-methylpiperazin-1-yl)methanone;
(R/S)-(3-(4-(2-amino-6-isopropylpyrimidin-4-yl)piperazin-2-yl)-4-bromophenyl)(4-methylpiperazin-1-yl)methanone;
(S/R)-(3-(4-(2-amino-6-isopropylpyrimidin-4-yl)piperazin-2-yl)-4-bromophenyl)(4-methylpiperazin-1-yl)methanone;
7-(3-(imidazo[1,2-a]pyridin-3-yl)piperidin-1-yl)-5-isopropyl-3H-imidazo[4,5-b]pyridine;
(R/S)-3-(4-(2-amino-6-isopropylpyrimidin-4-yl)piperazin-2-yl)-4-bromo-N-(2-(dimethylamino)ethyl)benzamide;
(S/R)-3-(4-(2-amino-6-isopropylpyrimidin-4-yl)piperazin-2-yl)-4-bromo-N-(2-(dimethylamino)ethyl)benzamide;
4-isopropyl-6-(3-(pyrazolo[1,5-a]pyridin-3-yl)piperazin-1-yl)pyrimidin-2-amine;
(R/S)-6-(3-(2-bromo-5-ethoxyphenyl)piperazin-1-yl)pyrimidine-2,4-diamine;
(S/R)-6-(3-(2-bromo-5-ethoxyphenyl)piperazin-1-yl)pyrimidine-2,4-diamine;
(R/S)-4-(3-(2-bromo-5-ethoxyphenyl)piperazin-1-yl)-6-isopropylpyrimidin-2-amine;
(S/R)-4-(3-(2-bromo-5-ethoxyphenyl)piperazin-1-yl)-6-isopropylpyrimidin-2-amine;
(R/S)-4-ethyl-6-(3-(imidazo[1,2-a]pyridin-3-yl)piperazin-1-yl)pyrimidin-2-amine;
(S/R)-4-ethyl-6-(3-(imidazo[1,2-a]pyridin-3-yl)piperazin-1-yl)pyrimidin-2-amine;
(R/S)-(1-(3-(4-(2-amino-6-isopropylpyrimidin-4-yl)piperazin-2-yl)-4-bromophenyl)azetidin-3-yl)methanol;
(S/R)-(1-(3-(4-(2-amino-6-isopropylpyrimidin-4-yl)piperazin-2-yl)-4-bromophenyl)azetidin-3-yl)methanol;
4-(3-(imidazo[1,2-a]pyridin-3-yl)piperazin-1-yl)-6-isopropylpyrimidin-2-amine;
4-(3-(2-bromo-5-(piperazin-1-yl)phenyl)piperazin-1-yl)-6-isopropylpyrimidin-2-amine;
(R/S)-7-(3-(imidazo[1,2-a]pyridin-3-yl)piperazin-1-yl)-5-isopropyl-3H-imidazo[4,5-b]pyridine;
(S/R)-7-(3-(imidazo[1,2-a]pyridin-3-yl)piperazin-1-yl)-5-isopropyl-3H-imidazo[4,5-b]pyridine;
3-(1-(6-isopropyl-1H-pyrrolo[2,3-b]pyridin-4-yl)piperidin-3-yl)imidazo[1,2-a]pyridine;
(R/S)-3-(4-(6-isopropyl-1H-pyrrolo[2,3-b]pyridin-4-yl)piperazin-2-yl)imidazo[1,2-a]pyridine;
(S/R)-3-(4-(6-isopropyl-1H-pyrrolo[2,3-b]pyridin-4-yl)piperazin-2-yl)imidazo[1,2-a]pyridine;
6-(3-(imidazo[1,2-a]pyridin-3-yl)piperazin-1-yl)-2-isopropyl-9H-purine;
4-(3-(imidazo[1,2-a]pyridin-3-yl)piperidin-1-yl)-2-isopropyl-7H-pyrrolo[2,3-d]pyrimidine;
4-(3-(imidazo[1,2-a]pyridin-3-yl)piperazin-1-yl)-2-isopropyl-7H-pyrrolo[2,3-d]pyrimidine;
6-(3-(imidazo[1,2-a]pyridin-3-yl)piperidin-1-yl)-2-isopropyl-9H-purine;
1-(3-(4-(2,6-diaminopyrimidin-4-yl)piperazin-2-yl)-4-(trifluoromethyl)benzoyl)azetidine-3-carbonitrile;
(3-(4-(2,6-diaminopyrimidin-4-yl)piperazin-2-yl)-4-(trifluoromethyl)phenyl)(4-methoxypiperidin-1-yl)methanone;
(S)-(3-(4-(2,6-diaminopyrimidin-4-yl)piperazin-2-yl)-4-(trifluoromethyl)phenyl)(piperidin-1-yl)methanone;
(R)-(3-(4-(2,6-diaminopyrimidin-4-yl)piperazin-2-yl)-4-(trifluoromethyl)phenyl)(piperidin-1-yl)methanone;
(S)-(3-(4-(2,6-diaminopyrimidin-4-yl)piperazin-2-yl)-4-(trifluoromethyl)phenyl)(4,4-difluoropiperidin-1-yl)methanone;
(R)-(3-(4-(2,6-diaminopyrimidin-4-yl)piperazin-2-yl)-4-(trifluoromethyl)phenyl)(4,4-difluoropiperidin-1-yl)methanone;
(S)-(3-(4-(2,6-diaminopyrimidin-4-yl)piperazin-2-yl)-4-(trifluoromethyl)phenyl)(morpholino)methanone; and
(R)-(3-(4-(2,6-diaminopyrimidin-4-yl)piperazin-2-yl)-4-(trifluoromethyl)phenyl)(morpholino)methanone.
   21. A compound chosen from Formula (2) or a tautomer, stereoisomer or a mixture of stereoisomers, or a pharmaceutically acceptable salt, or hydrate, or deuterated derivative thereof:

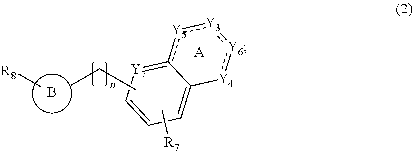

wherein:
Y₃ is chosen from CH, CH₂, N, CO, and NH;
Y₄ is chosen from CH, N, and CH₂;
Y₅ is chosen from CH, CH₂, CNHR₁₀, CO, N, 5 membered heteroaryl rings comprising two nitrogen atoms and three carbon atoms wherein the heteroaryl ring is optionally substituted with a methyl, and NH, wherein R₁₀ is chosen from H and C₁-C₄ alkyl;
Y₆ is chosen from CH and CH₂;
Y₇ is chosen from CH, N, and C—OH;
R₇ is absent or chosen from OH, halogen, 5 membered heteroaryl rings comprising two nitrogen atoms and three carbon atoms wherein the heteroaryl ring is optionally substituted with a methyl, and methyl;
ring A is an aromatic or non-aromatic 6-membered heterocyclic ring;
n is 0 or 1;
ring system B is chosen from:
   5 to 6 membered rings optionally comprising up to three nitrogen atoms, a sulfur atom, and/or an oxygen atom,
   bicyclic 9 or 10 membered rings comprising at least one aromatic ring, optionally comprising up to three nitrogen atoms, a sulfur atom, and/or a carbonyl, and
   bicyclic 7 or 8 membered aliphatic rings comprising an amine, and
wherein ring system B is optionally substituted with up to two halogens, a methyl, and/or NH₂;
R₈ is absent or chosen from:
   aliphatic amines comprising up to two nitrogen atoms, up to 7 carbon atoms, and optionally an alkoxy,
   N-substituted amides wherein at least one N-substituent comprises up to 7 carbon atoms and, optionally, an amine, a hydroxyl, and/or an alkoxy, and
   C-substituted carbonyls wherein the C-substituent comprises up to 6 carbon atoms and a tertiary amine;
and wherein Formula (2) encompasses all tautomers, stereoisomers, isotopes, pharmaceutically acceptable salts, and mixtures thereof.
22. The compound according to embodiment 21, wherein the compound is a compound of Formula (2a):

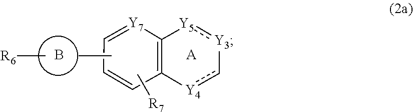

wherein:
Y₃ is chosen from N, NH, CH, CO, and C—NH₂;
Y₄ is chosen from N and CH;
Y₅ is chosen from N, NH, CH, CO, and C—NH₂;
at least one of Y₃, Y₄, and Y₅ is N;
each dashed bond in ring A independently represents a single or double bond, and R₇ is absent or chosen from OH, halogen, and methyl;
and wherein Formula (2a) encompasses all tautomers, stereoisomers, deuterated derivatives, pharmaceutically acceptable salts, and mixtures thereof.
23. The compound according to embodiment 22, wherein the compound is a compound of Formula (2aa):

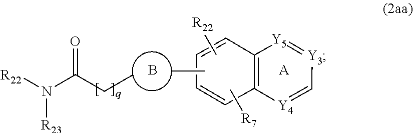

wherein:
q is 0 or 1;
R₂₂ is chosen from H and a C₁-C₃ alkyl;
R₂₃ consists of H, C, optionally N, and optionally O; and
R₂₃ comprises up to 7 carbon atoms and, optionally, an amine, a hydroxyl, and/or an alkoxy;
ring A is an aromatic 6-membered heterocyclic ring;
and wherein Formula (2aa) encompasses all tautomers, stereoisomers, deuterated derivatives, pharmaceutically acceptable salts, and mixtures thereof.
24. The compound according to any one of embodiments 21 to 23, wherein ring system B consists of C, H, optionally N, optionally S, optionally F, and optionally Cl.
25. The compound according to embodiment 24, wherein ring system B is chosen from:

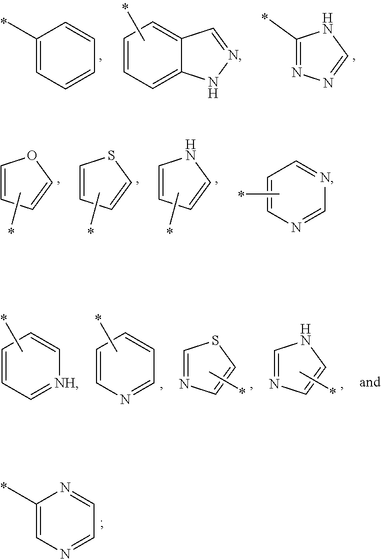

wherein each asterisk (*) denotes a point of attachment within Formula (2), Formula (2a), or Formula (2aa).
26. The compound according to embodiment 25, wherein the compound is Formula (2b):

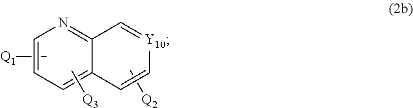

wherein:
Y₁₀ is N or CH;
Q₁ is chosen from a 5 to 6 membered rings optionally comprising up to two nitrogen atoms and a bicyclic 7 or 8 membered aliphatic rings comprising an amine; and
Q₂ and Q₃ are each independently a 5 membered heteroaryl ring comprising two nitrogen atoms and three carbon atoms wherein each heteroaryl ring is optionally substituted with a methyl;
and wherein Formula (2b) encompasses all tautomers, stereoisomers, deuterated derivatives, pharmaceutically acceptable salts, and mixtures thereof.
27. The compound according to embodiment 26, wherein the compound is chosen from:

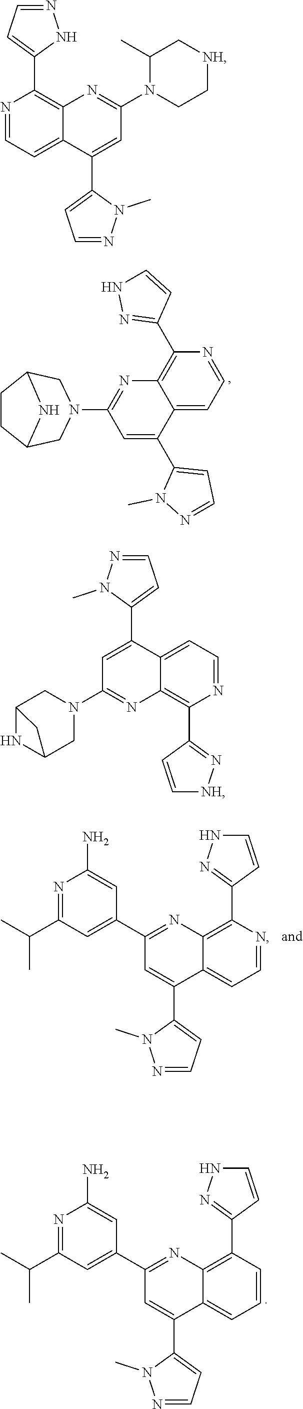

28. The compound according to embodiment 22, wherein the compound is chosen from:
5-(1-aminoisoquinolin-7-yl)-1,2-dihydro-3H-indazol-3-one;
N1-((5-(1-aminoisoquinolin-7-yl)-1H-indazol-3-yl)methyl)-N2,N2-dimethylethane-1,2-diamine;
5-(1-aminoisoquinolin-7-yl)-N-(2-(dimethylamino)ethyl)-1H-pyrrolo[2,3-b]pyridine-3-carboxamide;
5-(1-aminoisoquinolin-7-yl)-N-(1,4-dimethylpiperidin-4-yl)-1H-indazole-3-carboxamide;
4-(2-amino-8-hydroxyquinolin-6-yl)-N-(3-(dimethylamino)propyl)benzamide;
N-(3-(dimethylamino)propyl)-5-(8-hydroxyquinolin-6-yl)-4H-1,2,4-triazole-3-carboxamide;
N-(3-(dimethylamino)propyl)-5-(8-hydroxyquinolin-6-yl)thiophene-3-carboxamide;
N-((1,3-cis)-3-(dimethylamino)cyclobutyl)-5-(8-hydroxyquinolin-6-yl)thiophene-3-carboxamide;
4-(4-amino-8-hydroxyquinolin-6-yl)-N-(3-(dimethylamino)propyl)benzamide;
(3-(1-amino-5-hydroxyisoquinolin-7-yl)phenyl)(pyrrolidin-1-yl)methanone;
4-(4-amino-8-hydroxyquinazolin-6-yl)-N-(2-(dimethylamino)ethyl)benzamide;
N-(2-(dimethylamino)ethyl)-5-(8-hydroxyquinolin-6-yl)thiophene-3-carboxamide;
N-(3-(dimethylamino)propyl)-4-(8-hydroxyquinolin-6-yl)-1H-pyrrole-2-carboxamide;
N-(2-(dimethylamino)ethyl)-4-(8-hydroxyquinazolin-6-yl)benzamide;
N-(3-(dimethylamino)propyl)-5-(8-hydroxyquinolin-6-yl)pyrimidine-2-carboxamide;
N-(3-(dimethylamino)propyl)-5-(8-hydroxyquinolin-6-yl)thiazole-2-carboxamide;
N-(3-(dimethylamino)propyl)-4-(8-hydroxy-3-oxo-3,4-dihydroquinoxalin-6-yl)benzamide;
N-(3-(dimethylamino)propyl)-2-(8-hydroxyquinolin-6-yl)thiazole-5-carboxamide;
N-(3-(dimethylamino)propyl)-5-(8-hydroxyquinolin-6-yl)-1H-imidazole-2-carboxamide;
N-(3-(dimethylamino)propyl)-5-(8-hydroxyquinolin-6-yl)pyrazine-2-carboxamide;
N-(3-(dimethylamino)propyl)-2-(8-hydroxyquinolin-6-yl)-1H-imidazole-4-carboxamide;
6-(3-(pyrrolidine-1-carbonyl)phenyl)-3,4-dihydroisoquinolin-1 (2H)-one;
N-((1,3-cis)-3-(dimethylamino)cyclobutyl)-4-(8-hydroxyquinolin-6-yl)benzamide;
N-((1,3-trans)-3-(dimethylamino)cyclobutyl)-4-(8-hydroxyquinolin-6-yl)benzamide;
N-(3-(dimethylamino)propyl)-2-(8-hydroxyquinolin-6-yl)pyrimidine-5-carboxamide;
N-(3-(dimethylamino)propyl)-4-(8-hydroxyquinolin-6-yl)furan-2-carboxamide;
N-(3-(dimethylamino)propyl)-4-(8-hydroxy-1,2,3,4-tetrahydroquinolin-6-yl)benzamide;
N-(3-(dimethylamino)propyl)-4-(8-hydroxyquinoxalin-6-yl)benzamide;
N-((1s,4s)-4-(dimethylamino)cyclohexyl)-4-(8-hydroxyquinolin-6-yl)benzamide;
N-((1,4-trans)-4-(dimethylamino)cyclohexyl)-4-(8-hydroxyquinolin-6-yl)benzamide;
N-(3-(dimethylamino)propyl)-2-(4-(8-hydroxyquinolin-6-yl)phenyl)acetamide;
N-(4-(dimethylamino)butyl)-4-(8-hydroxyquinolin-6-yl)benzamide;
4-(8-hydroxyquinolin-6-yl)-N-((1-methylpiperidin-3-yl)methyl)benzamide;
4-(8-hydroxyquinolin-6-yl)-N-(1-methylazetidin-3-yl)benzamide;
4-(8-hydroxyquinolin-6-yl)-N-((1-methylpyrrolidin-3-yl)methyl)benzamide;
(3-(8-hydroxyquinolin-6-yl)phenyl)(pyrrolidin-1-yl)methanone;
N-(3-(dimethylamino)propyl)-6-(8-hydroxyquinolin-6-yl)nicotinamide;
N-(3-(dimethylamino)propyl)-5-(8-hydroxyquinolin-6-yl)picolinamide;
N-(3-(dimethylamino)propyl)-5-(8-hydroxyquinolin-6-yl)thiophene-2-carboxamide;
4-(8-hydroxyquinolin-6-yl)-N-(1-methylpiperidin-4-yl)benzamide;
4-(8-hydroxyquinolin-6-yl)-N-((1-methylazetidin-3-yl)methyl)benzamide;
N-(2-(dimethylamino)ethyl)-4-(8-hydroxyquinolin-6-yl)benzamide;
4-(8-hydroxyquinolin-6-yl)-N-((1-methylpiperidin-4-yl)methyl)benzamide;
(4-(8-hydroxyquinolin-6-yl)phenyl)(pyrrolidin-1-yl)methanone;
4-(8-hydroxyquinolin-6-yl)-N-propylbenzamide;
4-(8-hydroxyquinolin-6-yl)-N-(3-(methylamino)propyl)benzamide;
N-(3-(dimethylamino)propyl)-4-(4-hydroxy-1,5-naphthyridin-2-yl)benzamide;
N-(3-(dimethylamino)propyl)-4-(8-hydroxyquinolin-6-yl)cyclohex-3-ene-1-carboxamide;
N-(3-(dimethylamino)propyl)-4-(8-hydroxyquinolin-6-yl)-3,6-dihydropyridine-1(2H)-carboxamide;
N-(3-(dimethylamino)propyl)-2-(4-(8-hydroxyquinolin-6-yl)-3,6-dihydropyridin-1(2H)-yl)acetamide;
N-(3-(dimethylamino)propyl)-4-(7-fluoro-8-hydroxyquinolin-6-yl)benzamide;
4-(dimethylamino)-1-(4-(8-hydroxyquinolin-6-yl)-3,6-dihydropyridin-1(2H)-yl)butan-1-one;
2-chloro-N-(3-(dimethylamino)propyl)-3-fluoro-4-(8-hydroxyquinolin-6-yl)benzamide;
N-(3-(dimethylamino)propyl)-4-(8-hydroxyquinolin-6-yl)-2-methylbenzamide;
2-amino-N-(3-(dimethylamino)propyl)-4-(8-hydroxyquinolin-6-yl)benzamide;
2-bromo-N-(3-(dimethylamino)propyl)-4-(8-hydroxyquinolin-6-yl)benzamide;
N-(3-(dimethylamino)propyl)-2,6-difluoro-4-(8-hydroxyquinolin-6-yl)benzamide;
2-chloro-N-(3-(dimethylamino)propyl)-5-fluoro-4-(8-hydroxyquinolin-6-yl)benzamide;
N-(3-(dimethylamino)propyl)-2,5-difluoro-4-(8-hydroxyquinolin-6-yl)benzamide;
3-chloro-N-(3-(dimethylamino)propyl)-4-(8-hydroxyquinolin-6-yl)benzamide;
2,6-dichloro-N-(3-(dimethylamino)propyl)-4-(8-hydroxyquinolin-6-yl)benzamide;
N-(3-(dimethylamino)propyl)-4-(8-hydroxy-5-methylquinolin-6-yl)benzamide;
N-(3-(dimethylamino)propyl)-4-(8-hydroxyquinolin-6-yl)-3-methylbenzamide;
4-(7-chloro-8-hydroxyquinolin-6-yl)-N-(3-(dimethylamino)propyl)benzamide;
2,5-dichloro-N-(3-(dimethylamino)propyl)-4-(8-hydroxyquinolin-6-yl)benzamide;
3-bromo-N-(3-(dimethylamino)propyl)-4-(8-hydroxyquinolin-6-yl)benzamide;
6-(1,2,3,6-tetrahydropyridin-4-yl)quinolin-8-ol;
N-(3-(dimethylamino)propyl)-3-fluoro-4-(8-hydroxyquinolin-6-yl)benzamide;
5-(1-aminoisoquinolin-7-yl)-N-((2-methoxypyridin-4-yl)methyl)-1H-indazole-3-carboxamide;
5-(1-aminoisoquinolin-7-yl)-N-(2-(dimethylamino)ethyl)-1H-pyrazolo[4,3-b]pyridine-3-carboxamide;
5-(1-aminoisoquinolin-7-yl)-N-((1-methyl-1H-pyrazol-4-yl)methyl)-1H-indazole-3-carboxamide;
5-(4-aminoquinazolin-6-yl)-N-(2-(dimethylamino)ethyl)-1H-indazole-3-carboxamide;
5-(4-aminoquinazolin-6-yl)-N-(3-(dimethylamino)propyl)-1H-indazole-3-carboxamide;
5-(1-aminoisoquinolin-7-yl)-N-(2-hydroxyethyl)-1H-indazole-3-carboxamide;
(5-(1-aminoisoquinolin-7-yl)-1H-indazol-3-yl)(4-methoxypiperidin-1-yl)methanone;
5-(1-aminoisoquinolin-7-yl)-N-(((1,4-cis)-4-hydroxycyclohexyl)methyl)-1H-indazole-3-carboxamide;
5-(1-aminoisoquinolin-7-yl)-N-((1-methylazetidin-3-yl)methyl)-1H-indazole-3-carboxamide;
(5-(1-aminoisoquinolin-7-yl)-1H-indazol-3-yl)(4-(dimethylamino)piperidin-1-yl)methanone;
(5-(1-aminoisoquinolin-7-yl)-1H-indazol-3-yl)(4-hydroxypiperidin-1-yl)methanone;
5-(1-aminoisoquinolin-7-yl)-N-((1-methylpiperidin-4-yl)methyl)-1H-indazole-3-carboxamide;
5-(1-aminoisoquinolin-7-yl)-N-(1-methylpiperidin-4-yl)-1H-indazole-3-carboxamide;
(5-(1-aminoisoquinolin-7-yl)-1H-indazol-3-yl)(4-aminopiperidin-1-yl)methanone;
3-(8-amino-1,7-naphthyridin-2-yl)-N-(3-(dimethylamino)propyl)benzamide;
5-(1-aminoisoquinolin-7-yl)-N-(2-(dimethylamino)ethyl)-1H-indazole-3-carboxamide;
(4-(1-aminoisoquinolin-7-yl)-5,6,7,8-tetrahydronaphthalen-2-yl)(pyrrolidin-1-yl)methanone;
(3-((1-aminoisoquinolin-7-yl)methyl)phenyl)(pyrrolidin-1-yl)methanone;
7-(3-(2-(4-methylpiperazin-1-yl)ethyl)phenyl)isoquinolin-1-amine;
3-(1-aminoisoquinolin-7-yl)-N-(1-methylpiperidin-4-yl)benzamide;
(3-(1-aminoisoquinolin-7-yl)phenyl)(4-methoxypiperidin-1-yl)methanone;
3-(1-aminoisoquinolin-7-yl)-N-((1-methylazetidin-3-yl)methyl)benzamide;
3-(1-aminoisoquinolin-7-yl)-N-(3-(dimethylamino)propyl)benzamide;
(4-(1-aminoisoquinolin-7-yl)thiophen-2-yl)(pyrrolidin-1-yl)methanone;
6-(3-(3-(dimethylamino)pyrrolidine-1-carbonyl)phenyl)isoquinolin-1 (2H)-one;
(3-(1-aminoisoquinolin-7-yl)phenyl)(4-methylpiperazin-1-yl)methanone;
6-(3-(pyrrolidine-1-carbonyl)phenyl)isoquinolin-1 (2H)-one;
(3-(1-aminoisoquinolin-7-yl)phenyl)(pyrrolidin-1-yl)methanone;
(1-(1-aminoisoquinolin-7-yl)pyrrolidin-3-yl)(pyrrolidin-1-yl)methanone;
5-(1-aminoisoquinolin-7-yl)-N-(2-(dimethylamino)ethyl)benzo[b]thiophene-3-carboxamide;
1′-amino-N-(2-(dimethylamino)ethyl)-[6,7′-biisoquinoline]-3-carboxamide;
3-(1-aminoisoquinolin-7-yl)-N-(3-(dimethylamino)propyl)benzo[b]thiophene-6-carboxamide;
7-(1-(4-(dimethylamino)butyl)-1H-benzo[d]imidazol-6-yl)isoquinolin-1-amine;
3-(1-aminoisoquinolin-7-yl)-N-(3-(dimethylamino)propyl)-1H-indole-6-carboxamide;
5-(1-aminoisoquinolin-7-yl)-N-(2-(dimethylamino)ethyl)-2-methyl-2H-indazole-3-carboxamide;
7-(1H-indazol-5-yl)isoquinolin-1-amine;
7-(1H-benzo[d]imidazol-5-yl)isoquinolin-1-amine;
4-(1-methyl-1H-pyrazol-5-yl)-2-(2-methylpiperazin-1-yl)-8-(1H-pyrazol-5-yl)-1,7-naphthyridine;
2-(3,8-diazabicyclo[3.2.1]octan-3-yl)-4-(1-methyl-1H-pyrazol-5-yl)-8-(1H-pyrazol-3-yl)-1,7-naphthyridine;
3-(4-(1-methyl-1H-pyrazol-5-yl)-8-(1H-pyrazol-3-yl)-1,7-naphthyridin-2-yl)-3,6-diazabicyclo[3.1.1]heptane;
6-isopropyl-4-(4-(1-methyl-1H-pyrazol-5-yl)-8-(1H-pyrazol-3-yl)-1,7-naphthyridin-2-yl)pyridin-2-amine; and
6-isopropyl-4-(4-(1-methyl-1H-pyrazol-5-yl)-8-(1H-pyrazol-3-yl)quinolin-2-yl)pyridin-2-amine.
   29. A pharmaceutical composition comprising the compound according to any one of embodiments 1 to 28 and at least one additional component chosen from pharmaceutically acceptable carriers, pharmaceutically acceptable vehicles, and pharmaceutically acceptable excipients.
   30. The pharmaceutical composition according to embodiment 29, wherein the compound is present in a therapeutically effective amount.
   31. A method of treating cancer in a subject in need thereof, comprising administering to the subject an effective amount of the compound according to any one of embodiments 1 to 28 or of the pharmaceutical composition according to embodiment 29 or 30.
   32. The method according to embodiment 31, wherein the cancer is breast cancer.
   33. The method according to embodiment 31, wherein the cancer is pancreatic cancer.
   34. The method according to embodiment 31, wherein the cancer is colorectal cancer.
   35. A use of the compound according to any one of embodiments 1 to 28 or of the pharmaceutical composition according to embodiment 29 or 30, in the preparation of a medicament.
   36. A method of inhibiting E3 ligase in cells, comprising treating cells with the compound of any one of embodiments 1 to 28 or of the pharmaceutical composition according to embodiment 29 or 30.
   37. The method according to embodiment 36, wherein the cancer is selected from breast cancer, lung cancer, pancreatic cancer, colorectal cancer, gall bladder cancer, thyroid cancer, bile duct cancer, ovarian cancer, endometrial cancer, prostate cancer, and esophageal cancer.
   38. The method according to embodiment 36, further comprising administering to the subject in combination with an anti-cancer agent.

### EXAMPLES

The examples and preparations provided below further illustrate and exemplify the compounds as disclosed herein and methods of preparing such compounds. It is to be understood that the scope of the present disclosure is not limited in any way by the scope of the following examples and preparations.

The chemical entities described herein can be synthesized according to one or more illustrative schemes herein and/or techniques well known in the art. Unless specified to the contrary, the reactions described herein take place at atmospheric pressure, generally within a temperature range from about −78° C. to about 200° C. Further, except as otherwise specified, reaction times and conditions are intended to be approximate, e.g., taking place at about atmospheric pressure within a temperature range of about −78° C. to about 200° C. over a period that can be, for example, about 1 to about 24 h; reactions left to run overnight in some embodiments can average a period of about 16 h.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. See, e.g., Carey et al. Advanced Organic Chemistry, 3^{rd} Ed., 1990 New York: Plenum Press; Mundy et al., Name Reaction and Reagents in Organic Synthesis, 2^{nd} Ed., 2005 Hoboken, NJ: J. Wiley & Sons. Specific illustrations of suitable separation and isolation procedures are given by reference to the examples hereinbelow. However, other equivalent separation or isolation procedures can also be used.

In all of the methods, it is well understood that protecting groups for sensitive or reactive groups may be employed where necessary, in accordance with general principles of chemistry. Protecting groups are manipulated according to standard methods of organic synthesis (T. W. Greene and P. G. M. Wuts (1999) Protective Groups in Organic Synthesis, 3^{rd} Ed., John Wiley & Sons). These groups may be removed at a convenient stage of the compound synthesis using methods that are readily apparent to those skilled in the art.

When desired, the (R)- and (S)-isomers of the nonlimiting exemplary compounds, if present, can be resolved by methods known to those skilled in the art, for example, by formation of diastereoisomeric salts or complexes which can be separated, e.g., by crystallization; via formation of diastereoisomeric derivatives which can be separated, e.g., by crystallization, gas-liquid or liquid chromatography; selective reaction of one enantiomer with an enantiomer-specific reagent, e.g., enzymatic oxidation or reduction, followed by separation of the modified and unmodified enantiomers; or gas-liquid or liquid chromatography in a chiral environment, e.g., on a chiral support, such as silica with a bound chiral ligand or in the presence of a chiral solvent. Alternatively, a specific enantiomer can be synthesized by asymmetric synthesis using optically active reagents, substrates, catalysts or solvents, or by converting one enantiomer to the other by asymmetric transformation.

The compounds described herein can be optionally contacted with a pharmaceutically acceptable acid to form the corresponding acid addition salts. Also, the compounds described herein can be optionally contacted with a pharmaceutically acceptable base to form the corresponding basic addition salts.

In some embodiments, disclosed compounds can generally be synthesized by an appropriate combination of generally well-known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Millipore Sigma or can be readily prepared by those skilled in the art using commonly employed synthetic methodology.

The discussion below is offered to illustrate certain of the diverse methods available for use in making the disclosed compounds and is not intended to limit the scope of reactions or reaction sequences that can be used in preparing the compounds provided herein. The skilled artisan will understand that standard atom valencies apply to all compounds disclosed herein in genus or named compound for unless otherwise specified.

The following abbreviations have the definitions set forth below:
ACN acetonitrile
Boc tert-Butyloxycarbonyl
B₂pin₂ Bis(pinacolato)diboron
DAST Diethylaminosulfur trifluoride
DHP 3,4-Dihydropyran
DMAP 4-dimethylaminopyridine
DMF dimethylformamide
EA ethyl acetate
DMSO Dimethyl sulfoxide
EDCI 1-Ethyl-3-(3-dimethylaminopropyl)carbodiimide
LC-MS liquid chromatography/mass spectrometry
h hour
HOBt Hydroxybenzotriazole
LAH Lithium aluminium hydride
min minute
NMR nuclear magnetic resonance
dppfCl2 1,1′-bis(diphenylphosphino)ferrocene
PE petroleum ether
NBS N-Bromosuccinimide
NIS N-lodosuccinimide
Prep-TLC preparative thin layer chromatography
PyBop benzotriazol-1-yloxytripyrrolidinophosphonium hexafluorophosphate
SFC Supercritical fluid chromatography
T₃P Propanephosphonic acid anhydride
TEA triethylamine
Tf trifluoromethylsufonyl
TFA trifluoroacetic acid
THF tetrahydrofuran
TMS Trimethylsilyl
TsOH p-toluenesulfonic acid

### General Synthetic Schemes

The claimed compounds can be prepared according to the following schemes. The following schemes represent the general methods used in preparing these compounds. However, the synthesis of these compounds is not limited to these representative methods, as they can also be prepared through various other methods by those skilled in the art of synthetic chemistry.

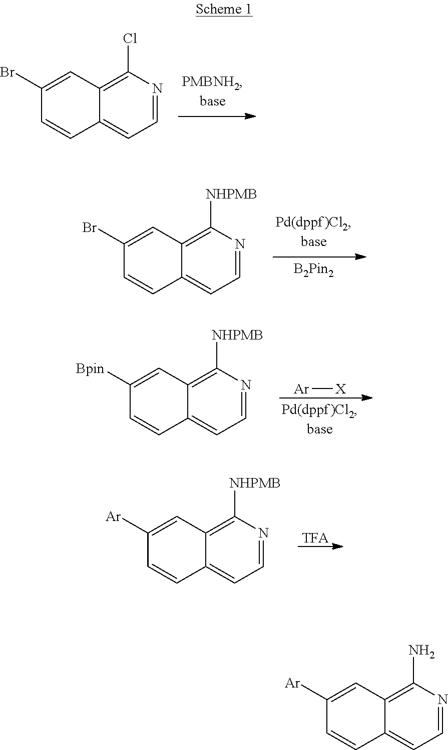

Compounds 1, 3, 8, 353, 355, 356, 357, 358, 359, 360, 361, and 362 can be prepared via a similar procedure to the one shown in Scheme 1.

TABLE 3
Characterization of selected compounds

| Compound | | |
|---|---|---|
| No. | LC-MS | ¹H NMR |
| 1 | [M + H]⁺ = | ¹H NMR (DMSO-d₆, 500 MHz) δ 11.52 (br, 1H), 8.65 (s, |
| | 277.0 | 1H), 8.14 (s, 1H), 8.11-8.09 (m, 2H), 7.86-7.83 (m, 2H), |
| | | 7.73 (d, J = 5.0 Hz, 1H), 7.64 (br, 2H), 7.42 (d, J = 7.0 Hz, |
| | | 1H), 7.03 (d, J = 5.0 Hz, 1H) |
| 3 | [M + H]⁺ = | ¹H NMR (CD₃OD, 500 MHz) δ 8.79 (s, 1H), 8.37 (d, J = 8.5 |
| | 361.0 | Hz, 1H), 8.30 (s, 1H), 8.01 (d, J = 8.5 Hz, 1H), 7.92 (d, J = |
| | | 8.0 Hz, 1H), 7.70 (d, J = 8.5 Hz, 1H), 7.57 (d, J = 7.0 Hz, |
| | | 1H), 7.26 (d, J = 7.0 Hz, 1H), 4.45 (s, 2H), 3.17-3.14 (m, |
| | | 2H), 3.12-3.09 (m, 2H), 2.73 (s, 6H) |
| 8 | M + H]⁺ = | ¹H NMR (CD₃OD, 400 MHz) δ 8.95 (d, J = 2.4 Hz, 1H), 8.82 |
| | 375.4 | (s, 1H), 8.78 (d, J = 2.4 Hz, 1H), 8.37 (dd, J = 1.6, 8.4 Hz, |
| | | 1H), 8.17 (s, 1H), 8.08 (d, J = 8.4 Hz, 1H), 7.60 (d, J = 7.2 |
| | | Hz, 1H), 7.31 (d, J = 7.2 Hz, 1H), 3.80 (t, J = 5.6 Hz, 2H), |
| | | 3.42 (t, J = 5.6 Hz, 2H), 3.02 (s, 6H) |
| 353 | [M + H]⁺ = | ¹H NMR (DMSO-d₆, 400 MHz) δ 8.56 (s, 1H), 8.16 (s, 1H), |
| | 318.1 | 8.04-7.90 (m, 3H), 7.85-7.74 (m, 2H), 7.60-7.54 (m, 1H), |
| | | 7.53-7.48 (m, 1H), 7.04-6.90 (m, 3H), 3.53-3.49 (m, 2H), |
| | | 3.46-3.43 (m, 2H), 1.95-1.79 (m, 4H) |
| 355 | [M + H]⁺ = | ¹H NMR (DMSO-d₆, 400 MHz) δ 8.81 (s, 1H), 8.55 (s, 1H), |
| | 391.0 | 8.44 (t, J = 5.6 Hz, 1H), 8.37 (s, 1H), 8.18 (d, J = 8.4 Hz, |
| | | 1H), 7.97 (d, J = 8.4 Hz, 1H), 7.94-7.89 (m, 1H), 7.84-7.79 |
| | | (m, 2H), 6.96-6.89 (m, 3H), 3.41 (dd, J = 6.8 Hz, 2H). 2.45 |
| | | (t, J = 6.8 Hz, 2H), 2.21 (s, 6H) |
| 356 | [M + H]⁺ = | ¹H NMR (CD₃OD, 400 MHz) δ 9.34 (s, 1H), 8.65 (d, J = 2.8 |
| | 386.1 | Hz, 2H), 8.51 (s, 1H), 8.36-8.25 (m, 2H), 8.18 (dd, J = 1.6, |
| | | 8.6 Hz, 1H), 7.91 (d, J = 8.4 Hz, 1H), 7.81 (d, J = 6.0 Hz, |
| | | 1H), 7.07 (d, J = 5.6 Hz, 1H), 3.67 (t, J = 6.8 Hz, 2H), 2.68 |
| | | (t, J = 6.8 Hz, 2H), 2.38 (s, 6H), |
| 357 | [M + H]⁺ = | ¹H NMR (CD₃OD, 500 MHz) δ 8.68 (s, 1H), 8.55 (s, 1H), |
| | 405.0 | 8.21 (d, J = 8.0 Hz, 1H), 8.08-8.02 (m, 3H), 7.95 (d, J = 9.0 |
| | | Hz, 1H), 7.63 (d, J = 7.0 Hz, 1H), 7.29 (d, J = 6.5 Hz, 1H), |
| | | 3.55 (t, J = 7.0 Hz, 2H), 3.23 (t, J = 7.5 Hz, 2H), 2.93 (s, |
| | | 6H), 2.11-2.05 (m, 2H) |
| 358 | [M + H]⁺ = | |
| | 360.5 | |
| 359 | LC-MS: | ¹H NMR (CDCl₃, 500 MHz) δ 8.05 (d, J = 8.5 Hz, 2H), |
| | [M + H]⁺ = | 7.97-7.93 (m, 2H), 7.89 (d, J = 8.5 Hz, 1H), 7.81 (d, J = 8.5 |
| | 388.0 | Hz, 1H), 7.69 (dd, J = 8.5, 1.5 Hz, 1H), 7.10 (d, J = 6.0 Hz, |
| | | 1H), 5.39 (br, 2H), 4.53 (s, 3H), 3.64 (q, J = 5.5 Hz, 2H), |
| | | 2.64 (t, J = 5.5 Hz, 2H), 2.34 (s, 6H) |
| 360 | [M + H]⁺ = | ¹H NMR (CDCl₃, 500 MHz) δ 8.05 (d, J = 8.5 Hz, 2H), |
| | 389.0 | 7.97-7.93 (m, 2H), 7.89 (d, J = 8.5 Hz, 1H), 7.81 (d, J = 8.5 |
| | | Hz, 1H), 7.69 (dd, J = 8.5, 1.5 Hz, 1H), 7.10 (d, J = 6.0 Hz, |
| | | 1H), 5.39 (br, 2H), 4.53 (s, 3H), 3.64 (q, J = 5.5 Hz, 2H), |
| | | 2.64 (t, J = 5.5 Hz, 2H), 2.34 (s, 6H) |
| 361 | [M + H]⁺ = | ¹H NMR (CD₃OD, 500 MHz) δ 8.58 (s, 1H), 8.20 (s, 1H), |
| | 261.0 | 8.17 (dd, J = 8.5, 1.5 Hz, 1H), 8.15 (s, 1H), 7.90 (d, J = 8.5 |
| | | Hz, 1H), 7.87 (dd, J = 8.5, 1.5 Hz, 1H), 7.69 (d, J = 9.0 Hz, |
| | | 1H), 7.66 (d, J = 6.0 Hz, 1H), 7.13 (d, J = 6.5 Hz, 1H) |
| 362 | LC-MS: | ¹H NMR (CD₃OD, 500 MHz) δ 8.55 (s, 1H), 8.20 (s, 1H), |
| | [M + H]⁺ = | 8.16-8.13 (m, 2H), 7.89-7.86 (m, 2H), 7.70-7.66 (m, 2H), |
| | 261.0 | 7.11 (d, J = 6.5 Hz, 1H) |

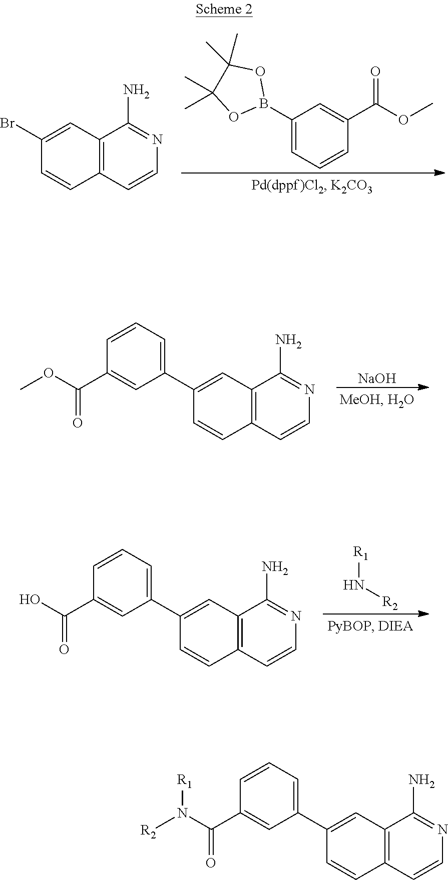

Compounds 345, 346, 347, 348, 351, and 353 can be prepared via a similar procedure to the one shown in Scheme 2.

TABLE 4
Characterization of selected compounds

| Compound | | |
|---|---|---|
| No. | LC-MS | ¹H NMR |
| 345 | [M + H]⁺ = | ¹H NMR (DMSO-d₆, 400 MHz) |
| | 361.2 | δ 8.58 (Br, 1H), 8.36 (d, J = 7.6 Hz, 1H), 8.23 (s, 1H), |
| | | 8.05-7.95 (m, 2H), 7.87-7.76 (m, 3H), 7.59 (t, J = 7.6 Hz, |
| | | 1H), 6.99-6.88 (m, 3H), 3.84-3.69 (m, 1H), 2.79 (d, J = |
| | | 11.6 Hz, 2H), 2.17 (s, 3H), 2.01-1.91 (m, 2H), 1.83-1.74 |
| | | (m, 2H), 1.67-1.54 (m, 2H) |
| 346 | [M + H]⁺ = | ¹H NMR (DMSO-d₆, 400 MHz) δ 8.55 (s, 1H), 8.01-7.99 |
| | 362.2 | (m, 1H), 7.93 (d, J = 8 Hz, 1H), 7.87 (s, 1H), 7.81-7.77 (m, |
| | | 2H), 7.57 (t, J = 8.0 Hz, 1H), 7.38 (d, J = 7.6 Hz, 1H), |
| | | 6.93-6.92 (m, 3H), 3.97 (m, 1H), 3.51 (m, 1H), 3.48-3.43 |
| | | (m, 1H), 3.26 (m, 5H), 1.91-1.81 (m, 2H), 1.49-1.45 (m, 2H) |
| 347 | [M + H]⁺ = | ¹H NMR (DMSO-d₆, 400 MHz) δ 8.69-8.62 (m, 1H) 8.58- |
| | 347.0 | 8.52 (m, 1H), 8.25 (s, 1H), 8.06-7.94 (m, 2H), 7.88-7.78 |
| | | (m, 3H), 7.60 (t, J = 7.6 Hz, 1H), 6.99-6.86 (m, 3H), 3.47 |
| | | (t, J = 6.4 Hz, 2H), 3.24 (d, J = 7.2 Hz, 2H), 2.98-2.90 (m, |
| | | 2H), 2.62-2.53 (m, 1H), 2.20 (s, 3H) |
| 348 | [M + H]⁺ = | ¹H NMR (CD₃CN, 400 MHz) δ 8.38 (s, 1H), 8.28 (s, 1H), |
| | 349.0 | 8.02 (dd, J = 8.4, 1.6 Hz, 2H), 7.95 (d, J = 8.0 Hz, 1H), |
| | | 7.90-7.77 (m, 3H), 7.60 (t, J = 7.6 Hz, 1H), 7.04 (d, J = 5.6 |
| | | Hz, 1H), 6.41-5.94 (m, 2H), 3.48 (q, J = 6.0 Hz, 2H), 2.88 |
| | | (t, J = 6.8 Hz, 2H), 2.59 (s, 6H), 2.48 (m, 2H) |
| 351 | [M + H]⁺ = | ¹H NMR (CD₃OD, 400 MHz) δ 8.75 (s, 1H), 8.33 (dd, J = |
| | 347.2 | 8.4, 1.6 Hz, 1H), 8.05 (d, J = 8.4 Hz, 1H), 8.00 (d, J = 7.6 |
| | | Hz, 1H), 7.95 (s, 1H) 7.72-7.65 (m, 1H), 7.59 (d, J = 6.8 |
| | | Hz, 2H), 7.29 (d, J = 7.2 Hz, 1H), 3.63-3.36 (m, 4H), 3.30- |
| | | 3.10 (m, 4H), 2.98 (s, 3H) |
| 353 | [M + H]⁺ = | ¹H NMR (DMSO-d₆, 400 MHz) δ 8.56 (s, 1H), 8.16 (s, 1H), |
| | 318.1 | 8.04-7.90 (m, 3H), 7.85-7.74 (m, 2H), 7.60-7.54 (m, 1H), |
| | | 7.53-7.48 (m, 1H), 7.04-6.90 (m, 3H), 3.53-3.49 (m, 2H), |
| | | 3.46-3.43 (m, 2H), 1.95-1.79 (m, 4H) |

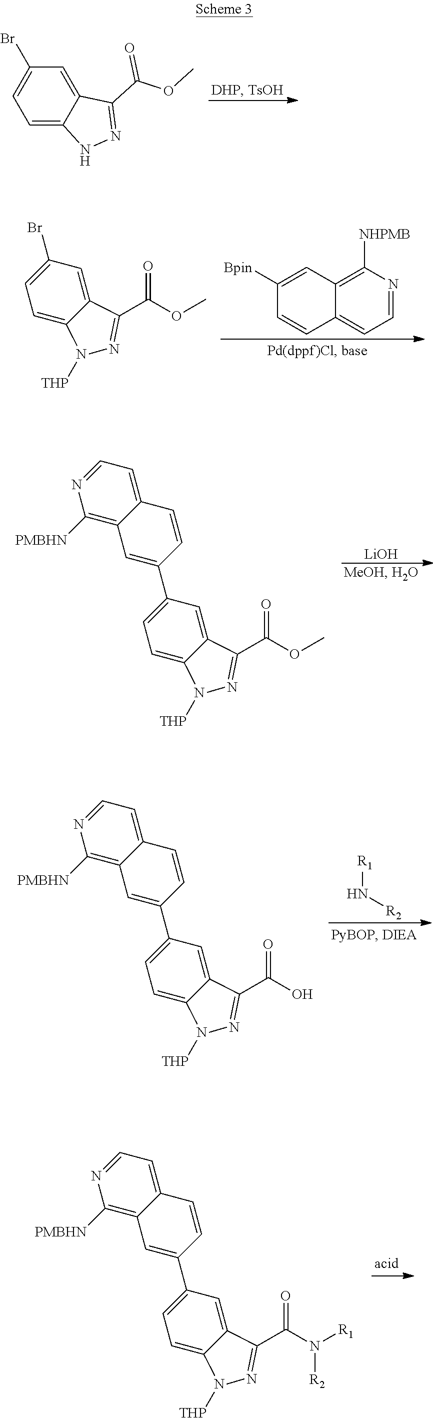

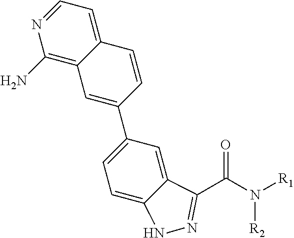

Compounds 10, 326, 327, 328, 329, 331, 332, 333, 334, 335, 336, 337, 338, 339, and 341 can be prepared via a similar procedure to the one shown in Scheme 3.

TABLE 5
Characterization of selected compounds

| Compound | | |
|---|---|---|
| No. | LC-MS | ¹H NMR |
| 10 | [M + H]⁺ = | ¹H NMR (DMSO-d₆, 400 MHz) δ 13.85 (d, J = 1.6 Hz, 1H), |
| | 415.4 | 13.37-13.01 (m, 1H), 9.89-9.46 (m, 1H), 9.24-9.05 (m, 1H), |
| | | 8.93 (s, 1H), 8.63-8.58 (m, 1H), 8.34 (d, J = 8.0 Hz, 1H), 8.07 |
| | | (d, J = 8.8 Hz, 1H), 7.98-7.93 (m, 2H), 7.82 (d, J = 8.8 Hz, |
| | | 1H), 7.71 (d, J = 6.8 Hz, 1H), 7.30 (d, J = 7.2 Hz, 1H), 3.14- |
| | | 3.06 (m, 2H), 2.84-2.82 (m, 1H), 2.79-2.75 (m, 3H), 2.57-2.55 |
| | | (m, 2H), 2.31-2.24 (m, 1H), 1.81-1.65 (m, 2H), 1.46 (s, 3H) |
| 326 | [M + H]⁺ = | ¹H NMR (DMSO-d₆, 400 MHz) δ 13.86 (s, 1H), 12.97 (m, 1H), |
| | 425.1 | 9.20-9.10 (m, 3H), 8.93 (s, 1H), 8.62 (s, 1H), 8.35 (dd, J = 1.6, |
| | | 8.4 Hz, 1H), 8.14-8.03 (m, 2H), 7.93 (dd, J = 2.0, 8.8 Hz, 1H), |
| | | 7.83 (d, J = 8.8 Hz, 1H), 7.68 (d, J = 6.8 Hz, 1H), 7.30 (d, J = |
| | | 6.8 Hz, 1H), 6.98 (dd, J = 1.2, 5.2 Hz, 1H), 6.75 (s, 1H), 4.50 |
| | | (d, J = 6.0 Hz, 2H), 3.83 (s, 3H) |
| 327 | [M + H]⁺ = | ¹H NMR (DMSO-d₆, 400 MHz) δ 14.21 (br, 1H), 13.71-13.19 |
| | 376.1 | (m, 1H), 9.58-9.39 (m, 1H), 9.33 (s, 1H), 9.27-9.17 (m, 2H), |
| | | 9.00-8.83 (m, 2H), 8.47-8.30 (m, 2H), 8.12 (d, J = 8.8 Hz, 1H), |
| | | 7.76 (d, J = 6.8 Hz, 1H), 7.32 (d, J = 6.8 Hz, 1H), 3.84 (d, J = |
| | | 6.0 Hz, 2H), 3.32-3.28 (m, 2H), 2.90 (s, 6H) |
| 328 | [M + H]⁺ = | ¹H NMR (DMSO-d₆, 400 MHz) δ 13.76 (s, 1H), 13.24 (br, 1H), |
| | 398.1 | 9.44-9.05 (m, 2H), 8.92 (s, 1H), 8.81 (s, 1H), 8.63 (s, 1H), |
| | | 8.34 (d, J = 1.6, 8.4 Hz, 1H), 8.07 (d, J = 8.4 Hz, 1H), 7.91 (d, |
| | | J = 1.6, 8.8 Hz, 1H), 7.79 (d, J = 8.4 Hz, 1H), 7.69 (d, J = 6.8 |
| | | Hz, 1H), 7.61 (s, 1H), 7.38 (s, 1H), 7.29 (d, J = 7.2 Hz, 1H), |
| | | 4.34 (d, J = 6.0 Hz, 2H), 3.78 (s, 3H) |
| 329 | [M + H]⁺ = | ¹H NMR (DMSO-d₆, 400 MHz) δ 13.91 (s, 1H), 9.98-9.52 (m, |
| | 376.2 | 2H), 9.50-9.29 (m, 1H), 8.86-8.75 (m, 3H), 8.59 (s, 1H), 8.37 |
| | | (dd, J = 1.6, 8.8 Hz, 1H), 7.93-7.86 (m, 2H), 7.86-7.78 (m, |
| | | 1H), 3.70-3.68 (m, 2H), 3.32 (s, 2H), 2.87 (s, 6H) |
| 331 | [M + H]⁺ = | ¹H NMR (CD₃OD, 400 MHz) δ 14.01-13.74 (m, 1H), 13.34- |
| | 348.3 | 12.95 (m, 1H), 9.30-9.01 (m, 2H), 8.91 (s, 1H), 8.62 (s, 1H), |
| | | 8.37-8.23 (m, 2H), 8.07 (d, J = 8.4 Hz, 1H), 7.96-7.89 (m, 1H), |
| | | 7.86-7.77 (m, 1H), 7.69 (d, J = 6.8 Hz, 1H), 7.28 (d, J = 7.2 |
| | | Hz, 1H), 4.86-4.72 (m, 1H), 3.60-3.50 (m, 2H), 3.45-3.40 (m, 2H) |
| 332 | [M + H]⁺ = | ¹H NMR (DMSO-d₆, 400 MHz) δ 13.68 (s, 1H), 13.20 (s, 1H), |
| | 402.0 | 9.25 (s, 2H), 8.92 (s, 1H), 8.42 (s, 1H), 8.35 (d, J = 1.6 Hz, |
| | | 1H), 8.07 (d, J = 8.4 Hz, 1H), 7.94 (d, J = 8.8 Hz, 1H), 7.80 (d, |
| | | J = 8.4 Hz, 1H), 7.69 (d, J = 6.8 Hz, 1H), 7.30 (d, J = 6.8 Hz, |
| | | 1H), 4.29-4.28 (m, 1H), 4.09-4.08 (m, 1H), 3.67 (t, J = 10.0 |
| | | Hz, 1H), 3.54-3.49 (m, 2H), 3.30 (s, 3H), 1.99-1.94 (m, 2H), |
| | | 1.53-1.52 (m, 2H) |
| 333 | [M + H]⁺ = | ¹H NMR (DMSO-d₆, 400 MHz) δ 13.90-13.88 (m, 1H), 13.61- |
| | 416.2 | 13.23 (m, 1H), 9.86-9.85 (m, 1H), 9.30-9.25 (m, 2H), 8.92- |
| | | 8.86 (m, 2H), 8.59 (s, 1H), 8.32 (d, J = 8.4 Hz, 1H), 8.07 (d, J = |
| | | 8.4 Hz, 1H), 7.93 (d, J = 8.8 Hz, 1H), 7.82 (d, J = 9.2 Hz, |
| | | 1H), 7.70 (d, J = 7.2 Hz, 1H), 7.29 (d, J = 3.6 Hz, 1H), 4.23- |
| | | 4.13 (m, 2H), 3.95-3.84 (m, 2H), 3.58-3.54 (m, 2H), 3.06-2.83 |
| | | (m, 1H), 2.81 (m, 3H) |
| 334 | [M + H]⁺ = | ¹H NMR (DMSO-d₆, 400 MHz) δ 13.90-13.88 (m, 1H), 13.61- |
| | 387.0 | 13.23 (m, 1H), 9.86-9.85 (m, 1H), 9.30-9.25 (m, 2H), 8.92- |
| | | 8.86 (m, 2H), 8.59 (s, 1H), 8.32 (d, J = 8.4 Hz, 1H), 8.07 (d, J = |
| | | 8.4 Hz, 1H), 7.93 (d, J = 8.8 Hz, 1H), 7.82 (d, J = 9.2 Hz, |
| | | 1H), 7.70 (d, J = 7.2 Hz, 1H), 7.29 (d, J = 3.6 Hz, 1H), 4.23- |
| | | 4.13 (m, 2H), 3.95-3.84 (m, 2H), 3.58-3.54 (m, 2H), 3.06-2.83 |
| | | (m, 1H), 2.81 (m, 3H) |
| 335 | [M + H]⁺ = | ¹H NMR (CD₃OD, 400 MHz) δ 8.79 (s, 1H), 8.46 (s, 1H), 8.36 |
| | 415.4 | (d, J = 6.4 Hz, 1H), 8.04 (d, J = 8.4 Hz, 1H), 7.94 (dd, J = 1.2, |
| | | 8.8 Hz, 1H), 7.78 (d, J = 8.0 Hz, 1H), 7.58 (d, J = 7.2 Hz, 1H), |
| | | 7.29 (d, J = 7.2 Hz, 1H), 5.32-5.31 (m, 1H), 5.03-5.01 (m, 1H), |
| | | 3.63-3.56 (m, 1H), 2.98-2.93 (m, 8H), 2.27-2.25 (m, 2H), 1.83- |
| | | 1.81 (m, 2H) |
| 336 | [M + H]⁺ = | ¹H NMR (DMSO-d₆, 400 MHz) δ 13.65 (s, 1H), 13.02 (s, 1H), |
| | 388.3 | 9.12 (s, 2H), 8.91 (s, 1H), 8.40 (s, 1H), 8.34 (d, J = 1.6 Hz, |
| | | 1H), 8.06 (d, J = 8.4 Hz, 1H), 7.92 (d, J = 1.6 Hz, 1H), 7.79 (d, |
| | | J = 8.8 Hz, 1H), 7.68 (d, J = 6.8 Hz, 1H), 7.28 (d, J = 6.8 Hz, |
| | | 1H), 4.81 (s, 1H), 4.40-4.31 (m, 1H), 4.19-4.16 (m, 1H), 3.82- |
| | | 3.78 (m, 1H), 3.61-3.52 (m, 1H), 1.84-1.81 (m, 2H), 1.47-1.43 |
| | | (m, 2H) |
| 337 | [M + H]⁺ = | ¹H NMR (DMSO-d₆, 400 MHz) δ 13.82 (s, 1H), 13.36 (s, 1H), |
| | 415.2 | 9.26 (s, 3H), 8.93 (s, 1H), 8.62 (t, J = 6.0 Hz, 1H), 8.62 (s, |
| | | 1H), 8.33 (d, J = 8.4 Hz, 1H), 8.08 (d, J = 8.4 Hz, 1H), 7.94 (d, |
| | | J = 10.4 Hz, 1H), 7.81 (d, J = 8.8 Hz, 1H), 7.71 (d, J = 7.2 Hz, |
| | | 1H), 7.30 (d, J = 6.8 Hz, 1H), 3.44 (t, J = 12.0 Hz, 2H), 3.26 (t, |
| | | J = 6.0 Hz, 2H), 2.91-2.90 (m, 2H), 2.76 (s, 3H), 1.91-1.83 (m, |
| | | 3H), 1.46-1.40 (m, 2H) |
| 338 | [M + H]⁺ = | ¹H NMR (DMSO-d₆, 400 MHz) δ 13.83 (s, 1H), 13.33-13.09 |
| | 401.3 | (m, 1H), 9.60-9.02 (m, 3H), 8.92 (s, 1H), 8.67 (d, J = 8.0 Hz, |
| | | 1H), 8.60 (s, 1H), 8.33 (d, J = 8.0 Hz, 1H), 8.08 (d, J = 8.4 Hz, |
| | | 1H), 7.92 (d, J = 1.2 Hz, 1H), 7.82 (d, J = 8.8 Hz, 1H), 7.71 (d, |
| | | J = 6.8 Hz, 1H), 7.30 (d, J = 6.8 Hz, 1H), 4.13-4.11 (m, 1H), |
| | | 3.51-3.49 (m, 2H), 3.18-3.08 (m, 2H), 2.79 (s, 3H), 2.13-2.02 |
| | | (m, 2H), 1.98-1.84 (m, 2H) |
| 339 | [M + H]⁺ = | ¹H NMR: (DMSO-d₆, 400 MHz) δ 13.74 (d, J = 4.8 Hz, 1H), |
| | 387.3 | 13.35-13.00 (m, 1H), 9.29-9.01 (m, 2H), 8.92 (s, 1H), 8.42 (s, |
| | | 1H), 8.35 (d, J = 8.8 Hz, 1H), 8.07 (d, J = 8.4 Hz, 1H), 7.96 |
| | | (dd, J = 1.6, 8.8 Hz, 4H), 7.81 (d, J = 8.8 Hz, 1H), 7.70 (d, J = |
| | | 6.8 Hz, 1H), 7.29 (d, J = 6.8 Hz, 1H), 4.88-4.64 (m, 2H), 3.29- |
| | | 3.27 (m, 2H), 3.00-2.92 (m, 1H), 2.09-1.99 (m, 2H), 1.61-1.50 |
| | | (m, 2H) |
| 341 | [M + H]⁺ = | ¹H NMR (CD₃OD, 400 MHz) δ 8.82 (s, 1H), 8.69 (s, 1H), 8.38 |
| | 375.2 | (dd, J = 1.6, 8.4 Hz, 1H), 8.06 (d, J = 8.4 Hz, 1H), 7.96 (dd, J = |
| | | 1.6, 8.4 Hz, 1H), 7.80 (d, J = 8.8 Hz, 1H), 7.60 (d, J = 6.8 |
| | | Hz, 1H), 7.30 (d, J = 7.2 Hz, 1H), 3.88 (t, J = 6.0 Hz, 2H), 3.47 |
| | | (t, J = 5.6 Hz, 2H), 3.04 (s, 6H) |

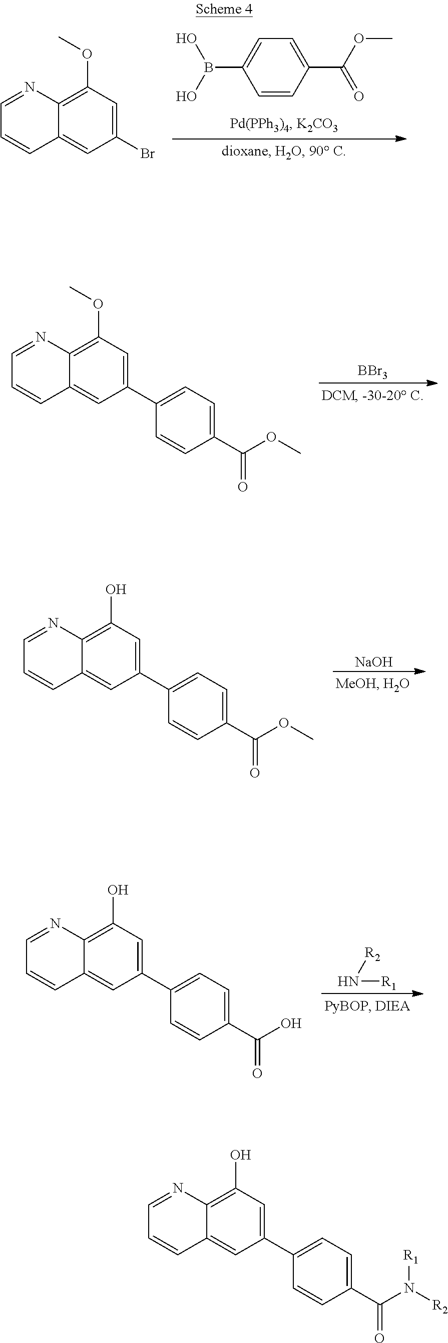

Compounds 279, 280, 286, 287, 289, 290, 291, 292, 297, 298, 299, 300, 302, and 303 can be prepared via a similar procedure to the one shown in Scheme 4.

TABLE 6
Characterization of selected compounds

| Compound | | |
|---|---|---|
| No. | LC-MS | ¹H NMR |
| 279 | [M + H]⁺ = | ¹H NMR (CD₃OD, 400 MHz) δ 9.03-8.86 (m, 2H), 8.13-7.88 (m, |
| | 362.3 | 6H), 7.67 (d, J = 2.8 Hz, 1H), 4.32-4.28 (m, 1H), 3.63-3.57 (m, |
| | | 1H), 2.92-2.89 (m, 2H), 2.90 (s, 6H), 2.47-2.42 (m, 2H) |
| 280 | [M + H]⁺ = | ¹H NMR (CD₃OD, 400 MHz) δ 9.00-8.92 (m, 2H), 8.04 (d, J = |
| | 362.4 | 8.0 Hz, 2H), 7.97-7.91 (m, 4H), 7.68-7.67 (m, 1H), 4.57-4.54 (m, |
| | | 1H), 4.01-3.98(m, 1H), 2.90 (s, 6H), 2.79-2.74 (m, 2H), 2.70- |
| | | 2.67 (m, 2H) |
| 286 | [M + H]⁺ = | ¹H NMR (DMSO-d₆, 400 MHz) δ 9.43 (s, 1H), 8.94-8.88 (m, |
| | 390.2 | 1H), 8.51 (d, J = 8.0 Hz, 1H), 8.19 (d, J = 6.0 Hz, 1H), 8.00-7.93 |
| | | (m, 2H), 7.92-7.87 (m, 2H), 7.83 (s, 1H), 7.67 (dd, J = 4.4, 8.4 |
| | | Hz, 1H), 7.50 (d, J = 1.6 Hz, 1H), 4.11 (d, J = 2.4 Hz, 1H), 3.26- |
| | | 3.19 (m, 1H), 2.78 (d, J = 5.2 Hz, 6H), 2.03 (d, J = 13.2 Hz, 2H), |
| | | 1.86-1.83 (m, 4H), 1.71-1.60 (m, 2H) |
| 287 | [M + H]⁺ = | ¹H NMR (CD₃OD, 400 MHz) δ 9.07-8.91 (m, 2H), 8.09-7.79 (m, |
| | 390.2 | 6H), 7.70 (s, 1H), 4.04-3.91 (m, 1H), 3.31-3.22 (m, 1H), 2.91 (s, |
| | | 6H), 2.33-2.14 (m, 4H), 1.82-1.67 (m, 2H), 1.65-1.50 (m, 2H) |
| 289 | [M + H]⁺ = | ¹H NMR (CD₃OD, 400 MHz) δ 9.22 (d, J = 8.0 Hz, 1H), 9.07 (dd, |
| | 364.0 | J = 1.2, 5.6 Hz, 1H), 8.15-8.04 (m, 4H), 7.94 (d, J = 8.8 Hz, 2H), |
| | | 7.79 (d, J = 1.6 Hz, 1H), 3.51 (t, J = 6.8 Hz, 2H), 3.24 (t, J = 7.6 |
| | | Hz, 2H), 2.93 (s, 6H), 1.88-1.74 (m, 4H) |
| 290 | [M + H]⁺ = | ¹H NMR (CD₃OD, 400 MHz) δ 8.81 (dd, J = 1.6, 4.4 Hz, 1H), |
| | 376.2 | 8.54 (s, 1H), 8.35 (dd, J = 1.6, 8.4 Hz, 1H), 8.00-7.92 (m, 2H), |
| | | 7.86 (d, J = 8.4 Hz, 2H), 7.70 (d, J = 2.0 Hz, 1H), 7.54 (dd, J = |
| | | 4.0, 8.0 Hz, 1H), 7.44 (d, J = 1.6 Hz, 1H), 3.37 (d, J = 6.4 Hz, |
| | | 2H), 3.27-3.25 (m, 1H), 3.18-3.14 (m, 1H), 2.60 (s, 3H), 2.55- |
| | | 2.47 (m, 1H), 2.40-2.28 (m, 1H), 2.11-2.08 (m, 1H), 1.91 (d, J = |
| | | 12.0 Hz, 2H), 1.78-1.66 (m, 1H), 1.30-1.19 (m, 1H) |
| 291 | [M + H]⁺ = | ¹H NMR (MeOD-d₄, 400 MHz) δ 8.97 (dd, J = 1.6, 4.8 Hz, 1H), |
| | 334.1 | 8.84 (d, J = 8.4 Hz, 1H), 8.06 (d, J = 6.8 Hz, 2H), 7.98-7.91 (m, |
| | | 3H), 7.88 (dd, J = 4.8, 8.4 Hz, 1H), 7.64 (d, J = 1.6 Hz, 1H), |
| | | 4.76-4.60 (m, 3H), 4.45-4.28 (m, 2H), 3.08-3.06 (m, 3H) |
| 292 | [M + H]⁺ = | ¹H NMR: (DMSO-d₆, 400 MHz) δ 8.86 (dd, J = 1.6, 4.0 Hz, 1H), |
| | 361.9 | 8.63 (t, J = 5.2 Hz, 1H), 8.40 (dd, J = 1.6, 8.4 Hz, 1H), 8.24 (s, |
| | | 1H), 8.00-7.94 (m, 2H), 7.91-7.86 (m, 2H), 7.79 (d, J = 1.6 Hz, |
| | | 1H), 7.59 (dd, J = 4.0, 8.4 Hz, 1H), 7.46 (d, J = 1.6 Hz, 1H), |
| | | 3.27 (t, J = 6.4 Hz, 2H), 2.66-2.62 (m, 2H), 2.48-2.40 (m, 3H), |
| | | 2.33 (s, 3H), 1.96-1.88 (m, 1H), 1.57-1.50 (m, 1H) |
| 297 | [M + H]⁺ = | ¹H NMR (CD₃OD, 400 MHz) δ 8.83 (dd, J = 1.6, 4.4 Hz, 1H), |
| | 362.2 | 8.48 (s, 1H), 8.37 (dd, J = 1.2, 8.4 Hz, 1H), 7.98 (d, J = 8.0 Hz, |
| | | 2H), 7.87 (d, J = 8.4 Hz, 2H), 7.71 (d, J = 2.0 Hz, 1H), 7.56 (dd, |
| | | J = 4.0, 8.4 Hz, 1H), 7.46 (d, J = 2.0 Hz, 1H), 4.19-4.13 (m, |
| | | 1H), 3.45 (d, J = 12.8 Hz, 2H), 3.03 (t, J = 11.2 Hz, 2H), 2.80 (s, |
| | | 3H), 2.22 (d, J = 12.0 Hz, 2H), 1.99-1.89 (m, 2H) |
| 298 | [M + H]⁺ = | ¹H NMR (DMSO-d₆, 400 MHz) δ 8.87 (dd, J = 1.2, 4.0 Hz, 1H), |
| | 347.9 | 8.66 (t, J = 5.6 Hz, 1H), 8.41 (dd, J = 1.2, 8.4 Hz, 1H), 8.26 (s, |
| | | 1H), 7.98 (d, J = 8.8 Hz, 2H), 7.90 (d, J = 8.8 Hz, 2H), 7.79 (s, |
| | | 1H), 7.60 (dd, J = 4.0, 8.0 Hz, 1H), 7.46 (d, J = 1.6 Hz, 1H), |
| | | 3.48-3.45 (m, 4H), 3.20-3.18 (m, 2H), 2.71-2.66 (m, 1H), 2.35 |
| | | (s, 3H) |
| 299 | [M + H]⁺ = | ¹H NMR (CD₃OD, 400 MHz) δ 8.81 (d, J = 3.6 Hz, 1H), 8.54 (s, |
| | 336.2 | 1H), 8.31 (d, J = 8.4 Hz, 1H), 7.91 (d, J = 7.6 Hz, 2H), 7.77 (d, J = |
| | | 8.0 Hz, 2H), 7.62 (s, 1H), 7.54 (dd, J = 4.0, 8.0 Hz, 1H), 7.39 |
| | | (s, 1H), 3.79 (t, J = 6.0 Hz, 2H), 3.30 (t, J = 6.0 Hz, 2H), 2.91 (s, 6H) |
| 300 | [M + H]⁺ = | ¹H NMR (DMSO-d₆, 400 MHz) δ 8.87 (dd, J = 1.2, 4.0 Hz, 1H), |
| | 376.2 | 8.57 (t, J = 5.6 Hz, 1H), 8.41 (dd, J = 1.2, 8.4 Hz, 1H), 8.23 (s, |
| | | 1H), 7.98 (d, J = 8.0 Hz, 2H), 7.89 (d, J = 8.4 Hz, 2H), 7.79 (d, J = |
| | | 1.2 Hz, 1H), 7.60 (dd, J = 4.0, 8.4 Hz, 1H), 7.46 (d, J = 1.2 |
| | | Hz, 1H), 3.19 (t, J = 6.0 Hz, 2H), 2.90-2.87 (m, 2H), 2.26 (s, |
| | | 3H), 2.07-2.00 (m, 2H), 1.72-1.61 (m, 2H), 1.59-1.58 (m, 1H), |
| | | 1.30-1.23 (m, 2H) |
| 302 | [M + H]⁺ = | ¹H NMR (DMSO-d₆, 400 MHz) δ 10.04 (s, 1H), 8.87 (d, J = 4.0 |
| | 307.0 | Hz, 1H), 8.54 (t, J = 4.8 Hz, 1H), 8.41 (d, J = 8.0 Hz, 1H), 7.98 |
| | | (d, J = 8.4 Hz, 2H), 7.89 (d, J = 8.0 Hz, 2H), 7.80 (s, 1H), 7.60 |
| | | (dd, J = 4.0, 8.4 Hz, 1H), 7.46 (s, 1H), 3.29-3.21 (m, 2H), 1.62- |
| | | 1.51 (m, 2H), 0.92 (t, J = 7.2 Hz, 3H) |
| 303 | [M + H]⁺ = | ¹H NMR (DMSO-d₆, 400 MHz) δ 8.86 (d, J = 2.4 Hz, 1H), 8.74 |
| | 336.0 | (t, J = 5.6 Hz, 1H), 8.41-8.37 (m, 2 H), 7.99 (d, J = 8.4 Hz, 2H), |
| | | 7.89 (d, J = 8.4 Hz, 2H), 7.78 (s, 1H), 7.61-7.46 (m, 21), 7.46 (s, |
| | | 1H), 3.37-3.33 (m, 2H), 2.80 (d, J = 7.2 Hz, 2H), 2.46 (s, 3H), |
| | | 1.85-1.78 (m, 2H) |

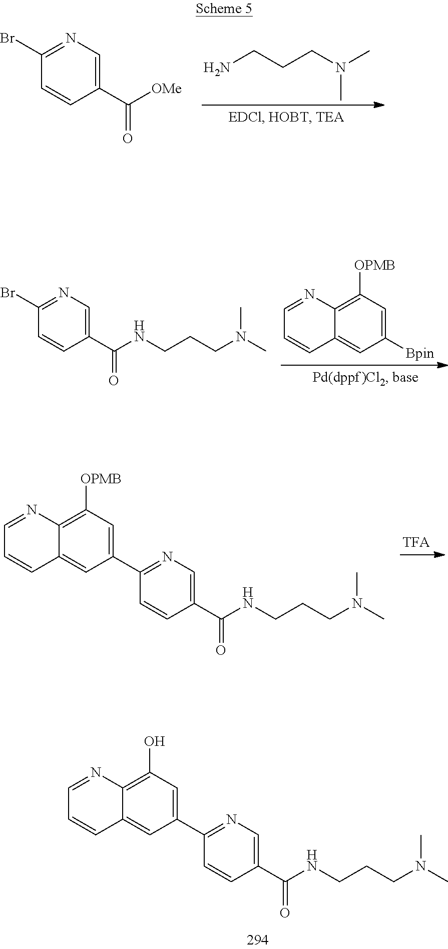

Compounds 264, 269, 271, 272, 274, 275, 276, 277, 281, 282, 283, 295, 296, 310, 311, 314, 315, 316, 317, 318, 320, 322, 323, and 325 can be prepared via a similar procedure to the one shown in Scheme 5.

TABLE 7
Characterization of selected compounds

| Compound | | |
|---|---|---|
| No. | LC-MS | ¹H NMR |
| 264 | [M + H]⁺ = | ¹H NMR (CD₃OD, 400 MHz) δ 8.94 (d, J = 5.2 Hz, 2H), 8.23 (s, 1H), |
| | 368.2 | 8.12 (s, 1H), 7.95 (d, J = 13.2 Hz 2H), 7.70 (s, 1H), 4.31-4.25 (m, |
| | | 1H), 3.60-3.52 (m, 1H), 2.86-2.84 (m, 8H), 2.51-2.45 (m, 2H) |
| 269 | [M + H]⁺ = | ¹H NMR (DMSO-d₆, 400 MHz) δ 8.84 (d, J = 6.0 Hz, 1H), 7.90-7.88 |
| | 339.2 | (m, 1H), 7.86 (s, 1H), 7.63 (s, 1H), 7.54 (s, 1H), 7.28 (d, J = 1.2 Hz, |
| | | 1H), 3.49 (t, J = 6.4 Hz, 2H), 3.21 (t, J = 7.2 Hz, 2H), 2.93 (s, 6H), |
| | | 2.08-2.01 (m, 2H) |
| 271 | [M + H]⁺ = | ¹H NMR (CD₃OD, 400 MHz) δ 9.30 (s, 2H), 8.87 (d, J = 2.8 Hz, 1H), |
| | 352.3 | 8.39 (d, J = 8.4 Hz, 1H), 7.82 (s, 1H), 7.58 (dd, J = 4.0, 8.0 Hz, 1H), |
| | | 7.49 (s, 1H), 3.52 (t, J = 6.8 Hz, 2H), 2.52-2.47 (m, 2H), 2.31 (s, 6H), |
| | | 1.91-1.88 (m, 2H) |
| 272 | [M + H]⁺ = | ¹H NMR (DMSO-d₆, 400 MHz) δ 10.40-10.04 (m, 1H), 8.98 (t, J = 5.6 |
| | 357.0 | Hz, 1H), 8.87 (dd, J = 4.4, 1.6 Hz, 1H), 8.51 (s, 1H), 8.38 (dd, J = 8.4, |
| | | 1.6 Hz, 1H), 8.18 (s, 1H), 7.83 (d, J = 1.6 Hz, 1H), 7.61 (dd, J = 8.4, |
| | | 4.4 Hz, 1H), 7.50 (d, J = 1.6 Hz, 1H), 3.33 (br s, 2H), 2.37-2.33 (m, |
| | | 2H), 2.20 (s, 6H), 1.70 (q, J = 7.2 Hz, 2H) |
| 274 | [M + H]⁺ = | ¹H NMR (DMSO-d₆, 400 MHz) δ 10.99-10.04 (m, 1H), 9.67-9.48 (m, |
| | 356.9 | 1H), 9.00-8.89 (m, 2H), 8.57-8.47 (m, 2H), 8.13 (d, J = 1.6 Hz, 1H), |
| | | 7.71-7.62 (m, 2H), 3.35 (q, J = 6.4 Hz, 2H), 3.15-3.06 (m, 2H), 2.79 |
| | | (d, J = 4.8 Hz, 6H), 1.96-1.85 (m, 2H) |
| 275 | [M + H]⁺ = | ¹H NMR (DMSO-d₆, 400 MHz) δ 9.37 (s, 1H), 8.85 (dd, J = 1.2, 4.4 |
| | 340.2 | Hz, 1H), 8.66 (t, J = 6.4 Hz, 1H), 8.46 (d, J = 8.4 Hz, 1H), 7.97 (d, J = |
| | | 1.2 Hz, 1H), 7.92 (s, 1H), 7.76-7.63 (m, 2H), 3.40-3.36 (m, J = 6.5 |
| | | Hz, 2H), 3.15-3.10 (m, 6H), 2.80 (d, J = 4.8 Hz, 6H), 1.97-1.87 (m, 2H) |
| 276 | [M + H]⁺ = | ¹H NMR (CD₃OD, 400 MHz) |
| | 352.0 | δ 9.32 (s, 2H), 8.88 (d, J = 4.6, 1.6 Hz, 1 H) 8.53 (s, 1H), 8.43 (dd, J = |
| | | 8.4, 1.6 Hz, 1H), 8.25 (d, J = 1.4 Hz, 1H), 7.94 (d, J = 2.0 Hz, 1H), |
| | | 7.59 (dd, J = 8.4, 4 Hz, 1H), 3.56 (t, J = 6.8 Hz, 2H), 3.02-2.94 (m, |
| | | 2H), 2.73 (s, 6H), 2.08-1.94 (m, 2H) |
| 277 | [M + H]⁺ = | ¹H NMR (DMSO-d₆, 400 MHz) δ 13.17 (br, 1H), 10.14 (br, 1H), 9.31 |
| | 340.0 | (br, 1H), 8.87 (dd, J = 4.0, 1.6 Hz, 1 H), 8.34 (dd, J = 8.4, 1.6 Hz, 1 |
| | | H), 8.27 (br s, 1H), 8.02 (s, 1H) 7.83-7.72 (m, 2 H), 7.61 (dd, J = 8.4, |
| | | 4.0 Hz, 1H) 3.35-3.30 (m, 2H) 3.12-3.08 (m, 2 H) 2.79 (d, J = 4.0 Hz, |
| | | 6 H) 1.97-1.80 (m, 2 H) |
| 281 | [M + H]⁺ = | 1H NMR (CD₃OD, 400 MHz) δ 8.78 (dd, J = 4.0, 1.2 Hz, 1H), 8.29 |
| | 356.3 | (dd, J = 8.4, 1.6 Hz, 1H), 8.03 (d, J = 1.2 Hz, 1H), 7.87 (d, J = 1.2 Hz, |
| | | 1H), 7.65 (d, J = 1.6 Hz, 1H), 7.52 (dd, J = 8.4, 4.4 Hz, 1H), 7.41 (d, J = |
| | | 2.0 Hz, 1H), 3.42 (t, J = 6.8 Hz, 2H), 2.48-2.44 (m, 2H), 1.80-1.87 |
| | | (m, 2 H) |
| 282 | [M + H]⁺ = | ¹H NMR (CD₃OD, 400 MHz) δ 9.27 (s, 2 H) 8.88 (dd, J = 4.19, 1.44 |
| | 352.1 | Hz, 1 H) 8.61 (s, 1 H) 8.51 (d, J = 1.25 Hz, 1 H) 8.42 (dd, J = 8.38, 1.38 |
| | | Hz, 1 H) 8.21 (d, J = 1.38 Hz, 1 H) 7.58 (dd, J = 8.38, 4.25 Hz, 1 H) |
| | | 3.54 (t, J = 6.63 Hz, 2 H) 3.08 (br t, J = 7.19 Hz, 2 H) 2.80 (s, 6 H) 1.99- |
| | | 2.08 (m, 2 H) |
| 283 | [M + H]⁺ = | ¹H NMR (DMSO-d₆, 400 MHz) δ 9.03-8.85 (m, 2H), 8.30 (s, 1H), |
| | 340.1 | 7.98-7.88 (m, 2H), 7.67 (s, 1H), 7.60 (d, J = 1.2 Hz, 1H), 3.52 (t, J = |
| | | 6.4 Hz, 2H), 3.28-3.20 (m, 2H), 2.94 (s, 6H), 2.14-2.02 (m, 2H) |
| 294 | [M + H]⁺ = | ¹H NMR (CD₃OD, 400 MHz) δ 9.18 (d, J = 2.0 Hz, 1H), 9.01 (dd, J = |
| | 351.1 | 1.2, 5.2 Hz, 1H), 8.93 (d, J = 8.0 Hz, 1H), 8.40 (dd, J = 2.4, 8.4 Hz, |
| | | 1H), 8.36 (d, J = 1.6 Hz, 1H), 8.23 (d, J = 8.4 Hz, 1H), 8.14 (d, J = 1.6 |
| | | Hz, 1H), 7.93 (dd, J = 5.2, 8.4 Hz, 1H), 3.57 (t, J = 6.4 Hz, 2H), 3.26 |
| | | (t, J = 3.2 Hz, 2H), 2.15-2.05 (m, 2H) |
| 295 | [M + H]⁺ = | ¹H NMR (DMSO-d₆, 400 MHz) δ 9.39 (s, 1H), 9.14-9.04 (m, 2H), |
| | 351.2 | 8.93 (dd, J = 1.6, 6.0 Hz, 1H), 8.51 (dd, J = 1.2, 8.4 Hz, 1H), 8.40 |
| | | (dd, J = 2.0, 10.4 Hz, 1H), 8.16 (d, J = 8.0 Hz, 1H), 7.92 (d, J = 1.6 |
| | | Hz, 1H), 7.68 (dd, J = 4.4, 8.4 Hz, 1H), 7.56 (d, J = 1.6 Hz, 1H), 3.41 |
| | | (q, J = 6.4 Hz, 2H), 3.20-3.05 (m, 2H), 2.79 (d, J = 4.8 Hz, 6H), 1.98- |
| | | 1.88 (m, 2H) |
| 296 | [M + H]⁺ = | ¹H NMR (CD₃OD, 400 MHz) δ 8.78 (d, J = 3.2 Hz, 1H), 8.53 (s, 1H), |
| | 356.3 | 8.29 (d, J = 7.2 Hz, 1H), 7.74-7.67 (m, 2H), 7.56-7.48 (m, 2H), 7.41 |
| | | (d, J = 1.6 Hz, 1H), 3.48 (t, J = 6.8 Hz, 2H), 3.09-3.01 (m, 2H), 2.79 |
| | | (s, 6H), 2.07-1.97 (m, 2H) |
| 310 | [M + H]⁺ = | ¹H NMR (CD₃OD, 500 MHz) δ 8.85 (dd, J = 4.5, 1.5 Hz, 1H), 8.35 |
| | 402.15 | (dd, J = 8.5, 1.5 Hz, 1H), 7.62 (t, J = 7.5 Hz, 1H), 7.59-7.55 (m, 2H), |
| | | 7.39 (d, J = 7.0 Hz, 1H), 7.29 (s, 1H), 3.45 (t, J = 7.0 Hz, 2H), 2.56 (t, |
| | | J = 8.0 Hz, 2H), 2.36 (s, 6H), 1.90-1.84 (m, 2H) |
| 311 | [M + H]⁺ = | ¹H NMR (CD₃OD, 500 MHz) δ 8.79 (dd, J = 4.0, 1.5 Hz, 1H), 8.32 |
| | 364.2 | (dd, J = 8.5, 2.0 Hz, 1H), 7.74-7.71 (m, 2H), 7.64 (d, J = 1.5 Hz, 1H), |
| | | 7.52 (dd, J = 8.0, 4.0 Hz, 1H), 7.41 (d, J = 1.5 Hz, 1H), 7.39 (d, J = |
| | | 8.0 Hz, 1H), 3.43 (t, J = 6.5 Hz, 2H), 2.50 (t, J = 7.5 Hz, 2H), 2.47 (s, |
| | | 3H), 2.32 (s, 6H), 1.88-1.82 (m, 2H) |
| 314 | [M + H]⁺ = | ¹H NMR (CD₃OD, 500 MHz) δ 8.84 (dd, J = 4.0, 1.5 Hz, 1H), 8.36 |
| | 386.15 | (dd, J = 8.0, 1.5 Hz, 1H), 7.71 (d, J = 2.0 Hz, 1H), 7.56 (dd, J = 8.0 |
| | | Hz, 4.0 Hz, 1H), 7.50 (d, J = 9.0 Hz, 2H), 7.39 (d, J = 1.5 Hz, 1H), |
| | | 3.53 (t, J = 7.0 Hz, 2H), 3.18 (t, J = 8.0 Hz, 2H), 2.89 (s, 6H), 2.08- |
| | | 2.02 (m, 2H) |
| 315 | [M + H]⁺ = | ¹H NMR (CD₃OD, 500 MHz) δ 8.84 (dd, J = 4.0, 1.5 Hz, 1H), 8.34 (d, |
| | 402.15 | J = 8.0 Hz, 1H), 7.71 (d, J = 6.5 Hz, 1H), 7.58 (s, 1H), 7.55 (dd, J = |
| | | 8.0, 4.0 Hz, 1H), 7.38 (d, J = 10.0 Hz, 1H), 7.29 (s, 1H), 3.44 (t, J = |
| | | 7.0 Hz, 2H), 2.51 (t, J = 7.5 Hz, 2H), 2.32 (s, 6H), 1.88-1.82 (m, 2H) |
| 316 | [M + H]⁺ = | ¹H NMR (CD₃OD, 500 MHz) δ 8.85 (dd, J = 4.5, 1.5 Hz, 1H), 8.35 |
| | 386.15 | (dd, J = 8.5, 1.5 Hz, 1H), 7.62 (s, 1H), 7.60 (dd, J = 10.5, 6.0 Hz, 1H), |
| | | 7.56 (dd, J = 8.5, 4.5 Hz, 1H), 7.51 (dd, J = 11.0, 6.0 Hz, 1H), 7.33 (s, |
| | | 1H), 3.46 (t, J = 6.5 Hz, 2H), 2.47 (t, J = 7.5 Hz, 2H), 2.29 (s, 6H), |
| | | 1.87-1.81 (m, 2H) |
| 317 | [M + H]⁺ = | ¹H NMR (CD₃OD, 500 MHz) δ 8.83 (d, J = 1.5 Hz, 1H), 8.31 (dd, J = |
| | 384.20 | 8.5, 1.5 Hz, 1H), 8.00 (d, J = 1.5 Hz, 1H), 7.84 (dd, J = 8.0, 2.0 Hz, |
| | | 1H), 7.57 (d, J = 8.0 Hz, 1H), 7.54 (dd, J = 8.0, 4.0 Hz, 1H), 7.41 (d, J = |
| | | 2.0 Hz, 1H), 7.18 (d, J = 1.5 Hz, 1H), 3.44 (t, J = 7.0 Hz, 2H), 2.44 |
| | | (t, J = 7.5 Hz, 2H), 2.28 (s, 6H), 1.87-1.80 (m, 2H) |
| 318 | [M + H]⁺ = | ¹H NMR (CD₃OD, 500 MHz) δ 8.83 (dd, J = 9.5, 2.0 Hz, 1H), 8.36 |
| | 418.10 | (dd, J = 8.0, 1.5 Hz, 1H), 7.82 (s, 2H), 7.69 (d, J = 2.0 Hz, 1H), 7.55 |
| | | (dd, J = 8.5, 4.5 Hz, 1H), 7.37 (d, J = 2.0 Hz, 1H), 3.45 (t, J = 6.5 Hz, |
| | | 2H), 2.52 (t, J = 7.5 Hz, 2H), 2.30 (s, 6H), 1.89-1.83 (m, 2H) |
| 320 | [M + H]⁺ = | ¹H NMR (CD₃OD, 500 MHz) δ 9.03 (dd, J = 5.5, 1.5 Hz, 1H), 9.00 |
| | 364.20 | (dd, J = 8.5, 1.5 Hz, 1H), 8.00 (dd, J = 8.5, 5.0 Hz, 1H), 7.86 (d, J = |
| | | 1.5 Hz, 1H), 7.80 (dd, J = 8.0, 2.0 Hz, 1H), 7.67 (d, J = 1.5 Hz, 1H), |
| | | 7.44 (d, J = 8.0 Hz, 1H), 7.38 (d, J = 1.5 Hz, 1H), 3.53 (t, J = 6.5 Hz, |
| | | 2H), 3.23 (t, J = 7.5 Hz, 2H), 2.93 (s, 6H), 2.38 (s, 3H), 2.10-2.04 (m, 2H) |
| 322 | [M + H]⁺ = | ¹H NMR (CD₃OD, 500 MHz) δ 8.86 (d, J = 4.0 Hz, 1H), 8.54 (s, |
| | 418.10 | 0.5H), 8.33 (d, J = 8.5 Hz, 1H), 7.67 (s, 1H), 7.61 (s, 1H), 7.56 (dd, J = |
| | | 8.5, 4.5 Hz, 1H), 7.43 (d, J = 1.5 Hz, 1H), 7.17 (s, 1H), 3.47 (t, J = |
| | | 6.5 Hz, 2H), 2.85 (t, J = 8.0 Hz, 2H), 2.60 (s, 6H), 1.98-1.92 (m, 2H) |
| 323 | [M + H]⁺ = | ¹H NMR (CD₃OD, 500 MHz) δ 9.00 (d, J = 5.0 Hz, 1H), 8.84 (d, J = |
| | 428.15 | 8.5 Hz, 1H), 8.26 (s, 1H), 7.96 (d, J = 7.5 Hz, 1H), 7.90 (dd, J = 8.5, |
| | | 5.0 Hz, 1H), 7.65 (s, 1H), 7.61 (d, J = 8.0 Hz, 1H), 7.38 (s, 1H), 3.53 |
| | | (t, J = 6.5 Hz, 2H), 3.23 (t, J = 8.0 Hz, 2H), 2.94 (s, 6H), 2.10-2.03 |
| | | (m, 2H) |
| 325 | [M + H]⁺ = | ¹H NMR (CD₃OD, 500 MHz) δ 9.17 (d, J = 8.0 Hz, 1H), 9.09 (d, J = |
| | 368.15 | 5.0 Hz, 1H), 8.14-8.08 (m, 1H), 8.01 (s, 1H), 7.90 (d, J = 8.0 Hz, 1H), |
| | | 7.85-7.80 (m, 2H), 7.70 (s, 1H), 3.56 (t, J = 6.5 Hz, 2H), 3.26 (t, J = |
| | | 7.5 Hz, 2H), 2.96 (s, 6H), 2.13-2.07 (m, 2H) |

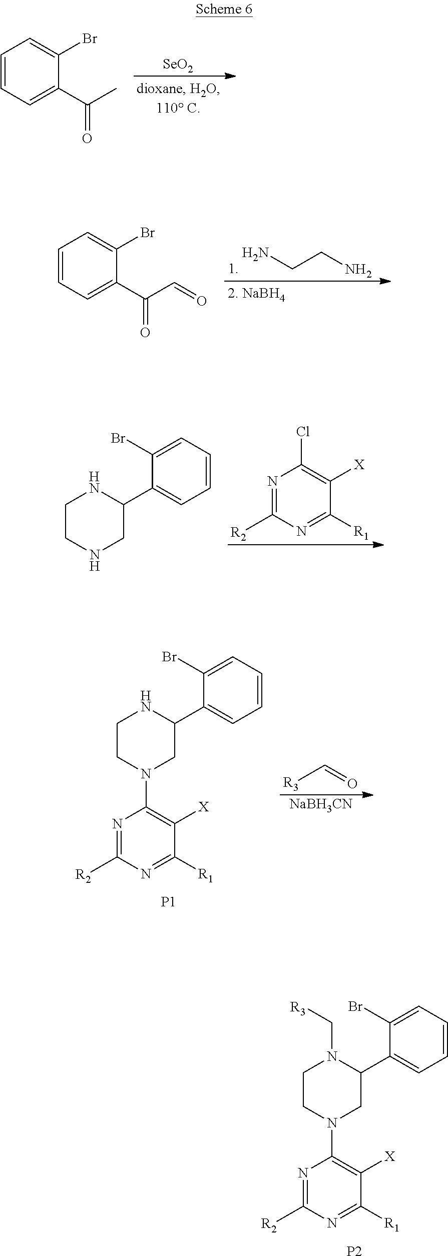

Compounds 11, 12, 13, 14, 34, 35, 42, 43, 44, 45, 54, 56, 57, 75, 76, 99, 100, 101, 110, 111, 112, 121, 122, 128, 129, 143, 144, 154, 155, 160, 161, 165, 167, 168, 169, 170, 171, 172, 173, 174, 175, 176, 177, and 183 can be prepared via a similar procedure to the one shown in Scheme 6.

TABLE 8
Characterization of selected compounds

| Compound | | | |
|---|---|---|---|
| No. | LC-MS | ¹H NMR | Chiral Separation Data |
| 11 | [M + H]⁺ = | ¹H NMR (DMSO-d₆, 400 MHz) δ | SFC peak 2 (Retention |
| | 417.1 | 8.02 (s, 1H), 7.89 (d, J = 8.0 Hz, | time 1.868 min, DAICEL |
| | | 1H), 7.62 (dd, J = 2.0, 8.4 Hz, 1H), | CHIRALPAK AD |
| | | 5.72 (s, 2H), 5.46 (s, 2H), 5.11 (s, | (250 mm*30 mm, 10 um)) |
| | | 1H), 4.19 (d, J = 12.0 Hz, 1H), | |
| | | 4.12 (d, J = 8.4 Hz, 1H), 4.02 (dd, | |
| | | J = 2.8, 10.4 Hz, 1H), 3.06-3.03 | |
| | | (m, 2H), 2.77-2.74 (m, 2H), 2.39- | |
| | | 2.33 (m, 1H) | |
| 12 | [M + H]⁺ = | | SFC peak 1 (Retention |
| | 417.1 | | time 1.582 min, DAICEL |
| | | | CHIRALPAK AD |
| | | | (250 mm*30 mm, 10 um)) |
| 13 | [M + H]⁺ = | ¹H NMR (DMSO-d₆, 400 MHz) δ | SFC peak 2 (Retention |
| | 444.2 | 8.02 (d, J = 2.0 Hz, 1H), 7.90 (d, J = | time 1.354 min, DAICEL |
| | | 8.4 Hz, 1H), 7.63 (dd, J = 2.0, | CHIRALPAK AS |
| | | 8.4 Hz, 1H), 5.97-5.94 (m, 3H), | (250 mm*30 mm, 10 um)) |
| | | 4.41-4.38 (m, 2H), 4.01 (dd, J = | |
| | | 2.8, 10.4 Hz, 1H), 3.08-3.06 (m, | |
| | | 2H), 2.90-2.84 (m, 1H), 2.77-2.74 | |
| | | (m, 1H), 2.62-2.58 (m, 1H), 2.49- | |
| | | 2.48 (m, 1H), 1.13 (d, J = 6.8 Hz, 6H) | |
| 14 | [M + H]⁺ = | | SFC peak 1 (Retention |
| | 444.2 | | time 1.106 min, DAICEL |
| | | | CHIRALPAK AS |
| | | | (250 mm*30 mm, 10 um)) |
| 34 | [M + H]⁺ = | ¹H NMR (DMSO-d₆, 400 MHz) δ | SFC peak 1 (Retention |
| | 358.1 | 12.80 (br, 1H), 8.06 (s, 1H), 7.95 | time 0.930 min, DAICEL |
| | | (d, J = 6.0 Hz, 1H), 7.76 (dd, J = | CHIRALPAK |
| | | 1.6, 8.0 Hz, 1H), 7.63 (dd, J = 1.2, | AD(250 mm*30 mm, 10 um)) |
| | | 8.0 Hz, 1H), 7.47-7.37 (m, 1H), | |
| | | 7.25 (dt, J = 2.0, 8.0 Hz, 1H), 6.53 | |
| | | (d, J = 6.0 Hz, 1H), 4.94-4.75 (m, | |
| | | 2H), 4.17 (dd, J = 2.4, 10.4 Hz, | |
| | | 1H), 3.13-3.03 (m, 2H), 3.02-2.92 | |
| | | (m, 2H), 2.71 (dd, J = 10.4, 12.4 | |
| | | Hz, 1H) | |
| 35 | [M + H]⁺ = | | SFC peak 2 (Retention |
| | 358.1 | | time 1.303 min, DAICEL |
| | | | CHIRALPAK |
| | | | AD(250 mm*30 mm, 10 um)) |
| 42 | [M + H]⁺ = | ¹H NMR (CD₃OD, 400 MHz) δ | SFC peak 2 (Retention |
| | 373.0 | 7.70 (dd, J = 1.2, 7.6 Hz, 1H), | time 2.459 min, |
| | | 7.66-7.61 (m, 1H), 7.44 (t, J = 7.6 | Phenomenex-Cellulose-2 |
| | | Hz, 1H), 7.29-7.21 (m, 2H), 6.18 | (250 mm*30 mm, 10 um)) |
| | | (d, J = 3.2 Hz, 1H), 4.67-4.61 (m, | |
| | | 1H), 4.56-4.49 (m, 1H), 4.34-4.29 | |
| | | (m, 1H), 3.29-3.20 (m, 2H), 3.18- | |
| | | 3.09 (m, 1H), 3.01 (dd, J = 10.8, | |
| | | 12.8 Hz, 1H) | |
| 43 | [M + H]⁺ = | | SFC peak 1 (Retention |
| | 373.0 | | time 1.097 min, |
| | | | Phenomenex-Cellulose-2 |
| | | | (250 mm*30 mm, 10 um)) |
| 44 | [M + H]⁺ = | | SFC peak 2 (Retention |
| | 432.9 | | time 2.015 min, DAICEL |
| | | | CHIRALCEL OD |
| | | | (250 mm*30 mm, 10 um)) |
| 45 | M + H]⁺ = | ¹H NMR (CD₃CN, 400 MHz) δ 7.83 | SFC peak 1 (Retention |
| | 432.9 | (d, J = 7.6 Hz, 2H), 7.70 (d, J = 8.0 | time 1.894 min, DAICEL |
| | | Hz, 1H), 7.48 (t, J = 7.2 Hz, 1H), | CHIRALCEL OD |
| | | 7.34-7.26 (m, 1H), 6.38-6.19 (m, | (250 mm*30 mm, 10 um)) |
| | | 2H), 5.51 (s, 1H), 4.14-4.05 (m, | |
| | | 1H), 3.71-3.67 (m, 1H), 3.55-3.46 | |
| | | (m, 2H), 3.43-3.29 (m, 1H), 3.18- | |
| | | 3.04 (m, 1H), 2.73-2.68 (m, 1H), | |
| | | 2.65-2.60 (m, 1H), 2.56-2.51 (m, | |
| | | 2H), 2.40-2.35 (m, 2H), 0.88-0.80 | |
| | | (m, 2H), 0.64-0.54 (m, 2H) | |
| 54 | [M + H]⁺ = | ¹H NMR (DMSO-d₆, 400 MHz) δ | |
| | 339.2 | 7.98 (d, J = 7.8 Hz, 1H), 7.74-7.65 | |
| | | (m, 2H), 7.54-7.46 (m, 1H), 5.66 | |
| | | (m, 2H), 5.42 (m, 2H), 5.01 (m, | |
| | | 1H), 4.13-3.99 (m, 2H), 3.92 (d, J = | |
| | | 9.6 Hz, 1H), 3.03-3.01 (m, 1H), | |
| | | 2.89 (m, 1H), 2.78-2.68 (m, 2H) | |
| 56 | [M + H]⁺ = | | SFC peak 2 (Retention |
| | 373.1 | | time 0.982 min, DAICEL |
| | | | CHIRALCEL AY-H |
| | | | (250 mm*30 mm, 10 um)) |
| 57 | [M + H]⁺ = | ¹H NMR (DMSO-d₆, 400 MHz) δ | SFC peak 1 (Retention |
| | 373.1 | 10.84 (s, 1H), 7.76 (dd, J = 1.6, | time 0.727 min, DAICEL |
| | | 7.6 Hz, 1H), 7.63 (dd, J = 1.2, 7.6 | CHIRALCEL AY-H |
| | | Hz, 1H), 7.50-7.34 (m, 1H), 7.25 | (250 mm*30 mm, 10 um)) |
| | | (dt, J = 1.6, 7.6 Hz, 1H), 6.74 (dd, | |
| | | J = 2.4, 3.6 Hz, 1H), 6.32 (dd, J = | |
| | | 2.0, 3.6 Hz, 1H), 5.51 (s, 2 H), | |
| | | 4.62 (d, J = 11.6 Hz, 1H), 4.54 (d, | |
| | | J = 12.4 Hz, 1H), 4.07 (dd, J = 2.8, | |
| | | 10.4 Hz, 1H), 3.11 (d, J = 11.6 Hz, | |
| | | 1H), 2.99 (dt, J = 2.7, 12.0 Hz, | |
| | | 1H), 2.91-2.82 (m, 1H), 2.62 (dd, J = | |
| | | 10.4, 12.4 Hz, 1H) | |
| 75 | [M + H]⁺ = | ¹H NMR (DMSO-d₆, 400 MHz) δ | SFC peak 2 (Retention |
| | 375.9 | 7.75-7.70 (m, 1H), 7.62 (d, J = 8.0 | time 1.867 min, DAICEL |
| | | Hz, 1H), 7.41 (t, J = 7.6 Hz, 1H), | CHIRALCEL |
| | | 7.27-7.21 (m, 1H), 6.14-6.01 (m, | OD(250 mm*30 mm, 10 um)) |
| | | 2H), 5.47 (s, 1H), 4.43-4.27 (m, | |
| | | 1H), 4.19-4.06 (m, 1H), 3.99-3.90 | |
| | | (m, 1H), 3.08-3.01 (m, 1H), 2.85- | |
| | | 2.71-(m, 2H), 2.66-2.63 (m, 1H), | |
| | | 2.40-2.35 (m, 1H), 1.15-1.10 (m, 6H) | |
| 76 | [M + H]⁺ = | ¹H NMR (DMSO-d₆, 400 MHz) δ | SFC peak 1 (Retention |
| | 375.9 | 7.75-7.70 (m, 1H), 7.62 (d, J = 8.0 | time 1.759 min, DAICEL |
| | | Hz, 1H), 7.41 (t, J = 7.6 Hz, 1H), | CHIRALCEL |
| | | 7.27-7.21 (m, 1H), 6.14-6.01 (m, | OD(250 mm*30 mm, 10 um)) |
| | | 2H), 5.47 (s, 1H), 4.43-4.27 (m, | |
| | | 1H), 4.19-4.06 (m, 1H), 3.99-3.90 | |
| | | (m, 1H), 3.08-3.01 (m, 1H), 2.85- | |
| | | 2.71-(m, 2H), 2.66-2.63 (m, 1H), | |
| | | 2.40-2.35 (m, 1H), 1.15-1.10 (m, 6H) | |
| 99 | [M + H]⁺ = | | SFC peak 2 (Retention |
| | 417.0 | | time 1.703 min, |
| | | | Phenomenex-Cellulose-2 |
| | | | (250 mm*50 mm, 10 um))) |
| 100 | [M + H]⁺ = | ¹H NMR (DMSO-d₆, 400 MHz) δ | SFC peak 1 (Retention |
| | 417.0 | 7.63 (d, J = 7.6 Hz, 1H), 7.58 (dd, | time 1.112 min, |
| | | J = 7.6, 1.2 Hz, 1H), 7.43 (t, J = | Phenomenex-Cellulose-2 |
| | | 7.6 Hz, 1H), 7.28-7.21 (m, 1H), | (250 mm*50 mm, 10 um)) |
| | | 6.36-6.26 (m, 1H), 5.59 (s, 2H), | |
| | | 5.11 (s, 1H) 4.22-4.11 (m, 1H), | |
| | | 4.09-3.97 (m, 1H), 3.41 (dd, J = | |
| | | 10.4, 2.8 Hz, 1H), 3.06-2.95 (m, | |
| | | 3H), 2.92-2.82 (m, 1H), 2.49-2.43 | |
| | | (m, 1H), 2.25-2.16 (m, 1H), 1.97 | |
| | | (s, 3H), 1.01-0.90 (m, 1H), 0.43- | |
| | | 0.35 (m, 2H), 0.19 0.12 (m, 2H) | |
| 101 | [M + H]⁺ = | ¹H NMR (DMSO-d₆, 400 MHz) δ | |
| | 403.1 | 8.14 (s, 1H), 7.65-7.58 (m, 2H), | |
| | | 7.44 (t, J = 7.2 Hz, 1H), 7.27-7.23 | |
| | | (m, 1H), 6.48 (s, 1H), 5.59 (s, 2H), | |
| | | 5.28 (s, 1H), 4.26-4.23 (m, 1H), | |
| | | 4.09-4.06 (m, 1H), 3.44-3.40 (m, | |
| | | 1H), 3.00-2.90 (m, 2H), 2.59-2.56 | |
| | | (m, 1H), 2.42 (m, 1H), 2.25-2.19 | |
| | | (m, 1H), 1.97 (s, 3H), 0.65-0.61 | |
| | | (m, 2H), 0.41-0.38 (m, 2H) | |
| 110 | [M + H]⁺ = | ¹H NMR (DMSO-d6, 400 MHz) δ | |
| | 299.1 | 7.26-7.41 (m, 5H), 6.09 (d, J = 4.8 | |
| | | Hz, 1H), 5.51 (s, 2H), 5.00 (s, 1H) | |
| | | 4.08 (d, J = 12.0 Hz, 2H), 2.99- | |
| | | 2.84 (m, 3H), 2.67 (d, J = 4.8 Hz, | |
| | | 3H), 2.58 (dd, J = 12.8, 10.8 Hz, | |
| | | 1H), 2.23-2.11 (m, 1 H), 1.96 (s, 3 H) | |
| 111 | [M + H]⁺ = | | SFC peak 2 (Retention |
| | 402.7 | | time 1.936 min, DAICEL |
| | | | CHIRALCEL OD |
| | | | (250 mm*30 mm, 10 um)) |
| 112 | [M + H]⁺ = | ¹H NMR (CD₃OD, 400 MHz) δ | SFC peak 1 (Retention |
| | 402.7 | 8.49 (s, 1H), 7.65 (t, J = 8.8 Hz, | time 1.060 min, DAICEL |
| | | 2H), 7.42 (t, J = 7.6 Hz, 1H), 7.28- | CHIRALCEL OD |
| | | 7.22 (m, 1H), 4.93-4.89 (m, 1H), | (250 mm*30 mm, 10 um)) |
| | | 4.43 (t, J = 12.4 Hz, 2H), 4.22 (dd, | |
| | | J = 10.4, 2.4 Hz, 1H), 3.20 (d, J = | |
| | | 12.0 Hz, 1H), 3.13-3.04 (m, 3H), | |
| | | 3.00-2.92 (m, 1H), 2.82 (dd, J = | |
| | | 12.8, 10.8 Hz, 1H), 1.14-1.06 (m, | |
| | | 1H), 0.61-0.54 (m, 2H) | |
| 121 | [M + H]⁺ = | | SFC peak 2 (Retention |
| | 388.6 | | time 1.820 min, DAICEL |
| | | | CHIRALPAK AD |
| | | | (250 mm*30 mm, 10 um)) |
| 122 | [M + H]⁺ = | ¹H NMR (DMSO-d₆, 400 MHz) δ | SFC peak 1 (Retention |
| | 388.6 | 7.72 (dd, J = 1.6, 7.2 Hz, 1H), 7.61 | time 1.081 min, DAICEL |
| | | (dd, J = 7.6, 1.0 Hz, 1H), 7.40 (t, J = | CHIRALPAK AD |
| | | 7.6 Hz, 1H), 7.23 | (250 mm*30 mm, 10 um)) |
| | | (t, J = 7.6 Hz, 1H), 6.38 (d, J = 2.0 | |
| | | Hz, 1H), 5.46 (s, 2 H), 5.27 (s, 1 | |
| | | H), 4.28-4.12 (m, 2H), 3.96 (dd, J = | |
| | | 2.8, 10.0 Hz, 1H), 3.05-3.00 (m, | |
| | | 1H), 2.75 (d, J = 8.4 Hz, 2H), 2.43- | |
| | | 2.38 (m, 2H), 0.65-0.58 (m, 2H), | |
| | | 0.42-0.36 (m, 2H) | |
| 128 | [M + H]⁺ = | ¹H NMR (DMSO-d₆, 400 MHz) | SFC peak 2 (Retention |
| | 285.3 | δ 7.43 (d, J = 7.2 Hz, 2H), 7.34 (t, | time 2.128 min, DAICEL |
| | | J = 7.2 Hz, 2H), 7.28 (d, J = 7.2 | CHIRALPAK AY-H |
| | | Hz, 1H) 6.06 (d, J = 4.4 Hz, 1H), | (250 mm*30 mm, 10 um) |
| | | 5.47 (s, 2H), 4.99 (s, 1H), 4.16- | |
| | | 4.13 (m, 1H), 4.03-4.00 (m, 1H), | |
| | | 3.63 (dd, J = 2.8, 10.4 Hz, 1H), | |
| | | 3.04-2.97 (m, 1H), 2.77-2.70 (m, | |
| | | 2H), 2.67-2.66 (m, 4H) | |
| 129 | [M + H]⁺ = | | SFC peak 1 (Retention |
| | 285.3 | | time 1.983 min, DAICEL |
| | | | CHIRALPAK AY-H |
| | | | (250 mm*30 mm, 10 um) |
| 143 | [M + H]⁺ = | ¹H NMR (DMSO-d₆, 400 MHz) | SFC peak 2 (Retention |
| | 362.9 | δ7.65-7.55 (m, 2H), 7.43 (t, J = 7.6 | time 1.033 min, DAICEL |
| | | Hz, 1H), 7.28-7.20 (m, 1H), 5.71 | CHIRALCEL AD |
| | | (s, 2H), 5.49 (s, 2H), 5.08 (s, 1H), | (250 mm*30 mm, 10 um)) |
| | | 4.14-3.95 (m, 2H), 3.41 (dd, J = | |
| | | 10.4, 3.2 Hz, 1H), 2.98 (d, J = 11.6 | |
| | | Hz, 1H), 2.91-2.81 (m, 1H), 2.48- | |
| | | 2.39 (m, 1H), 2.25-2.17 (m, 1H), | |
| | | 1.97 (s, 3H) | |
| 144 | [M + H]⁺ = | ¹H NMR (DMSO-d₆, 400 MHz) δ | SFC peak 1 (Retention |
| | 362.8 | 7.66-7.55 (m, 2H), 7.43 (t, J = 7.2 | time 0.883 min, DAICEL |
| | | Hz, 1H), 7.28-7.20 (m, 1H), 5.71 | CHIRALCEL AD |
| | | (s, 2H). 5.49 (s, 2H), 5.08 (s, 1H), | (250 mm*30 mm, 10 um)) |
| | | 4.16-3.95 (m, 2H), 3.41 (dd, J = | |
| | | 10.4, 2.8 Hz, 1H), 2.98 (d, J = 11.2 | |
| | | Hz, 1H) 2.91-2.82 (m, 1H), 2.48- | |
| | | 2.40 (m, 1H), 2.25-2.17 (m, 1H), | |
| | | 1.97 (s, 3H) | |
| 154 | [M + H]⁺ = | ¹H NMR (DMSO-d₆, 400 MHz) δ | SFC peak 2 (Retention |
| | 377.2 | 7.65-7.58 (m, 2H), 7.44 (t, J = 8.8 | time 2.003 min, DAICEL |
| | | Hz, 1H), 7.25 (t, J = 8.8 Hz, 1H), | CHIRALCEL OD |
| | | 6.13-6.12 (m, 1H), 5.51 (s, 2H), | (250 mm*30 mm, 10 um)) |
| | | 5.05 (s, 1H), 4.19 (d, J = 12.4 Hz, | |
| | | 1H), 4.05 (d, J = 13.2 Hz, 1H), | |
| | | 3.42 (dd, J = 10.4, 3.2 Hz, 1H), | |
| | | 3.00-2.86 (m, 2H), 2.68 (d, J = 4.4 | |
| | | Hz, 3H), 2.49-2.47 (m, 1H), 2.25- | |
| | | 2.19 (m, 1H), 1.92 (s, 3H) | |
| 155 | [M + H]⁺ = | ¹H NMR (CD₃OD, 400 MHz) δ | SFC peak 1 (Retention |
| | 377.1 | 7.65-7.62 (m, 2H), 7.42 (t, J = 7.4 | time 1.775 min, DAICEL |
| | | Hz, 1H), 7.23 (t, J = 8.0 Hz, 1H), | CHIRALCEL OD |
| | | 5.16 (s, 1H), 4.36 (d, J = 13.2 Hz, | (250 mm*30 mm, 10 um)) |
| | | 1H), 4.13 (d, J = 13.2 Hz, 1H), | |
| | | 3.64 (dd, J = 10.4, 3.2 Hz, 1H), | |
| | | 3.12-3.01 (m, 2H), 2.81 (s, 3H), | |
| | | 2.75-2.72 (m, 1H), 2.40-2.37 (m, | |
| | | 1H), 2.10 (s, 3H), | |
| 160 | [M + H]⁺ = | ¹H NMR (CDCl₃, 400 MHz) δ 7.63- | SFC peak 2 (Retention |
| | 389.9 | 7.58 (m, 2H), 7.36 (d, J = 7.6 Hz, | time 1.364 min, DAICEL |
| | | 1H), 7.16 (d, J = 8.0 Hz, 1H), 5.91 | CHIRALCEL AS |
| | | (s, 1H), 4.66 (s, 2H), 4.47 (d, J = | (250 mm*30 mm, 10 um)) |
| | | 14.0 Hz, 1H), 4.18 (d, J = 14.0 Hz, | |
| | | 1H), 3.60-3.56 (m, 1H), 3.12-3.10 | |
| | | (m, 1H), 3.02-3.00 (m, 1H), 2.76- | |
| | | 2.67 (m, 2H), 2.39-2.38 (m, 1H), | |
| | | 2.10 (s, 3H), 1.21 (d, J = 6.8 Hz, 6H) | |
| 161 | [M + H]⁺ = | | SFC peak 1 (Retention |
| | 389.9 | | time 1.198 min, DAICEL |
| | | | CHIRALCEL AS |
| | | | (250 mm*30 mm, 10 um)) |
| 165 | [M + H]⁺ = | ¹H NMR (DMSO-d₆, 400 MHz) δ | |
| | 298.2 | 8.20 (s, 2H), 7.47 (d, J = 7.2 Hz, | |
| | | 2H), 7.41-7.34 (m, 2H), 7.34-7.29 | |
| | | (m, 1H), 6.55 (br, 2H), 6.04 (s, | |
| | | 1H), 4.40 (d, J = 11.2 Hz, 1H), | |
| | | 4.28 (d, J = 11.2 Hz, 1H), 3.76 (dd, | |
| | | J = 2.8, 10.8 Hz, 1H), 3.10 (d, J = | |
| | | 11.6 Hz, 1H), 2.94 (dt, J = 2.4, | |
| | | 12.4 Hz, 1H), 2.85-2.71 (m, 2H), | |
| | | 2.69-2.63 (m, 1H), 1.15 (d, J = 6.8 | |
| | | Hz, 6H) | |
| 167 | [M + H]⁺ = | ¹H NMR (DMSO-d₆, 400 MHz) δ | |
| | 363.1 | 7.71 (dd, J = 7.6, 1.6 Hz, 1H), 7.61 | |
| | | (dd, J = 8.0, 1.2 Hz, 1H), 7.43-7.37 | |
| | | (m, 1H), 7.25-7.23 (m, 1H), 6.10 | |
| | | (q, J = 4.4 Hz, 1H), 5.47 (s, 2H), | |
| | | 5.03 (s, 1H), 4.19-4.11 (m, 2 H), | |
| | | 3.97-3.93 (m, 1H), 3.06-2.99 (m, | |
| | | 1H), 2.92-2.81 (m, 1H), 2.76-2.70 | |
| | | (m, 2H), 2.67 (d, J = 4.4 Hz, 3H), | |
| | | 2.38-2.32 (m, 1H) | |
| 168 | [M + H]⁺ = | ¹H NMR (CD₃OD, 400 MHz) δ | |
| | 326.2 | 8.54 (s, 1H), 7.53 (dd, J = 6.0, 3.2 | |
| | | Hz, 1H), 7.26-7.20 (m, 3H), 6.03 | |
| | | (s, 1H), 4.52-4.36 (m, 2H), 4.00 | |
| | | (dd, J = 10.6, 2.8 Hz, 1H), 3.17- | |
| | | 3.05 (m, 2H), 2.97-2.85 (m, 2H), | |
| | | 2.77 (q, J = 7.2 Hz, 2H), 2.72-2.65 | |
| | | (m, 1H), 1.27-1.21 (m, 9H) | |
| 169 | [M + H]⁺ = | ¹H NMR (DMSO-d₆, 400 MHz) δ | |
| | 394.2 | 7.56 (d, J = 7.6 Hz, 1H), 7.48-7.46 | |
| | | (m, 1H), 7.38-7.27 (m, 1H), 5.93 | |
| | | (s, 1H), 5.89 (s, 2H), 4.41-4.20 (m, | |
| | | 2H), 4.00 (dd, J = 2.4, 10.0 Hz, | |
| | | 1H), 3.05 (d, J = 10.8 Hz, 1H), | |
| | | 2.98-2.96 (m, 1H), 2.88-2.72 (m, | |
| | | 2H), 2.65-2.57 (m, 1H), 2.49-2.44 | |
| | | (m, 1H), 1.13 (d, J = 6.8 Hz, 6H) | |
| 170 | [M + H]⁺ = | ¹H NMR (CD₃OD, 400 MHz) δ | |
| | 412.0 | 7.72-7.58 (m, 2H), 6.03 (s, 1H), | |
| | | 4.55-4.38 (m, 2H), 4.13-4.06 (m, | |
| | | 1H), 3.19-3.12 (m, 1H), 3.07-2.87 | |
| | | (m, 2H), 2.71-2.62 (m, 2H), 1.23 | |
| | | (d, J = 7.6 Hz, 6H) | |
| 171 | [M + H]⁺ = | ¹H NMR (CD₃OD, 400 MHz) δ | |
| | 396.0 | 7.71 (dd, J = 6.4, 8.8 Hz, 1H), 7.46 | |
| | | (dd, J = 2.8, 8.4 Hz, 1H), 7.23-7.21 | |
| | | (m, 1H), 6.03 (s, 1H), 4.56-4.39 | |
| | | (m, 2H), 4.15 (dd, J = 2.8, 10.4 Hz, | |
| | | 1H), 3.20-3.12 (m, 1H), 3.07-2.87 | |
| | | (m, 2H), 2.78-2.62 (m, 2H), 1.23 | |
| | | (d, J = 7.6 Hz, 6H) | |
| 172 | [M + H]⁺ = | ¹H NMR (CD₃OD, 400 MHz) δ | |
| | 348.9 | 7.64-7.60 (m, 2H) 7.41 (t, J = 7.6 | |
| | | Hz, 1H) 7.23-7.19 (m, 1H) 5.29 (s, | |
| | | 1H) 4.34-4.18 (m, 3H) 3.20-3.08 | |
| | | (m, 1H) 3.03-2.85 (m, 2H) 2.68 | |
| | | (dd, J = 12.8, 10.4 Hz, 1H) | |
| 173 | [M + H]⁺ = | ¹H NMR (CD₃OD, 400 MHz) δ | |
| | 394.0 | 7.71 (dd, J = 6.4, 8.8 Hz, 1H), 7.46 | |
| | | (dd, J = 2.8, 8.4 Hz, 1H), 7.23-7.21 | |
| | | (m, 1H), 6.03 (s, 1H), 4.56-4.39 | |
| | | (m, 2H), 4.15 (dd, J = 2.8, 10.4 Hz, | |
| | | 1H), 3.20-3.12 (m, 1H), 3.07-2.87 | |
| | | (m, 2H), 2.78-2.62 (m, 2H), 1.23 | |
| | | (d, J = 7.6 Hz, 6H) | |
| 174 | [M + H]⁺ = | ¹H NMR (CD₃OD, 400 MHz) δ | |
| | 394.0 | 7.65 (dd, J = 5.2, 8.8 Hz, 1H), 7.49 | |
| | | (dd, J = 3.2, 10.0 Hz, 1H), 7.06- | |
| | | 7.01 (m, 1H), 6.04 (s, 1H), 4.53- | |
| | | 4.44 (m, 2H), 4.13 (dd, J = 2.0, 6.4 | |
| | | Hz, 1H), 3.20-3.13 (m, 1H), 3.06- | |
| | | 2.87 (m, 2H), 2.79-2.59 (m, 2H), | |
| | | 1.23 (d, J = 7.6 Hz, 6H) | |
| 175 | [M + H]⁺ = | ¹H NMR (CD₃OD, 400 MHz) δ | |
| | 332.1 | 7.67 (dd, J = 1.6, 7.6 Hz, 1H), 7.46 | |
| | | (dd, J = 1.6, 7.6 Hz, 1H), 7.41-7.28 | |
| | | (m, 2H), 6.03 (s, 1H), 4.47 (dd, J = | |
| | | 14.4, 18.8 Hz, 2H), 4.22 (dd, J = | |
| | | 2.8, 10.8 Hz, 1H), 3.20-3.14 (m, | |
| | | 1H), 3.07-2.90 (m, 2H), 2.77-2.74 | |
| | | (m, 1H), 2.70-2.66 (m, 1H), 1.24 | |
| | | (s, 3H), 1.22 (s, 3H) | |
| 176 | [M + H]⁺ = | ¹H NMR (CD₃OD, 400 MHz) δ | |
| | 424.1 | 7.93 (dd, J = 0.8, 7.6 Hz, 1H), 7.63 | |
| | | (dd, J = 1.6, 8.0 Hz, 1H), 7.50-7.39 | |
| | | (m, 1H), 7.07 (m, 1H), 6.06 (s, | |
| | | 1H), 4.46 (t, J = 10.6 Hz, 2H), 4.03 | |
| | | (dd, J = 2.8, 10.4 Hz, 1H), 3.21- | |
| | | 3.14 (m, 1H), 3.05-2.89 (m, 2H), | |
| | | 2.75-2.64 (m, 2H), 1.24 (s, 3H), | |
| | | 1.22 (s, 3H) | |
| 177 | [M + H]⁺ = | ¹H NMR (DMSO-d₆, 400 MHz) δ | |
| | 312.2 | 8.19 (s, 2H), 7.57 (d, J = 6.8 Hz, | |
| | | 1H), 7.26-7.11 (m, 3H), 6.05 (s, | |
| | | 2H), 5.97 (s, 1H), 4.41-4.20 (m, | |
| | | 2H), 3.82 (dd, J = 2.8, 10.4 Hz, | |
| | | 1H), 3.06 (d, J = 11.2 Hz, 1H), | |
| | | 2.95-2.85 (m, 1H), 2.84-2.74 (m, | |
| | | 1H), 2.65-2.54 (m, 2H), 2.38 (s, | |
| | | 3H), 1.15 (s, 3H), 1.13 (s, 3H) | |
| 183 | [M + H]⁺ = | ¹H NMR (MeOD-d₄, 400 MHz) δ | |
| | 375.9 | 8.34 (br d, J = 1.6 Hz, 2H), 7.66 | |
| | | (ddd, J = 16.4, 8.0, 1.2 Hz, 2H), | |
| | | 7.43 (t, J = 7.2 Hz, 1H), 7.26 (td, J = | |
| | | 7.6, 1.6 Hz, 1H), 6.34 (s, 1H), | |
| | | 4.21 (dd, J = 10.8, 2.8 Hz, 1H), | |
| | | 3.30-3.13 (m, 4H), 3.01-2.81 (m, | |
| | | 3H), 1.31 (d, J = 7.2 Hz, 6H) | |

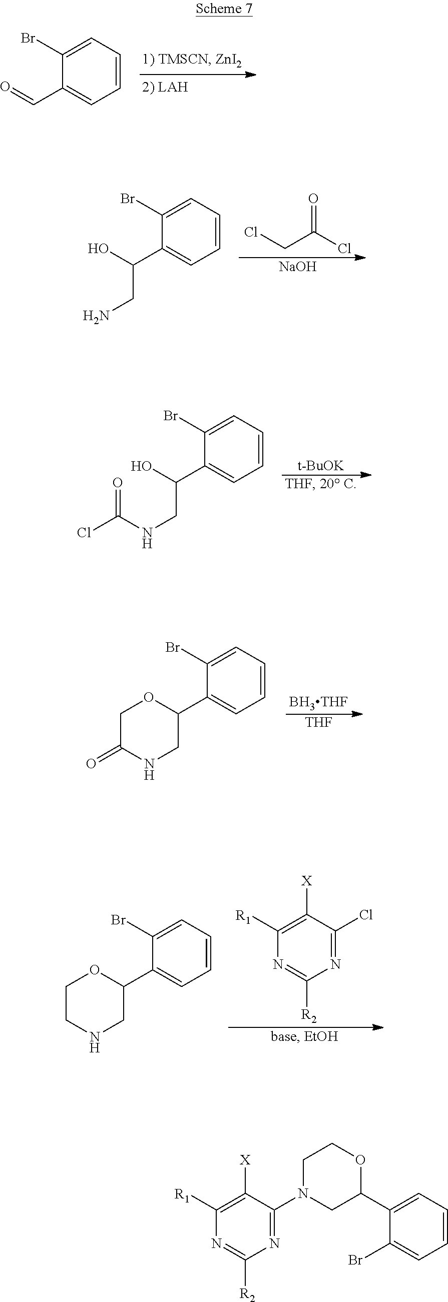

Compounds 29, 39, 40, 46, 47, 48, 49, 72, 73, 95, 96, 108, and 109 can be prepared via a similar procedure to the one shown in Scheme 7.

TABLE 9
Characterization of selected compounds

| Compound | | | |
|---|---|---|---|
| No. | LC-MS | ¹H NMR | Chiral Separation Data |
| 29 | [M + H]⁺ = | ¹H NMR (CD₃CN, 400 MHz) δ 7.84 (d, | |
| | 340.2 | J = 7.6 Hz, 1H), 7.77-7.66 (m, 2H), | |
| | | 7.53 (t, J = 7.6 Hz, 1H), 6.51-6.25 (m, | |
| | | 4H), 5.27 (s, 1H), 4.79 (d, J = 10.4 | |
| | | Hz, 1H), 4.80-4.07 (m, 3H), 3.79-3.69 | |
| | | (m, 1H), 3.29-3.14 (m, 1H), 3.02-2.88 | |
| | | (m, 1H) | |
| 39 | [M + H]⁺ = | | SFC peak 2 (Retention |
| | 374.1 | | time 2.191 min, |
| | | | DAICEL CHIRALCEL |
| | | | OJ |
| | | | (250 mm*30 mm, 10 um)) |
| 40 | [M + H]⁺ = | ¹H NMR (DMSO-d₆, 400 MHz) δ | SFC peak 1 (Retention |
| | 374.1 | 10.89 (br, 1H), 7.70-7.57 (m, 2H), | time 2.050 min, |
| | | 7.46 (t, J = 7.6 Hz, 1H), 7.30 (dt, J = | DAICEL CHIRALCEL |
| | | 1.4, 7.6 Hz, 1H), 6.80-6.74 (m, 1H), | OJ |
| | | 6.35 (dd, J = 1.6, 3.6 Hz, 1H), 5.58 | (250 mm*30 mm, 10 um)) |
| | | (s, 2H), 4.79 (dd, J = 2.4, 10.0 Hz, | |
| | | 1H), 4.71 (d, J = 12.4 Hz, 1H), 4.48 | |
| | | (d, J = 12.4 Hz, 1H), 4.14 (dd, J = | |
| | | 2.0, 11.2 Hz, 1H), 3.80 (dt, J = 2.4, | |
| | | 11.6 Hz, 1H), 3.21-3.14 (m, 1H), 2.79 | |
| | | (dd, J = 10.4, 12.8 Hz, 1H) | |
| 46 | [M + H]⁺ = | ¹H NMR (DMSO-d₆, 400 MHz) δ | SFC peak 2 (Retention |
| | 373.9 | 10.90 (br s, 1H), 7.66-7.62 (m, 2H), | time 2.009 min, |
| | | 7.47 (dt, J = 1.2, 7.6 Hz, 1H), 7.34- | DAICEL CHIRALCEL |
| | | 7.28 (m, 2H), 6.05 (d, J = 3.2 Hz, | AD |
| | | 1H), 5.43-5.32 (m, 2H), 4.81 (dd, J = | (250 mm*30 mm, 10 um)) |
| | | 2.4, 10.4 Hz, 1H), 4.44 (br d, J = 13.2 | |
| | | Hz, 1H), 4.31 (br d, J = 12.8 Hz, 1H), | |
| | | 4.12 (dd, J = 2.4, 11.6 Hz, 1H), 3.89 | |
| | | (dt, J = 2.8, 11.6 Hz, 1H), 3.18 (dt, J = | |
| | | 2.8, 12.4 Hz, 1H), 2.85 (dd, J = | |
| | | 10.8, 13.2 Hz, 1H) | |
| 47 | [M + H]⁺ = | | SFC peak 1 (Retention |
| | 373.9 | | time 1.705 min, |
| | | | DAICEL CHIRALCEL |
| | | | AD |
| | | | (250 mm*30 mm, 10 um)) |
| 48 | [M + H]⁺ = | ¹H NMR (DMSO-d₆, 400 MHz) δ 7.62 | SFC peak 1 (Retention |
| | 367.9 | (dd, J = 0.8, 8.0 Hz, 1H), 7.57 (dd, J = | time 2.250 min, |
| | | 1.2, 8.0 Hz, 1H), 7.47-7.40 (m, 1H), | DAICEL CHIRALCEL |
| | | 7.28 (dt, J = 1.6, 7.6 Hz, 1H), 6.11 (s, | OJ-H |
| | | 2H), 5.55 (s, 2H), 4.76 (dd, J = 2.0, | (250 mm*30 mm, 10 um)) |
| | | 10.4 Hz, 1H), 4.24 (d, J = 13.2 Hz, | |
| | | 1H), 4.08-3.98 (m, 2H), 3.78 (dt, J = | |
| | | 2.4, 12.0 Hz, 1H), 3.04-2.95 (m, 1H), | |
| | | 2.66-2.60 (m, 1H) | |
| 49 | [M + H]⁺ = | ¹H NMR (DMSO-d₆, 400 MHz) δ 7.62 | SFC peak 2 (Retention |
| | 367.6 | (dd, J = 0.8, 8.0 Hz, 1H), 7.57 (dd, J = | time 2.630 min, |
| | | 1.6, 7.6 Hz, 1H), 7.44 (t, J = 7.6 Hz, | DAICEL CHIRALCEL |
| | | 1H), 7.28 (dt, J = 1.6, 7.6 Hz, 1H), | OJ-H |
| | | 6.10 (s, 2H), 5.55 (s, 2H), 4.76 (dd, J = | (250 mm*30 mm, 10 um)) |
| | | 2.0, 10.0 Hz, 1H), 4.23 (d, J = 13.2 | |
| | | Hz, 1H), 4.10-3.96 (m, 2H), 3.82-3.73 | |
| | | (m, 1H), 3.00 (dt, J = 3.2, 12.4 Hz, | |
| | | 1H), 2.66-2.60 (m, 1H) | |
| 72 | [M + H]⁺ = | ¹H NMR (DMSO-d₆, 400 MHz) δ 7.64 | SFC peak 2 (Retention |
| | 376.8 | (dd, J = 0.8, 8.0 Hz, 1H), 7.59 (dd, J = | time 1.573 min, |
| | | 1.6, 7.6 Hz, 1H), 7.48-7.42 (m, 1H), | DAICEL CHIRALCEL |
| | | 7.32-7.26 (m, 1H), 6.15 (s, 2H), 5.50 | OJ |
| | | (s, 1H), 4.68 (dd, J = 2.4, 10.4 Hz, | (250 mm*30 mm, 10 um)) |
| | | 1H), 4.48 (d, J = 12.8 Hz, 1H), 4.13- | |
| | | 4.03 (m, 2H), 3.76-3.68 (m, 1H), 2.97 | |
| | | (dt, J = 3.6, 12.4 Hz, 1H), 2.67-2.65 | |
| | | (m, 1H), 2.60-2.58 (m, 1H), 1.16-1.10 | |
| | | (m, 6H) | |
| 73 | [M + H]⁺ = | ¹H NMR (DMSO-d₆, 400 MHz) δ 7.64 | SFC peak 1 (Retention |
| | 376.8 | (dd, J = 0.8, 8.0 Hz, 1H), 7.59 (dd, J = | time 1.482 min, |
| | | 1.6, 7.6 Hz, 1H), 7.47-7.42 (m, 1H), | DAICEL CHIRALCEL |
| | | 7.32-7.26 (m, 1H), 6.16 (s, 2H), 5.50 | OJ |
| | | (s, 1H), 4.72-4.65 (m, 1H), 4.48 (d, J = | (250 mm*30 mm, 10 um)) |
| | | 12.8 Hz, 1H), 4.13-4.02 (m, 2H), | |
| | | 3.76-3.68 (m, 1H), 2.97 (dt, J = 3.6, | |
| | | 12.4 Hz, 1H), 2.70-2.62 (m, 1H), | |
| | | 2.60-2.53 (m, 1H), 1.16-1.10 (m, 6H) | |
| 95 | [M + H]⁺ = | ¹H NMR (DMSO-d₆, 400 MHz) δ | SFC peak 1 (Retention |
| | 312.2 | 7.47-7.27 (m, 5H), 6.43 (br d, J = 2.0 | time 1.629 min, |
| | | Hz, 1H), 5.53 (s, 2H), 5.25 (s, 1H), | DAICEL CHIRALCEL |
| | | 4.48 (dd, J = 10.4, 2.0 Hz, 1H), 4.25 | OD |
| | | (d, J = 13.6 Hz, 1H), 4.08-3.94 (m, | (250 mm*30 mm, 10 um)) |
| | | 2H), 3.69-3.63 (m, 1H), 2.95-2.85 (m, | |
| | | 1H), 2.69-2.60 (m, 1H), 2.42-2.38 (m, | |
| | | 1H), 0.63 (dd, J = 6.4, 1.6 Hz, 2H), | |
| | | 0.46-0.33 (m, 2H) | |
| 96 | [M + H]⁺ = | ¹H NMR (DMSO-d₆, 400 MHz) δ | SFC peak 2 (Retention |
| | 312.2 | 7.45-7.29 (m, 5H), 6.43 (d, J = 2.0 | time 2.098 min, |
| | | Hz, 1H), 5.25 (s, 1H), 5.53 (s, 2H), | DAICEL CHIRALCEL |
| | | 4.48 (dd, J = 10.4, 2.4 Hz, 1H), 4.25 | OD |
| | | (d, J = 13.2 Hz, 1H), 4.07-3.96 (m, | (250 mm*30 mm, 10 um)) |
| | | 2H), 3.69-36.3 (m, 1H), 2.95-2.85 (m, | |
| | | 1H), 2.69-2.62 (m, 1H), 2.45-2.43 (m, | |
| | | 1H), 0.67-0.59 (m, 2H), 0.43-0.35 (m, 2H) | |
| 108 | [M + H]⁺ = | ¹H NMR (DMSO-d₆, 400 MHz) δ 7.42- | SFC peak 2 (Retention |
| | 286.2 | 7.31 (m, 5H), 6.17-6.16 (m, 1H), 5.59 | time 1.724 min, |
| | | (s, 2H), 5.05 (s, 1H), 4.48-4.44 (m, | Phenomenex- |
| | | 1H), 4.21 (d, J = 12.8 Hz, 1H), 4.05- | Cellulose-2 |
| | | 3.96 (m, 2H), 3.64 (dt, J = 2.4, 11.6 | (250 mm*30 mm, 10 um)) |
| | | Hz, 1H), 2.86 (dt, J = 2.8, 12.4 Hz, | |
| | | 1H), 2.68 (d, J = 4.8 Hz, 3H), 2.64- | |
| | | 2.58 (m, 1H) | |
| 109 | [M + H]⁺ = | ¹H NMR (DMSO-d₆, 400 MHz) δ 7.40- | SFC peak 1 (Retention |
| | 286.2 | 7.33 (m, 5H), 6.13 (s, 1H), 5.54 (s, | time 1.051 min, |
| | | 2H), 5.04 (s, 1H), 4.46 (d, J = 9.6 Hz, | Phenomenex- |
| | | 1H), 4.21 (d, J = 12.0 Hz, 1H), 4.04- | Cellulose-2 |
| | | 3.96 (m, 2H), 3.64 (t, J = 10.0 Hz, | (250 mm*30 mm, 10 um)) |
| | | 1H), 2.85 (t, J = 10.8 Hz, 1H), 2.68 (s, | |
| | | 3H), 2.63-2.60 (m, 1H) | |

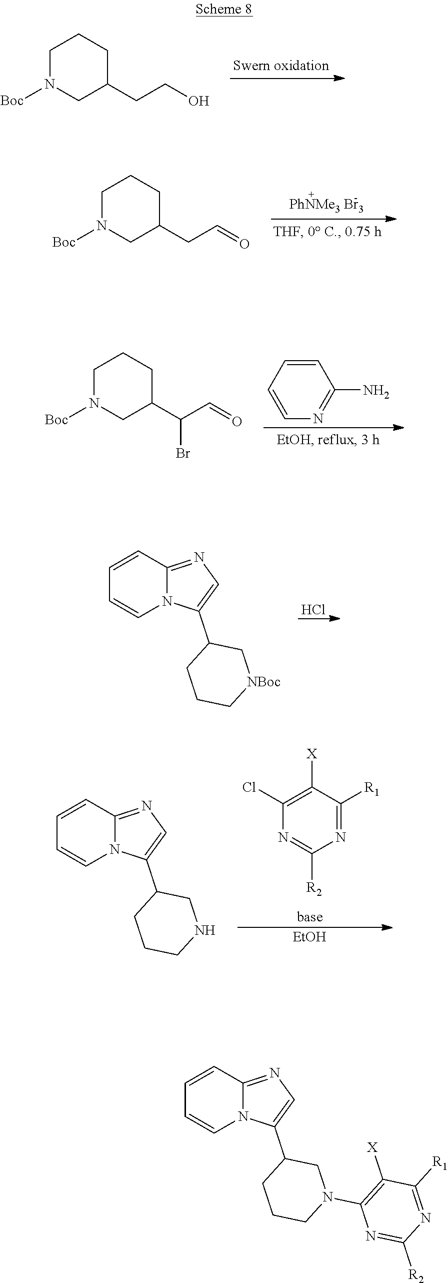

Compounds 19, 62, 90, 91, 107, 123, 124, 125, 126, 141, 142, 178, 239, 241, 242, 245, 253, 255, and 258 can be prepared via a similar procedure to the one shown in Scheme 8.

TABLE 10
Characterization of selected compounds

| Compound | | | |
|---|---|---|---|
| No. | LC-MS | ¹H NMR | Chiral Separation Data |
| 19 | [M + H]⁺ = | ¹H NMR (MeOD-d₄, 400 MHz) δ 9.30- | |
| | 361.4 | 9.18 (m, 1H), 8.23 (s, 1H), 8.05-8.00 | |
| | | (m, 2H), 7.98-7.93 (m, 1H), 7.63 (t, J = | |
| | | 6.8 Hz, 1H), 6.82 (s, 1H), 4.82-4.48 | |
| | | (m, 2H), 3.70-3.51 (m, 3H), 3.21-3.16 | |
| | | (m, 1H), 2.46-2.38 (m, 1H), 2.21-2.11 | |
| | | (m, 2H), 2.04-1.94 (m, 1H), 1.43 (dd, | |
| | | J = 1.2, 8.0 Hz, 6H) | |
| 62 | [M + H]⁺ = | ¹H NMR (DMSO-d₆, 400 MHz) δ | |
| | 391.3 | 15.58-12.94 (m, 1H), 9.15-9.14 (m, | |
| | | 1H), 8.40 (s, 1H), 8.15-8.13 (m, 2H), | |
| | | 8.02-7.94 (m, 2H), 7.61-7.58 (m, 1H), | |
| | | 6.98-6.96 m, 1H), 5.65-5.19 (m, 1H), | |
| | | 4.69-4.65 (m, 1H), 3.64-3.61 (m, 2H), | |
| | | 3.46 (t, J = 12.0 Hz, 1H), 2.27-2.24 | |
| | | (m, 1H), 1.83-1.80 (m, 2H), 1.82 (d, J = | |
| | | 12.3 Hz, 1H) | |
| 90 | [M + H]⁺ = | ¹H NMR (DMSO-d₆, 400 MHz) δ | |
| | 319.2 | 11.73 (s, 1H), 8.57 (d, J = 6.8 Hz, | |
| | | 1H), 8.24 (s, 1H), 7.57 (d, J = 8.8 Hz, | |
| | | 1H), 7.47 (s, 1H), 7.26-7.21 (m, 1H), | |
| | | 7.20-7.19 (m, 1H), 7.00-6.95 (m, 1H), | |
| | | 6.57-6.56 (m, 1H), 4.92 (d, J = 12.0 | |
| | | Hz, 1H), 4.64 (d, J = 14.0 Hz, 1H), | |
| | | 3.41-3.34 (m, 1H), 3.30-3.16 (m, 2H), | |
| | | 2.21-2.17 (m, 1H), 1.97-1.83 (m, 2H), | |
| | | 1.77-1.65 (m, 1H) | |
| 91 | [M + H]⁺ = | ¹H NMR (CD₃OD, 400 MHz) δ 8.85 | |
| | 318.1 | (d, J = 7.2 Hz, 1H), 8.04-7.92 (m, | |
| | | 4H), 7.53 (t, J = 6.8 Hz, 1H), 7.31 (d, | |
| | | J = 3.6 Hz, 1H), 6.91-6.84 (m, 2H), | |
| | | 4.70 (d, J = 13.6 Hz, 1H), 4.52 (d, J = | |
| | | 13.6 Hz, 1H), 3.81-3.75 (m, 1H), | |
| | | 3.71-3.61 (m, 2H), 2.48-2.40 (m, 1H), | |
| | | 2.13-1.99 (m, 3H) | |
| 107 | [M + H]⁺ = | ¹H NMR (DMSO-d₆, 400 MHz) δ 8.47 | |
| | 350.0 | (d, J = 6.8 Hz, 1H), 7.56 (d, J = 9.2 | |
| | | Hz, 1H), 7.44 (s, 1H), 7.26-7.18 (m, | |
| | | 1H), 7.02-6.93 (m, 1H), 6.38 (d, J = | |
| | | 2.4 Hz, 1H), 5.49 (s, 2H), 5.29 (s, | |
| | | 1H), 4.55 (d, J = 12.4 Hz, 1H), 4.12 | |
| | | (d, J = 13.4 Hz, 1H), 3.17-3.05 (m, | |
| | | 1H), 3.03-2.93 (m, 1H), 2.93-2.85 (m, | |
| | | 1H), 2.45-2.41 (m, 1H), 2.18-2.10 (m, | |
| | | 1H), 1.87-1.76 (m, 1H), 1.76-1.69 (m, | |
| | | 1H), 1.69-1.64 (m, 1H), 0.68-0.58 (m, | |
| | | 2H), 0.45-0.34 (m, 2H) | |
| 123 | | | SFC peak 2 (Retention |
| | | | time 2.413 min, |
| | | | DAICEL CHIRALPAK |
| | | | IG |
| | | | (250 mm*30 mm, 10 um)) |
| 124 | [M + H]⁺ = | ¹H NMR (CD₃OD, 400 MHz) δ 8.86 | SFC peak 1 (Retention |
| | 338.3 | (d, J = 6.62 Hz, 1H), 8.05-7.96 (m, | time 1.905 min, |
| | | 3H), 7.58 (t, J = 7.2 Hz, 1H), 4.90- | DAICEL CHIRALPAK |
| | | 4.87 (m, 1H), 4.37-3.97 (m, 2H), | IG |
| | | 3.48-3.43 (m, 1H), 3.42-3.34 (m, 2H), | (250 mm*30 mm, 10 um)) |
| | | 2.90 (d, J = 2.8 Hz, 6H), 2.30-2.32 | |
| | | (m, 1H), 2.06-2.06 (m, 2H), 1.88-1.82 | |
| | | (m, 1H) | |
| 125 | [M + H]⁺ = | ¹H NMR (CD₃OD, 400 MHz) δ 8.52 | SFC peak 2 (Retention |
| | 351.2 | (s, 1H), 7.57 (d, J = 9.2, 1H), 7.46 (s, | time 2.534 min, |
| | | 1H), 7.37-7.31 (m, 1H), 6.98 (t, J = | DAICEL CHIRALPAK |
| | | 6.8, 1H), 5.53 (s, 1H), 4.92-4.88 (m, | IG |
| | | 1H), 4.20 (d, J = 8.0 Hz, 1H), 3.26- | (250 mm*30 mm, 10 um)) |
| | | 3.17 (m, 2H), 3.08-3.06 (m, 1H), 2.85 | |
| | | (s, 3H), 2.83-2.78 (m, 1H), 2.32-2.24 | |
| | | (m, 1H), 2.12-2.00 (m, 1H), 1.95-1.87 | |
| | | (m, 1H), 1.85-1.74 (m, 1H), 1.29 (d, J = | |
| | | 6.8 Hz, 3H), 1.23 (d, J = 6.8 Hz, 3H) | |
| 126 | [M + H]⁺ = | | SFC peak 1 (Retention |
| | 351.2 | | time 2.151 min, |
| | | | DAICEL CHIRALPAK |
| | | | IG |
| | | | (250 mm*30 mm, 10 um) |
| 141 | [M + H]⁺ = | | SFC peak 2 (Retention |
| | 337.3 | | time 1.432 min, |
| | | | Phenomenex- |
| | | | Cellulose-2 |
| | | | (250 mm*30 mm, 10 um)) |
| 142 | [M + H]⁺ = | ¹H NMR (CD₃OD, 400 MHz) δ 8.46 | SFC peak 1 (Retention |
| | 337.3 | (d, J = 6.4 Hz, 1H), 7.58 (d, J = 9.2 | time 1.017 min, |
| | | Hz, 1H), 7.47 (s, 1H), 7.38-7.34 (m, | Phenomenex- |
| | | 1H), 7.01 (t, J = 6.4 Hz, 1H), 5.72 (s, | Cellulose-2 |
| | | 1H), 4.82 (d, J = 11.6 Hz, 1H), 4.15 | (250 mm*30 mm, 10 um)) |
| | | (d, J = 11.6 Hz, 1H), 3.29-3.14 (m, | |
| | | 3H), 2.89-2.79 (m, 1H), 2.31-2.29 (m, | |
| | | 1H), 2.12-2.00 (m, 1H), 1.99-1.88 (m, | |
| | | 1H), 1.85-1.73 (m, 1H), 1.27 (d, J = | |
| | | 6.8 Hz, 3H), 1.24 (d, J = 6.8 Hz, 3H), | |
| | | LC-MS: [M + H]⁺ = 337.3 | |
| 178 | [M + H]⁺ = | ¹H NMR (DMSO-d₆, 400 MHz) δ 8.46 | |
| | 337.1 | (d, J = 6.8 Hz, 1H), 8.18 (s, 1H), 7.57 | |
| | | (d, J = 9.2 Hz, 1H), 7.44 (s, 1H), | |
| | | 7.25-7.21 (m, 1H), 7.00 (t, J = 6.8 Hz, | |
| | | 1H), 5.98-5.95 (m, 3H), 4.67-4.63 (m, | |
| | | 1H), 4.27-4.22 (m, 1H), 3.17-3.16 (m, | |
| | | 1H), 3.04-2.99 (m, 2H), 2.62-2.60 (m, | |
| | | 1H), 2.18-2.15 (m, 1H), 1.79-1.74 (m, | |
| | | 2H), 1.74-1.73 (m, 1H), 1.15-1.13 (d, | |
| | | J = 6.4 Hz, 6H) | |
| 239 | [M + H]⁺ = | ¹H NMR (CDCl₃, 500 MHz) δ 8.96 (d, | |
| | 338.0 | J = 1.5 Hz, 1H), 7.89 (d, J = 6.5 Hz, | |
| | | 1H), 7.48 (s, 1H), 7.46 (d, J = 6.5 Hz, | |
| | | 1H), 5.32 (s, 1H), 4.65 (s, 2H), 4.47 | |
| | | (s, 2H), 4.39 (d, J = 13.0 Hz, 1H), | |
| | | 4.13 (d, J = 13.0 Hz, 1H), 3.23 (dd, J = | |
| | | 13.0, 10.0 Hz, 1H), 3.07-3.00 (m, | |
| | | 2H), 2.22-2.17 (m, 1H), 1.95-1.86 (m, | |
| | | 1H), 1.79-1.75 (m, 1H), 1.69-1.63 (m, | |
| | | 1H) | |
| 241 | [M + H]⁺ = | ¹H NMR (CDCl₃, 500 MHz) δ 8.50 | |
| | 311.0 | (dd, J = 9.0, 2.0 Hz, 1H), 8.36 (dd, J = | |
| | | 7.0, 2.0 Hz, 1H), 7.38 (s, 1H), 6.83 | |
| | | (dd, J = 6.5, 4.0 Hz, 1H), 5.28 (s, 1H), | |
| | | 4.54 (s, 2H), 4.40-4.39 (m, 1H), 4.37 | |
| | | (s, 2H), 4.16 (d, J = 13.0 Hz, 1H), | |
| | | 3.28 (dd, J = 13.0, 10.0 Hz, 1H), | |
| | | 3.11-3.01 (m, 2H), 2.24-2.18 (m, 1H), | |
| | | 2.03-1.92 (m, 1H), 1.78-1.75 (m, 2H) | |
| 242 | [M + H]⁺ = | ¹H NMR (CD₃OD, 500 MHz) δ 8.53 | |
| | 373.0 | (dd, J = 5.0, 2.0 Hz, 1H), 7.53 (s, 1H), | |
| | | 7.28-7.23 (m, 1H), 6.03 (s, 1H), 4.70 | |
| | | (d, J = 12.5 Hz, 1H), 4.18 (d, J = 13.5 | |
| | | Hz, 1H), 3.27-3.21 (m, 1H), 3.19-3.08 | |
| | | (m, 2H), 2.71-2.62 (m, 1H), 2.27-2.23 | |
| | | (m, 1H), 2.01-1.93 (m, 1H), 1.89-1.84 | |
| | | (m, 1H), 1.78-1.71 (m, 1H), 1.21 (dd, | |
| | | J = 7.0, 3.0 Hz, 6H) | |
| 245 | [M + H]⁺ = | ¹H NMR (CDCl₃, 500 MHz) δ 8.59 | |
| | 338.0 | (dd, J = 7.0, 2.0 Hz, 1H), 8.56 (dd, J = | |
| | | 4.0, 2.0 Hz, 1H), 7.63 (s, 1H), 6.93 | |
| | | (dd, J = 7.0, 4.0 Hz, 1H), 5.91 (s, 1H), | |
| | | 4.89 (s, 2H), 4.85 (d, J = 13.0 Hz, | |
| | | 1H), 4.15 (d, J = 14.0 Hz, 1H), 3.15- | |
| | | 3.09 (m, 1H), 3.07-3.00 (m, 1H), 2.86 | |
| | | (dd, J = 13.0, 11.0 Hz, 1H), 2.77-2.66 | |
| | | (m, 1H), 2.29-2.25 (m, 1H), 2.02-1.96 | |
| | | (m, 1H), 1.95-1.88 (m, 1H), 1.76-1.67 | |
| | | (m, 1H), 1.24 (d, J = 7.0 Hz, 6H) | |
| 253 | [M + H]⁺ = | ¹H NMR (DMSO-d₆, 500 MHz) δ 8.49 | |
| | 323.15 | (d, J = 6.5 Hz, 1H), 7.56 (d, J = 9.0 | |
| | | Hz, 1H), 7.47 (s, 1H), 7.28-7.24 (m, | |
| | | 1H), 6.98-6.94 (m, 1H), 5.90 (s, 2H), | |
| | | 5.67 (s, 1H), 4.05-3.82 (m, 3H), 3.70- | |
| | | 3.58 (m, 3H), 2.60-2.55 (m, 1H), | |
| | | 2.19-2.09 (m, 1H), 1.13 (d, J = 6.5 | |
| | | Hz, 6H) | |
| 255 | [M + H]⁺ = | ¹H NMR (DMSO-d₆, 500 MHz) δ 8.48 | |
| | 296.0 | (d, J = 6.5 Hz, 1H), 7.55 (d, J = 9.0 | |
| | | Hz, 1H), 7.44 (s, 1H), 7.25-7.21 (m, | |
| | | 1H), 6.96-6.93 (m, 1H), 5.62 (s, 2H), | |
| | | 5.42 (s, 2H), 4.84 (s, 1H), 3.96-3.90 | |
| | | (m, 1H), 3.86-3.79 (m, 1H), 3.55-3.46 | |
| | | (m, 1H), 3.41-3.39(m, 2H), 2.47-2.43 | |
| | | (m, 1H), 2.14-2.06 (m, 1H) | |
| 258 | [M + H]⁺ = | ¹H NMR (CDCl₃, 500 MHz) δ 8.96 (d, | |
| | 338.0 | J = 1.5 Hz, 1H), 7.89 (d, J = 6.5 Hz, | |
| | | 1H), 7.48 (s, 1H), 7.46 (d, J = 6.5 Hz, | |
| | | 1H), 5.32 (s, 1H), 4.65 (s, 2H), 4.47 | |
| | | (s, 2H), 4.39 (d, J = 13.0 Hz, 1H), | |
| | | 4.13 (d, J = 13.0 Hz, 1H), 3.23 (dd, J = | |
| | | 13.0, 10.0 Hz, 1H), 3.07-3.00 (m, | |
| | | 2H), 2.22-2.17 (m, 1H), 1.95-1.86 (m, | |
| | | 1H), 1.79-1.75 (m, 1H), 1.69-1.63 (m, | |
| | | 1H) | |

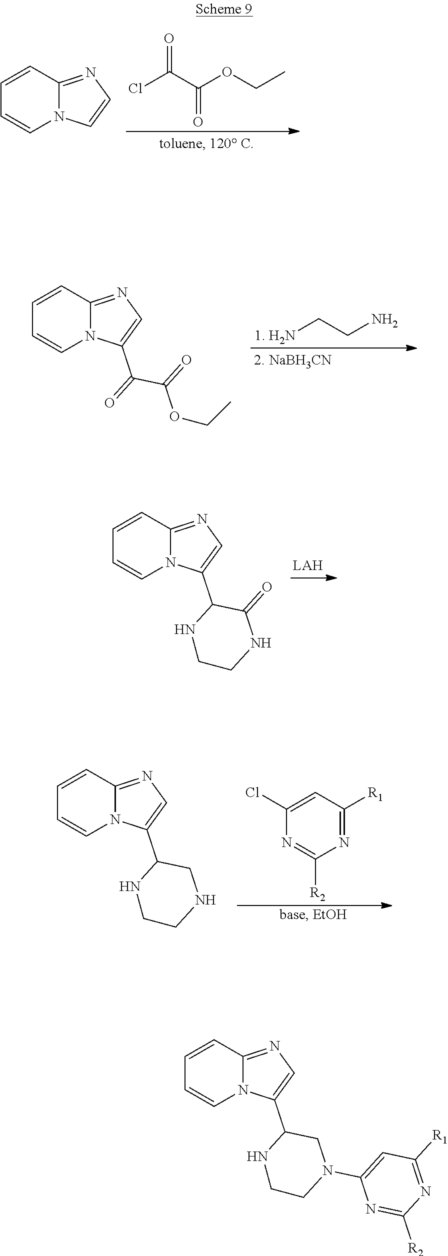

Compounds 22, 27, 28, 32, 55, 92, 103, and 104 can be prepared via a similar procedure to the one shown in Scheme 9.

TABLE 11
Characterization of selected compounds

| Compound | | | |
|---|---|---|---|
| No. | LC-MS | ¹H NMR | Chiral Separation Data |
| 22 | [M + H]⁺ = | ¹H NMR (CDCl₃, 500 MHz) δ 8.45 (d, J = | |
| | 338.0 | 7.0 Hz, 1H), 7.99 (s, 1H), 7.72 (dt, J = | |
| | | 9.0, 2.0 Hz, 1H), 7.13-7.09 (m, 1H), 6.76 | |
| | | (td, J = 7.0, 1.5 Hz, 1H), 5.84 (s, 1H), | |
| | | 4.75 (br, 2H), 4.36 (d, J = 13.0 Hz, 1H), | |
| | | 4.29 (s, J = 12.5 Hz, 1H), 4.11 (dd, J = | |
| | | 10.5, 3.5 Hz, 1H), 3.21-3.17 (m, 1H), | |
| | | 3.10-2.96 (m, 3H), 2.71-2.64 (m, 1H), | |
| | | 1.21 (d, J = 7.0 Hz, 6H) | |
| 27 | [M + H]⁺ = | ¹H NMR (CD₃OD, 400 MHz) δ 8.60 (d, J = | SFC peak 1 |
| | 323.7 | 7.2 Hz, 1H), 7.64-7.52 (m, 2H), 7.37 (dd, | (Retention time |
| | | J = 7.6, 8.4 Hz, 1H), 7.01 (t, J = 6.8 Hz, | 1.946 min, DAICEL |
| | | 1H), 6.30 (s, 1H), 4.73-4.62 (m, 1H), | CHIRALPAK |
| | | 4.41-4.25 (m, 2H), 3.56 (dd, J = 10.0, | AS(250 mm*30 mm, |
| | | 13.2 Hz, 1H), 3.39-3.34 (m, 1H), 3.14- | 10 um)) |
| | | 3.11 (m, 1H), 3.02-2.95 (m, 1H), 2.57 (q, | |
| | | J = 7.6 Hz, 2H), 1.27 (t, J = 7.6 Hz, 3H) | |
| 28 | [M + H]⁺ = | | SFC peak 2 |
| | 323.7 | | (Retention time |
| | | | 2.244 min, DAICEL |
| | | | CHIRALPAK |
| | | | AS(250 mm*30 mm, |
| | | | 10 um)) |
| 32 | [M + H]⁺ = | ¹H NMR (CD₃OD, 400 MHz) δ 8.62-8.49 | |
| | 338.2 | (m, 1H), 7.64-7.51 (m, 2H), 7.40-7.29 (m, | |
| | | 1H), 7.00 (t, J = 6.8 Hz, 1H), 6.07 (s, 1H), | |
| | | 4.67-4.56 (m, 1H), 4.32 (dd, J = 9.6, 2.8 | |
| | | Hz, 1H), 4.24-4.11 (m, 1H) 3.40-3.35 (m, | |
| | | 1H), 3.25-3.18 (m, 1H), 3.15-3.05 (m, | |
| | | 1H), 3.01-2.91 (m, 1H) 2.76-2.58 (m, 1H) | |
| | | 1.23 (d, J = 7.2 Hz, 6H) | |
| 55 | [M + H]⁺ = | ¹H NMR (DMSO-d₆, 400 MHz) δ 10.83 (br | |
| | 335.2 | s, 1H), 8.65 (d, J = 6.8 Hz, 1H), 7.75-7.40 | |
| | | (m, 2H), 7.32-7.18 (m, 2H), 6.93 (dt, J = | |
| | | 1.1, 6.8 Hz, 1H), 6.03 (d, J = 2.8 Hz, 1H), | |
| | | 5.35 (s, 2H), 4.45 (d, J = 12.4 Hz, 1H), | |
| | | 4.32 (dd, J = 2.8, 10.0 Hz, 1H), 4.22 (d, J = | |
| | | 12.0 Hz, 1H), 3.46-3.39 (m, 1H), 3.23- | |
| | | 3.15 (m, 1H), 3.03-2.91 (m, 2H) | |
| 92 | [M + H]⁺ = | ¹H NMR (CD₃OD, 400 MHz) δ 3.50-3.60 | |
| | 333.2 | (m, 2H), 3.30-3.29 (m, 2H), 3.07 (s, 3H), | |
| | | 2.93 (s, 3H), 2.69-2.77 (m, 1H), 2.41-2.49 | |
| | | (m, 5 H), 1.91 (d, J = 12.8 Hz, 1H), 1.77- | |
| | | 1.73 (m, 3H), 1.52-1.65 (m, 4H), 1.10- | |
| | | 1.19 (m, 1H) | |
| 103 | [M + H]⁺ = | ¹H NMR (DMSO-d₆ 400 MHz) δ 8.63-8.58 | SFC peak 2 |
| | 336.0 | (m, 1H), 7.58-7.50 (m, 2H), 7.27-7.20 (m, | (Retention time |
| | | 1H), 6.95-6.86 (m, 1H), 6.06 (s, 1H), 5.82 | 2.242 min, DAICEL |
| | | (s, 2H), 4.44-4.34 (m, 1H), 4.18 (d, J = | CHIRALCEL AS |
| | | 9.2 Hz, 1H), 4.12-4.03 (m, 1H), 3.27 (d, J = | (250 mm*30 mm, 10 um)) |
| | | 2.0 Hz, 1H), 3.04-2.97 (m, 1H), 2.96- | |
| | | 2.90 (m, 1H), 2.83-2.76 (m, 1H), 1.77- | |
| | | 1.67 (m, 1H), 0.88-0.84 (m, 2H), 0.79- | |
| | | 0.73 (m, 2H) | |
| 104 | [M + H]⁺ = | ¹H NMR (DMSO-d₆, 400 MHz) δ 8.61 (d, | SFC peak 1 |
| | 336.0 | J = 6.8 Hz, 1H), 7.58-7.50 (m, 2H), 7.27- | (Retention time |
| | | 7.20 (m, 1H), 6.95-6.86 (m, 1H), 6.08- | 1.942 min, DAICEL |
| | | 6.04 (m, 1H), 5.86-5.78 (m, 2H), 4.44- | CHIRALCEL AS |
| | | 4.34 (m, 1H), 4.23-4.14 (m, 1H), 4.12- | (250 mm*30 mm, 10 um)) |
| | | 4.02 (m, 1H), 3.29-3.25 (m, 1H), 3.04- | |
| | | 2.98 (m, 1H), 2.93 (d, J = 12.8 Hz, 1H), | |
| | | 2.83-2.78 (m, 1H), 1.76-1.70 (m, 1H), | |
| | | 0.88-0.84 (m, 2H), 0.79-0.73 (m, 2H) | |

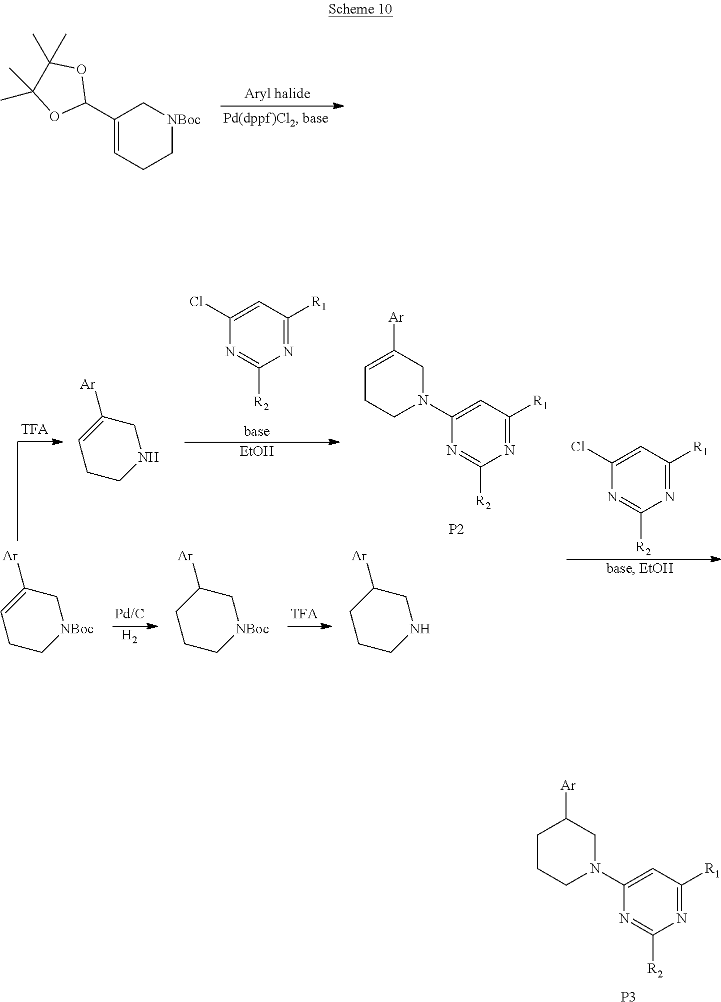

Compounds 225, 230, 232, 235, 237, 238, 240, 246, 247, 248, 249, and 250 can be prepared via a similar procedure to the one shown in Scheme 10.

TABLE 12
Characterization of selected compounds

| Compound | | |
|---|---|---|
| No. | LC-MS | ¹H NMR |
| 225 | [M + H]⁺ = | ¹H NMR (CDCl₃, 500 MHz) δ 8.03 (s. 1H), 7.85 (d, J = 7.0 |
| | 310.0 | Hz, 1H), 7.43 (d, J = 9.0 Hz, 1H), 6.63 (dd, J = 9.5, 6.5 Hz, |
| | | 1H), 6.51 (t, J = 6.5 Hz, 1H), 5.17 (s, 1H), 4.91 (s, 2H), 4.78 |
| | | (s, 2H), 4.41-4.36 (m, 2H), 3.17-3.08 (m, 2H), 2.89 (td, J = |
| | | 13.0, 3.0 Hz, 1H), 2.07-2.01 (m, 1H), 1.88-1.83 (m, 1H), |
| | | 1.71-1.61 (m, 1H), 1.35 (d, J = 6.5 Hz, 1H) |
| 230 | [M + H]⁺ = | ¹H NMR (CD₃OD, 500 MHz) δ 7.60 (d, J = 7.5 Hz, 1H), 7.35 |
| | 373.0 | (t, J = 7.5 Hz, 1H), 7.30-7.27 (m, 1H), 7.21 (t, J = 7.5 Hz, |
| | | 1H), 5.96 (s, 1H), 5.84-5.81 (m, 1H), 4.23 (s, 2H), 3.82 (t, J = |
| | | 6.0 Hz, 2H), 2.68-2.62 (m, 1H), 2.38-2.35 (m, 2H), 1.21 (d, |
| | | J = 6.5 Hz, 6H) |
| 232 | [M + H]⁺ = | ¹H NMR (CDCl₃, 500 MHz) δ 7.77 (d, J = 8.0 Hz, 1H), 7.42- |
| | 351.0 | 7.35 (m, 2H), 7.14 (t, J = 7.0 Hz, 1H), 5.89 (s, 1H), 4.66 (s, |
| | | 3H), 4.49 (d, J = 13.0 Hz, 1H), 4.04 (s, 3H), 3.29-3.23 (m, |
| | | 1H), 3.15 (t, J = 12.5 Hz, 1H), 2.93 (td, J = 13.0, 2.5 Hz, |
| | | 1H), 2.69-2.63 (m, 1H), 2.26 (d, J = 13.5 Hz, 1H), 2.05-1.98 |
| | | (m, 1H), 1.90-1.87 (m, 1H), 1.75-1.68 (m, 1H), 1.21 (dd, J = |
| | | 7.0, 3.0 Hz, 6H) |
| 235 | [M + H]⁺ = | ¹H NMR (CDCl₃, 500 MHz) δ 8.05 (s, 1H), 7.86 (d, J = 7.0 |
| | 337.0 | Hz, 1H), 7.44 (d, J = 9.5 Hz, 1H), 6.66-6.62 (m, 1H), 6.54- |
| | | 6.50 (m, 1H), 5.87 (s, 1H), 4.69 (br, 2H), 4.51-4.45 (m, 2H), |
| | | 3.19-3.08 (m, 2H), 2.95-2.88 (m, 1H), 2.69-2.63 (m, 1H), |
| | | 2.11-2.05 (m, 2H), 1.89-1.86 (m, 1H), 1.72-1.66 (m, 1H), |
| | | 1.21 (dd, J = 7.0, 1.5 Hz, 6H) |
| 237 | [M + H]⁺ = | ¹H NMR (CDCl₃, 500 MHz) δ 8.07 (s, 1H), 7.89 (d, J = 7.0 |
| | 308.20 | Hz, 1H), 7.66 (d, J = 9.0 Hz, 1H), 6.74 (dd, J = 9.5, 6.5 Hz, |
| | | 1H), 6.56 (t, J = 7.0 Hz, 1H), 6.38-6.36 (m, 1H), 5.32 (s, |
| | | 1H), 4.57 (s, 2H), 4.48 (s, 2H), 4.37 (s, 2H), 3.84 (t, J = 6.0 |
| | | Hz, 2H), 2.43 (s, 2H) |
| 238 | [M + H]⁺ = | ¹H NMR (CDCl₃, 500 MHz) δ 8.08 (s, 1H), 7.90 (d, J = 7.5 |
| | 335.20 | Hz, 1H), 7.66 (d, J = 9.5 Hz, 1H), 6.74 (dd, J = 9.5, 6.5 Hz, |
| | | 1H), 6.56 (t, J = 7.0 Hz, 1H), 6.39-6.36 (m, 1H), 5.97 (s, |
| | | 1H), 4.71 (s, 2H), 4.57 (s, 2H), 3.87 (t, J = 6.0 Hz, 2H), 2.72- |
| | | 2.65 (m, 1H), 2.46-2.42 (m, 2H), 1.22 (d, J = 7.0 Hz, 6H) |
| 240 | [M + H]⁺ = | ¹H NMR (CDCl₃, 500 MHz) δ 8.45 (d, J = 7.0 Hz, 1H), 7.84 |
| | 337.0 | (s, 1H), 7.51 (d, J = 9.0 Hz, 1H), 7.13 (t, J = 7.5 Hz, 1H), |
| | | 6.77 (t, J = 7.0 Hz, 1H), 5.87 (s, 1H), 5.01-3.88 (m, 4H), |
| | | 3.18-2.81 (m, 4H), 2.22 (d, J = 13.0 Hz, 1H), 1.98-1.85 (m, |
| | | 2H), 1.76-1.67 (m, 1H), 1.28 (d, J = 7.0 Hz, 6H) |
| 246 | [M + H]⁺ = | ¹H NMR (CDCl₃, 500 MHz) δ 9.99 (br, 1H), 7.81 (d, J = 8.5 |
| | 310.0 | Hz, 1H), 7.46 (d, J = 8.5 Hz, 1H), 7.39 (t, J = 8.0 Hz, 1H), |
| | | 7.17 (t, J = 7.5 Hz, 1H), 5.21 (s, 1H), 4.63-4.55 (m, 3H), |
| | | 4.43-4.35 (m, 3H), 3.31-3.26 (m, 1H), 3.14 (t, J = 12.0 Hz, |
| | | 1H), 2.91 (t, J = 13.0 Hz, 1H), 2.28 (d, J = 13.5 Hz, 1H), |
| | | 2.05-1.97 (m, 2H) |
| 247 | [M + H]⁺ = | ¹H NMR (CD₃OD, 500 MHz) δ 7.64 (d, J = 8.0 Hz, 1H), 7.33 |
| | 350.0 | (d, J = 8.5 Hz, 1H), 7.17 (t, J = 8.0 Hz, 1H), 7.05 (t, J = 7.5 |
| | | Hz, 1H), 7.02 (s, 1H), 5.99 (s, 1H), 4.61 (d, J = 13.0 Hz, |
| | | 1H), 4.40 (d, J = 13.5 Hz, 1H), 3.76 (s, 3H), 3.07-2.81 (m, |
| | | 3H), 2.68-2.62 (m, 1H), 2.21-2.18 (m, 1H), 1.95-1.81 (m, |
| | | 2H), 1.73-1.65 (m, 1H), 1.22 (t, J = 7.0 Hz, 6H) |
| 248 | [M + H]⁺ = | ¹H NMR (CD₃OD, 500 MHz) δ 7.65 (d, J = 8.0 Hz, 1H), 7.32 |
| | 323.0 | (d, J = 8.0 Hz, 1H), 7.16 (t, J = 7.5 Hz, 1H), 7.04 (t, J = 7.5 |
| | | Hz, 1H), 7.00 (s, 1H), 5.28 (s, 1H), 4.45 (d, J = 13.0 Hz, |
| | | 1H), 4.26 (d, J = 13.0 Hz, 1H), 3.75 (s, 3H), 3.04-2.98 (m, |
| | | 1H), 2.94-2.84 (m, 2H), 2.19-2.14 (m, 1H), 1.87-1.78 (m, |
| | | 2H), 1.73-1.67 (m, 1H) |
| 249 | [M + H]⁺ = | ¹H NMR (CD₃OD, 500 MHz) δ 7.64 (d, J = 7.5 Hz, 1H), 7.34 |
| | 336.0 | (d, J = 8.0 Hz, 1H), 7.11-7.07 (m, 2H), 7.04-7.00 (m, 1H), |
| | | 5.98 (s, 1H), 4.63-4.59 (m, 1H), 4.41-4.38 (m, 1H), 3.07- |
| | | 2.97 (m, 3H), 2.68-2.62 (m, 1H), 2.23-2.18 (m, 1H), 1.94- |
| | | 1.84 (m, 2H), 1.74-1.68 (m, 1H), 1.22 (t, J = 7.0 Hz, 6H) |
| 250 | [M + H]⁺ = | ¹H NMR (CD₃OD, 500 MHz) δ 7.64 (d, J = 8.0 Hz, 1H), 7.33 |
| | 309.0 | (d, J = 8.0 Hz, 1H), 7.10-7.06 (m, 2H), 7.02 (t, J = 8.0 Hz, |
| | | 1H), 5.28 (s, 1H), 4.46 (d, J = 13.0 Hz, 1H), 4.27 (d, J = 13.5 |
| | | Hz, 1H), 3.05-2.99 (m, 1H), 2.95-2.87 (m, 2H), 2.21-2.17 (m, |
| | | 1H), 1.89-1.79 (m, 2H), 1.74-1.67 (m, 1H) |

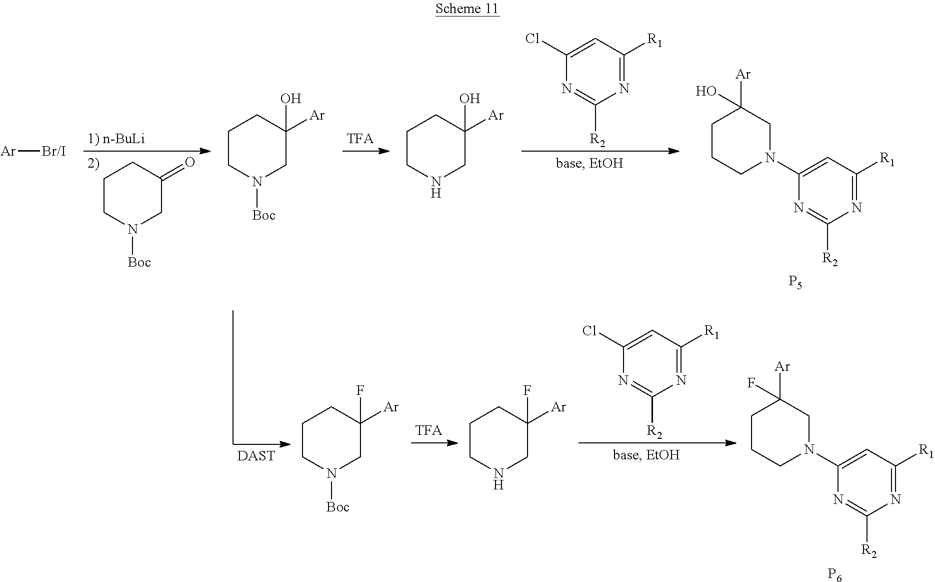

Compounds 224, 226, 228, 231, 233, 234, 236, and 243 can be prepared via a similar procedure to the one shown in Scheme 11.

TABLE 13
Characterization of selected compounds

| Compound | | |
|---|---|---|
| No. | LC-MS | ¹H NMR |
| 224 | [M + H]⁺ = | ¹H NMR (CDCl₃, 500 MHz) δ 7.95 (s, 1H), 7.83 (d, J = 9.0 |
| | 353.0 | Hz, 1H), 7.79 (d, J = 7.5 Hz, 1H), 6.58 (dd, J = 9.0, 6.5 Hz, |
| | | 1H), 6.46 (t, J = 7.5 Hz, 1H), 5.86 (s, 1H), 4.64 (br, 2H), |
| | | 4.54 (d, J = 13.5 Hz, 1H), 4.20 (d, J = 13.5 Hz, 1H), 3.42 |
| | | (d, J = 13.5 Hz, 1H), 3.36 (br, 1H), 2.98 (td, J = 13.0, 3.0 |
| | | Hz, 1H), 2.62-2.55 (m, 1H), 2.26-2.19 (m, 1H), 2.04-2.00 |
| | | (m, 1H), 1.96-1.90 (m, 1H), 1.68-1.63 (m, 1H), 1.13 (d, J = |
| | | 7.0 Hz, 6H) |
| 226 | [M + H]⁺ = | ¹H NMR (CDCl₃, 500 MHz) δ 8.13 (d, J = 8.0 Hz, 1H), 7.58 |
| | 407.05 | (d, J = 8.0 Hz, 1H), 7.38 (t, J = 7.0 Hz, 1H), 7.13 (t, J = 7.5 |
| | | Hz, 1H), 5.82 (s, 1H), 4.91-4.62 (m, 2H), 4.43-4.32 (m, 2H), |
| | | 3.70-3.61 (m, 1H), 3.49-3.43 (m, 1H), 3.36-3.25 (m, 1H), |
| | | 2.75-2.62 (m, 2H), 2.20-2.11 (m, 1H), 2.05-1.92 (m, 1H), |
| | | 1.85-1.75 (m, 3H), 1.24 (d, J = 7.0 Hz, 6H) |
| 228 | [M + H]⁺ = | ¹H NMR (CDCl₃, 500 MHz) δ 7.70 (dd, J = 8.0, 2.0 Hz, 1H), |
| | 368.0 | 7.62 (d, J = 8.0 Hz, 1H), 7.37 (t, J = 8.0 Hz, 1H), 7.18 (t, J = |
| | | 8.5 Hz, 1H), 5.18 (s, 1H), 4.51 (s, 2H), 4.43-4.37 (m, 1H), |
| | | 4.34 (s, 2H), 3.99 (dd, J = 37.0, 14.5 Hz, 1H), 3.04 (t, J = |
| | | 12.0 Hz, 1H), 2.96-2.83 (m, 1H), 2.09-1.97 (m, 2H), 1.89- |
| | | 1.78 (m, 1H), 1.21-1.17 (m, 1H) |
| 231 | [M + H]⁺ = | ¹H NMR (CDCl₃, 500 MHz) δ 7.65 (dd, J = 8.0, 1.5 Hz, 1H), |
| | 379.0 | 7.54 (d, J = 8.5 Hz, 1H), 7.33 (t, J = 8.0 Hz, 1H), 7.20 (t, J = |
| | | 7.5 Hz, 1H), 5.66 (s, 1H), 4.69 (br, 2H), 4.25 (br, 1H), 3.92 |
| | | (d, J = 12.0 Hz, 1H), 3.73-3.70 (m, 2H), 3.06 (s, 1H), 2.76- |
| | | 2.65 (m, 2H), 2.49 (s, 1H), 1.23 (d, J = 7.0 Hz, 6H) |
| 233 | [M + H]⁺ = | ¹H NMR (CDCl₃, 500 MHz) 7.70 (dd, J = 8.0, 2.0 Hz, 1H), |
| | 395.0 | 7.63 (d, J = 8.0 Hz, 1H), 7.38 (td, J = 8.0, 1.5 Hz, 1H), 7.20 |
| | | (td, J = 7.5, 2.0 Hz, 1H), 5.88 (s, 1H), 4.61 (s, 2H), 4.56- |
| | | 4.38 (m, 2H), 3.99 (dd, J = 37.0, 14.0 Hz, 1H), 3.05 (t, J = |
| | | 12.0 Hz, 1H), 2.97-2.82 (m, 1H), 2.70-2.62 (m, 1H), 2.08- |
| | | 2.00 (m, 2H), 1.79-1.76 (m, 1H), 1.21 (d, J = 7.0 Hz, 6H) |
| 234 | [M + H]⁺ = | ¹H NMR (CDCl₃, 500 MHz) δ 7.87 (d, J = 8.0 Hz, 1H), 7.60 |
| | 364.0 | (d, J = 7.5 Hz, 1H), 7.34 (t, J = 8.0 Hz, 1H), 7.13 (t, J = 8.0 |
| | | Hz, 1H), 5.25 (s, 1H), 4.54 (s, 1H), 4.51 (s, 2H), 4.37 (s, |
| | | 2H), 3.99 (d, J = 13.5 Hz, 1H), 3.90 (d, J = 13.5 Hz, 1H), |
| | | 3.13 (td, J = 13.0, 3.0 Hz, 1H), 2.71 (td, J = 13.0, 4.5 Hz, |
| | | 1H), 2.07-1.99 (m, 1H), 1.88 (d, J = 12.0 Hz, 1H), 1.66-1.60 |
| | | (m, 2H) |
| 236 | [M + H]⁺ = | ¹H NMR (CDCl₃, 500 MHz) δ 7.85 (dd, J = 8.0, 1.5 Hz, 1H), |
| | 391.0 | 7.61 (d, J = 8.0 Hz, 1H), 7.35 (t, J = 7.5 Hz, 1H), 7.14 (td, J = |
| | | 7.5, 1.5 Hz, 1H), 5.95 (s, 1H), 4.66-4.60 (m, 3H), 4.14 (d, |
| | | J = 14.0 Hz, 1H), 3.89 (d, J = 14.0 Hz, 1H), 3.71 (s, 1H), |
| | | 3.14 (td, J = 13.0, 3.0 Hz, 1H), 2.73-2.62 (m, 2H), 2.09- |
| | | 1.99 (m, 1H), 1.95 (d, J = 13.5 Hz, 1H), 1.69-1.66 (m, 1H), |
| | | 1.21 (d, J = 7.0 Hz, 6H) |
| 243 | [M + H]⁺ = | ¹H NMR (DMSO-d₆, 500 MHz) δ 8.79 (d, J = 7.0 Hz, 1H), |
| | 353.0 | 7.54 (d, J = 9.0 Hz, 1H), 7.41 (s, 1H), 7.23 (t, J = 8.0 Hz, |
| | | 1H), 6.94 (t, J = 7.0 Hz, 1H), 5.83-5.78 (m, 3H), 4.03 (d, J = |
| | | 13.5 Hz, 1H), 3.85 (d, J = 13.5 Hz, 1H), 3.64-3.55 (m, 3H), |
| | | 2.54-2.52 (m, 1H), 2.31-2.23 (m, 1H), 2.08-2.01 (m, 1H), |
| | | 1.85-1.78 (m, 1H), 1.50-1.42 (m, 1H), 1.07 (dd, J = 7.0, 3.5 |
| | | Hz, 6H) |

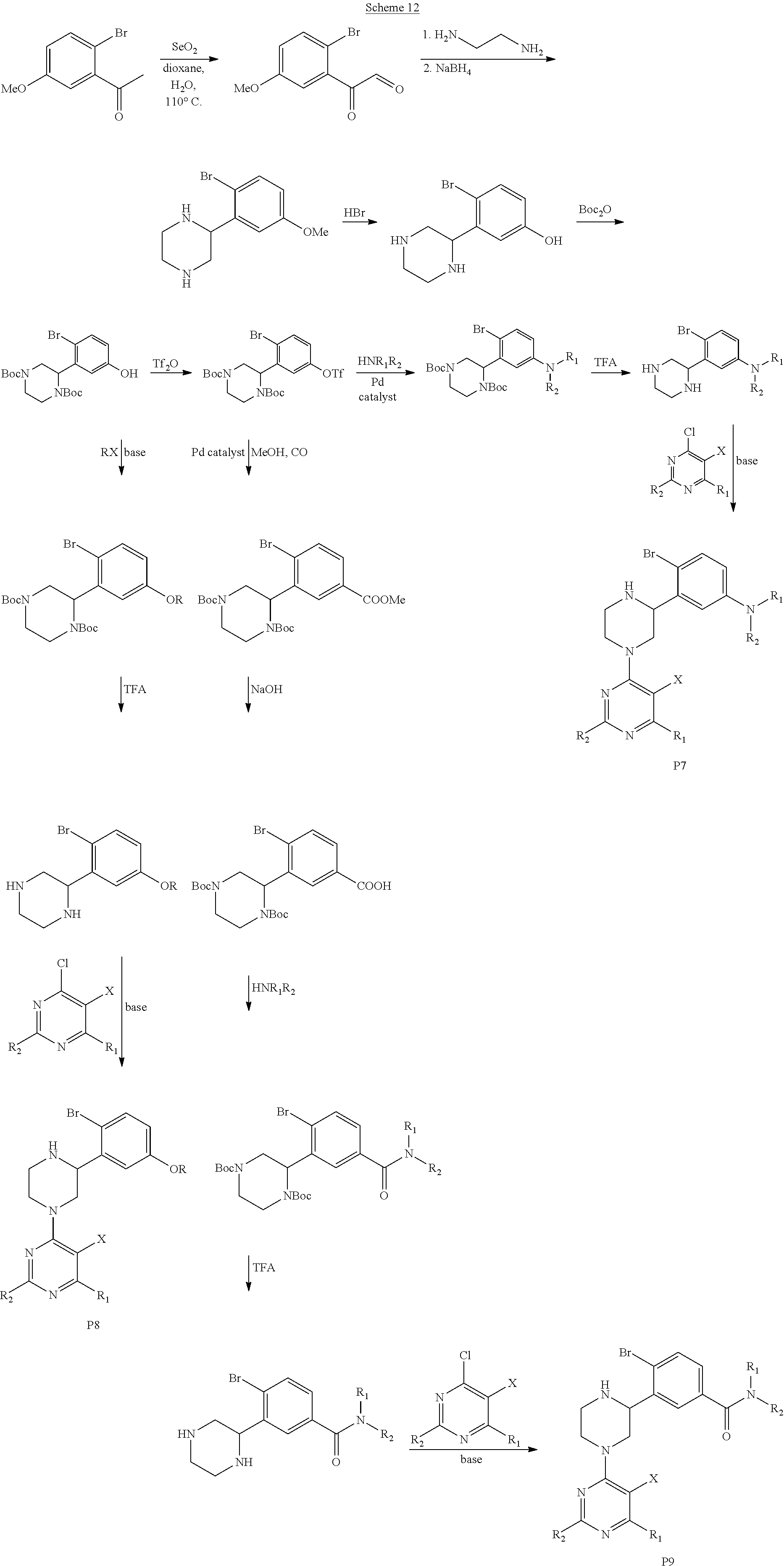

Compounds 9, 15, 16, 17, 18, 20, 21, 23, 24, 25, 26, 30, 31, 33, 50, 51, 377, 378, 379, 380, 381, 382, 383, and 384 can be prepared via a similar procedure to the one shown in Scheme 12.

TABLE 14
Characterization of selected compounds

| Compound | | | |
|---|---|---|---|
| No. | LC-MS | ¹H NMR | Chiral Separation Data |
| 9 | [M + H]⁺ = | ¹H NMR: (DMSO-d₆, 400 MHz) δ | |
| | 463.2 | 8.50 (t, J = 5.6 Hz, 1H), 8.17 (d, J = | |
| | | 2.0 Hz, 1H), 7.73-7.66 (m, 2H), | |
| | | 5.72 (s, 2H), 5.48 (s, 2H), 5.10 (s, | |
| | | 1H), 4.14 (dd, J = 2.4, 10.4 Hz, | |
| | | 2H), 3.99 (dd, J = 2.4, 10.0 Hz, | |
| | | 1H), 3.39-3.36 (m, 2H), 3.08-3.04 | |
| | | (m, 1H), 2.78-2.74 (m, 2H), 2.46- | |
| | | 2.43 (m, 1H), 2.43-2.40 (m, 2H), | |
| | | 2.20 (s, 6H) | |
| 15 | [M + H]⁺ = | ¹H NMR (DMSO-d₆, 400 MHz) δ | SFC peak 2 (Retention |
| | 475.2 | 7.70-7.68 (m, 2H), 7.24 (dd, J = | time 0.905 min, DAICEL |
| | | 2.4, 8.4 Hz, 1H), 5.70 (s, 2H), 5.45 | CHIRALPAK OD |
| | | (s, 2H), 5.09 (s, 1H), 4.16-4.07 (m, | (250 mm*30 mm, 10 um)) |
| | | 2H), 3.97 (dd, J = 2.4, 10.0 Hz, | |
| | | 1H), 3.62-3.60 (s, 2H), 3.28-3.23 | |
| | | (m, 2H), 3.03-2.98 (m, 1H), 2.75- | |
| | | 2.67 (m, 2H), 2.37-2.32 (m, 3H), | |
| | | 2.26-2.24 (m, 2H), 2.19 (s, 3H) | |
| 16 | [M + H]⁺ = | | SFC peak 1 (Retention |
| | 475.2 | | time 0.552 min, DAICEL |
| | | | CHIRALPAK OD |
| | | | (250 mm*30 mm, 10 um)) |
| 17 | [M + H]⁺ = | ¹H NMR (CD₃OD, 400 MHz) δ | SFC peak 2 (Retention |
| | 502.2 | 7.65-7.62 (m, 2H), 7.19 (dd, J = | time 2.236 min, DAICEL |
| | | 2.0, 8.0 Hz, 1H), 5.95 (s, 1H), 4.43 | CHIRALPAK |
| | | (d, J = 12.4 Hz, 1H), 4.33 (d, J = | AS(250 mm*30 mm, 10 um)) |
| | | 12.4 Hz, 1H), 4.09 (dd, J = 2.8, | |
| | | 10.4 Hz, 1H), 3.69-3.68 (m, 2H), | |
| | | 3.36-3.35 (m, 2H), 3.05 (d, J = | |
| | | 12.0 Hz, 1H), 2.99-2.91 (m, 1H), | |
| | | 2.83-2.82 (m, 1H), 2.68-2.56 (m, | |
| | | 2H), 2.43-2.32 (m, 4H), 2.23 (s, | |
| | | 3H), 1.24 (d, J = 6.8 Hz, 6H) | |
| 18 | [M + H]⁺ = | | SFC peak 1 (Retention |
| | 502.2 | | time 1.887 min, DAICEL |
| | | | CHIRALPAK |
| | | | AS(250 mm*30 mm, 10 um)) |
| 20 | [M + H]⁺ = | | SFC peak 2 (Retention |
| | 490.2 | | time 1.436 min, DAICEL |
| | | | CHIRALPAK IC |
| | | | (250 mm*30 mm, 10 um)) |
| 21 | [M + H]⁺ = | ¹H NMR (DMSO-d₆, 400 MHz) δ | SFC peak 1 (Retention |
| | 490.2 | 8.57 (s, 1H), 8.19 (s, 1H), 7.75- | time 0.893 min, DAICEL |
| | | 7.70 (m, 2H), 5.96-5.95 (m, 3H), | CHIRALPAK IC |
| | | 4.35-4.33 (m, 2H), 4.00-3.98 (m, | (250 mm*30 mm, 10 um)) |
| | | 1H), 3.41-3.38 (m, 2H), 3.08 (d, J = | |
| | | 11.2 Hz, 1H), 2.87-2.80 (m, 1H), | |
| | | 2.78-2.75 (m, 1H), 2.63-2.58 (m, | |
| | | 4H), 2.29 (s, 6H), 1.13 (d, J = 7.2 | |
| | | Hz, 6H) | |
| 23 | [M + H]⁺ = | | SFC peak 2 (Retention |
| | 393.0 | | time 2.119 min, DAICEL |
| | | | CHIRALPAK |
| | | | AD(250 mm*30 mm, 10 um)) |
| 24 | [M + H]⁺ = | ¹H NMR (DMSO-d₆, 400 MHz) δ | SFC peak 1 (Retention |
| | 393.0 | 7.48 (d, J = 8.8 Hz, 1H), 7.25 (d, J = | time 0.943 min, DAICEL |
| | | 3.2 Hz, 1H), 6.81 (dd, J = 3.2, | CHIRALPAK |
| | | 8.8 Hz, 1H), 5.71 (s, 2H), 5.47 (s, | AD(250 mm*30 mm, 10 um)) |
| | | 2H), 5.08 (s, 1H), 4.15-4.05 (m, | |
| | | 2H), 4.05-3.97 (m, 2H), 3.89 (dd, J = | |
| | | 2.4, 10.0 Hz, 1H), 3.05-2.99 (m, | |
| | | 1H), 2.79-2.68 (m, 2H), 2.55-2.50 | |
| | | (m, 1H), 2.39-2.33 (m, 1H), 1.32 | |
| | | (t, J = 6.8 Hz, 3H) | |
| 25 | [M + H]⁺ = | | SFC peak 2 (Retention |
| | 420.1 | | time 1.993 min, DAICEL |
| | | | CHIRALPAK |
| | | | AD(250 mm*30 mm, 10 um)) |
| 26 | [M + H]⁺ = | ¹H NMR (DMSO-d₆, 400 MHz) δ | SFC peak 1 (Retention |
| | 420.1 | 7.49 (d, J = 8.8 Hz, 1H), 7.26 (d, J = | time 1.831 min, DAICEL |
| | | 3.2 Hz, 1H), 6.82 (dd, J = 3.2, | CHIRALPAK |
| | | 8.8 Hz, 1H), 5.91-5.88 (m, 3H), | AD(250 mm*30 mm, 10 um)) |
| | | 4.35-4.20 (m, 2H), 4.07-3.97 (m, | |
| | | 2H), 3.87 (dd, J = 2.4, 10.4 Hz, | |
| | | 1H), 3.03 (d, J = 10.8 Hz, 1H), | |
| | | 2.92 (s, 1H), 2.84-2.70 (m, 2H), | |
| | | 2.61-2.52 (m, 2H), 1.32 (t, J = 6.8 | |
| | | Hz, 3H), 1.12 (d, J = 6.8 Hz, 6H) | |
| 30 | [M + H]⁺ = | | SFC peak 2 (Retention |
| | 461.2 | | time 2.019 min, DAICEL |
| | | | CHIRALPAK |
| | | | AS(250 mm*30 mm, 10 um)) |
| 31 | [M + H]⁺ = | ¹H NMR: (DMSO-d₆, 400 MHz) δ | SFC peak 1 (Retention |
| | 461.2 | 7.33 (d, J = 8.8 Hz, 1H), 6.74 (d, J = | time 1.795 min, DAICEL |
| | | 2.8 Hz, 1H), 6.29 (dd, J = 2.8, | CHIRALPAK |
| | | 8.4 Hz, 1H), 5.91 (s, 1H), 5.89 (s, | AS(250 mm*30 mm, 10 um)) |
| | | 2H), 4.75 (t, J = 5.2 Hz, 1H), 4.35- | |
| | | 4.27 (m, 2H), 3.86-3.83 (m, 3H), | |
| | | 3.58-3.52 (m, 4H), 3.04 (d, J = | |
| | | 12.4 Hz, 1H), 2.83-2.76 (m, 4H), | |
| | | 2.58-2.46 (m, 1H), 2.47-2.42 (m, | |
| | | 1H), 1.13 (d, J = 6.8 Hz, 6H) | |
| 33 | LC-MS: | ¹H NMR (CD₃OD, 400 MHz) | |
| | [M + H]⁺ = | δ 7.39 (d, J = 8.8 Hz, 1H), 7.24 (d, | |
| | 462.2 | J = 2.8 Hz, 1H), 6.82 (dd, J = 2.8, | |
| | | 8.8 Hz, 1H), 5.27 (s, 1H), 4.23 (d, | |
| | | J = 10.4 Hz, 2H), 4.11 (dd, J = 2.8, | |
| | | 10.0 Hz, 1H), 3.80-3.71 (m, 2H), | |
| | | 3.44 (d, J = 6.4 Hz, 2H), 3.13-3.08 | |
| | | (m, 1H), 2.99-2.86 (m, 2H), 2.76- | |
| | | 2.65 (m, 3H), 1.84 (d, J = 12.4 Hz, | |
| | | 2H), 1.68-1.56 (m, 1H), 1.38 (m, | |
| | | 2H) | |
| 50 | [M + H]⁺ = | | SFC peak 2 (Retention |
| | 447.0 | | time 1.101 min, DAICEL |
| | | | CHIRALCEL |
| | | | OD(250 mm*30 mm, 10 um)) |
| 51 | [M + H]⁺ = | ¹H NMR (DMSO-d₆, 400 MHz) δ | SFC peak 1 (Retention |
| | 447.0 | 7.37 (d, J = 8.8 Hz, 1H), 7.26-7.22 | time 0.625 min, DAICEL |
| | | (m, 1H), 6.81 (dd, J = 3.2, 9.2 Hz, | CHIRALCEL |
| | | 1H), 5.69 (s, 2H), 5.47-5.40 (m, | OD(250 mm*30 mm, 10 um)) |
| | | 2H), 5.07 (s, 1H), 4.14-3.99 (m, | |
| | | 2H), 3.89-3.79 (m, 1H), 3.16-3.08 | |
| | | (m, 4H), 3.05-2.98 (m, 1H), 2.87- | |
| | | 2.79 (m, 1H), 2.74-2.65 (m, 2H), | |
| | | 2.46-2.42 (m, 4H), 2.35-2.32 (m, | |
| | | 1H), 2.21 (s, 3H) | |
| 377 | | ¹H NMR (400 MHz, DMSO-d₆) δ | |
| | | 8.18 (s, 1H), 7.80 (d, J = 8.0 Hz, | |
| | | 1H), 7.71 (d, J = 8.0 Hz, 1H), 5.67 | |
| | | (s, 2H), 5.42 (s, 2H), 5.02 (s, 1H), | |
| | | 4.54-4.48 (m, 2H), 4.41-4.32 (m, | |
| | | 1H), 4.26-4.18 (m, 1H), 4.12-4.06 | |
| | | (m, 2H), 3.95-3.86 (m, 2H), 3.04- | |
| | | 3.01 (m, 2H), 2.76 (t, J = 8.4 Hz, | |
| | | 2H), 2.54-2.53 (m, 1H). | |
| 378 | | ¹H NMR (400 MHz, CDCl₃) δ 8.01 | |
| | | (s, 1H), 7.71 (d, J = 8.0 Hz, 1H), | |
| | | 7.42 (d, J = 8.0 Hz, 1H), 5.12 (s, | |
| | | 1H), 4.53 (s, 2H), 4.35 (S, 2H), | |
| | | 4.29 (d, J = 9.6 Hz, 1H), 4.19 (d, J = | |
| | | 9.2 Hz, 2H), 4.00 (s, 1H), 3.55- | |
| | | 3.49 (m, 3H), 3.37 (s, 3H), 3.24- | |
| | | 3.11 (m, 2H), 2.99-2.97 (m, 2H), | |
| | | 2.73-2.72 (m, 1H), 2.02-1.95 (m, | |
| | | 1H), 1.79-1.67 (m, 2H), 1.63-1.51 | |
| | | (m, 1H). | |
| 379 | | ¹H NMR (400 MHz, DMSO-d₆) δ | |
| | | 7.94 (s, 1H), 7.78 (d, J = 8.0 Hz, | |
| | | 1H), 7.48 (d, J = 8.0 Hz, 1H), 5.67 | |
| | | (s, 2H), 5.41 (s, 2H), 5.02 (s, 1H), | |
| | | 4.10-4.04 (m, 2H), 3.95-3.92 (m, | |
| | | 1H), 3.61-3.57 (m, 2H), 3.27-3.20 | |
| | | (m, 2H), 3.03-2.98 (m, 2H), 2.75- | |
| | | 2.72 (m, 2H), 1.62-1.54 (m, 4H), | |
| | | 1.45 (s, 2H). | |
| 380 | | ¹H NMR (400 MHz, DMSO-d₆) δ | |
| | | 7.94 (s, 1H), 7.78 (d, J = 8.0 Hz, | |
| | | 1H), 7.48 (d, J = 7.6 Hz, 1H), 5.67 | |
| | | (s, 2H), 5.42 (s, 2H), 5.02 (s, 1H), | |
| | | 4.10-4.04 (m, 2H), 3.93 (d, J = 9.2 | |
| | | Hz, 1H), 3.61 (s, 2H), 3.23-3.22 | |
| | | (m, 2H), 3.02 (d, J = 8.0 Hz, 2H), | |
| | | 2.75-2.67 (m, 2H), 1.62-1.57 (m, | |
| | | 4H), 1.45 (s, 2H). | |
| 381 | | ¹H NMR (400 MHz, DMSO-d₆) δ | |
| | | 8.02 (s, 1H), 7.80 (d, J = 8.0 Hz, | |
| | | 1H), 7.57 (d, J = 8.4 Hz, 1H), 5.88 | |
| | | (s, 2H), 5.68 (s, 2H), 5.04 (s, 1H), | |
| | | 4.00 (t, J = 9.2 Hz, 2H), 3.94-3.91 | |
| | | (m, 1H), 3.76-3.73 (m, 2H), 3.37- | |
| | | 3.35 (m, 2H), 3.04 (d, J = 9.6 Hz, | |
| | | 1H), 2.78-2.70 (m, 2H), 2.57 (t, J = | |
| | | 11.2 Hz, 1H), 2.09-2.01 (m, 4H). | |
| 382 | | ¹H NMR (400 MHz, DMSO-d₆) δ | |
| | | 8.02 (s, 1H), 7.80 (d, J = 8.0 Hz, | |
| | | 1H), 7.57 (d, J = 8.0 Hz, 1H), 5.69 | |
| | | (s, 2H), 5.43 (s, 2H), 5.02 (s, 1H), | |
| | | 4.10-4.05 (m, 2H), 3.94 (d, J = | |
| | | 10.4 Hz, 1H), 3.77-3.73 (s, 2H), | |
| | | 3.40-3.35 (m, 2H), 3.04-3.01 (m, | |
| | | 2H), 2.76-2.73 (m, 2H), 2.56-2.53 | |
| | | (m, 1H), 2.09-2.03 (m, 4H). | |
| 383 | | ¹H NMR (400 MHz, DMSO-d₆) δ | |
| | | 7.98 (s, 1H), 7.79 (d, J = 8.4 Hz, | |
| | | 1H), 7.52 (d, J = 8.4 Hz, 1H), 5.67 | |
| | | (s, 2H), 5.41 (s, 2H), 5.02 (s, 1H), | |
| | | 4.13-4.01 (m, 2H), 3.94 (d, J = 9.6 | |
| | | Hz, 1H), 3.79-3.45 (m, 8H), 3.08- | |
| | | 2.96 (m, 2H), 2.78-2.69 (m, 2H). | |
| 384 | | ¹H NMR (400 MHz, DMSO-d₆) δ | |
| | | 7.98 (s, 1H), 7.79 (d, J = 8.0 Hz, | |
| | | 1H), 7.53 (d, J = 8.0 Hz, 1H), 5.68 | |
| | | (s, 2H), 5.43 (s, 2H), 5.02 (s, 1H), | |
| | | 4.11-4.04 (m, 2H), 3.94 (br d, J = | |
| | | 9.6 Hz, 1H), 3.69-3.51 (m, 8H), | |
| | | 3.07-2.95 (m, 2H), 2.79-2.67 (m, | |
| | | 2H). | |

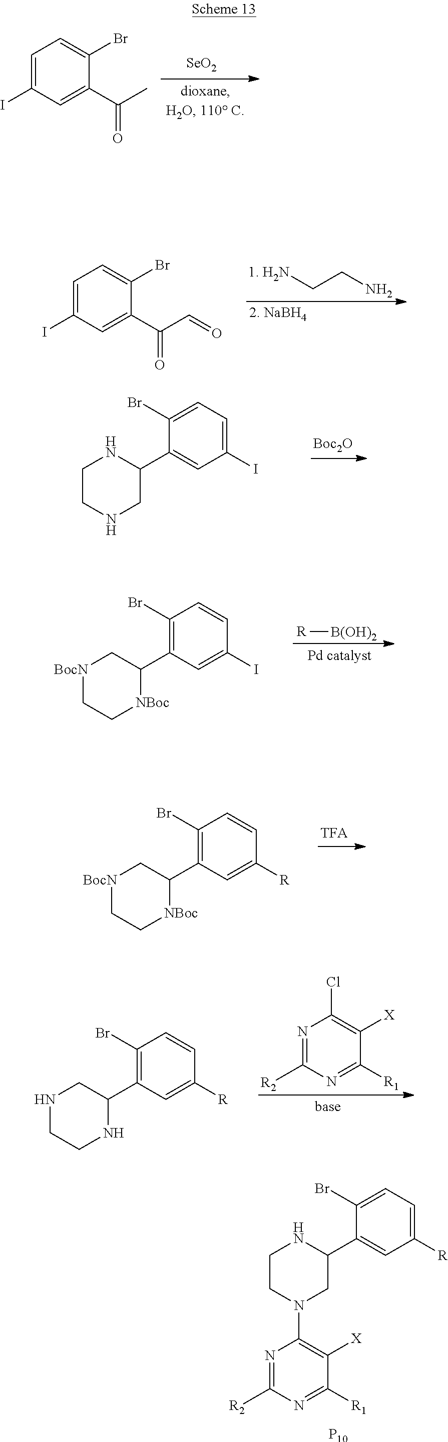

Compounds 4-7 can be prepared via a similar procedure to the one shown in Scheme 13.

TABLE 15
Characterization of selected compounds

| Compound | | | |
|---|---|---|---|
| No. | LC-MS | ¹H NMR | Chiral Separation Data |
| 4 | [M + H]⁺ = | | SFC peak 2 (Retention |
| | 443.2 | | time 2.264 min, |
| | | | DAICEL CHIRALPAK |
| | | | AS |
| | | | (250 mm*30 mm, 10 um)) |
| 5 | [M + H]⁺ = | ¹H NMR (DMSO-d₆, 400 MHz) δ 7.71 | SFC peak 1 (Retention |
| | 443.2 | (d, J = 2.4 Hz, 1H), 7.62 (d, J = 8.4 | time 1.980 min, |
| | | Hz, 1H), 7.57 (s, 1H), 7.27-7.23 (m, | DAICEL CHIRALPAK |
| | | 1H), 5.70 (s, 2H), 5.45 (s, 2H), 5.10 | AS |
| | | (s, 1H), 4.18-4.08 (m, 2H), 4.00-3.95 | (250 mm*30 mm, 10 um)) |
| | | (m, 1H), 3.78 (s, 3H), 3.05-3.01 (m, | |
| | | 1H), 2.99-2.91 (m, 1H), 2.77-2.71 (m, | |
| | | 2H), 2.45-2.40 (m, 1H), 2.36 (s, 3H) | |
| 6 | [M + H]⁺ = | | SFC peak 2 (Retention |
| | 470.2 | | time 1.466 min, |
| | | | DAICEL CHIRALPAK |
| | | | AD |
| | | | (250 mm*30 mm, 10 um)) |
| 7 | [M + H]⁺ = | ¹H NMR: (DMSO-d₆, 400 MHz) δ 7.70 | SFC peak 1 (Retention |
| | 470.2 | (s, 1H), 7.62 (d, J = 8.0 Hz, 1H), 7.57 | time 0.966 min, |
| | | (s, 1H), 7.25 (dd, J = 2.4, 8.4 Hz, 1H), | DAICEL CHIRALPAK |
| | | 5.93 (s, 1H), 5.89 (s, 2H), 4.43-4.18 | AD |
| | | (m, 2H), 3.98-3.93 (m, 1H), 3.78 (s, | (250 mm*30 mm, 10 um)) |
| | | 3H), 3.10-2.96 (m, 2H), 2.85-2.75 (m, | |
| | | 2H), 2.59-2.58 (m, 1H), 2.36 (s, 3H), | |
| | | 1.12 (d, J = 6.8 Hz, 6H) | |

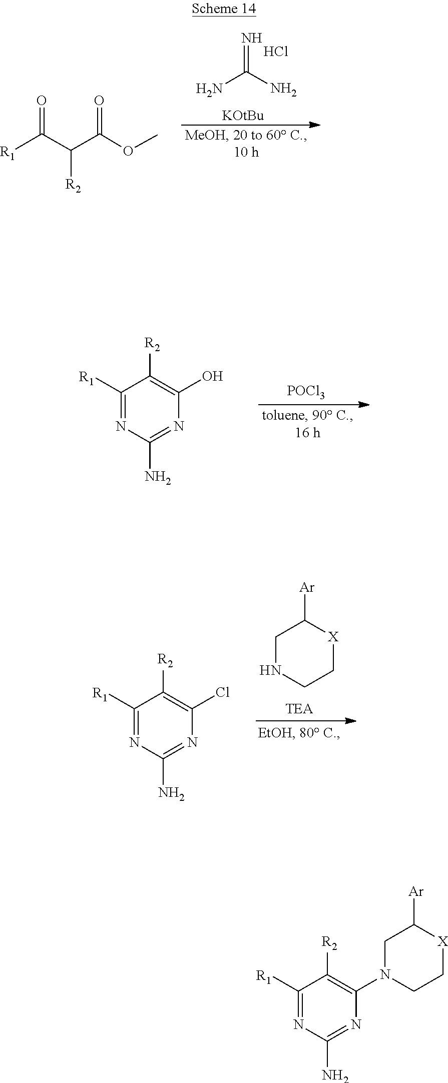

Compounds 184, 185, 186, 189, 191, 196, 199, 201, 208, 211, 212, 215, and 219 can be prepared via a similar procedure to the one shown in Scheme 14.

TABLE 16
Characterization of selected compounds

| Compound | | |
|---|---|---|
| No. | LC-MS | ¹H NMR |
| 184 | [M + H]⁺ = | ¹H NMR (MeOD-d₄, 400 MHz) δ 8.60-8.05 (m, 2H), 7.66 (d, |
| | 389.9 | J = 8.0 Hz, 2H), 7.49-7.39 (m, 1H), 7.27 (td, J = 7.6, 1.6 |
| | | Hz, 1H), 4.59 (q, J = 13.2 Hz, 2H), 4.41 (dd, J = 10.8, 2.4 |
| | | Hz, 1H), 4.11-3.97 (m, 4H), 3.26-3.16 (m, 3H), 3.02-2.94 |
| | | (m, 1H), 2.75 (s, 2H) |
| 185 | [M + H]⁺ = | ¹H NMR (DMSO-d₆, 400 MHz) δ 14.10-13.54 (m, 1H), 8.57- |
| | 305.2 | 7.88 (m, 1H), 6.10 (s, 2H), 4.44-4.34 (d, J = 12.0 Hz, 1H), |
| | | 4.15 (s, 2H), 4.03-3.97 (m, 1H), 3.94-3.93 (m, 1H), 3.84 (s, |
| | | 2H), 3.10-3.02 (m, 2H), 3.00-2.94 (m, 1H), 2.78-2.50 (m, |
| | | 1H) |
| 186 | [M + H]⁺ = | ¹H NMR (DMSO-d₆, 400 MHz) δ 8.51 (d, J = 6.8 Hz, 1H), |
| | 353.1 | 7.56 (d, J = 9.2 Hz, 1H), 7.45 (s, 1H), 7.27-7.19 (m, 1H), |
| | | 6.99 (t, J = 6.8 Hz, 1H), 6.10 (s, 2H), 4.56 (d, J = 12.8 Hz, |
| | | 1H), 4.28-4.06 (m, 3H), 3.93-3.77 (m, 2H), 3.30-3.20 (m, |
| | | 1H), 3.09 (t, J = 12.0 Hz, 1H) 3.03-2.94 (m, 1H), 2.16 (d, J = |
| | | 12.0 Hz, 1H) |
| 189 | [M + H]⁺ = | ¹H NMR (DMSO-d₆, 400 MHz) δ 7.73 (dd, J = 8.0, 1.6 Hz, |
| | 391.9 | 1H), 7.61 (dd, J = 8.0, 1.2 Hz, 1H), 7.41 (t, J = 7.6 Hz, 1H). |
| | | 7.23 (td, J = 7.6, 1.6 Hz, 1H), 6.10 (s, 2H) 4.27-4.15 (m, |
| | | 3H), 4.10-4.00 (m, 2H), 3.92-3.86 (m, 1H), 3.84-3.78 (m, |
| | | 1H), 3.04 (br d, J = 10.0 Hz, 1H), 2.89-2.83 (m, 2H), 2.56 |
| | | (br s, 2H) |
| 191 | [M + H]⁺ = | ¹H NMR (DMSO-d₆, 400 MHz) δ 8.58 (d, J = 6.8 Hz, 1H), |
| | 354.1 | 7.76 (d, J = 9.6 Hz, 1H), 7.44-7.29 (m, 1H), 7.11-6.95 (m, |
| | | 1H), 5.98 (s, 2H), 4.62 (d, J = 12.8 Hz, 1H), 4.36 (d, J = |
| | | 13.2 Hz, 1H), 3.51-3.46 (m, 1H), 3.28-3.22 (m, 1H), 3.19- |
| | | 3.12 (m, 2H), 3.10-3.02 (m, 1H), 3.00-2.91 (m, 2H), 2.20 |
| | | (d, J = 12.8 Hz, 1H), 1.98-1.80 (m, 2H), 1.73-1.59 (m, 1H) |
| 196 | [M + H]⁺ = | ¹H NMR (CD₃OD, 400 MHz) δ 7.56-7.53 (m, 1H), 6.20 (s, |
| | 301.3 | 1H), 4.51 (s, 2H), 4.05-3.90 (m, 3H), 3.79-3.64 (m, 1H), |
| | | 3.09-3.03 (m, 3H), 2.68 (t, J = 6.0 Hz, 2H), 2.13-2.06 (m, |
| | | 1H), 1.69-1.66 (m, 3H) |
| 199 | [M + H]⁺ = | ¹H NMR (CD₃OD, 400 MHz) δ 7.61 (d, J = 2.4 Hz, 1H), |
| | 303.1 | 6.28 (d, J = 2.4 Hz, 1H), 4.86-4.45 (m, 2H), 4.38-4.32 (m, |
| | | 2H), 4.12-4.10 (m, 2H), 3.36-3.35 (m, 1H), 3.34-3.28 (m, |
| | | 1H), 3.05-3.01 (m, 1H), 2.22-2.13 (m, 1H), 1.98-1.82 (m, |
| | | 2H), 1.79-1.72(m, 1H) |
| 201 | [M + H]⁺ = | ¹H NMR (DMSO-d₆, 400 MHz) δ 8.17 (s, 1H), 7.26 (s, 1H), |
| | 308.1 | 6.72 (s, 1H), 6.03 (s, 2H), 4.23-4.03 (m, 4H), 3.83 (s, 2H), |
| | | 2.90-2.77 (m, 1H), 2.58-2.55 (m, 1H), 2.10 (t, J = 7.2 Hz, |
| | | 2H), 1.81 (d, J = 12.0 Hz, 1H), 1.69-1.58 (m, 1H), 1.52-1.32 |
| | | (m, 4H), 1.21-1.05 (m, 1H) |
| 208 | [M + H]⁺ = | ¹H NMR(DMSO-d₆, 400 MHz) δ 13.95 (s, 1H), 7.81 (s, 2H), |
| | 317.0 | 7.58 (d, J = 2.0 Hz, 1H), 6.16 (d, J = 2.0 Hz, 1H), 4.37 (d, J = |
| | | 12.4 Hz, 1H), 4.15 (d, J = 13.2 Hz, 1H), 3.72-3.55 (m, |
| | | 2H), 3.20-3.06 (m, 2H), 3.01-2.86 (m, 5H), 2.13-2.02 (m, |
| | | 1H), 1.86-1.60 (m, 3H) |
| 211 | [M + H]⁺ = | ¹H NMR(CD₃OD, 400 MHz) δ 7.66-7.43 (m, 1H), 6.23 (s, |
| | 285.3 | 1H), 5.97 (s, 1H), 4.53 (d, J = 12.8 Hz, 1H), 4.43-4.24 (m, |
| | | 1H), 3.09-2.81 (m, 3H), 2.21-2.10 (m, 1H), 1.86-1.73 (m, |
| | | 3H), 1.69-1.54 (m, 1H), 0.99-0.84 (m, 4H) |
| 212 | [M + H]⁺ = | ¹H NMR (CD₃OD, 400 MHz) δ 7.64-7.42 (m, 1H), 6.22 (s, |
| | 299.2 | 1H), 6.00 (s, 1H), 4.55 (d, J = 12.0 Hz, 1H), 4.42-4.26 (m, |
| | | 1H), 3.38-3.32 (m, 1H), 3.05-2.93 (m, 2H), 2.92-2.83 (m, |
| | | 1H), 2.30-2.10 (m, 5H), 2.07-1.97 (m, 1H), 1.88-1.74 (m, |
| | | 3H), 1.67-1.54 (m, 1H) |
| 215 | [M + H]⁺ = | ¹H NMR(CD₃OD, 400 MHz) δ 7.60 (d, J = 2.0 Hz, 1H), 6.27 |
| | 285.4 | (d, J = 2.0 Hz, 1H), 5.24-5.08 (m, 1H), 4.6-4.36 (m, 1H), |
| | | 3.32-3.12 (m, 2H), 3.10-2.98 (m, 3H), 2.86 (t, J = 7.8 Hz, |
| | | 2H), 2.24-2.12 (m, 3H), 1.99-1.83 (m, 2H), 1.78-1.62 (m, |
| | | 1H) |
| 219 | [M + H]⁺ = | ¹H NMR(CD₃OD, 400 MHz) δ 7.59 (d, J = 2.4 Hz, 1H), |
| | 301.3 | 6.26 (d, J = 2.4 Hz, 1H), 4.74-4.62 (m, 1H), 4.56 (d, J = 2.0 |
| | | Hz, 2H), 4.52-4.40 (m, 1H), 3.96-3.84 (m, 1H), 3.81-3.78 |
| | | (m, 1H), 3.26-3.21 (m, 2H), 3.18-3.05 (m, 1H), 2.80-2.56 |
| | | (m, 2H), 2.24-2.12 (m, 1H), 2.00-1.64 (m, 3H) |

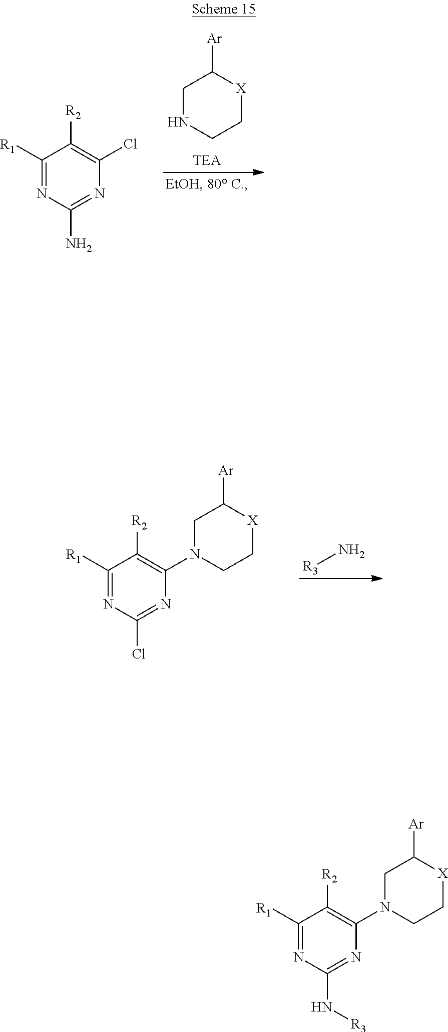

Compounds 71, 77, 78, 85, 113, 114, 117, 118, 127, 134, 150, 151, 152, and 53 can be prepared via a similar procedure to the one shown in Scheme 15.

TABLE 17
Characterization of selected compounds

| Compound | | | |
|---|---|---|---|
| No. | LC-MS | ¹H NMR | Chiral Separation Data |
| 71 | [M + H]⁺ = | ¹H NMR: (DMSO-d₆, 400 MHz) δ 7.66- | |
| | 402.8 | 7.56 (m, 2H), 7.43 (t, J = 7.2 Hz, 1H), | |
| | | 7.27-7.20 (m, 1H), 6.13 (d, J = 3.6 Hz, | |
| | | 1H), 5.75 (s, 2H), 5.11 (s, 1H), 4.12 (d, J = | |
| | | 11.2 Hz, 2H), 3.41 (dd, J = 3.2, 10.4 | |
| | | Hz, 1H), 3.01-2.84 (m, 2H), 2.63-2.59 (m, | |
| | | 1H), 2.48-2.44 (m, 1H), 2.22 (dt, J = 2.8, | |
| | | 11.6 Hz, 1H), 1.97 (s, 3H), 0.53-0.50 (m, | |
| | | 2H), 0.40-0.32 (m, 2H) | |
| 77 | [M + H]⁺ = | ¹H NMR (DMSO-d₆, 400 MHz) δ 7.64 (d, | SFC peak 2 |
| | 403.8 | J = 7.8 Hz, 1H), 7.57 (d, J = 6.8 Hz, 1H), | (Retention time |
| | | 7.44 (t, J = 7.6 Hz, 1H), 7.29 (t, J = 6.8 | 2.31 min, DAICEL |
| | | Hz, 1H), 6.39-6.25 (m, 1H), 5.57-5.48 (m, | CHIRALCEL |
| | | 2H), 5.12 (s, 1H), 4.70-4.64 (m, 1H), | OD(250 mm*30 mm, |
| | | 4.27-4.22 (m, 1H), 4.08-4.02 (m, 2H), | 10 um)) |
| | | 3.69 (d, J = 11.6 Hz, 1 H), 3.04 (t, J = 6.0 | |
| | | Hz, 2H), 2.89-2.83 (m, 1H), 2.58-2.55 (m, | |
| | | 1H), 1.00-0.94 (m, 1H), 0.42-0.36 (m, | |
| | | 2H), 0.18-0.13 (m, 2H) | |
| 78 | [M + H]⁺ = | ¹H NMR (DMSO-d₆, 400 MHz) | SFC peak 1 |
| | 403.8 | δ 7.64 (dd, J = 8.0, 1.2 Hz, 1H), 7.57 (dd, | (Retention time |
| | | J = 7.6, 1.6 Hz, 1H), 7.47-7.41 (m, 1H), | 1.95 min, DAICEL |
| | | 7.31-7.25 (m, 1H), 6.31 (br s, 1H), 5.54 | CHIRALCEL |
| | | (s, 2H) 5.12 (s, 1H) 4.68 (dd, J = 10.0, | OD(250 mm*30 mm, |
| | | 2.4 Hz, 1H), 4.25 (d, J = 12.8 Hz, 1H), | 10 um)) |
| | | 4.10-4.00 (m, 2H), 3.72-3.63 (m, 1H), | |
| | | 3.04 (t, J = 6.0 Hz, 2H), 2.90-2.82 (m, | |
| | | 1H), 2.61-2.58 (m, 1H), 1.02-0.92 (m, | |
| | | 1H), 0.42-0.36 (m, 2H), 0.2-0.10 (m, 2H) | |
| 85 | [M + H]⁺ = | ¹H NMR (DMSO-d₆, 400 MHz) δ 8.54 (s, | |
| | 362.1 | 1H), 7.60 (d, J = 8.0 Hz, 1H), 7.42-7.41 | |
| | | (m, 1 H), 7.39-7.33 (m, 1H), 7.15 (t, J = | |
| | | 8.0 Hz, 1H), 4.88-4.87 (m, 1H), 4.54-4.32 | |
| | | (m, 2H), 3.23-3.17 (m, 1H), 3.03-2.99 (m, | |
| | | 1H), 2.94 (d, J = 12.4 Hz, 1H), 2.89 (s, | |
| | | 3H), 2.04-2.02 (m, 1H), 1.93-1.83 (m, | |
| | | 2H), 1.71-1.61 (m, 1H) | |
| 113 | [M + H]⁺ = | | SFC peak 2 |
| | 363.0 | | (Retention time |
| | | | 2.497 min, DAICEL |
| | | | CHIRALCEL AD |
| | | | (250 mm*30 mm, 10 um)) |
| 114 | [M + H]⁺ = | ¹H NMR (DMSO-d₆, 400 MHz) δ 7.72 (d, | SFC peak 1 |
| | 363.0 | J = 7.6 Hz, 1H), 7.62 (d, J = 8.0 Hz, 1H), | (Retention time |
| | | 7.41 (t, J = 7.2 Hz, 1H), 7.28-7.20 (m, | 2.272 min, DAICEL |
| | | 1H), 6.09-5.97 (m, 1H), 5.95-5.79 (m, | CHIRALCEL AD |
| | | 2H), 5.09 (s, 1H), 4.37-4.21 (m, 1H), | (250 mm*30 mm, 10 um)) |
| | | 4.14-4.04 (m, 1H), 4.03-3.92 (m, 1H), | |
| | | 3.06 (d, J = 8.4 Hz, 1H), 2.79 (d, J = 8.0 | |
| | | Hz, 2H), 2.69 (d, J = 4.8 Hz, 3H), 2.44- | |
| | | 2.33 (m, 2H) | |
| 117 | [M + H]⁺ = | ¹H NMR (CD₃OD, 400 MHz) δ 8.48-8.42 | SFC peak 2 |
| | 324.0 | (m, 1H), 7.55 (d, J = 8.8 Hz, 1H), 7.47- | (Retention time |
| | | 7.42 (m, 1H), 7.35-7.29 (m, 1H), 7.02- | 2.141 min, DAICEL |
| | | 6.95 (m, 1H), 4.94 (s, 1H),, 4.76 (d, J = | CHIRALCEL OD |
| | | 0.8 Hz, 1H), 4.13-4.05 (m, 1H), 3.19-3.13 | (250 mm*30 mm, 10 um)) |
| | | (m, 1H), 3.12-2.99 (m, 1H), 2.98-2.91 (m, | |
| | | 1H), 2.87 (s, 3H), 2.29-2.21 (m, 1H), | |
| | | 2.03-1.94 (m, 1 H), 1.89-1.75 (m, 2H) | |
| 118 | [M + H]⁺ = | ¹H NMR (CD₃OD, 400 MHz) δ 8.44 (d, J = | SFC peak 1 |
| | 324.1 | 6.8 Hz, 1H), 7.58-7.53 (m, 1H), 7.45 (s, | (Retention time |
| | | 1H), 7.35-7.29 (m, 1H), 7.02-6.97 (m, | 2.024 min, DAICEL |
| | | 1H), 4.94 (s, 1H), 4.78-4.74 (m, 1H), | CHIRALCEL OD |
| | | 4.13-4.05 (m, 1H), 3.22-3.12 (m, 2H), | (250 mm*30 mm, 10 um)) |
| | | 3.11-3.03 (m, 1H), 2.98 (dd, J = 13.2, | |
| | | 10.4 Hz, 1H), 2.87 (s, 3H), 2.29-2.23 (m, | |
| | | 1H), 2.04-1.95 (m, 1H), 1.92-1.75 (m, | |
| | | 2H), LC-MS: [M + H]⁺ = 324.1 | |
| 127 | [M + H]⁺ = | ¹H NMR (CD₃OD, 400 MHz) δ 8.44 (d, J = | |
| | 381.2 | 6.0 Hz, 1H), 7.58 (d, J = 9.2 Hz, 1H), 7.47 | |
| | | (s, 1 H), 7.39-7.31 (m, 1 H), 7.04 (t, J = | |
| | | 6.8 Hz, 1H), 5.99 (s, 1H), 4.84-4.77 (m, | |
| | | 1H), 4.27-4.14 (m, 1H), 3.74-3.66 (m, | |
| | | 2H), 3.55-3.45 (m, 2H), 3.29-3.19 (m, | |
| | | 2H), 3.17-3.08 (m, 1H), 2.73-2.61 (m, | |
| | | 1H), 2.33-2.25 (m, 1H), 2.09-1.97 (m, 1 | |
| | | H), 1.96-1.87 (m, 1H), 1.85-1.69 (m, 1H), | |
| | | 1.24-1.21 (m, 6H) | |
| 134 | [M + H]⁺ = | ¹H NMR (DMSO-d₆, 400 MHz) δ 8.44 (d, | |
| | 351.3 | J = 6.8 Hz, 1H), 8.23 (s, 1H), 7.56 (d, J = | |
| | | 9.2 Hz, 1H), 7.45 (s, 1H), 7.26-7.18 (m, | |
| | | 1H), 6.93 (t, J = 6.8 Hz, 1H), 6.43-6.27 | |
| | | (m, 1H), 5.94 (s, 1H), 4.73 (d, J = 4.0 Hz, | |
| | | 1H), 4.29-4.12 (m, 1H), 3.14 (m, 1H), | |
| | | 3.06 (t, J = 11.6 Hz, 1H), 2.99-2.89 (m, | |
| | | 1H), 2.76 (d, J = 4.8 Hz, 3H), 2.63-2.57 | |
| | | (m, 1H), 2.15 (d, J = 14.0 Hz, 1H), 1.91- | |
| | | 1.82 (m, 1H), 1.80-1.74 (m, 1H), 1.64- | |
| | | 1.57 (m, 1H), 1.16-1.13 (m, 6H) | |
| 150 | [M + H]⁺ = | ¹H NMR (CD₃CN, 400 MHz) δ 7.73-7.71 | SFC peak 1 |
| | 418.1 | (m, 1H), 7.62-7.59 (m, 1H), 7.41-7.37 (m, | (Retention time |
| | | 1H), 7.22-7.20 (m, 1H), 5.89 (s, 1H), | 1.109 min, DAICEL |
| | | 4.97-4.96 (m, 1H), 4.52-4.49 (m, 1H), | CHIRALCEL OJ |
| | | 4.30-4.28 (m, 1H), 4.08-4.05 (m, 2H), | (250 mm*30 mm, 10 um)) |
| | | 3.11-3.08 (m, 1H), 2.95-2.83 (m, 2H), | |
| | | 2.64-2.53 (m, 2H), 1.16-1.13 (m, 12H), | |
| | | LC-MS: [M + H]⁺ = 418.1 | |
| 151 | [M + H]⁺ = | ¹H NMR (CD₃CN, 400 MHz) δ 7.73-7.71 | SFC peak 2 |
| | 418.2 | (m, 1H), 7.60 (d, J = 8.0 Hz, 1H), 7.39 (t, | (Retention time |
| | | J = 7.6 Hz, 1H), 7.22-7.20 (m, 1H), 5.90 | 1.441 min, DAICEL |
| | | (s, 1H), 4.99-4.97 (m, 1H), 4.52-4.49 (m, | CHIRALCEL OJ |
| | | 1H), 4.30-4.28 (m, 1H), 4.10-4.02 (m, | (250 mm*30 mm, 10 um)) |
| | | 2H), 3.11-3.07 (m, 1H), 2.95-2.83 (m, | |
| | | 2H), 2.64-2.53 (m, 2H), 1.16-1.13 (m, | |
| | | 12H) | |
| 152 | [M + H]⁺ = | | SFC peak 2 |
| | 390.2 | | (Retention time |
| | | | 1.622 min, DAICEL |
| | | | CHIRALCEL OJ |
| | | | (250 mm*30 mm, 10 um)) |
| 153 | [M + H]⁺ = | ¹H NMR (CD₃CN, 400 MHz) δ 7.72 (d, J = | SFC peak 1 |
| | 390.2 | 7.6 Hz, 1H), 7.60 (d, J = 8.0 Hz, 1H), | (Retention time |
| | | 7.40 (t, J = 7.6 Hz, 1H), 7.20 (t, J = 7.6 | 1.333 min, DAICEL |
| | | Hz, 1H), 5.91 (s, 1H), 5.12 (s, 1H), 4.63- | CHIRALCEL OJ |
| | | 4.45 (m, 1H), 4.32 (d, J = 10.4 Hz, 1H), | (250 mm*30 mm, 10 um)) |
| | | 4.07 (dd, J = 2.4, 10.0 Hz, 1H), 3.10 (d, J = | |
| | | 10.0 Hz, 1H), 2.97-2.85 (m, 2H), 2.83 | |
| | | (d, J = 5.2 Hz, 3H), 2.66-2.53 (m, 2H), | |
| | | 1.16 (d, J = 6.8 Hz, 6H) | |

### Preparation of Intermediates

### Preparation of 3-(piperazin-2-yl)imidazo[1,2-a]pyridine, Int 1-1

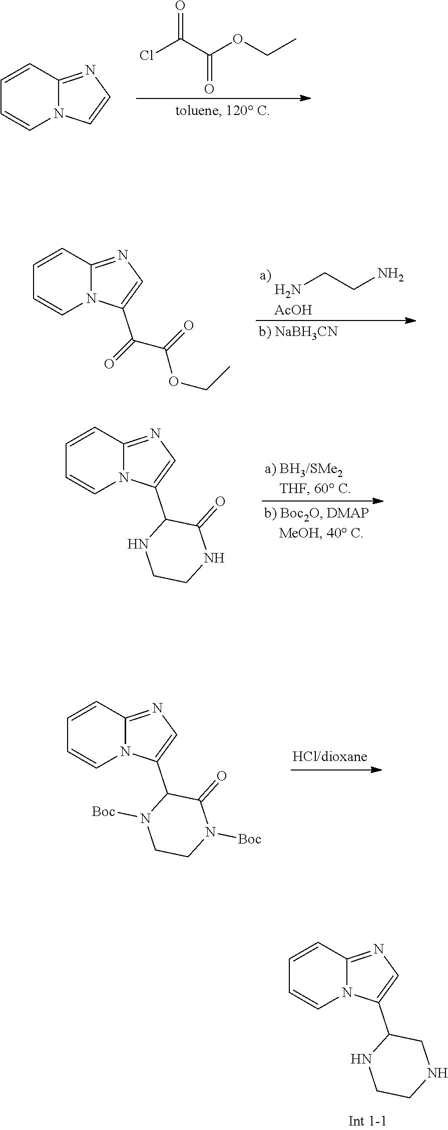

### Step 1: Preparation of Ethyl 2-(imidazo[1,2-a]pyridin-3-yl)-2-oxoacetate

A mixture of imidazo[1,2-a]pyridine (35 g, 296.27 mmol) and ethyl 2-chloro-2-oxo-acetate (80.90 g, 592.54 mmol) in toluene (900 mL) was stirred at 120° C. for 16 h. LC-MS showed 72% peak area with the desired mass. After cooling to room temperature, MeOH (100 mL) was added to the reaction mixture, then the resulted mixture was stirred for 0.5 h. After that, the resulted mixture was diluted with ethyl acetate (3 L), neutralized with saturated NaHCO₃ solution (1 L). The organic layer was separated and washed with brine (1 L×2) (Note: a small quantity of pale-yellow solid emerged during neutralization which was the desired product), dried over Na₂SO₄, filtered and concentrated under reduced pressure to give a residue which was re-crystallized with Petroleum ether/Ethyl acetate=2/1 (V/V=3/1, 500 mL) to give pure ethyl 2-(imidazo[1,2-a]pyridin-3-yl)-2-oxoacetate (60 g) as a gray solid. ¹H NMR (DMSO-d₆, 400 MHz) δ 9.53 (d, J=6.8 Hz, 1H), 8.75 (s, 1H), 7.95 (d, J=8.8 Hz, 1H), 7.82-7.77 (m, 1H), 7.43-7.40 (m, 1H), 4.39 (q, J=7.2 Hz, 2H), 1.35 (t, J=7.2 Hz, 3H).

### Step 2: Preparation of 3-(imidazo[1,2-a]pyridin-3-yl)piperazin-2-one

To a solution of ethyl 2-imidazo[1,2-a]pyridin-3-yl-2-oxo-acetate (33 g, 151.23 mmol) in EtOH (400 mL) was added CH₃COOH (9.08 g, 151.23 mmol, 8.65 mL) and ethane-1,2-diamine (10.00 g, 166.35 mmol, 11.13 mL) at 20° C. The reaction mixture was stirred at the same temperature for 1 h before NaBH₃CN (57.02 g, 907.39 mmol) was added portion-wise. The resulted mixture was heated to 80° C. and stirred for 16 h. LC-MS showed 85% peak area with the desired mass. The reaction mixture was concentrated under reduced pressure to give a residue which was further purified by MPLC (SiO₂, DCM/MeOH=10:1), and then MPLC (SiO₂, ethyl acetate (NH₃·H₂O 5%)/MeOH=10:1) to afford 3-(imidazo[1,2-a]pyridin-3-yl)piperazin-2-one (120 g) as a yellow solid. ¹H NMR (DMSO-d₆, 400 MHz) δ 8.45 (d, J=6.8 Hz, 1H), 8.11-8.04 (m, 1H), 7.60 (d, J=9.2 Hz, 1H), 7.36-7.28 (m, 1H), 6.99 (t, J=6.8 Hz, 1H), 5.04 (s, 1H), 3.46-3.36 (m 1H), 3.30-3.21 (m 1H), 3.09-3.02 (m 1H), 2.97-3.94 (m, 1H).

### Step 3: Preparation of Di-Tert-Butyl 2-(imidazo[1,2-a]pyridin-3-yl)piperazine-1,4-dicarboxylate

To a solution of 3-imidazo[1,2-a]pyridin-3-ylpiperazin-2-one (20 g, 92.49 mmol) in THF (200 mL) was added Me₂S—BH₃ (10 M, 46.25 mL) at 0° C. and then the mixture was warmed to 60° C. and stirred for 16 h. LC-MS indicated 25% peak area of the desired mass. The reaction mixture was quenched with t-BuOH (300 mL) at room temperature and concentrated under reduced pressure to afford 3-(piperazin-2-yl)imidazo[1,2-a]pyridine (50 g, crude) as a gray solid which was used in next step without further purification.

To a mixture of tert-butyl 3-imidazo[1,2-a]pyridin-3-ylpiperazine-1-carboxylate (1.8 g, 5.95 mmol) in MeOH (20 mL) was added Boc₂O (2.60 g, 11.91 mmol, 2.74 mL). The mixture was stirred at 40° C. for 32 h, then DMAP (72.73 mg, 0.595 mmol) was added. The reaction mixture was stirred at 40° C. for 16 h. LC-MS showed 36% peak area with the desired mass. The reaction mixture was concentrated under reduced pressure to give a residue which was further purified by flash chromatography (SiO₂, Ethyl acetate/Petroleum=1/1) to afford di-tert-butyl 2-imidazo[1,2-a]pyridin-3-ylpiperazine-1,4-dicarboxylate (500 mg) as a white solid.

### Step 4: Preparation of 3-(piperazin-2-yl)imidazo[1,2-a]pyridine

Di-tert-butyl 2-imidazo[1,2-a]pyridin-3-ylpiperazine-1,4-dicarboxylate (1.4 g, 3.48 mmol) was added in a solution of HCl in dioxane (15 mL). The mixture was stirred at 20° C. for 0.5 h. LC-MS showed 98% peak area with the desired mass. The reaction mixture was concentrated under reduced pressure to afford Int 1-1 (1.1 g, crude, 3HCl) without further purification. 1H NMR (CD₃OD, 400 MHz) δ 9.88 (d, J=7.2 Hz, 1H), 9.49 (s, 1H), 8.90-8.80 (m, 2H), 8.49-8.45 (m, 1H), 6.48 (dd, J=4.0, 10.8 Hz, 1H), 4.67-4.55 (m, 2H), 4.48-4.39 (m, 3H), 4.36-4.25 (m, 1H).

### Preparation of 3-(piperidin-3-yl)imidazo[1,2-a]pyridine, Int 1-2

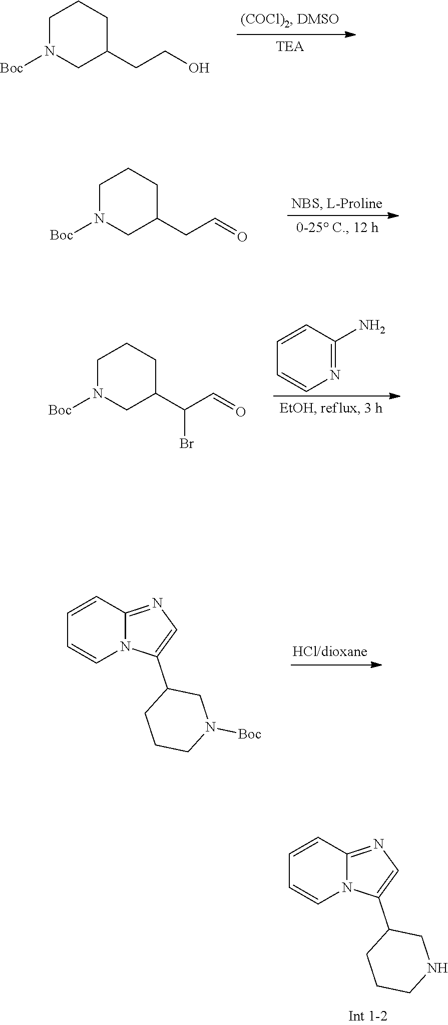

### Step 1: Preparation of Tert-Butyl 3-(2-oxoethyl)piperidine-1-carboxylate

To a solution of oxalyl chloride (14.50 g, 114.25 mmol, 10.00 mL) in DCM (200 mL) was added DMSO (14.99 g, 191.88 mmol, 14.99 mL) dropwise at −78° C. under N₂. The temperature was maintained and the reaction mixture was stirred for further 30 min, then a solution of tert-butyl 3-(2-hydroxyethyl)piperidine-1-carboxylate (10 g, 43.61 mmol) in DCM (20 mL) was added dropwise. After that, TEA (17.65 g, 174.43 mmol, 24.28 mL) was added dropwise and the reaction mixture was stirred at −78° C. for 1 h, warmed to 20° C. and stirred for 1 h. TLC (Petroleum ether/Ethyl acetate=3/1) analysis indicated formation of a major new spot (Rf=0.6). The reaction mixture was diluted with DCM (200 mL) and washed with 1 N HCl (200 mL×2). The organic layer was separated, dried over Na₂SO₄, filtered and concentrated under reduced pressure to give tert-butyl 3-(2-oxoethyl)piperidine-1-carboxylate (10 g, crude) as yellow oil.

### Step 2: Preparation of Tert-Butyl 3-(1-bromo-2-oxoethyl)piperidine-1-carboxylate

To a mixture of tert-butyl 3-(2-oxoethyl)piperidine-1-carboxylate (11 g, 48.39 mmol) in DCM (110 mL) was added NBS (8.61 g, 48.39 mmol), L-proline (1.11 g, 9.68 mmol) at 0° C. The reaction mixture was warmed to 20° C. and stirred at the same temperature for 10 h. TLC analysis (Petroleum ether/Ethyl acetate=3/1) indicated formation of a major new spot (Rf=0.7). The reaction mixture was diluted with water (100 mL), extracted with DCM (100 mL×3). The organic layer was separated, dried over Na₂SO₄, filtered and concentrated under reduced pressure to give tert-butyl 3-(1-bromo-2-oxoethyl)piperidine-1-carboxylate (14.2 g, crude) as yellow oil.

### Step 3: Preparation of Tert-Butyl 3-(imidazo[1,2-a]pyridin-3-yl)piperidine-1-carboxylate

A mixture of tert-butyl 3-(1-bromo-2-oxoethyl)piperidine-1-carboxylate (14.2 g, 46.38 mmol), pyridin-2-amine (3.06 g, 32.46 mmol) was dissolved in EtOH (140 mL). The mixture was stirred at 80° C. for 10 h. LC-MS showed about 61% peak area with the desired mass. The reaction mixture was concentrated under vacuum to give a residue which was further purified by prep-HPLC (ammonia hydroxide condition) to give tert-butyl 3-(imidazo[1,2-a]pyridin-3-yl)piperidine-1-carboxylate (4.4 g) as yellow oil.

### Step 4: Preparation of 3-(piperidin-3-yl)imidazo[1,2-a]pyridine

To a mixture of tert-butyl 3-(imidazo[1,2-a]pyridin-3-yl)piperidine-1-carboxylate (4.4 g, 14.60 mmol) in DCM (30 mL) was added HCl/dioxane (4 M, 40 mL). The mixture was stirred at 20° C. for 1 h. LC-MS showed a single peak with the desired mass. The reaction mixture was concentrated under vacuum to give 3-(piperidin-3-yl)imidazo[1,2-a]pyridine (4.3 g, crude, 2HCl) as a brown solid.

### Preparation of 2-(imidazo[1,2-a]pyridin-3-yl)morpholine, Int 1-3

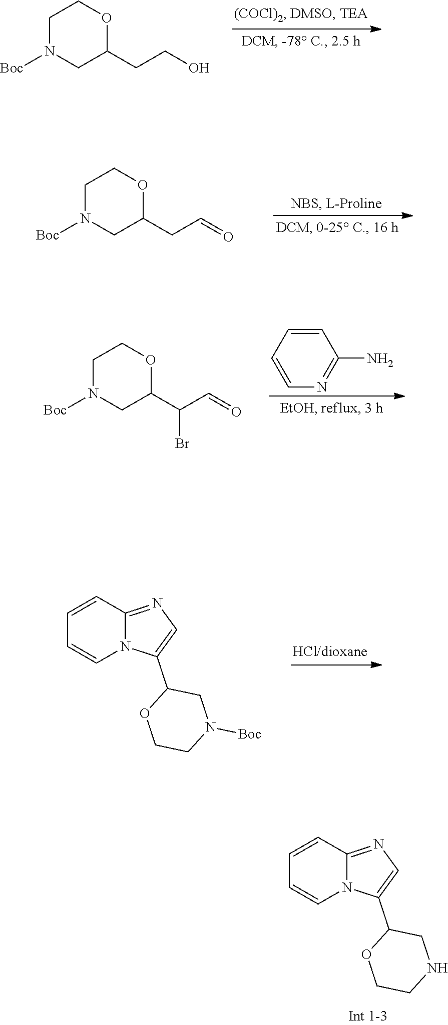

### Step 1: Preparation of Tert-Butyl 2-(2-oxoethyl)morpholine-4-carboxylate

To a solution of oxalyl chloride (2.88 g, 22.66 mmol, 1.98 mL) in DCM (40 mL) was added a solution of DMSO (2.97 g, 38.05 mmol, 2.97 mL) in DCM (2 mL) dropwise at −78° C. The mixture was stirred for 2 h at the same temperature. Then, a solution of tert-butyl 2-(2-hydroxyethyl)morpholine-4-carboxylate (2 g, 8.65 mmol) in DCM (2 mL) was added dropwise and the reaction mixture was stirred for 0.5 h. After that, a solution of TEA (3.50 g, 34.59 mmol, 4.81 mL) in DCM (2 mL) was added at −78° C. The mixture was stirred for 0.5 h while maintaining the same temperature, and then warmed to 25° C. and stirred for 1 h. TLC analysis (Petroleum ether/Ethyl acetate=3/1) showed formation of one new spot (Rf=0.43). The reaction mixture was washed with water (30 mL), 1 N HCl solution (30 mL) and brine (30 mL). The organic layer was separated, dried over Na₂SO₄, filtered and concentrated to give tert-butyl 2-(2-oxoethyl)morpholine-4-carboxylate (1.5 g) as yellow oil.

### Step 2: Preparation of Tert-Butyl 2-(1-bromo-2-oxoethyl)morpholine-4-carboxylate

To a solution of tert-butyl 2-(2-oxoethyl)morpholine-4-carboxylate (1.5 g, 6.54 mmol) in DCM (20 mL) was added NBS (1.16 g, 6.54 mmol) and L-proline (150.65 mg, 1.31 mmol) at 0° C. The reaction mixture was warmed to 25° C. and stirred for 16 h. The reaction was washed with water (30 mL) and brine (30 mL). The organic layer was separated, dried over Na₂SO₄, filtered and concentrated to give tert-butyl 2-(1-bromo-2-oxo-ethyl)morpholine-4-carboxylate (1.4 g, crude) as yellow oil.

### Step 3: Preparation of Tert-Butyl 2-(imidazo[1,2-a]pyridin-3-yl)morpholine-4-carboxylate

To a solution of pyridin-2-amine (427.56 mg, 4.54 mmol) in EtOH (20 mL) was added tert-butyl 2-(1-bromo-2-oxo-ethyl)morpholine-4-carboxylate (1.4 g, 4.54 mmol). The reaction was stirred at 80° C. for 3 h. LC-MS showed 35% peak area with the desired mass. The reaction mixture was concentrated to give a residue which was further purified by column chromatography (SiO₂, Dichloromethane/Methanol=19/1) to give tert-butyl 2-(imidazo[1,2-a]pyridin-3-yl)morpholine-4-carboxylate (600 mg) as a yellow solid.

### Step 4: Preparation of 2-(imidazo[1,2-a]pyridin-3-yl)morpholine

A mixture of tert-butyl 2-imidazo[1,2-a]pyridin-3-ylmorpholine-4-carboxylate (600 mg, 1.98 mmol) in HCl/dioxane (4 M, 10 mL) was stirred at 25° C. for 2 h. LC-MS showed 80% peak area with the desired mass. The reaction mixture was concentrated to give 2-(imidazo[1,2-a]pyridin-3-yl)morpholine (546 mg, 2HCl) as brown oil without further purification.

### Preparation of 8-((4-methoxybenzyl)oxy)-6-(4,4,5,5-tetramethyl-1,3,2-dioxaborolan-2-yl)quinoline, Int 1-4

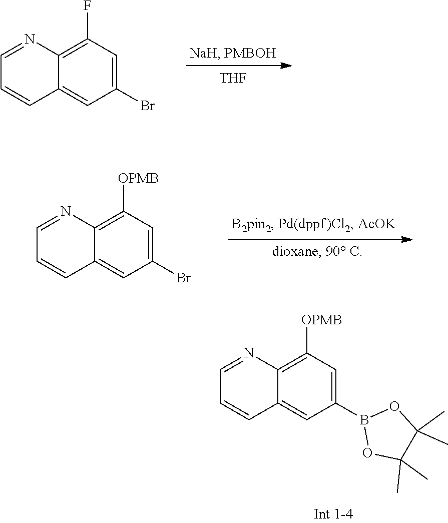

### Step 1: Preparation of 6-bromo-8-((4-methoxybenzyl)oxy)quinoline

To a mixture of (4-methoxyphenyl)methanol (12.22 g, 88.48 mmol) in THF (100 mL) was added NaH (3.54 g, 88.48 mmol, 60% purity) portionwise at 0° C. under N₂ atmosphere. The reaction mixture was stirred at 0° C. for 30 min. A solution of 6-bromo-8-fluoroquinoline (10 g, 44.24 mmol) in THF was added and then the reaction mixture was warmed to 20° C. and stirred for 16 h. LC-MS showed 53% of peak area with the desired mass. The reaction mixture was poured into saturated solution of NH₄Cl (500 mL), extracted with ethyl acetate (800 mL×2). The combined organic layers were washed with brine (500 mL×2), dried over Na₂SO₄, filtered and concentrated under reduced pressure to give a residue which was triturated with ethyl acetate (100 mL) at 20° C. for 30 min and filtered to afforded 6-bromo-8-((4-methoxybenzyl)oxy)quinoline (8.2 g) as a white solid. ¹H NMR (DMSO-d₆, 400 MHz) δ 8.86-8.85 (m, 1H), 8.30-8.28 (m, 1H), 7.80 (s, 1H), 8.00-7.58 (m, 1H), 7.46 (d, J=8.80 Hz, 2H), 7.42 (d, J=2.00 Hz 1H), 7.00-6.98 (m, 2H), 5.24 (s, 2H) 3.78 (s, 3H).

### Step 2: Preparation of 8-((4-methoxybenzyl)oxy)-6-(4,4,5,5-tetramethyl-1,3,2-dioxaborolan-2-yl)quinoline

6-bromo-8-((4-methoxybenzyl)oxy)quinoline (8.1 g, 23.53 mmol), 4,4,4′,4′,5,5,5′,5′-octamethyl-2,2′-bi(1,3,2-dioxaborolane) (7.17 g, 28.24 mmol), AcOK (2.77 g, 28.24 mmol), Pd(dppf)Cl₂ (1.72 g, 2.35 mmol) dioxane (80 mL) were added to an oven-dried round bottom flask and blanketed with nitrogen by performing three cycles of evacuation/backfill. The reaction mixture was heated to 90° C. and stirred at the same temperature for 16 h under N₂ atmosphere. LC-MS showed 38% of peak area with the desired mass. After cooling to room temperature, the reaction mixture was poured into water (200 mL), extracted with ethyl acetate (280 mL×2). The combined organic layers were washed with brine (100 mL×2), dried over Na₂SO₄, filtered and concentrated to give a residue which was further purified by column chromatography (SiO₂, Petroleum ether/Ethyl acetate=3/1) to afford Int 1-4 (8 g) as a yellow solid. ¹H NMR (CD₃OD, 400 MHz) δ 8.83-8.82 (m, 1H), 8.37-8.34 (m, 1H), 7.94 (s, 1H), 7.58-7.53 (m, 2H), 7.49-7.46 (m, 2H), 6.94-6.91 (m, 2H), 5.31 (s, 2H), 3.79 (d, J=3.20 Hz, 3H), 1.39 (s, 12H).

### Preparation of 4-(8-hydroxyquinolin-6-yl)benzoic Acid, Int 1-5

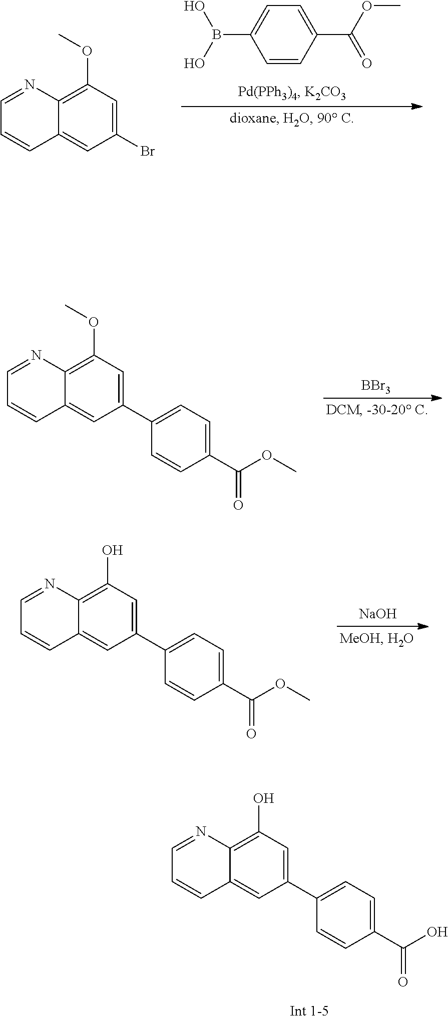

### Step 1: Preparation of Methyl 4-(8-methoxyquinolin-6-yl)benzoate

To a mixture of 6-bromo-8-methoxy-quinoline (2 g, 8.40 mmol), (4-methoxycarbonylphenyl)boronic acid (1.81 g, 10.08 mmol) in dioxane (20 mL), H₂O (2 mL) was added Pd(PPh₃)₄ (970.73 mg, 840.05 umol), K₂CO₃ (2.32 g, 16.80 mmol). The suspension was degassed under vacuum and purged with N₂ several times. The mixture was stirred at 90° C. for 4 h under N₂ atmosphere. LC-MS showed about 40% peak area of the desired mass. The reaction mixture was concentrated under vacuum to give a residue which was further purified by column chromatography (SiO₂, Petroleum ether/Ethyl acetate=1/1) to afford methyl 4-(8-methoxyquinolin-6-yl)benzoate (2.85 g) as a yellow solid.

### Step 2: Preparation of Methyl 4-(8-hydroxyquinolin-6-yl)benzoate

Methyl 4-(8-methoxy-6-quinolyl)benzoate (2.85 g, 9.70 mmol) was dissolved in DCM (29 mL). The mixture was degassed under vacuum and purged with N₂ several times and then cooled to −30° C. BBr₃ (9.72 g, 38.80 mmol) was added slowly at the same temperature and then stirred at 20° C. for 16 h. LC-MS showed about 51% peak area of the desired mass. The reaction mixture was filtered under vacuum and the resulting filtrate was concentrated under vacuum to give methyl 4-(8-hydroxyquinolin-6-yl)benzoate (1.2 g, crude) as a yellow solid without further purification.

### Step 3: Preparation of 4-(8-hydroxyquinolin-6-yl)benzoic Acid

To a mixture of methyl 4-(8-hydroxy-6-quinolyl)benzoate (1.54 g, 5.51 mmol) in MeOH (15 mL), H₂O (3 mL) was added NaOH (881.60 mg, 22.04 mmol). The reaction mixture was stirred at 40° C. for 16 h. LC-MS showed about 88% peak area of the desired mass. The mixture was diluted with H₂O (20 mL), adjusted to pH=3 with HCl (12 N). The resulting mixture was filtered and the filtrate was concentrated under vacuum to give a 4-(8-hydroxyquinolin-6-yl)benzoic acid (1.2 g) as a yellow solid. ¹H NMR (DMSO-d₆, 400 MHz) δ 9.07-8.99 (m, 1H), 8.94 (d, J=8.0 Hz, 1H), 8.11 (d, J=8.4 Hz, 2H), 8.06 (d, J=1.2 Hz, 1H), 7.97-7.90 (m, 3H), 7.74 (d, J=1.2 Hz, 1H).

### Preparation of 2-(2-bromophenyl)piperazine, Int 1-6

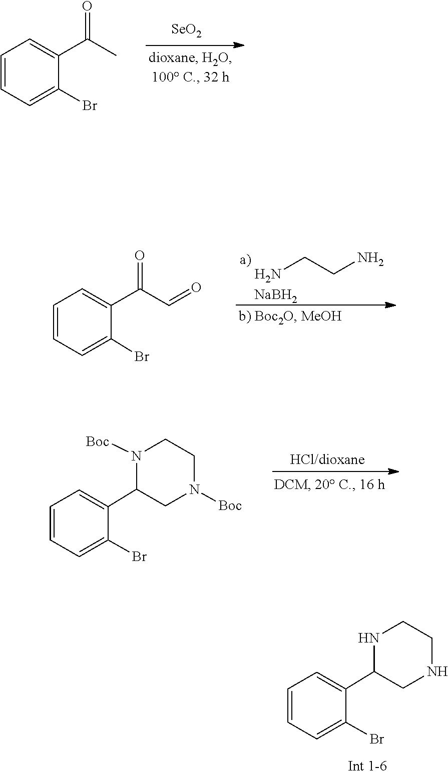

### Step 1: Preparation of 2-(2-bromophenyl)-2-oxoacetaldehyde

To a mixture of 1-(2-bromophenyl)ethanone (50 g, 251.20 mmol, 33.78 mL) in dioxane (500 mL) and H₂O (50 mL) was added SeO₂ (30.66 g, 276.32 mmol, 30.06 mL). The mixture was stirred at 100° C. for 32 h. TLC (Petroleum ether/Ethyl acetate=3/1) analysis indicated formation of one major new spot (Rt=0.3 for product). The reaction mixture was filtered and the filtrate was concentrated under reduced pressure to afford 2-(2-bromophenyl)-2-oxoacetaldehyde (50 g, crude) as yellow oil.

### Step 2: Preparation of Di-Tert-Butyl 2-(2-bromophenyl)piperazine-1,4-dicarboxylate

To a solution of 2-(2-bromophenyl)-2-oxoacetaldehyde (50 g, 234.71 mmol) in MeOH (500 mL) and THF (500 mL) was added ethane-1,2-diamine (16.93 g, 281.65 mmol, 18.85 mL) at 0° C. under N₂ atmosphere. The reaction mixture was stirred at the same temperature for 3 h. NaBH₄ (19.54 g, 516.36 mmol) was added at 0° C. portion-wise and the reaction mixture was stirred at 25° C. for 16 h. LC-MS showed 50% peak area with the desired mass. The reaction mixture was slowly acidified with HCl (1 M) to pH around 8 and then was concentrated under reduced pressure to give the crude product which was triturated with 800 mL solvent (Dichloromethane:Methanol=8:1) at 20° C. for 30 min. The mixture was filtered and the filtrate was concentrated under reduced pressure to afford 2-(2-bromophenyl)piperazine (60 g, crude) as yellow oil.

To a mixture of crude 2-(2-bromophenyl)piperazine (25 g, 103.68 mmol) in MeOH (250 mL) was added Boc₂O (90.51 g, 414.72 mmol, 95.27 mL). The mixture was stirred at 20° C. for 16 h. TLC analysis (Petroleum ether/Ethyl acetate=3/1) indicated formation of one major new spot (Rf=0.45 for product). The residue was purified by column chromatography (SiO₂, Petroleum ether/Ethyl acetate=10/1 to 3/1) to afford di-tert-butyl 2-(2-bromophenyl)piperazine-1,4-dicarboxylate (19.5 g) as yellow oil.

### Step 3: Preparation of 2-(2-bromophenyl)piperazine

To a mixture of di-tert-butyl 2-(2-bromophenyl)piperazine-1,4-dicarboxylate (19.5 g, 44.18 mmol) in DCM (200 mL) was added HCl/dioxane (4 M, 200 mL). The mixture was stirred at 20° C. for 16 h. LC-MS showed 94% peak area of the desired mass. The reaction mixture was concentrated under reduced pressure to afford 2-(2-bromophenyl)piperazine (18.6 g, crude, 2HCl) as a white solid without further purification. ¹H NMR (DMSO-d₆, 400 MHz) δ 7.93 (d, J=5.6 Hz, 1H), 7.83-7.70 (m, 1H,) 7.59 (s, 1H), 7.46 (s, 1H), 5.26 (d, J=10.0 Hz, 1H), 3.91-3.70 (m, 6H).

### Preparation of 3-(2-bromophenyl)piperidine, Int 1-7

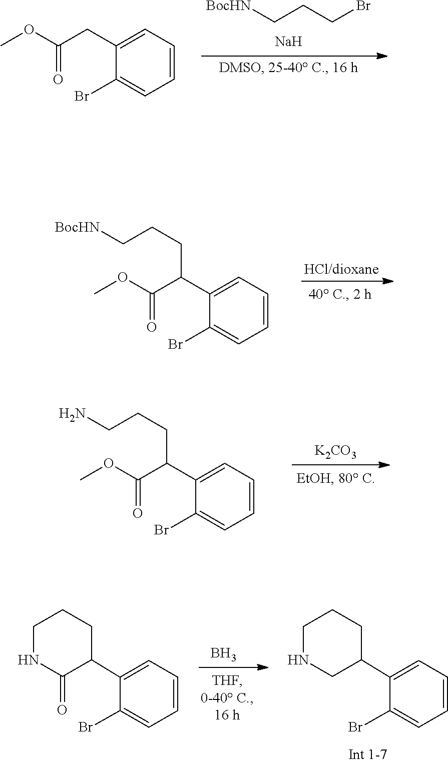

### Step 1: Preparation of Methyl 2-(2-bromophenyl)-5-((tert-butoxycarbonyl)amino)pentanoate

To a solution of methyl 2-(2-bromophenyl)acetate (6 g, 26.19 mmol) in DMSO (12 mL) was added NaH (1.12 g, 28.03 mmol, 60% purity) under N₂ atmosphere and stirred at 25° C. for 20 min. A solution of tert-butyl (3-bromopropyl)carbamate (6.24 g, 26.19 mmol) in DMSO (12 mL) was added drop-wise to the mixture. The reaction mixture was stirred at 40° C. for 2 h. LC-MS showed 70% peak area of the desired mass. After cooling to room temperature, the reaction was quenched with saturated NH₄Cl (200 mL) and extracted with ethyl acetate (150 mL×3). The organic layer was separated, washed with brine (100 mL×2), dried over Na₂SO₄, filtered and concentrated to give methyl 2-(2-bromophenyl)-5-((tert-butoxycarbonyl)amino)pentanoate (9.5 g) as yellow oil.

### Step 2: Preparation of Methyl 5-amino-2-(2-bromophenyl)pentanoate

A mixture of methyl 2-(2-bromophenyl)-5-((tert-butoxycarbonyl)amino)pentanoate (9.5 g, 24.59 mmol) in HCl/dioxane (4 M, 18.45 mL) was stirred at 40° C. for 0.5 h. LC-MS showed 94% peak area of the desired mass. The reaction mixture was concentrated to give methyl 5-amino-2-(2-bromophenyl)pentanoate (8.83 g, crude, HCl) as a white solid without further purification.

### Step 3: Preparation of 3-(2-bromophenyl)piperidin-2-one

To a solution of methyl 5-amino-2-(2-bromophenyl)pentanoate (8.83 g, 27.37 mmol, HCl) in EtOH (80 mL) was added K₂CO₃ (5.67 g, 41.05 mmol). The reaction mixture was stirred at 80° C. for 16 h. LC-MS showed 97% peak area with the desired mass. The reaction mixture was filtered through celite and the filtrate was concentrated to give a residue which was triturated with H₂O (40 mL) and filtered to give 3-(2-bromophenyl)piperidin-2-one (6 g) as a white solid. ¹H NMR (DMSO-d₆, 400 MHz) δ 7.74 (s, 1H), 7.65-7.50 (m, 1H), 7.36-7.28 (m, 1H), 7.24 (d, J=7.6, 1.6 Hz, 1H), 7.20-7.12 (m, 1H), 3.80 (dd, J=9.2, 6.4 Hz, 1H), 3.31-3.21 (m, 2H), 2.09-1.93 (m, 1H). 1.86-1.70 (m, 3H).

### Step 4: Preparation of 3-(2-bromophenyl)piperidine

To a solution of 3-(2-bromophenyl)piperidin-2-one (6 g, 23.61 mmol) in THF (180 mL) was added borane (1 M in THF, 118.05 mL) at 0° C. under N₂. The reaction mixture was stirred at 40° C. for 16 h. LC-MS showed 72% peak area of the desired mass. The reaction mixture was quenched with t-BuOH (50 mL) and then concentrated to give 3-(2-bromophenyl)piperidine (16 g, crude) as colorless oil.

### Preparation of 2-(2-bromophenyl)morpholine, Int 1-8

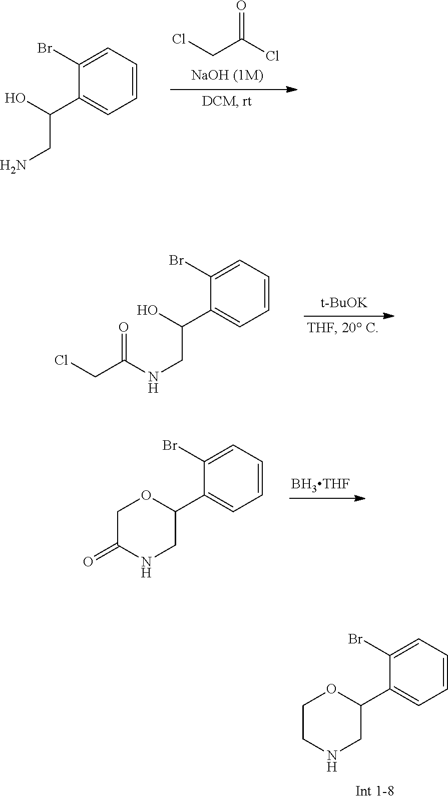

### Step 1: Preparation of N-(2-(2-bromophenyl)-2-hydroxyethyl)-2-chloroacetamide

To a solution of 2-amino-1-(2-bromophenyl)ethanol (1 g, 4.63 mmol) and NaOH (1 M, 5.55 mL) in DCM (10 mL) was added 2-chloroacetyl chloride (784.05 mg, 6.94 mmol, 552.15 uL) dropwise at 0° C. under N₂ atmosphere. The reaction mixture was stirred at 25° C. for 1 h. LC-MS showed 33% peak area of the desired mass. The reaction mixture was poured into water (30 mL), extracted with DCM (80 mL×2). The combined organic layers were washed with brine (30 mL×2), dried over Na₂SO₄, filtered and concentrated to give a crude residue which was further purified by column chromatography (SiO₂, Petroleum ether/Ethyl acetate=2:1) to afford N-(2-(2-bromophenyl)-2-hydroxyethyl)-2-chloroacetamide (790 mg) as a white solid.

### Step 2: Preparation of 6-(2-bromophenyl)morpholin-3-one

To a solution of N-[2-(2-bromophenyl)-2-hydroxy-ethyl]-2-chloro-acetamide (390 mg, 1.33 mmol) in THF (20 mL) was added t-BuOK (179.50 mg, 1.60 mmol). The mixture was stirred at 25° C. for 16 h. LC-MS showed 78% peak area of the desired mass. The reaction mixture was poured into water (40 mL), extracted with ethyl acetate (40 mL×2). The combined organic layers were washed with brine (20 mL×2), dried over Na₂SO₄, filtered and concentrated to give a crude residue which was further purified by column chromatography (SiO₂, Petroleum ether/Ethyl acetate=2/1) to afford 6-(2-bromophenyl)morpholin-3-one (450 mg) as a white solid. ¹H NMR (CDCl₃, 400 MHz)

δ 7.60-7.55 (m, 2H), 7.39-7.38 (m, 1H), 7.27-7.21 (m, 1H), 6.91 (s, 1H), 5.07 (dd, J=10.4, 3.2 Hz, 1H), 4.51-4.36 (m, 2H), 3.75-3.67 (m, 1H), 3.34 (dd, J=12.4, 10.8 Hz, 1H).

### Step 3: Preparation of 6-(2-bromophenyl)morpholin-3-one

To a solution of 6-(2-bromophenyl)morpholin-3-one (250 mg, 976.20 umol) in THF (3 mL) was added borane/tetrahydrofuran (1 M, 4.88 mL) in one portion at 0° C. The mixture was stirred at 40° C. for 18 h. LC-MS showed a peak area (17%) of the desired mass and 65% of m/z=180.2. The reaction mixture was stirred for another 16 h. LC-MS showed a peak area (21%) of the desired mass and 59% of m/z=312.4. The mixture was quenched with t-BuOH (20 mL) and then concentrated under reduced pressure to give 2-(2-bromophenyl)morpholine (450 mg) as a white solid.

### Preparation of Di-Tert-Butyl 2-(2-bromo-5-iodophenyl)piperazine-1,4-dicarboxylate, Int 1-9

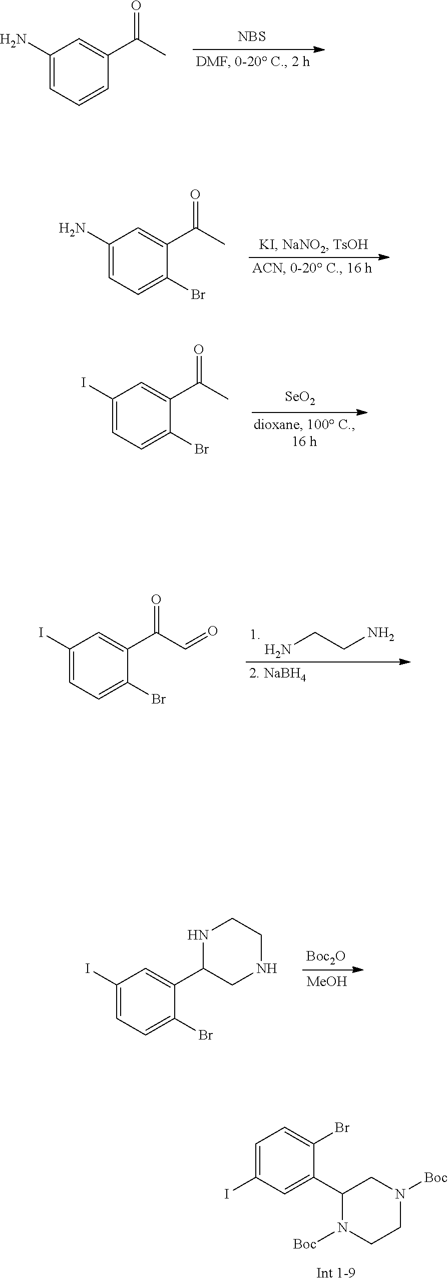

### Step 1: Preparation of 1-(5-amino-2-bromophenyl)ethan-1-one

A solution of 1-(3-aminophenyl)ethanone (50 g, 369.92 mmol) in DMF (200 mL) was added a solution of NBS (65.84 g, 369.92 mmol) in DMF (300 mL) at 0° C. over a period of 1 h. The mixture was stirred at 25° C. for 1 h. LC-MS showed 81% peak area of the desired mass. The reaction mixture was poured into water (500 mL), extracted with ethyl acetate (500 mL×3). The combined organic layers were washed with brine (500 mL×2), dried over anhydrous sodium sulfate, filtered and concentrated under reduced pressure to give a residue which was further purified by column chromatography (SiO₂, Petroleum ether/Ethyl acetate=3/1) to afford 1-(5-amino-2-bromophenyl)ethanone (65 g) as a yellow solid.

### Step 2: Preparation of 1-(2-bromo-5-iodophenyl)ethan-1-one

To a mixture of 1-(5-amino-2-bromophenyl)ethanone (10 g, 46.72 mmol) in acetonitrile (1000 mL) was added TsOH·H₂O (26.66 g, 140.15 mmol). To the mixture was added KI (19.39 g, 116.79 mmol) and NaNO₂ (6.45 g, 93.43 mmol). The mixture was stirred at 20° C. for 16 h. LC-MS showed 81% peak area of the desired mass. The reaction mixture was poured into water (500 mL), adjusted to pH 8-10 by saturated NaHCO₃ and decolorized by the addition of Na₂SO₃ solution. The resulted mixture was extracted with ethyl acetate (800 mL×3). The combined organic layers were washed with brine (500 mL×3), dried over anhydrous sodium sulfate, filtered and concentrated under reduced pressure to give a crude residue which was further purified by column chromatography (SiO₂, Petroleum ether/Ethyl acetate=5/1) to afford 1-(2-bromo-5-iodophenyl)ethanone (31.5 g) as a white solid.

### Step 3: Preparation of 2-(2-bromo-5-iodophenyl)-2-oxoacetaldehyde

To a mixture of 1-(2-bromo-5-iodophenyl)ethanone (31.5 g, 96.94 mmol) in dioxane (300 mL) and H₂O (30 mL) was added SeO₂ (10.76 g, 96.94 mmol). The mixture was stirred at 100° C. for 16 h. TLC analysis (Petroleum ether/Ethyl acetate=3/1) indicated formation of one major new spot (Rt=0.25 for product). After cooling to room temperature, the reaction mixture was poured into brine (200 mL), extracted with ethyl acetate (250 mL×3). The combined organic layers were washed with brine (200 mL×3), dried over anhydrous sodium sulfate, filtered and concentrated under reduced pressure to afford 2-(2-bromo-5-iodophenyl)-2-oxoacetaldehyde (30 g, crude) as brown oil without further purification.

### Step 4: Preparation of 2-(2-bromo-5-iodophenyl)piperazine

To a solution of 2-(2-bromo-5-iodophenyl)-2-oxoacetaldehyde (70 g, 206.54 mmol) in MeOH (700 mL) and THF (700 mL) was added slowly ethane-1,2-diamine (12.41 g, 206.54 mmol, 13.82 mL) in portions at 0° C. under N₂ and the mixture was stirred at 0° C. for 3 h. NaBH₄ was slowly added slowly at 0° C. under N₂ and then the reaction mixture was warmed to 20° C. and stirred for 16 h. LC-MS showed 60% peak area with the desired mass. The reaction mixture was acidified with HCl (1 M, to pH˜8-9), concentrated under reduced pressure to give the crude product which was triturated with Dichloromethane/Methanol (8/1, 800 mL) at 20° C. for 30 min. Then the suspension was filtered and the filtrate was concentrated under reduced pressure to afford 2-(2-bromo-5-iodophenyl)piperazine (70 g, 40% purity) as brown oil.

### Step 5: Preparation of Di-Tert-Butyl 2-(2-bromo-5-iodophenyl)piperazine-1,4-dicarboxylate

To a mixture of 2-(2-bromo-5-iodophenyl)piperazine (70 g, 76.29 mmol, 40% purity) in MeOH (700 mL) was Boc₂O (66.60 g, 305.16 mmol) dropwise. The mixture was stirred at 40° C. for 16 h. TLC (Petroleum ether/Ethyl acetate=2/1) indicated formation of one major new spot (Rt=0.45 for product). After cooling to room temperature, the reaction mixture was concentrated under reduced pressure to give a residue which was further purified by column chromatography (SiO₂, Petroleum ether/Ethyl acetate=5/1) to afford di-tert-butyl 2-(2-bromo-5-iodophenyl)piperazine-1,4-dicarboxylate (33 g) as a white solid. ¹H NMR (DMSO-d₆, 400 MHz) δ 7.60-7.53 (m, 1H), 7.51-7.37 (m, 2H), 4.94 (d, J=4.8 Hz, 1H), 3.86-3.56 (m, 5H), 3.31-3.20 (m, 1H), 1.36-1.12 (m, 18H).

### Preparation of Di-Tert-Butyl 2-(2-bromo-5-(((trifluoromethyl)sulfonyl)oxy)phenyl)piperazine-1,4-dicarboxylate, Int 1-10

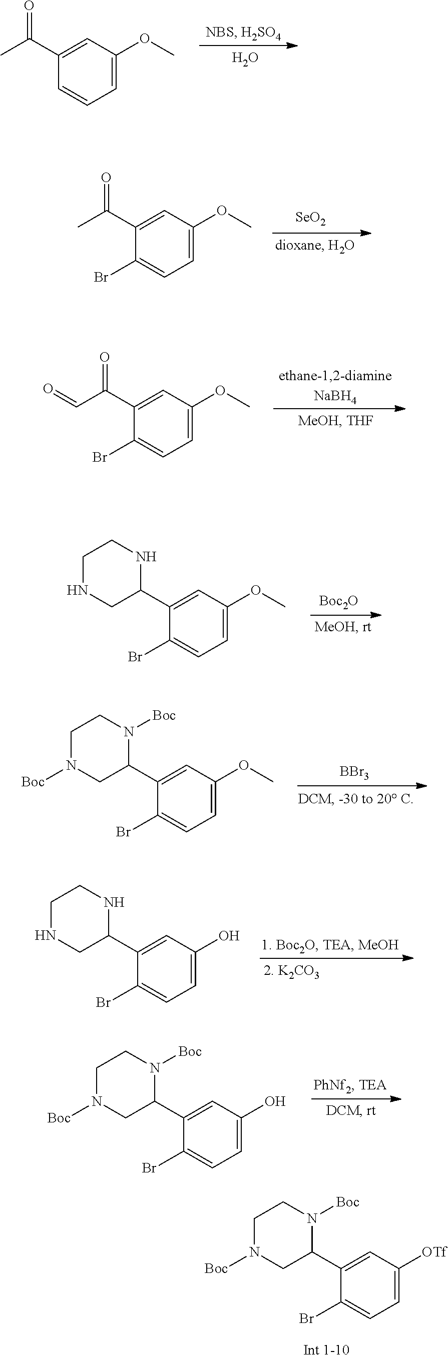

### Step 1: Preparation of 1-(2-bromo-5-methoxyphenyl)ethan-1-one

To a solution of 1-(3-methoxyphenyl)ethanone (25 g, 166.47 mmol, 22.94 mL) in H₂O (1665 mL) was added NBS (31.11 g, 174.80 mmol). The mixture was stirred at 60° C., then H₂SO₄ (81.64 g, 332.95 mmol, 12 M, 44.37 mL) was added dropwise and the resulting mixture was stirred at 60° C. for 5 h. LC-MS showed about 91% peak area of the desired mass. The reaction mixture was extracted with ethyl acetate (2 L×2) and washed with brine (2 L×2). The organic layers were combined, dried over Na₂SO₄ and concentrated under reduced pressure to give a crude residue which was triturated with ethanol (300 mL) for 30 min at −78° C. and immediately filtered to give 1-(2-bromo-5-methoxy-phenyl)ethanone (45 g) as a red liquid.

### Step 2: Preparation of 2-(2-bromo-5-methoxyphenyl)-2-oxoacetaldehyde

A mixture of SeO₂ (23.98 g, 216.09 mmol, 23.51 mL) in dioxane (450 mL) and H₂O (45 mL) was stirred at 55° C. for 5 min. 1-(2-bromo-5-methoxyphenyl)ethanone (45 g, 196.45 mmol) was added and the reaction mixture was stirred at 100° C. for 32 h. TLC (Petroleum ether/Ethyl acetate=3/1) indicated full consumption of the methyl ketone material and formation of a new spot (Rf=0.3). The reaction mixture was filtered and the mother liquor was washed with brine (1 L×2), dried over Na₂SO₄, filtered and concentrated under reduced pressure to give 2-(2-bromo-5-methoxyphenyl)-2-oxoacetaldehyde (45 g, crude) as brown oil without further purification.

### Step 3: Preparation of 2-(2-bromo-5-methoxyphenyl)piperazine

To a solution of 2-(2-bromo-5-methoxyphenyl)-2-oxoacetaldehyde (99 g, 407.32 mmol) in MeOH (800 mL), THF (800 mL) was added ethane-1,2-diamine (29.37 g, 488.78 mmol, 32.71 mL) at 0° C. under N₂ and the mixture was stirred at 0° C. for 3 h. NaBH₄ (33.90 g, 896.10 mmol) was added in portions at 0° C. and the reaction mixture was stirred at 25° C. for 16 h. LC-MS showed about 44% peak area of the desired mass. The reaction mixture was slowly acidified with HCl (1 M, to pH˜8-9), concentrated under reduced pressure to remove solvent and then lyophilized to afford a crude residue which was triturated with 1 L solvent (Dichloromethane/Methanol=8/1) at 20° C. for 30 min. The resulting mixture was filtered and the filtrate was concentrated under reduced pressure to give 2-(2-bromo-5-methoxyphenyl)piperazine (100 g, crude) as brown oil.

### Step 4: Preparation of Di-Tert-Butyl 2-(2-bromo-5-methoxyphenyl)piperazine-1,4-dicarboxylate

To a solution of 2-(2-bromo-5-methoxyphenyl)piperazine (100 g, 368.80 mmol) in MeOH (1 L) was added Boc₂O (241.47 g, 1.11 mol, 254.17 mL). The mixture was stirred at 30° C. for 16 h. LC-MS showed about 40% peak area of the desired mass. The reaction mixture was concentrated under reduced pressure to give a crude residue which was further purified by column chromatography (SiO₂, Petroleum ether/Ethyl acetate=3/1) to afford di-tert-butyl 2-(2-bromo-5-methoxyphenyl)piperazine-1,4-dicarboxylate (48 g) as yellow oil. ¹H NMR (DMSO-d₆, 400 MHz) δ 7.58-7.44 (m, 1H), 6.89-6.81 (m, 1H), 6.76 (s, 1H), 5.06-4.97 (m, 1H), 3.92-3.79 (m, 2H), 3.71 (s, 3H), 3.37 (s, 1H), 3.30-3.19 (m, 1H), 2.98-2.88 (m, 2H), 2.02-1.95 (m, 1H), 1.37 (s, 9H), 1.18 (s, 9H).

### Step 5: Preparation of 4-bromo-3-(piperazin-2-yl)phenol

A solution of di-tert-butyl 2-(2-bromo-5-methoxyphenyl)piperazine-1,4-dicarboxylate (13 g, 27.58 mmol) in DCM (130 mL) was cooled to −30° C. A solution BBr₃ (27.64 g, 110.31 mmol, 10.63 mL) in DCM (13 mL) was added dropwise. The reaction mixture was stirred at 25° C. for 16 h under N₂. LC-MS showed about 79% peak area of the desired mass. The reaction mixture was filtered and the solid was concentrated under reduced pressure to afford 4-bromo-3-(piperazin-2-yl)phenol (15 g, crude, HBr salt form) as a yellow solid.

### Step 6: Preparation of Di-Tert-Butyl 2-(2-bromo-5-hydroxyphenyl)piperazine-1,4-dicarboxylate

To a solution of 4-bromo-3-(piperazin-2-yl)phenol (34 g, 81.16 mmol, 2HBr) in MeOH (340 mL) was added TEA (41.06 g, 405.78 mmol, 56.48 mL), followed by slow addition of Boc₂O (53.14 g, 243.47 mmol, 55.93 mL). The mixture was stirred at 25° C. for 16 h. LC-MS showed about 38% peak area of the desired mass. The reaction mixture was concentrated under reduced pressure to give a crude residue which was redissolved in MeOH (340 mL). After that, K₂CO₃ (33.65 g, 243.47 mmol) was added and the reaction mixture was stirred at 50° C. for 16 h. LC-MS showed about 68% peak area of the desired mass. The reaction mixture was concentrated under reduced pressure to remove solvent. The resulting residue was diluted with water (300 mL), adjusted to pH=6 with 1 N HCl, and extracted with ethyl acetate (300 mL×3). The combined organic layers were washed with brine (200 mL), dried over Na₂SO₄, filtered and concentrated under reduced pressure to give a crude residue which was further purified by column chromatography (SiO₂, Petroleum ether/Ethyl acetate=99/1 to 3/1) to afford di-tert-butyl 2-(2-bromo-5-hydroxyphenyl)piperazine-1,4-dicarboxylate (27 g) as a brown solid. ¹H NMR (DMSO-d₆, 400 MHz) δ 9.63 (s, 1H), 7.41-7.31 (m, 1H), 6.73-6.65 (m, 1H), 6.60 (dd, J=2.8, 8.8 Hz, 1H), 4.97 (t, J=4.8 Hz, 1H), 3.96-3.75 (m, 2H), 3.72-3.69 (m, 2H), 3.31-3.13 (m, 2H), 1.39-1.34 (m, 3H), 1.27-1.09 (m, 15H).

### Step 7: Preparation of Di-Tert-Butyl 2-(2-bromo-5-(((trifluoromethyl)sulfonyl)oxy)phenyl)piperazine-1,4-dicarboxylate

To a solution of di-tert-butyl 2-(2-bromo-5-hydroxyphenyl)piperazine-1,4-dicarboxylate (15 g, 32.80 mmol) in DCM (75 mL) was added TEA (8.30 g, 81.99 mmol, 11.41 mL) and 1,1,1-trifluoro-N-phenyl-N-(trifluoromethylsulfonyl)methanesulfonamide (16.52 g, 46.24 mmol). The mixture was stirred at 20° C. for 16 h. LC-MS showed about 40% peak area of the desired mass. The reaction mixture was concentrated under reduced pressure to give a crude residue which was further purified by column chromatography (SiO₂, Petroleum ether/Ethyl acetate=99/1 to 75/25) to afford Int 1-10 (11.2 g) as a yellow oil. ¹H NMR (DMSO-d₆, 400 MHz) δ 7.89 (d, J=8.8 Hz, 1H), 7.48-7.39 (m, 1H), 7.36-7.24 (m, 1H), 5.06 (s, 1H), 3.85-3.60 (m, 4H), 3.45-3.35 (m, 1H), 3.35-3.29 (m, 1H), 1.41-1.04 (m, 18H).

### Preparation of N-(4-methoxybenzyl)-7-(4,4,5,5-tetramethyl-1,3,2-dioxaborolan-2-yl)isoquinolin-1-amine, Int 1-11

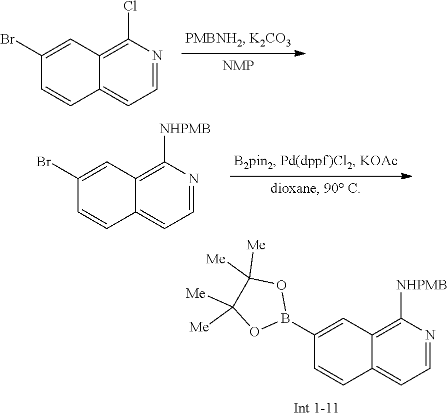

### Step 1: Preparation of 7-bromo-N-(4-methoxybenzyl)isoquinolin-1-amine

To a solution of 7-bromo-1-chloroisoquinoline (10 g, 41.24 mmol) in NMP (100 mL) was added K₂CO₃ (11.40 g, 82.47 mmol), (4-methoxyphenyl)methanamine (8.49 g, 61.86 mmol, 8.00 mL). The mixture was stirred at 120° C. for 16 h. LC-MS showed about 68% peak area of the desired mass. After cooling to room temperature, the reaction mixture was diluted with H₂O (200 mL) and then extracted with EtOAc (200 mL×3). The combined organic layers were washed with brine (100 mL×3), dried over Na₂SO₄, filtered and concentrated under reduced pressure to give a crude residue which was further purified by column chromatography (SiO₂, Petroleum ether/Ethyl acetate=3/1) to give 7-bromo-N-(4-methoxybenzyl)isoquinolin-1-amine (9.5 g) as a white solid.

### Step 2: Preparation of N-(4-methoxybenzyl)-7-(4,4,5,5-tetramethyl-1,3,2-dioxaborolan-2-yl)isoquinolin-1-amine

To a mixture of 7-bromo-N-(4-methoxybenzyl)isoquinolin-1-amine (9.5 g, 27.68 mmol), 4,4,4′,4′,5,5,5′,5′-octamethyl-2,2′-bi(1,3,2-dioxaborolane) (7.73 g, 30.45 mmol) in dioxane (100 mL) was added Pd(dppf)Cl₂ (2.03 g, 2.77 mmol), KOAc (6.79 g, 69.20 mmol). The mixture was stirred at 90° C. for 2 h under N₂. LC-MS showed 90% peak area of the desired mass. After cooling to room temperature, the reaction mixture was diluted with H₂O (100 mL) and then extracted with EtOAc (100 mL×3). The combined organic layers were washed with brine (80 mL×2) and concentrated under reduced pressure to give a residue which was further purified by column chromatography (SiO₂, Petroleum ether/Ethyl acetate=3/1) to give N-(4-methoxybenzyl)-7-(4,4,5,5-tetramethyl-1,3,2-dioxaborolan-2-yl)isoquinolin-1-amine (11 g) as brown oil. ¹H NMR (DMSO-d₆, 400 MHz) δ 8.60 (s, 1H), 8.21 (t, J=6.0 Hz, 1H), 7.86 (d, J=5.6 Hz, 1H), 7.82 (d, J=8.0 Hz, 1H), 7.65 (d, J=8.0 Hz, 1H), 7.30 (d, J=8.8 Hz, 2H), 6.86-6.83 (m, 3H), 4.64 (d, J=5.6 Hz, 2H), 3.70 (s, 3H), 1.34 (s, 12H).

### Preparation of N-(4-methoxybenzyl)-7-(4,4,5,5-tetramethyl-1,3,2-dioxaborolan-2-yl)isoquinolin-1-amine, Int 1-12

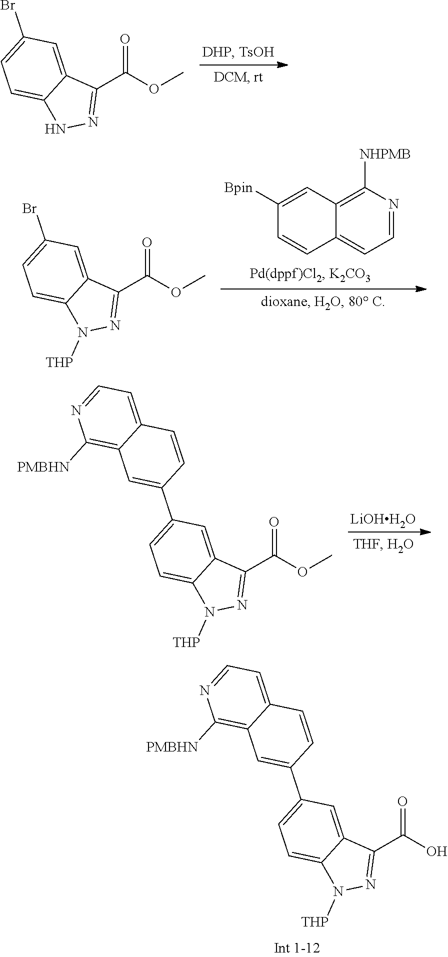

### Step 1: Preparation of Methyl 5-bromo-1-(tetrahydro-2H-pyran-2-yl)-1H-indazole-3-carboxylate

To a mixture of methyl 5-bromo-1H-indazole-3-carboxylate (9 g, 35.28 mmol) in DCM (100 mL) was added DHP (4.45 g, 52.93 mmol, 4.84 mL) and TsOH·H₂O (1.34 g, 7.06 mmol). The mixture was stirred at 30° C. for 16 h. LC-MS showed about 74% peak area of the desired mass. The reaction mixture was diluted with H₂O (100 mL) and then extracted with dichloromethane (100 mL×3). The combined organic layers were washed with brine (100 mL×2), dried over Na₂SO₄, filtered and concentrated under reduced pressure to give a residue. The residue was purified by column chromatography (SiO₂, Petroleum ether/Ethyl acetate=2/1) to give methyl 5-bromo-1-(tetrahydro-2H-pyran-2-yl)-1H-indazole-3-carboxylate (12 g) as a white solid.

### Step 2: Preparation of Methyl 5-(1-((4-methoxybenzyl)amino)isoquinolin-7-yl)-1-(tetrahydro-2H-pyran-2-yl)-1H-indazole-3-carboxylate

To a mixture of N-(4-methoxybenzyl)-7-(4,4,5,5-tetramethyl-1,3,2-dioxaborolan-2-yl)isoquinolin-1-amine (11 g, 28.18 mmol), methyl 5-bromo-1-(tetrahydro-2H-pyran-2-yl)-1H-indazole-3-carboxylate (9.56 g, 28.18 mmol) in dioxane (110 mL), H₂O (11 mL) was added Pd(dppf)Cl₂ (2.06 g, 2.82 mmol), K₂CO₃ (11.69 g, 84.55 mmol). The mixture was stirred at 80° C. for 16 h under N₂. LC-MS showed about 63% peak of the desired mass. The reaction mixture was concentrated under reduced pressure to give a residue. The residue was purified by column chromatography (SiO₂, Petroleum ether/Ethyl acetate=2/1) to give methyl 5-(1-((4-methoxybenzyl)amino)isoquinolin-7-yl)-1-(tetrahydro-2H-pyran-2-yl)-1H-indazole-3-carboxylate (15 g) as brown oil.

### Step 3: Preparation of 5-(1-((4-methoxybenzyl)amino)isoquinolin-7-yl)-1-(tetrahydro-2H-pyran-2-yl)-1H-indazole-3-carboxylic acid

To a solution of methyl 5-(1-((4-methoxybenzyl)amino)isoquinolin-7-yl)-1-(tetrahydro-2H-pyran-2-yl)-1H-indazole-3-carboxylate (15 g, 28.70 mmol) in THF (100 mL), H₂O (20 mL) was added LiOH·H₂O (3.61 g, 86.11 mmol). The mixture was stirred at 40° C. for 16 h. LC-MS showed about 94% of the desired mass. The reaction mixture was diluted with H₂O (10 mL), adjusted to pH=6 with HCl (1 N), and some solid was formed. The solid was collected by filtration and dried under vacuum to give 5-(1-((4-methoxybenzyl)amino)isoquinolin-7-yl)-1-(tetrahydro-2H-pyran-2-yl)-1H-indazole-3-carboxylic acid (10 g) as a yellow solid. ¹H NMR (DMSO-d₆, 400 MHz) δ 8.65 (s, 1H), 8.50 (s, 1H), 8.20 (s, 1H), 7.99-7.94 (m, 3H), 7.86-7.81 (m, 2H), 7.33 (d, J=8.4 Hz, 2H), 6.93 (d, J=5.6 Hz, 1H), 6.87 (d, J=8.0 Hz, 2H), 6.01 (d, J=8.4 Hz, 1H), 4.72 (d, J=5.2 Hz, 2H), 3.92 (d, J=11.2 Hz, 1H), 3.84-3.79 (m, 1H), 3.71 (s, 3H), 2.44-2.43 (m, 1H), 2.09-2.02 (m, 2H), 1.78-1.77 (m, 1H), 1.63 (m, 2H).

### Preparation of 3-(1-aminoisoquinolin-7-yl)benzoic acid, Int 1-13

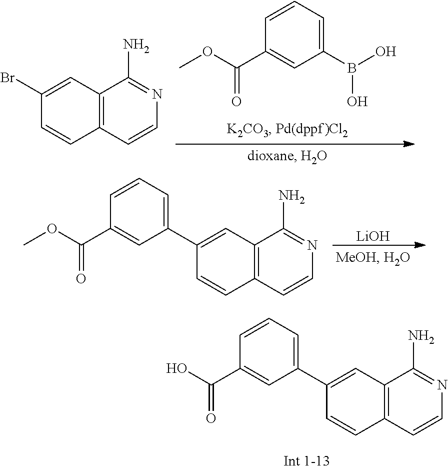

### Step 1: Preparation of methyl 3-(1-aminoisoquinolin-7-yl)benzoate

To a mixture of 7-bromoisoquinolin-1-amine (1.5 g, 6.72 mmol) and methyl 3-(4,4,5,5-tetramethyl-1,3,2-dioxaborolan-2-yl)benzoate (1.76 g, 6.72 mmol) in dioxane (15 mL) and H₂O (3 mL) was added Pd(dppf)Cl₂ (491.71 mg, 672.00 umol) and K₂CO₃ (1.86 g, 13.44 mmol) in one portion at 25° C. under N₂, and then the mixture was stirred at 80° C. for 16 h under N₂ atmosphere. LC-MS showed a peak area of 87% with the desired mass (M+H=279.1). After cooling to room temperature, the reaction mixture was concentrated under reduced pressure to give a residue. The residue was purified by column chromatography (SiO₂, Dichloromethane/Methanol=10/1) to afford methyl 3-(1-amino-7-isoquinolyl)benzoate (2 g, crude) as a black brown solid.

### Step 2: Preparation of 3-(1-aminoisoquinolin-7-yl)benzoic acid

To a solution of methyl 3-(1-amino-7-isoquinolyl)benzoate (1 g, 3.59 mmol) in MeOH (10 mL) and H₂O (2 mL) was added LiOH·H₂O (452.35 mg, 10.78 mmol). The mixture was stirred at 50° C. for 16 h. LC-MS showed a 90% peak area with the desired mass (M+H=364.9). The reaction mixture was poured into water and adjusted to pH=5 with HCl (1.0 M). Solid was formed and the solid (first batch product) was collected by filtration and the aqueous phase was purified by prep-HPLC (FA conditions) to afford 3-(1-amino-7-isoquinolyl)benzoic acid (700 mg) as a black brown solid. ¹H NMR (DMSO-d₆, 400 MHz) δ 8.59-8.54 (m, 1H), 8.43-8.33 (m, 1H), 8.13-8.04 (m, 1H), 8.01-7.92 (m, 2H), 7.84-7.76 (m, 2H), 7.69-7.58 (m, 1H), 7.03-6.91 (m, 3H).

### Preparation of Exemplary Compounds

### Example 1: Preparation of 1′-amino-N-(2-(dimethylamino)ethyl)-[6,7′-biisoquinoline]-3-carboxamide (Compound 356)

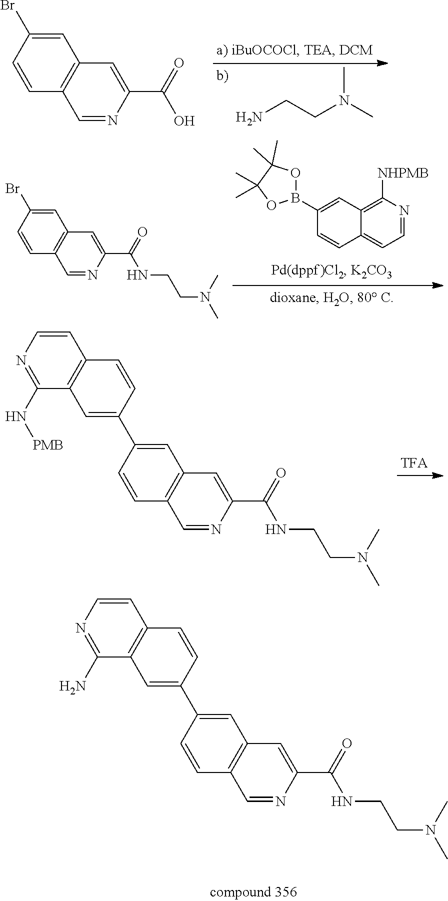

### Step 1: Preparation of 6-bromo-N-(2-(dimethylamino)ethyl)isoquinoline-3-carboxamide

To a solution of 6-bromoisoquinoline-3-carboxylic acid (250 mg, 991.81 umol) in DMF (2 mL) was added PyBOP (774.20 mg, 1.49 mmol), EDCl (570.40 mg, 2.98 mmol) and N′,N′-dimethylethane-1,2-diamine (87.43 mg, 991.81 umol). The mixture was stirred at 20° C. for 16 h. LC-MS showed peak area of 75% with the desired mass. The reaction mixture was poured into water (30 mL), extracted with ethyl acetate (30 mL×3). The combined organic layers were washed with brine (20 mL), dried over Na₂SO₄, filtered and concentrated under reduced pressure to give a residue. The residue was purified by column chromatography (SiO₂, DCM/MeOH=I/O to 1/1) to afford 6-bromo-N-[2-(dimethylamino)ethyl]isoquinoline-3-carboxamide (200 mg) as a white solid.

### Step 2: Preparation of N-(2-(dimethylamino)ethyl)-1′-((4-methoxybenzyl)amino)-[6,7′-biisoquinoline]-3-carboxamide

A mixture of 6-bromo-N-[2-(dimethylamino)ethyl]isoquinoline-3-carboxamide (150 mg, 465.55 umol), N-[(4-methoxyphenyl)methyl]-7-(4,4,5,5-tetramethyl-1,3,2-dioxaborolan-2-yl)isoquinolin-1-amine (181.70 mg, 465.55 umol), K₂CO₃ (193.03 mg, 1.40 mmol), Pd(dppf)Cl₂ (34.06 mg, 46.55 umol) in dioxane (2 mL) and H₂O (0.4 mL) was degassed and purged with N₂ for 3 times, and then the mixture was stirred at 80° C. for 16 h under N₂ atmosphere. LC-MS showed a peak area of 87% with the desired mass. The reaction mixture was poured into water (50 ml), extracted with ethyl acetate (30 mL×3). The combined organic layers were washed with brine (20 mL), dried over Na₂SO₄, filtered and concentrated. The residue was purified by column chromatography (SiO₂, Petroleum ether/Ethyl acetate=I/O to 0/1) to afford N-[2-(dimethylamino)ethyl]-6-[1-[(4-methoxyphenyl)methylamino]-7-isoquinolyl]isoquinoline-3-carboxamide (120 mg) as a yellow solid.

### Step 3: Preparation of 1′-amino-N-(2-(dimethylamino)ethyl)-[6,7′-biisoquinoline]-3-carboxamide

A solution of N-[2-(dimethylamino)ethyl]-6-[1-[(4-methoxyphenyl)methylamino]-7-isoquinolyl]isoquinoline-3-carboxamide (120 mg, 237.34 umol) in TFA (2 mL) was stirred at 40° C. for 16 h. LC-MS showed a peak area of 83% with the desired mass. The reaction mixture was concentrated under reduced pressure to give a residue which was purified by prep-HPLC (TFA conditions) to afford (23 mg, purity 86%) as a white solid, which was further purified by prep-HPLC (basic conditions) to afford the compound 356 (2.2 mg) as a white solid. ¹H NMR (CD₃OD, 400 MHz) δ 9.34 (s, 1H), 8.65 (d, J=2.8 Hz, 2H), 8.51 (s, 1H), 8.36-8.25 (m, 2H), 8.18 (dd, J=1.6, 8.6 Hz, 1H), 7.91 (d, J=8.4 Hz, 1H), 7.81 (d, J=6.0 Hz, 1H), 7.07 (d, J=5.6 Hz, 1H), 3.67 (t, J=6.8 Hz, 2H), 2.68 (t, J=6.8 Hz, 2H), 2.38 (s, 6H), LC-MS: [M+H]+=386.1.

### Example 2: Preparation of 5-(1-aminoisoquinolin-7-yl)-N-((1-methylpiperidin-4-yl)methyl)-1H-indazole-3-carboxamide (Compound 337)

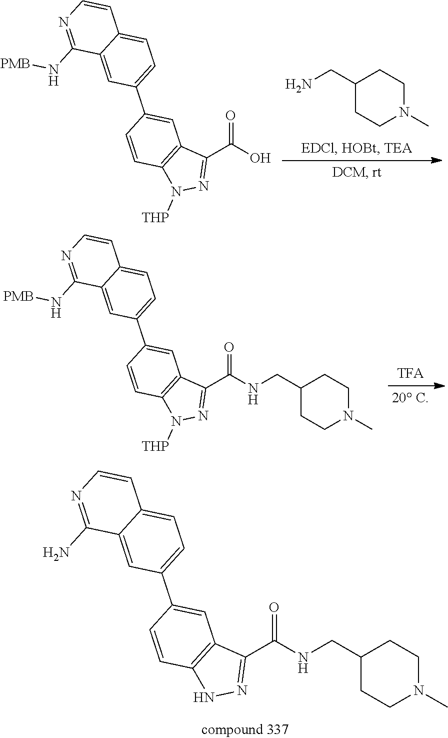

### Step 1: Preparation of 5-(1-((4-methoxybenzyl)amino)isoquinolin-7-yl)-N-((1-methylpiperidin-4-yl)methyl)-1-(tetrahydro-2H-pyran-2-yl)-1H-indazole-3-carboxamide

To a mixture of 5-(1-((4-methoxybenzyl)amino)isoquinolin-7-yl)-1-(tetrahydro-2H-pyran-2-yl)-1H-indazole-3-carboxylic acid (150 mg, 294.95 umol), (1-methylpiperidin-4-yl)methanamine (45.38 mg, 353.94 umol) in DCM (1.5 mL) was added TEA (119.38 mg, 1.18 mmol), EDCl (84.81 mg, 442.42 umol) and HOBt (47.82 mg, 353.94 umol). The mixture was stirred at 20° C. for 16 h. LC-MS showed peak area of 27% with the desired mass. The reaction mixture was concentrated under reduced to give 5-(1-((4-methoxybenzyl)amino)isoquinolin-7-yl)-N-((1-methylpiperidin-4-yl)methyl)-1-(tetrahydro-2H-pyran-2-yl)-1H-indazole-3-carboxamide (300 mg, crude) as a yellow solid.

### Step 2: Preparation of 5-(1-aminoisoquinolin-7-yl)-N-((1-methylpiperidin-4-yl)methyl)-1H-indazole-3-carboxamide

A mixture of 5-(1-((4-methoxybenzyl)amino)isoquinolin-7-yl)-N-((1-methylpiperidin-4-yl)methyl)-1-(tetrahydro-2H-pyran-2-yl)-1H-indazole-3-carboxamide (300 mg, 484.84 umol) was dissolved in TFA (4.62 g, 40.52 mmol, 3 mL). The mixture was stirred at 20° C. for 2 h. LC-MS showed peak area of 25% with the desired mass. The reaction mixture was concentrated under reduced pressure to remove solvent to give a residue which was purified by prep-HPLC (TFA conditions) to give compound 337 (29.5 mg) as a white solid. ¹H NMR (DMSO-d₆, 400 MHz) δ 13.82 (s, 1H), 13.36 (s, 1H), 9.26 (s, 3H), 8.93 (s, 1H), 8.62 (t, J=6.0 Hz, 1H), 8.62 (s, 1H), 8.33 (d, J=8.4 Hz, 1H), 8.08 (d, J=8.4 Hz, 1H), 7.94 (d, J=10.4 Hz, 1H), 7.81 (d, J=8.8 Hz, 1H), 7.71 (d, J=7.2 Hz, 1H), 7.30 (d, J=6.8 Hz, 1H), 3.44 (t, J=12.0 Hz, 2H), 3.26 (t, J=6.0 Hz, 2H), 2.91-2.90 (m, 2H), 2.76 (s, 3H), 1.91-1.83 (m, 3H), 1.46-1.40 (m, 2H), LC-MS: [M+H]+=415.2.

### Example 3: Preparation of 4-(3-(1H-pyrazol-5-yl)piperidin-1-yl)-6-cyclopropylpyrimidin-2-amine (Compound 211)

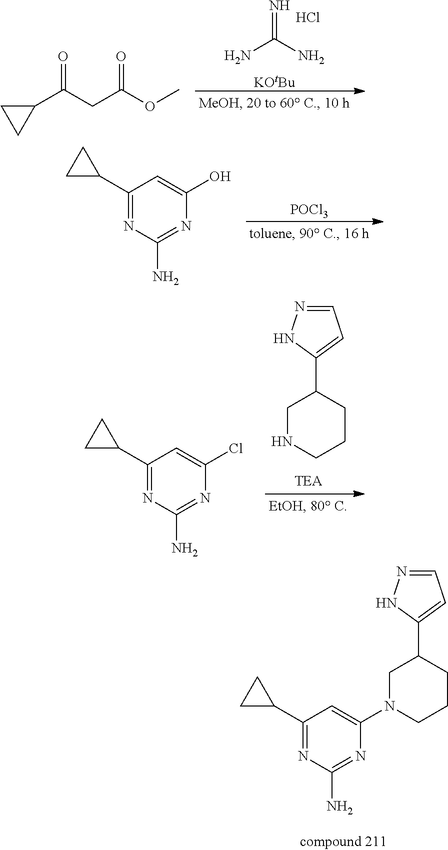

### Step 1: Preparation of 2-amino-6-cyclopropylpyrimidin-4-ol

To a mixture of methyl 3-cyclopropyl-3-oxo-propanoate (2 g, 14.07 mmol), guanidine (1.61 g, 16.88 mmol, HCl) in MeOH (20 mL) was added t-BuOK (8.53 g, 75.97 mmol). The mixture was stirred at 20° C. for 15 min and was stirred at 60° C. for 5 min. Then the mixture was stirred at 20° C. for 10 hr. LC-MS showed about 7.6% peak area with the desired mass. The reaction mixture was filtered and the filtrate was concentrated under vacuum to give a residue dried by azeotroping with toluene (30 mL×3) to afford 2-amino-6-cyclopropylpyrimidin-4-ol (500 mg, crude) as white solid.

### Step 2: Preparation of 4-chloro-6-cyclopropylpyrimidin-2-amine

To a mixture of 2-amino-6-cyclopropyl-pyrimidin-4-ol (500 mg, 3.31 mmol) in toluene (4 mL) was added POCl₃ (2.54 g, 16.54 mmol, 1.54 mL). The mixture was stirred at 90° C. for 10 hr. The reaction mixture was concentrated under reduced pressure to afford 4-chloro-6-cyclopropylpyrimidin-2-amine (500 mg, crude) as brown oil.

### Step 3: Preparation of 4-(3-(1H-pyrazol-5-yl)piperidin-1-yl)-6-cyclopropylpyrimidin-2-amine

To a mixture of 4-chloro-6-cyclopropyl-pyrimidin-2-amine (100 mg, 589.58 umol), 3-(1H-pyrazol-5-yl)piperidine (170.23 mg, 907.05 umol, HCl salt) in EtOH (1 mL) was added TEA (734.27 mg, 7.26 mmol, 1.01 mL). The mixture was stirred at 80° C. for 10 hr. LC-MS showed about peak area of 24% with the desired mass. The reaction mixture was concentrated under reduced pressure to give a residue which was purified by prep-HPLC (NH₄OH conditions) to afford compound 211 (10.6 mg) as a white solid. ¹H NMR (CD₃OD, 400 MHz) δ 7.66-7.43 (m, 1H), 6.23 (s, 1H), 5.97 (s, 1H), 4.53 (d, J=12.8 Hz, 1H), 4.43-4.24 (m, 1H), 3.09-2.81 (m, 3H), 2.21-2.10 (m, 1H), 1.86-1.73 (m, 3H), 1.69-1.54 (m, 1H), 0.99-0.84 (m, 4H), LC-MS: [M+H]+=285.2.

### Example 4: Preparation of 4-(3-(1H-pyrazol-5-yl)piperidin-1-yl)-6,7-dihydro-5H-cyclopenta[d]pyrimidin-2-amine (Compound 195)

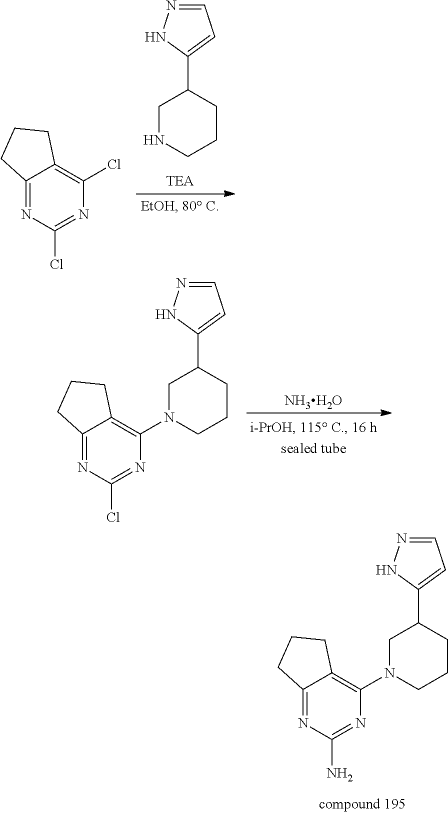 [SMILES: Clc1nc(Cl)c2c(n1)CCC2 ; c1cc(C2CCCNC2)[nH]n1 ; Clc1nc2c(c(N3CCCC(c4ccn[nH]4)C3)n1)CCC2 ; "NH3·H2O" ; Nc1nc2c(c(N3CCCC(c4ccn[nH]4)C3)n1)CCC2]

### Step 1: 4-(3-(1H-pyrazol-5-yl)piperidin-1-yl)-2-chloro-6,7-dihydro-5H-cyclopenta[d]pyrimidine

To a solution of 2,4-dichloro-6,7-dihydro-5H-cyclopenta[d]pyrimidine (168.78 mg, 892.81 umol), 3-(1H-pyrazol-5-yl)piperidine (150 mg, 992.01 umol) in EtOH (1.5 mL) was added TEA (351.33 mg, 3.47 mmol). The mixture was stirred at 80° C. for 16 h. LC-MS showed peak area of 94% with the desired mass. The reaction mixture was cooled to room temperature, concentrated under reduced pressure to give a residue which was purified by prep-TLC (Petroleum ether/Ethyl acetate=1/1, Rf=0.3) to give 4-(3-(1H-pyrazol-5-yl)piperidin-1-yl)-2-chloro-6,7-dihydro-5H-cyclopenta[d]pyrimidine (180 mg, 60%) as a white solid. ¹H NMR (CDCl₃, 400 MHz) δ 8.08-7.81 (m, 1H), 7.48 (d, J=1.2 Hz, 1H), 6.10 (d, J=2.0 Hz, 1H), 4.48 (d, J=14.4 Hz, 1H), 4.34 (d, J=14.4 Hz, 1H), 3.17 (t, J=10.4 Hz, 1H), 3.14-3.04 (m, 1H), 2.93-2.89 (m, 3H), 2.28-2.74 (m, 2H), 2.18-2.12 (m, 1H), 1.99-1.96 (m, 2H), 1.78-1.74 (m, 2H), 1.61-1.58 (m, 1H).

### Step 2: 4-(3-(1H-pyrazol-5-yl)piperidin-1-yl)-6,7-dihydro-5H-cyclopenta[d]pyrimidin-2-amine

A mixture of 4-(3-(1H-pyrazol-5-yl)piperidin-1-yl)-2-chloro-6,7-dihydro-5H-cyclopenta[d]pyrimidine (120 mg, 395.01 umol) was dissolved in a mixture of NH₃·H₂O (6 mL) and i-PrOH (2 mL). The mixture was stirred at 115° C. for 20 h in a sealed tube. The residue was cooled to room temperature, concentrated under vacuum to give a residue which was purified by prep-HPLC (TFA conditions) to give compound 195 (5 mg) as a white solid. ¹H NMR (METHANOL-d₄, 400 MHz) δ 7.60 (d, J=2.0 Hz, 1H), 6.27 (d, J=2.0 Hz, 1H), 5.24-5.08 (m, 1H), 4.6-4.36 (m, 1H), 3.32-3.12 (m, 2H), 3.10-2.98 (m, 3H), 2.86 (t, J=7.8 Hz, 2H), 2.24-2.12 (m, 3H), 1.99-1.83 (m, 2H), 1.78-1.62 (m, 1H), LC-MS: [M+H]+=285.4.

### Example 5: Preparation of (3-(1-aminoisoquinolin-7-yl)phenyl)(4-methoxypiperidin-1-yl)methanone (Compound 346)

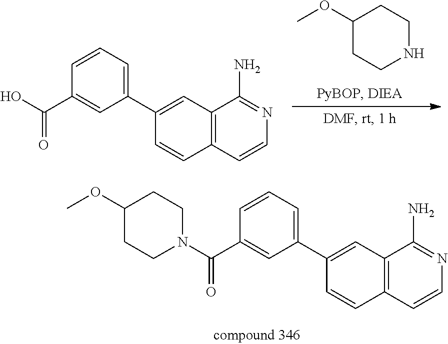

### Step 1: Preparation of (3-(1-aminoisoquinolin-7-yl)phenyl)(4-methoxypiperidin-1-yl)methanone

To a solution of 3-(1-aminoisoquinolin-7-yl)benzoic acid (100 mg, 378.39 umol) in DMF (1 mL) was added 4-methoxypiperidine (43.58 mg, 378.39 umol), PyBOP (295.37 mg, 567.58 umol) and DIEA (195.62 mg, 1.51 mmol, 263.63 uL). The mixture was stirred at 20° C. for 2 h. LC-MS showed peak area of 70% with the desired mass The mixture was filtered and concentrated under reduced pressure to give a residue which was purified by prep-HPLC (NH₄HCO₃ condition) to afford (3-(1-aminoisoquinolin-7-yl)phenyl)(4-methoxypiperidin-1-yl)methanone (26.0 mg) as a yellow solid. ¹H NMR (DMSO-d₆, 400 MHz) δ 8.55 (s, 1H), 8.01-7.99 (m, 1H), 7.93 (d, J=8 Hz, 1H), 7.87 (s, 1H), 7.81-7.77 (m, 2H), 7.57 (t, J=8.0 Hz, 1H), 7.38 (d, J=7.6 Hz, 1H), 6.93-6.92 (m, 3H), 3.97 (m, 1H), 3.51 (m, 1H), 3.48-3.43 (m, 1H), 3.26 (m, 5H), 1.91-1.81 (m, 2H), 1.49-1.45 (m, 2H), LC-MS: [M+H]+=362.2.

### Example 6: Preparation of (4-(8-hydroxyquinolin-6-yl)-N-((1-methylazetidin-3-yl)methyl)benzamide (Compound 298)

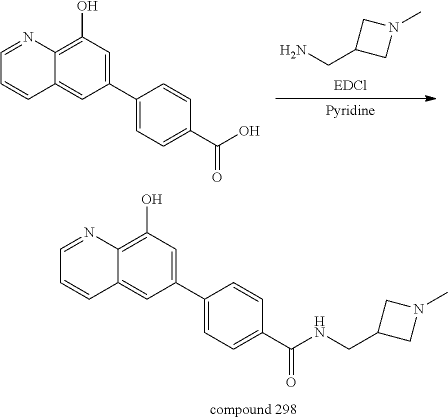

### Step 1: Preparation of 4-(8-hydroxyquinolin-6-yl)-N-((1-methylazetidin-3-yl)methyl)benzamide

To a mixture of 4-(8-hydroxy-6-quinolyl)benzoic acid (50 mg, 188.49 umol), (1-methylazetidin-3-yl)methanamine (32.62 mg, 188.49 umol, 2HCl) in Pyridine (0.5 mL) was added EDCl (39.75 mg, 207.34 umol). The mixture was stirred at 20° C. for 1 h. LC-MS showed peak area of 67% with the desired mass. The reaction mixture was concentrated under reduced pressure to give a residue which was purified by prep-HPLC (FA conditions) to afford compound 298 (1.6 mg, 2.02%, FA salt) as a white solid.

¹H NMR (DMSO-d₆, 400 MHz) δ 8.87 (dd, J=1.2, 4.0 Hz, 1H), 8.66 (t, J=5.6 Hz, 1H), 8.41 (dd, J=1.2, 8.4 Hz, 1H), 8.26 (s, 1H), 7.98 (d, J=8.8 Hz, 2H), 7.90 (d, J=8.8 Hz, 2H), 7.79 (s, 1H), 7.60 (dd, J=4.0, 8.0 Hz, 1H), 7.46 (d, J=1.6 Hz, 1H), 3.48-3.45 (m, 4H), 3.20-3.18 (m, 2H), 2.71-2.66 (m, 1H), 2.35 (s, 3H), LC-MS: [M+H]+=347.9.

### Example 7: Preparation of N-(3-(dimethylamino)propyl)-5-(8-hydroxyquinolin-6-yl)thiophene-3-carboxamide (Compound 281)

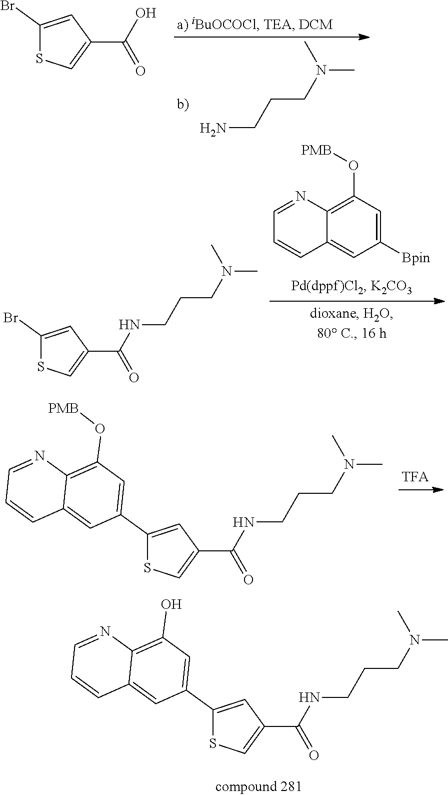

### Step 1: Preparation of 5-bromo-N-(3-(dimethylamino)propyl)thiophene-3-carboxamide

A mixture of 5-bromothiophene-3-carboxylic acid (1 g, 4.83 mmol), isobutyl carbonochloridate (659.65 mg, 4.83 mmol), TEA (1.47 g, 14.49 mmol) in THF (20 mL) was degassed and purged with N₂ for 3 times, and then the mixture was stirred at 0° C. for 1 h. Then, N′,N′-dimethylpropane-1,3-diamine (493.51 mg, 4.83 mmol) was added. The mixture was stirred at 25° C. for 16 h. LC-MS showed a major peak (85%) with the desired mass. The reaction mixture was poured into water (50 ml), extracted with DCM (50 mL×2). The combined organic layers were washed with brine (50 mL×2), dried over Na₂SO₄, filtered and concentrated under reduced pressure to give a residue which was purified by column chromatography (SiO₂, Dichloromethane/Methanol=10/1) to afford 5-bromo-N-[3-(dimethylamino)propyl]thiophene-3-carboxamide (0.95 g) as brown oil. ¹H NMR (CDCl₃, 400 MHz) δ 8.55-8.39 (m, 1H) 7.71 (d, J=1.6 Hz, 1H) 7.26 (d, J=1.6 Hz, 1H) 3.54-3.50 (m, 2H) 2.53-2.49 (m, 2H) 2.32 (s, 6H) 1.76-1.71 (m, 2H).

### Step 2: Preparation of N-(3-(dimethylamino)propyl)-5-(8-((4-methoxybenzyl)oxy)quinolin-6-yl)thiophene-3-carboxamide

To a mixture of 5-bromo-N-[3-(dimethylamino)propyl]thiophene-3-carboxamide (100 mg, 343.40 umol) and 8-[(4-methoxyphenyl)methoxy]-6-(4,4,5,5-tetramethyl-1,3,2-dioxaborolan-2-yl)quinoline (134.36 mg, 343.40 umol) in dioxane (1.5 mL) and H₂O (0.3 mL) was added Pd(dppf)Cl₂ (25.13 mg, 34.34 umol) and K₂CO₃ (142.38 mg, 1.03 mmol) in one portion at 25° C., and then the mixture was stirred at 80° C. for 16 h under N₂ atmosphere. LC-MS showed peak area of 48% with the desired mass. The reaction mixture was concentrated under reduced pressure to give a residue which was purified by prep-TLC (Dichloromethane/Methanol=10/1) to afford N-[3-(dimethylamino)propyl]-5-[8-[(4-methoxyphenyl)methoxy]-6-quinolyl]thiophene-3-carboxamide (60 mg) as a brown oil. ¹H NMR (CD₃OD, 400 MHz) δ 8.77 (dd, J=4.4, 1.6 Hz, 1H), 8.33 (dd, J=8.4, 1.6 Hz, 1H), 8.04 (d, J=1.2 Hz, 1H), 7.89 (d, J=1.2 Hz, 1H), 7.73 (d, J=1.6 Hz, 1H), 7.56 (dd, J=8.4, 4.31 Hz, 1H), 7.54-7.50 (m, 3H), 6.95 (d, J=8.8 Hz, 2H), 5.40 (s, 2H), 3.79 (s, 3H), 3.42 (t, J=6.8 Hz, 2H), 2.47-2.42 (m, 2H), 2.28 (s, 6H), 1.86-1.80 (m, 2H).

### Step 3: Preparation of N-(3-(dimethylamino)propyl)-5-(8-hydroxyquinolin-6-yl)thiophene-3-carboxamide

To a solution of N-[3-(dimethylamino)propyl]-5-[8-[(4-methoxyphenyl)methoxy]-6-quinolyl]thiophene-3-carboxamide (60 mg, 126.16 umol) in DCM (1 mL) was added TFA (1 mL). The mixture was stirred at 25° C. for 1 h. LC-MS showed peak area of 86% with the desired mass. The reaction mixture was concentrated under reduced pressure to give a residue which was purified by prep-HPLC (NH₄OH conditions) to afford N-[3-(dimethylamino)propyl]-5-(8-hydroxy-6-quinolyl)thiophene-3-carboxamide (3.3 mg) as a yellow solid. ¹H NMR (CD₃OD, 400 MHz) δ 8.78 (dd, J=4.0, 1.2 Hz, 1H), 8.29 (dd, J=8.4, 1.6 Hz, 1H), 8.03 (d, J=1.2 Hz, 1H), 7.87 (d, J=1.2 Hz, 1H), 7.65 (d, J=1.6 Hz, 1H), 7.52 (dd, J=8.4, 4.4 Hz, 1H), 7.41 (d, J=2.0 Hz, 1H), 3.42 (t, J=6.8 Hz, 2H), 2.48-2.44 (m, 2H), 1.80-1.87 (m, 2H), LC-MS: [M+H]+=356.3.

### Example 8: Preparation of R or S-4-(3-(2-bromophenyl)piperazin-1-yl)-6-isopropyl-N-methylpyrimidin-2-amine (Compound 153 and 154)

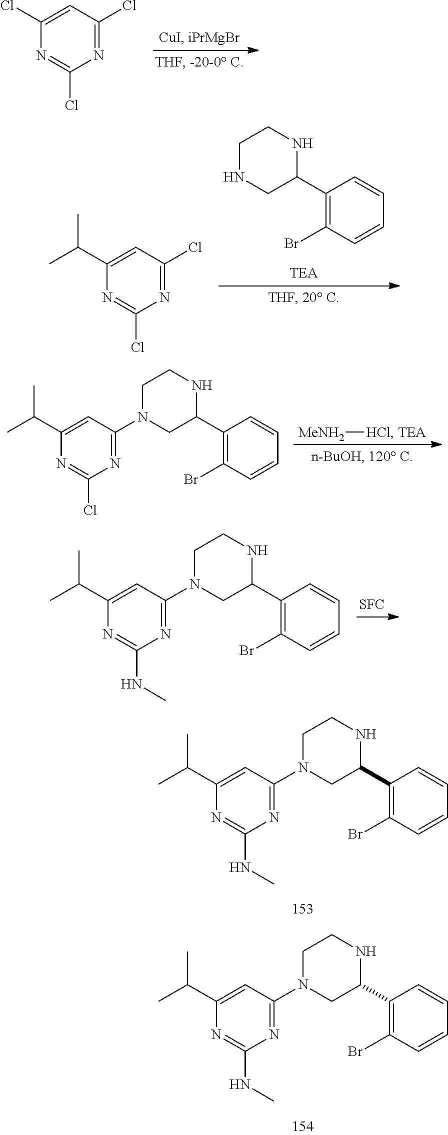

### Step 1: Preparation of 2,4-dichloro-6-isopropylpyrimidine

To a solution of 2,4,6-trichloropyrimidine (3 g, 16.36 mmol) in 2-MeTHF (150 mL) was added CuI (311.49 mg, 1.64 mmol) under N₂ and then the mixture was cooled to −10° C. and i-PrMgBr (1 M, 24.53 mL) was added dropwise at −10° C. The mixture was stirred at 20° C. for 16 h. LC-MS showed peak area of 33% with the desired mass. The mixture was quenched with NH₄Cl solution (50 mL) and extracted with EtOAc (100 mL×3). The combined organic layer was washed with brine (50 mL×3), dried over Na₂SO₄, filtered and concentrated under reduced pressure to give a residue which was purified by column chromatography (SiO₂, Petroleum ether/Ethyl acetate=100/3) to afford 2,4-dichloro-6-isopropylpyrimidine (1.6 g) as brown oil.

### Step 2: Preparation of 4-(3-(2-bromophenyl)piperazin-1-yl)-2-chloro-6-isopropylpyrimidine

To a solution of 2,4-dichloro-6-isopropylpyrimidine (1.3 g, 6.80 mmol) in THF (40 mL) was added 2-(2-bromophenyl)piperazine (1.97 g, 8.17 mmol) and TEA (2.07 g, 20.41 mmol). The mixture was stirred at 20° C. for 16 h. The reaction mixture was filtered under vacuum to give a filter head and a filtrate. The filtrate was diluted with H₂O (50 mL) and extracted with EtOAc (100 mL×3). The combined organic layers were washed with brine (50 mL), dried over Na₂SO₄, filtered and concentrated under reduced pressure to give a residue which was purified by column chromatography (SiO₂, Petroleum ether/Ethyl acetate=4/1) to afford 4-(3-(2-bromophenyl)piperazin-1-yl)-2-chloro-6-isopropylpyrimidine (6.65 g) as yellow oil.

### Step 3: Preparation of 4-(3-(2-bromophenyl)piperazin-1-yl)-6-isopropyl-N-methylpyrimidin-2-amine

To a solution of 4-(3-(2-bromophenyl)piperazin-1-yl)-2-chloro-6-isopropylpyrimidine (200 mg, 505.40 umol) and methanamine hydrochloride (34.12 mg, 505.40 umol) in n-BuOH (2 mL) was added TEA (255.71 mg, 2.53 mmol). The mixture was stirred at 120° C. for 16 h. LC-MS peak area of 71% with the desired mass. The mixture was filtered and concentrated under reduced pressure to give a residue which was purified by prep-HPLC (basic condition) to give 4-(3-(2-bromophenyl)piperazin-1-yl)-6-isopropyl-N-methylpyrimidin-2-amine (50 mg) as a white solid.

### Step 4: SFC Separation

4-(3-(2-bromophenyl)piperazin-1-yl)-6-isopropyl-N-methylpyrimidin-2-amine (50 mg) was separated by SFC (column: DAICEL CHIRALCEL OJ (250 mm*30 mm, 10 um); mobile phase: [0.1% NH3H2O IPA]; B %: 30%-30%, 5.45; 49 min) to afford:

Peak 1 (Retention time 1.333 min on SFC) compound 153 (17.7 mg) as a white solid, ¹H NMR (CD₃CN, 400 MHz) δ 7.72 (d, J=7.6 Hz, 1H), 7.60 (d, J=8.0 Hz, 1H), 7.40 (t, J=7.6 Hz, 1H), 7.20 (t, J=7.6 Hz, 1H), 5.91 (s, 1H), 5.12 (s, 1H), 4.63-4.45 (m, 1H), 4.32 (d, J=10.4 Hz, 1H), 4.07 (dd, J=2.4, 10.0 Hz, 1H), 3.10 (d, J=10.0 Hz, 1H), 2.97-2.85 (m, 2H), 2.83 (d, J=5.2 Hz, 3H), 2.66-2.53 (m, 2H), 1.16 (d, J=6.8 Hz, 6H), LC-MS: [M+H]+=390.2.

Peak 2 (Retention time 1.622 min on SFC) compound 154 (13.8 mg) as a white solid, ¹H NMR (CD₃CN, 400 MHz) δ 7.72 (d, J=7.6 Hz, 1H), 7.63-7.58 (m, 1H), 7.40 (t, J=7.6 Hz, 1H), 7.23-7.18 (m, 1H), 5.91 (s, 1H), 5.08 (d, J=1.8 Hz, 1H), 4.53 (dd, J=4.0, 10.0 Hz, 1H), 4.32 (d, J=8.4 Hz, 1H), 4.07 (dd, J=2.4, 10.4 Hz, 1H), 3.10 (d, J=10.0 Hz, 1H), 2.96-2.85 (m, 2H), 2.83 (d, J=4.8 Hz, 3H), 2.66-2.52 (m, 2H), 1.16 (d, J=6.8 Hz, 6H), LC-MS: [M+H]+=390.0.

### Example 9: Preparation of R or S-6-(2-(2-bromophenyl)morpholino)-N⁴-(cyclopropylmethyl)pyrimidine-2,4-diamine (Compound 77 and 78)

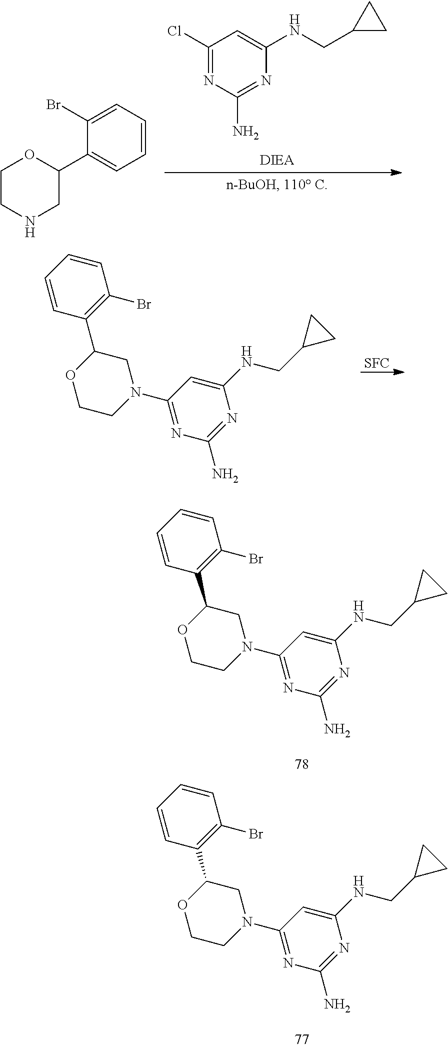

### Step 1: Preparation of 6-(2-(2-bromophenyl)morpholino)-N4-(cyclopropylmethyl)pyrimidine-2,4-diamine

To a solution of 2-(2-bromophenyl)morpholine (200 mg, 826.06 umol) and 6-chloro-N4-(cyclopropylmethyl)pyrimidine-2,4-diamine (164.10 mg, 826.06 umol) in n-BuOH (2 mL) was added DIEA (427.05 mg, 3.30 mmol). The mixture was stirred at 120° C. for 48 h. LC-MS showed a major peak (59%) with the desired mass. The reaction mixture was concentrated under reduced pressure to give a residue which was purified by prep-HPLC (basic conditions) to afford 6-(2-(2-bromophenyl)morpholino)-N4-(cyclopropylmethyl)pyrimidine-2,4-diamine (90 mg) as a yellow solid.

### Step 2: SFC Separation

6-(2-(2-bromophenyl)morpholino)-N4-(cyclopropylmethyl)pyrimidine-2,4-diamine (95 mg) was separated by SFC (column: DAICEL CHIRALCEL OD (250 mm*30 mm, 10 um); mobile phase: [0.1% NH3H2O IPA]; B %: 45%-45%, A2.8; 35 min) to afford:

Peak 1 (Retention time 1.950 min) as compound 78 (30 mg, 80% purity) which was further purified by prep-HPLC (basic conditions) to afford pure product as a white solid, ¹H NMR (DMSO-d₆, 400 MHz) δ 7.64 (dd, J=8.0, 1.2 Hz, 1H), 7.57 (dd, J=7.6, 1.6 Hz, 1H), 7.47-7.41 (m, 1H), 7.31-7.25 (m, 1H), 6.31 (br s, 1H), 5.54 (s, 2H) 5.12 (s, 1H) 4.68 (dd, J=10.0, 2.4 Hz, 1H), 4.25 (d, J=12.8 Hz, 1H), 4.10-4.00 (m, 2H), 3.72-3.63 (m, 1H), 3.04 (t, J=6.0 Hz, 2H), 2.90-2.82 (m, 1H), 2.61-2.58 (m, 1H), 1.02-0.92 (m, 1H), 0.42-0.36 (m, 2H), 0.2-0.10 (m, 2H), LC-MS: [M+H]+=404.8.

Peak 2 (Retention time 2.31 min) compound 77 (35 mg, 80% purity) which was further purified by prep-HPLC (basic conditions) to afford pure to afford pure product (14.2 mg) as a white solid, ¹H NMR (DMSO-d₆, 400 MHz) δ 7.64 (d, J=7.8 Hz, 1H), 7.57 (d, J=6.8 Hz, 1H), 7.44 (t, J=7.6 Hz, 1H), 7.29 (t, J=6.8 Hz, 1H), 6.39-6.25 (m, 1H), 5.57-5.48 (m, 2H), 5.12 (s, 1H), 4.70-4.64 (m, 1H), 4.27-4.22 (m, 1H), 4.08-4.02 (m, 2H), 3.69 (d, J=11.6 Hz, 1H), 3.04 (t, J=6.0 Hz, 2H), 2.89-2.83 (m, 1H), 2.58-2.55 (m, 1H), 1.00-0.94 (m, 1H), 0.42-0.36 (m, 2H), 0.18-0.13 (m, 2H), LC-MS: [M+H]+=404.8.

### Example 10: Preparation of R or S-6-(3-(imidazo[1,2-a]pyridin-3-yl)piperidin-1-yl)-2-isopropylpyrimidin-4-amine (Compound 141 and 142)

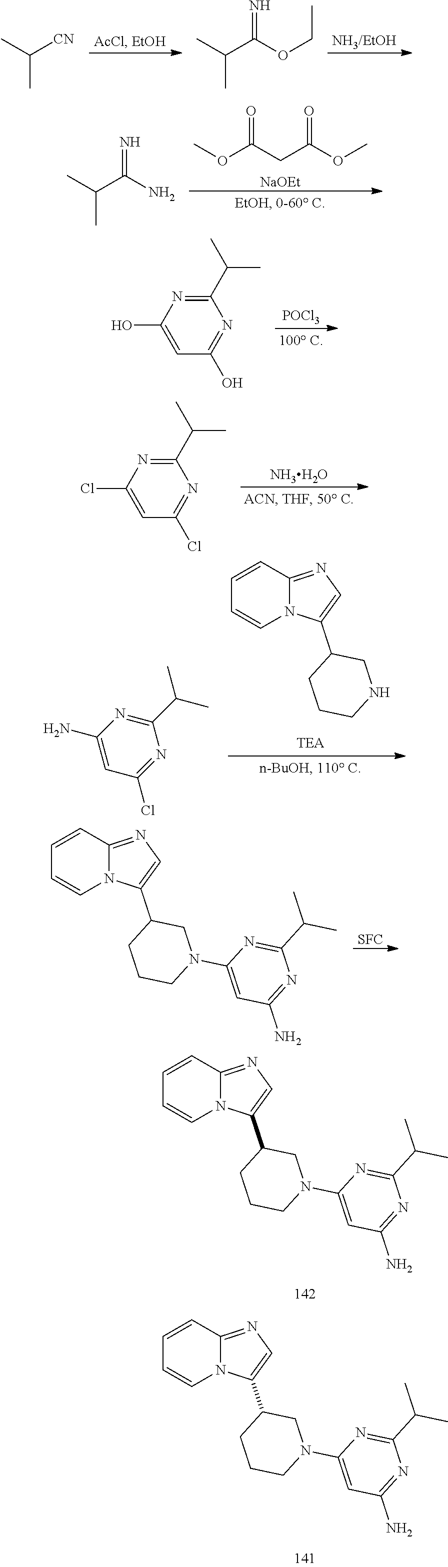

### Step 1: Preparation of Ethyl Isobutyrimidate

To a solution of isobutyronitrile (5.2 g, 75.25 mmol) in EtOH (50 mL) at 0° C. was added acetyl chloride (47.25 g, 601.98 mmol, 42.96 mL) dropwise. Once the addition was complete, the mixture was warmed to 20° C. and stirred at 20° C. for 16

The reaction mixture was concentrated under reduced pressure to give ethyl isobutyrimidate (10 g crude) as a white solid.

### Step 2: Preparation of Isobutyrimidamide

A solution of ethyl isobutyrimidate (10 g, 86.83 mmol) was added to a solution of NH3 in EtOH (20 mL). The mixture was stirred at 25° C. for 16 h. The reaction mixture was concentrated under reduced pressure to give a residue. The residue was dried over toluene (50 mL×3) to give isobutyrimidamide (8.7 g, crude) as a white solid.

### Step 3: Preparation of 2-Isopropylpyrimidine-4,6-Diol

To a solution of EtOH (30 mL) was added NaH (2.26 g, 56.58 mmol, 60% purity) slowly at 0° C. The solution was stirred at 20° C. for 30 min. To the solution was added isobutyrimidamide (3 g, 18.86 mmol) and dimethyl malonate (2.49 g, 18.86 mmol). The mixture was stirred at 80° C. for 4 h. The reaction mixture was adjusted pH to 7-8 with HCl (1 N, 40 mL). Solid was formed and the solid was collected by filtration. The solid was dried by azeotroping with toluene to give 2-isopropylpyrimidine-4,6-diol (2 g) as a yellow solid.

### Step 4: Preparation of 4,6-dichloro-2-isopropylpyrimidine

A solution of 2-isopropylpyrimidine-4,6-diol (500 mg, 3.24 mmol) was dissolved in POCl₃ (5 mL). The mixture was stirred at 100° C. for 16 h. TLC (Petroleum ether/Ethyl acetate=1/0) indicated formation of one new spot (Rf=0.24). After cooling to room temperature, the reaction mixture was concentrated under reduced pressure to remove solvent. The residue was slowly added to saturated solution of NaHCO₃ (50 mL) in a ice bath. The resulting mixture was extracted with Ethyl acetate (50 mL×3). The combined organic layers were washed with brine (50 mL), dried over Na₂SO₄, filtered and concentrated under reduced pressure to give 4,6-dichloro-2-isopropylpyrimidine (500 mg crude) as a yellow solid.

### Step 5: Preparation of 6-chloro-2-isopropylpyrimidin-4-amine

To a solution of 4,6-dichloro-2-isopropylpyrimidin (500 mg, 2.62 mmol) in ACN (1.5 mL) and THF (1.5 mL) was added NH₃·H₂O (2.73 g, 21.81 mmol, 3 mL). The mixture was stirred at 50° C. for 32 h. LC-MS showed peak area of 91% with the desired mass. After cooling to room temperature, the reaction mixture was concentrated under reduced pressure to remove solvent. The residue was dried over toluene (50 mL×3) to give 6-chloro-2-isopropylpyrimidin-4-amine (400 mg, crude) as an off-white solid.

### Step 6: Preparation of 6-(3-(imidazo[1,2-a]pyridin-3-yl)piperidin-1-yl)-2-isopropylpyrimidin-4-amine

To a solution of 6-chloro-2-isopropylpyrimidin-4-amine (237.86 mg, 1.39 mmol) and 3-(piperidin-3-yl)imidazo[1,2-a]pyridine (380 mg, 1.39 mmol, 2HCl) in n-BuOH (4 mL) was added TEA (701.19 mg, 6.93 mmol). The mixture was stirred at 110° C. for 32 h. LC-MS showed peak area of 58% with the desired mass. The reaction mixture was cooled to room temperature, concentrated under reduced pressure to remove solvent. The residue was purified by prep-HPLC (FA conditions) to afford 6-(3-(imidazo[1,2-a]pyridin-3-yl)piperidin-1-yl)-2-isopropylpyrimidin-4-amine (60 mg, FA salt) as a white solid.

### Step 7: SFC Separation

6-(3-(imidazo[1,2-a]pyridin-3-yl)piperidin-1-yl)-2-isopropylpyrimidin-4-amine (60 mg, FA) was separated by SFC (column: Phenomenex-Cellulose-2 (250 mm*30 mm, 10 um); mobile phase: [ACN/EtOH (0.1% NH3H2O)]; B %: 50%-50%, 4.3; 25 min) to afford:

Peak 1 (Retention time 1.017 min) as compound 142 (15.9 mg) as a yellow solid, ¹H NMR (MeOD-d₄, 400 MHz) δ 8.51 (d, J=6.8 Hz, 1H), 7.56 (d, J=9.2 Hz, 1H), 7.45 (s, 1H), 7.45-7.32 (m, 1H), 6.99 (t, J=6.0 Hz, 1H), 5.67 (s, 1H), 4.91-4.89 (m, 1H), 4.12 (d, J=13.6 Hz, 1H), 3.22-3.12 (m, 2H), 3.00-2.98 (m, 1H), 2.82-2.78 (m, 1H), 2.32-2.22 (m, 1H), 2.11-1.99 (m, 1H), 1.93-1.83 (m, 1H), 1.82-1.69 (m, 1H), 1.27 (d, J=6.8 Hz, 3H), 1.23 (d, J=6.8 Hz, 3H), LC-MS: [M+H]+=337.3.

Peak 2 (Retention time 1.432 min) as compound 141 (17.6 mg) as a yellow solid, ¹H NMR (MeOD-d₄, 400 MHz) δ 8.46 (d, J=6.4 Hz, 1H), 7.58 (d, J=9.2 Hz, 1H), 7.47 (s, 1H), 7.38-7.34 (m, 1H), 7.01 (t, J=6.4 Hz, 1H), 5.72 (s, 1H), 4.82 (d, J=11.6 Hz, 1H), 4.15 (d, J=11.6 Hz, 1H), 3.29-3.14 (m, 3H), 2.89-2.79 (m, 1H), 2.31-2.29 (m, 1H), 2.12-2.00 (m, 1H), 1.99-1.88 (m, 1H), 1.85-1.73 (m, 1H), 1.27 (d, J=6.8 Hz, 3H), 1.24 (d, J=6.8 Hz, 3H), LC-MS: [M+H]+=337.3.

### Example 11: Preparation of 4-(3-(imidazo[1,2-a]pyridin-3-yl)piperazin-1-yl)-6-isopropylpyrimidin-2-amine (Compound 32)

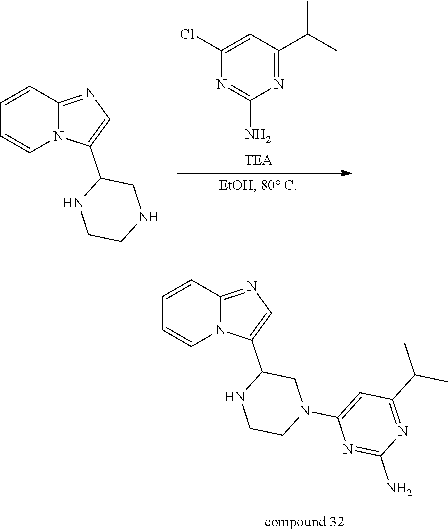 [SMILES: c1ccn2c(C3CNCCN3)cnc2c1 ; CC(C)c1cc(Cl)nc(N)n1 ; CC(C)c1cc(N2CCNC(c3cnc4ccccn34)C2)nc(N)n1]

### Step 1: Preparation of 4-(3-(imidazo[1,2-a]pyridin-3-yl)piperazin-1-yl)-6-isopropylpyrimidin-2-amine

To a mixture of 3-piperazin-2-ylimidazo[1,2-a]pyridine (400 mg, 1.28 mmol, 3 HCl) and 4-chloro-6-isopropyl-pyrimidin-2-amine (198.26 mg, 1.16 mmol) in EtOH (4 mL) was added TEA (714.34 mg, 7.06 mmol). The mixture was stirred at 80° C. for 10 h. LC-MS showed peak area of 76% with the desired mass. The reaction mixture was concentrated under reduced pressure to give a residue. The residue was purified by prep-HPLC (NH₄OH conditions) to afford 4-(3-imidazo[1,2-a]pyridin-3-ylpiperazin-1-yl)-6-isopropyl-pyrimidin-2-amine (90 mg) as a white solid, ¹H NMR (CD₃OD, 400 MHz) δ 8.62-8.49 (m, 1H), 7.64-7.51 (m, 2H), 7.40-7.29 (m, 1H), 7.00 (t, J=6.8 Hz, 1H), 6.07 (s, 1H), 4.67-4.56 (m, 1H), 4.32 (dd, J=9.6, 2.8 Hz, 1H), 4.24-4.11 (m, 1H) 3.40-3.35 (m, 1H), 3.25-3.18 (m, 1H), 3.15-3.05 (m, 1H), 3.01-2.91 (m, 1H) 2.76-2.58 (m, 1H) 1.23 (d, J=7.2 Hz, 6H), LC-MS: [M+H]+=338.2.

### Example 12: Preparation of 6-(3-(2-bromophenyl)piperidin-1-yl)-N²-methylpyrimidine-2,4-diamine (Compound 85)

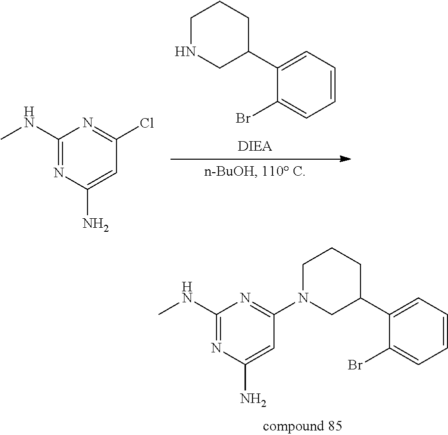

### Step 1: Preparation of 6-(3-(2-bromophenyl)piperidin-1-yl)-N2-methylpyrimidine-2,4-diamine

To a solution of 6-chloro-N2-methylpyrimidine-2,4-diamine (300 mg, 1.89 mmol) and 3-(2-bromophenyl)piperidine (963.60 mg, 1.32 mmol, 33% purity) in n-BuOH (1 mL) was added DIEA (488.97 mg, 3.78 mmol). The mixture was stirred at 110° C. for 16 h. LC-MS showed peak area of 13% with the desired mass. The mixture was filtered and concentrated under reduced pressure to give a residue. The residue was purified by prep-HPLC (FA condition) to afford 6-(3-(2-bromophenyl)piperidin-1-yl)-N2-methylpyrimidine-2,4-diamine (23.6 mg, FA salt) as a white solid, ¹H NMR (DMSO-d₆, 400 MHz) δ 8.54 (s, 1H), 7.60 (d, J=8.0 Hz, 1H), 7.42-7.41 (m, 1H), 7.39-7.33 (m, 1H), 7.15 (t, J=8.0 Hz, 1H), 4.88-4.87 (m, 1H), 4.54-4.32 (m, 2H), 3.23-3.17 (m, 1H), 3.03-2.99 (m, 1H), 2.94 (d, J=12.4 Hz, 1H), 2.89 (s, 3H), 2.04-2.02 (m, 1H), 1.93-1.83 (m, 2H), 1.71-1.61 (m, 1H), LC-MS: [M+H]+=364.1.

### Example 13: Preparation of N-(3-(dimethylamino)propyl)-4-(8-hydroxyquinoxalin-6-yl)benzamide (Compound 285)

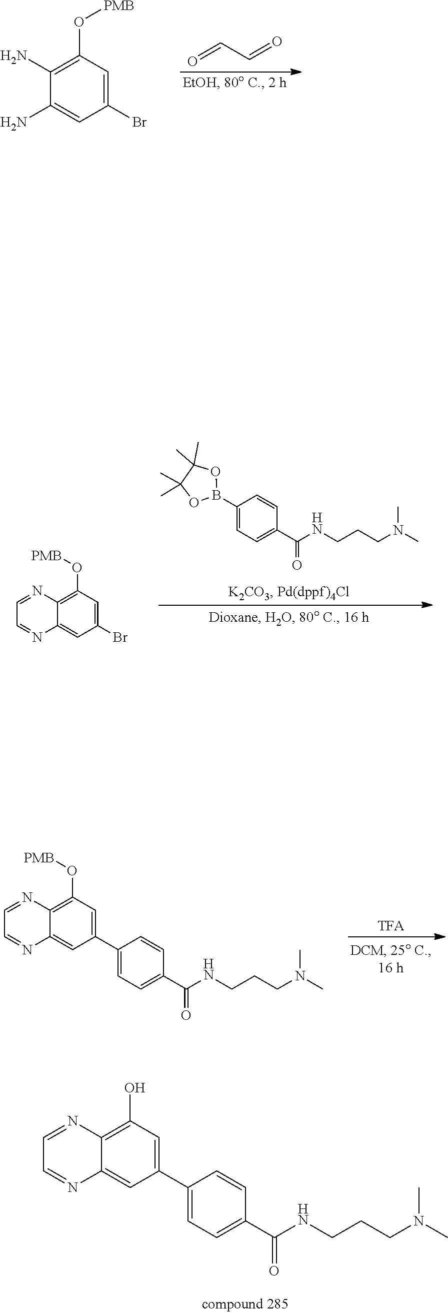

### Step1: Preparation of 7-bromo-5-((4-methoxybenzyl)oxy)quinoxaline

To a solution of 5-bromo-3-((4-methoxybenzyl)oxy)benzene-1,2-diamine (300 mg, 928.26 umol) in EtOH (3 mL) was added oxalaldehyde (64.65 mg, 1.11 mmol, 58.24 uL). The reaction was stirred at 80° C. for 2 h. LC-MS showed peak area of 63% with the desired mass. The reaction was concentrated to give 7-bromo-5-((4-methoxybenzyl)oxy)quinoxaline (260 mg) as a yellow solid. ¹H NMR (DMSO-d₆, 400 MHz) δ 9.00-8.85 (m, 2H), 7.87 (s, 1H), 7.61-7.52 (m, 1H), 7.47 (br d, J=8.4 Hz, 2H), 6.99 (br d, J=8.4 Hz, 2H), 5.28 (s, 2H), 3.77 (s, 3H).

### Step 2: Preparation of N-(3-(dimethylamino)propyl)-4-(8-((4-methoxybenzyl)oxy)quinoxalin-6-yl)benzamide

To a solution of 7-bromo-5-((4-methoxybenzyl)oxy)quinoxaline (100 mg, 289.70 umol,) and N-(3-(dimethylamino)propyl)-4-(4,4,5,5-tetramethyl-1,3,2-dioxaborolan-2-yl)benzamide (96.25 mg, 289.70 umol) in dioxane (1 mL) and H₂O (0.1 mL) was added K₂CO₃ (80.08 mg, 579.40 umol) and Pd(dppf)Cl₂ (21.20 mg, 28.97 umol). The reaction was stirred at 80° C. for 16 h. LC-MS showed peak area of 65% with the desired mass. The reaction was purified by prep-TLC (Dichloromethane:Methanol=5:1, 0.1% NH₃·H₂O) to give N-[3-(dimethylamino)propyl]-4-[8-[(4-methoxyphenyl)methoxy]quinoxalin-6-yl]benzamide (100 mg) as a brown solid.

### Step 3: Preparation of N-(3-(dimethylamino)propyl)-4-(8-hydroxyquinoxalin-6-yl)benzamide

To a solution of N-(3-(dimethylamino)propyl)-4-(8-((4-methoxybenzyl)oxy)quinoxalin-6-yl)benzamide (100 mg, 212.51 umol) in TFA (2 mL) and DCM (2 mL) was stirred at 25° C. for 16 h. LC-MS showed peak area of 58% with the desired mass. The reaction was concentrated to give a residue which was purified by prep-HPLC (FA condition) to give N-(3-(dimethylamino)propyl)-4-(8-hydroxyquinoxalin-6-yl)benzamide (26 mg, FA) as a yellow solid. ¹H NMR (DMSO-d₆, 400 MHz) δ 11.26-10.10 (m, 1H), 8.97 (d, J=1.6 Hz, 1H), 8.88 (d, J=1.6 Hz, 1H), 8.69-8.61 (m, 1H), 8.17 (s, 1H), 8.01-7.97 (m, 2H), 7.96-7.91 (m, 2H), 7.86 (d, J=2.0 Hz, 1H), 7.54 (d, J=2.0 Hz, 1H), 3.33 (br d, J=5.6 Hz, 2H), 2.60 (br t, J=7.2 Hz, 2H), 2.39 (s, 6H), 1.82-1.72 (m, 2H). LC-MS: [M+H]+=351.1.

### Example 14: Preparation of 4-(3-(2-bromo-3-fluorophenyl)piperazin-1-yl)-6-isopropylpyrimidin-2-amine (Compound 169)

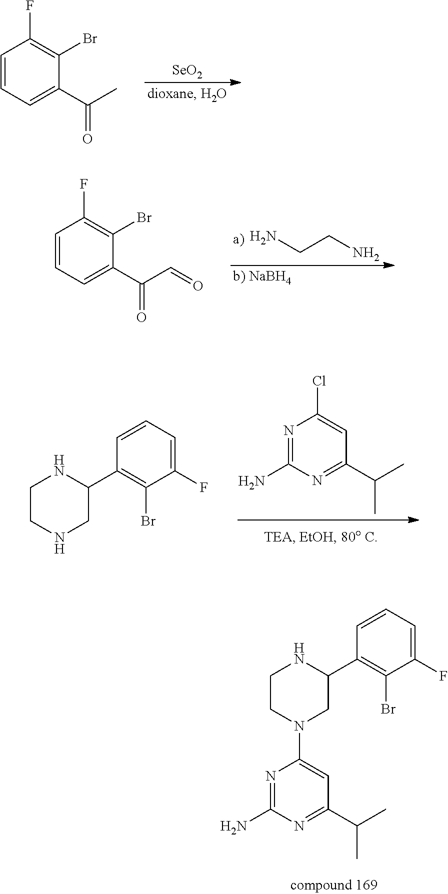

### Step1: Preparation of 2-(2-bromo-3-fluorophenyl)-2-oxoacetaldehyde

To a mixture of 1-(2-bromo-3-fluorophenyl)ethanone (200 mg, 921.51 umol) in dioxane (2 mL), H₂O (0.2 mL) was added SeO₂ (102.25 mg, 921.51 umol). The mixture was stirred at 100° C. for 10 h. TLC (Petroleum ether/Ethyl acetate=3/1) showed about 50% of new spot (Rf=0.2) was formed. The reaction mixture was filtered under vacuum to give a filter head and a filtrate. The filtrate was diluted with H₂O (20 mL) and extracted with EtOAc (20 mL×3). The combined organic layers were washed with brine (50 mL), dried over Na₂SO₄, filtered and concentrated under reduced pressure to give 2-(2-bromo-3-fluorophenyl)-2-oxoacetaldehyde (230 mg, crude) as yellow oil.

### Step 2: Preparation of 2-(2-bromo-3-fluorophenyl)piperazine

To a solution of 2-(2-bromo-3-fluorophenyl)-2-oxoacetaldehyde (230 mg, 995.59 umol) in MeOH (1.5 mL) and THF (1.5 mL) was added ethane-1,2-diamine (71.80 mg, 1.19 mmol) at 0° C. under N₂ and the mixture was stirred at 0° C. for 3 h. Then the reaction was added NaBH₄ (82.86 mg, 2.19 mmol) at 0° C. and stirred at 25° C. for 16 h. LC-MS showed peak area of 52% with the desired mass. The reaction mixture was quenched with HCl (1 N, 2 mL). The reaction mixture (pH=8-9) was stirred at 20° C. for 5 min. The resulting mixture was concentrated under vacuum to give a residue. The residue was lyophilized to remove water. Then the residue was triturated with DCM/MeOH (8/1; 100 mL) to give a filter head and a filtrate. The filtrate was concentrated under vacuum to give a 2-(2-bromo-3-fluorophenyl)piperazine (250 mg, crude) as yellow oil.

### Step 3: Preparation of 4-(3-(2-bromo-3-fluorophenyl)piperazin-1-yl)-6-isopropylpyrimidin-2-amine

To a mixtures of 2-(2-bromo-3-fluorophenyl)piperazine (250 mg, 964.81 umol), 4-chloro-6-isopropyl-pyrimidin-2-amine (115.91 mg, 675.37 umol) in EtOH (2 mL) was added TEA (292.89 mg, 2.89 mmol). The mixture was stirred at 80° C. for 10 h. LC-MS showed peak area of 64% with the desired mass. The mixture was cooled to room temperature, concentrated under vacuum to give a residue. The residue was purified by prep-HPLC (basic conditions) to afford compound 169 (72 mg, 18%) as a white solid. ¹H NMR (DMSO-d₆, 400 MHz) δ 7.56 (d, J=7.6 Hz, 1H), 7.48-7.46 (m, 1H), 7.38-7.27 (m, 1H), 5.93 (s, 1H), 5.89 (s, 2H), 4.41-4.20 (m, 2H), 4.00 (dd, J=2.4, 10.0 Hz, 1H), 3.05 (d, J=10.8 Hz, 1H), 2.98-2.96 (m, 1H), 2.88-2.72 (m, 2H), 2.65-2.57 (m, 1H), 2.49-2.44 (m, 1H), 1.13 (d, J=6.8 Hz, 6H), LC-MS: [M+H]+=394.2.

### Example 15: Preparation of 7-(3-(imidazo[1,2-a]pyridin-3-yl)piperidin-1-yl)-5-isopropyl-3H-imidazo[4,5-b]pyridine (Compound 19)

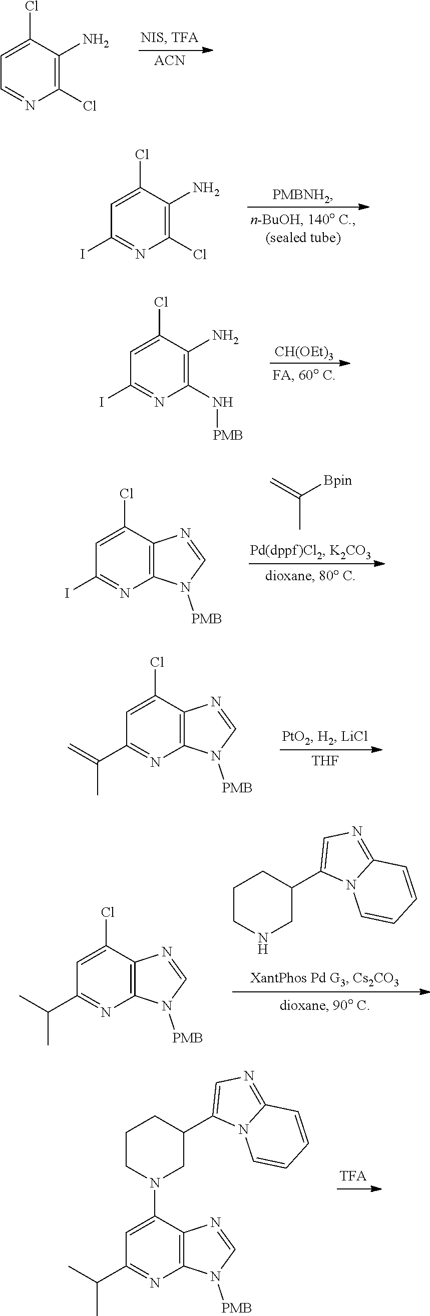

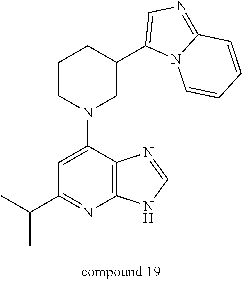 [SMILES: CC(C)c1cc(N2CCCC(c3cnc4ccccn34)C2)c2nc[nH]c2n1]

### Step1: Preparation of 2,4-dichloro-6-iodopyridin-3-amine

To a solution of 2,4-dichloropyridin-3-amine (4 g, 24.54 mmol) in ACN (40 mL) was added NIS (6.07 g, 26.99 mmol), TFA (839.41 mg, 7.36 mmol, 545.07 uL). The reaction mixture was stirred at 40° C. for 16 h. LC-MS showed peak area of 89% with the desired mass. The reaction mixture was poured into the saturated solution of Na₂SO₃ (100 ml), extracted with Ethyl acetate (50 mL×3). The combined organic layers were washed with brine (100 mL), dried over Na₂SO₄, filtered and concentrated to afford 2,4-dichloro-6-iodopyridin-3-amine (7 g) as a red solid. ¹H NMR (DMSO-d₆, 400 MHz) δ 7.75 (s, 1H), 6.01 (s, 2H).

### Step 2: Preparation of 4-chloro-6-iodo-N²-(4-methoxybenzyl)pyridine-2,3-diamine

To a solution of 2,4-dichloro-6-iodopyridin-3-amine (4 g, 13.85 mmol) in n-BuOH (24 mL) was added PMBNH₂ (9.50 g, 69.23 mmol). The mixture was stirred at 140° C. for 48 h. LC-MS showed peak area of 48% with the desired mass. The residue was cooled to room temperature, concentrated under vacuum to give a residue. The residue was purified by prep-HPLC (HCl conditions) to afford 4-chloro-6-iodo-N2-(4-methoxybenzyl)pyridine-2,3-diamine (1.9 g) as a red solid.

### Step 3: Preparation of 7-chloro-5-iodo-3-(4-methoxybenzyl)-3H-imidazo[4,5-b]pyridine

To a solution of 4-chloro-6-iodo-N2-(4-methoxybenzyl)pyridine-2,3-diamine (900 mg, 2.31 mmol) in HCOOH (4.5 mL) was added CH(OEt)₃ (1.03 g, 6.93 mmol, 1.15 mL). The mixture was stirred at 60° C. for 1.5 h. LC-MS showed peak area of 84% with the desired mass. The reaction mixture was cooled to room temperature, poured into the saturated solution of NaHCO₃ (20 ml), extracted with Ethyl acetate (30 mL×3). The combined organic layers were washed with brine (50 mL), dried over Na₂SO₄, filtered and concentrated under reduced pressure to afford 7-chloro-5-iodo-3-(4-methoxybenzyl)-3H-imidazo[4,5-b]pyridine (900 mg) as red oil.

### Step 4: Preparation of 7-chloro-3-(4-methoxybenzyl)-5-(prop-1-en-2-yl)-3H-imidazo[4,5-b]pyridine

To a solution of 7-chloro-5-iodo-3-(4-methoxybenzyl)-3H-imidazo[4,5-b]pyridine (900 mg, 2.25 mmol) and 4,4,5,5-tetramethyl-2-(prop-1-en-2-yl)-1,3,2-dioxaborolane (378.46 mg, 2.25 mmol) in dioxane (9 mL), H₂O (0.9 mL) was added Pd(dppf)Cl₂ (247.19 mg, 337.83 umol) and K₂CO₃ (778.16 mg, 5.63 mmol). The mixture was stirred at 80° C. for 16 h under N₂. LC-MS (showed about 67% peak of the desired mass. The residue was cooled to room temperature, purified by column chromatography (SiO₂, Petroleum ether/Ethyl acetate=100/0 to 1/1) to afford 7-chloro-3-(4-methoxybenzyl)-5-(prop-1-en-2-yl)-3H-imidazo[4,5-b]pyridine (500 mg) as a white solid.

### Step 5: Preparation of 7-chloro-5-isopropyl-3-(4-methoxybenzyl)-3H-imidazo[4,5-b]pyridine

To a solution of 7-chloro-3-(4-methoxybenzyl)-5-(prop-1-en-2-yl)-3H-imidazo[4,5-b]pyridine (500 mg, 1.59 mmol) in THF (2.5 mL) was added PtO₂ (100 mg, 440.37 umol), LiCl (202.64 mg, 4.78 mmol). The mixture was stirred at 25° C. for 2 h under H₂ (15 Psi). LC-MS showed peak area of 88% with the desired mass. The reaction mixture was filtered and the mother solution was concentrated under reduced pressure to afford 7-chloro-5-isopropyl-3-(4-methoxybenzyl)-3H-imidazo[4,5-b]pyridine (600 mg) as brown oil.

### Step 6: Preparation of 7-(3-(imidazo[1,2-a]pyridin-3-yl)piperidin-1-yl)-5-isopropyl-3-(4-methoxybenzyl)-3H-imidazo[4,5-b]pyridine

To a solution of 7-chloro-5-isopropyl-3-[(4-methoxyphenyl)methyl]imidazo[4,5-b]pyridine (300 mg, 949.98 umol), 3-(3-piperidyl)imidazo[1,2-a]pyridine (248.42 mg, 1.04 mmol, HCl) in dioxane (3 mL) was added XantPhos Pd G3 (90.09 mg, 95.00 umol), Cs₂CO₃ (1.08 g, 3.32 mmol). The mixture was stirred at 90° C. for 16 h under N₂. LC-MS showed peak area of 17% with the desired mass. The reaction mixture was cooled to room temperature, concentrated under reduced pressure to remove solvent. The residue was purified by prep-TLC (SiO₂, Petroleum ether/Ethyl acetate=0/1) to afford 7-(3-(imidazo[1,2-a]pyridin-3-yl)piperidin-1-yl)-5-isopropyl-3-(4-methoxybenzyl)-3H-imidazo[4,5-b]pyridine (50 mg) as yellow oil.

### Step 7: Preparation of 7-(3-(imidazo[1,2-a]pyridin-3-yl)piperidin-1-yl)-5-isopropyl-3H-imidazo[4,5-b]pyridine

To a solution of 7-(3-(imidazo[1,2-a]pyridin-3-yl)piperidin-1-yl)-5-isopropyl-3-(4-methoxybenzyl)-3H-imidazo[4,5-b]pyridine (50 mg, 104.04 umol) in TFA (1 mL) was stirred at 25° C. for 16 h. LC-MS showed peak area of 75% with the desired mass. The reaction mixture was concentrated under vacuum to give a residue. The residue was purified by prep-HPLC (TFA conditions) to afford compound 19 (24.2 mg, 2TFA salts) as a yellow solid. ¹H NMR (MeOD-d₄, 400 MHz) δ 9.30-9.18 (m, 1H), 8.23 (s, 1H), 8.05-8.00 (m, 2H), 7.98-7.93 (m, 1H), 7.63 (t, J=6.8 Hz, 1H), 6.82 (s, 1H), 4.82-4.48 (m, 2H), 3.70-3.51 (m, 3H), 3.21-3.16 (m, 1H), 2.46-2.38 (m, 1H), 2.21-2.11 (m, 2H), 2.04-1.94 (m, 1H), 1.43 (dd, J=1.2, 8.0 Hz, 6H), LC-MS: [M+H]+=361.4.

### Example 16: Preparation of 3-(1-aminoisoquinolin-7-yl)-N-(3-(dimethylamino)propyl)benzo[b]thiophene-6-carboxamide (Compound 357)

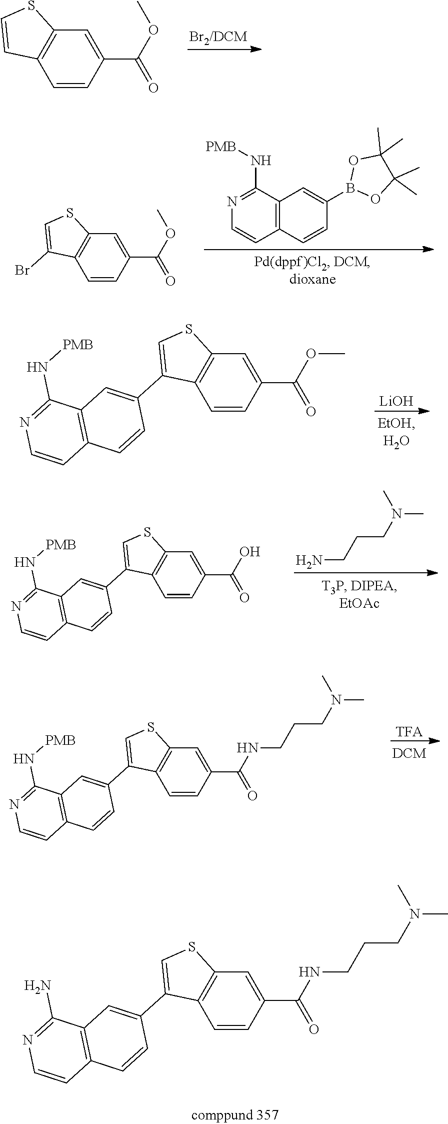

### Step1: Preparation of Methyl 3-bromobenzo[b]thiophene-6-carboxylate

Br₂ (499 mg, 3.121 mmol, 2 eq) was added dropwise to a mixture of methyl benzo[b]thiophene-6-carboxylate (300 mg, 1.561 mmol, 1 eq) in DCM (5 mL) at 0° C., then the mixture was allowed to stir for overnight. TLC (EA/PE=1:5) showed that the starting material was consumed completely and desired compound was detected. The solvent was concentrated under vacuum to give 3-bromobenzo[b]thiophene-6-carboxylate (350 mg, yield 82.72%) as a yellow solid which was used directly in next step without further purification. ¹H NMR (CDCl₃, 500 MHz) δ 8.58 (s, 1H), 8.12 (dd, J=8.0, 1.5 Hz, 1H), 7.87 (d, J=8.5 Hz, 1H), 7.63 (s, 1H), 3.97 (s, 3H).

### Step 2: Preparation of Methyl 3-(1-((4-methoxybenzyl)amino)isoquinolin-7-yl)benzo[b]thiophene-6-carboxylate

A mixture of methyl 3-bromobenzo[b]thiophene-6-carboxylate (400 mg, 1.446 mmol, 1 eq, purity 98%), N-(4-methoxybenzyl)-7-(4,4,5,5-tetramethyl-1,3,2-dioxaborolan-2-yl)isoquinolin-1-amine (846 mg, 2.2 mmol, 1.5 eq), Pd(dppf)Cl₂·DCM (120. mg, 0.145 mmol, 0.1 eq, purity 98%) and K₂CO₃ (605 mg, 4.3 mmol, 3 eq, purity 99%) in dioxane (10 mL) and H₂O (2 mL) was stirred at 100° C. under N₂ for 16 h. LC-MS showed that the starting material was consumed completely and desired compound was detected. The mixture was concentrated and the residue was purified by column chromatography on silica gel to give methyl 3-(1-((4-methoxybenzyl)amino)isoquinolin-7-yl)benzo[b]thiophene-6-carboxylate (250 mg, purity 90%, yield 34.24%) as a yellow solid.

### Step 3: Preparation of 3-(1-((4-methoxybenzyl)amino)isoquinolin-7-yl)benzo[b]thiophene-6-carboxylic Acid

A mixture of methyl 3-(1-((4-methoxybenzyl)amino)isoquinolin-7-yl)benzo[b]thiophene-6-carboxylate (250 mg, 0.550 mmol, 1 eq) and LiOH (52.7 mg, 2.27 mmol, 4 eq) in EtOH (5 mL) and H₂O (2 mL) was stirred at 40° C. for 2 h. LC-MS showed that the starting material was consumed completely and desired compound was detected. The mixture was concentrated and the residue was dissolved in water (5 mL), adjusted to pH 3˜4 with HCl (1 N), extracted with EtOAc (3×5 mL), the combined organic layer was dried over anhydrous Na₂SO₄, filtered and concentrated to give 3-(1-((4-methoxybenzyl)amino)isoquinolin-7-yl)benzo[b]thiophene-6-carboxylic acid (200 mg, purity 90%, yield 74.29%) as a white solid which was used directly in next step without further purification.

### Step 4: Preparation of N-(3-(dimethylamino)propyl)-3-(1-((4-methoxybenzyl)amino)isoquinolin-7-yl)benzo[b]thiophene-6-carboxamide

T₃P (346.7 mg, 0.545 mmol, 1.2 eq, purity 50%) was added to a mixture of 3-(1-((4-methoxybenzyl)amino)isoquinolin-7-yl)benzo[b]thiophene-6-carboxylic acid (200 mg, 0.454 mmol, 1 eq), N¹,N¹-dimethylpropane-1,3-diamine (55.7 mg, 0.545 mmol, 1.2 eq) and DIEA (176.0 mg, 1.362 mmol, 3 eq) in EtOAc (5 mL) at room temperature, then the mixture was stirred at this temperature for 10 h. LC-MS showed that the starting material was consumed completely and desired compound was detected. The mixture was concentrated and the residue was purified by column chromatography on silica gel to give the desired compound (150 mg, purity 90%, yield 56.67%) as yellow solid.

### Step 5: Preparation of 3-(1-aminoisoquinolin-7-yl)-N-(3-(dimethylamino)propyl)benzo[b]thiophene-6-carboxamide

The N-(3-(dimethylamino)propyl)-3-(1-((4-methoxybenzyl)amino)isoquinolin-7-yl)benzo[b]thiophene-6-carboxamide (150 mg, 0.282 mmol, 1 eq, purity 98.65%) in DCM (4.5 mL) and TFA (1 mL) was stirred at room temperature for 10 h. LC-MS showed that the starting material was consumed completely and desired compound was detected. The mixture was concentrated and the residue was purified by Prep-HPLC and lyophilized to give compound 357 (26.6 mg, purity 99.9%, yield 23.29%) as a white solid. ¹H NMR (CD₃OD, 500 MHz) δ 8.68 (s, 1H), 8.55 (s, 1H), 8.21 (d, J=8.0 Hz, 1H), 8.08-8.02 (m, 3H), 7.95 (d, J=9.0 Hz, 1H), 7.63 (d, J=7.0 Hz, 1H), 7.29 (d, J=6.5 Hz, 1H), 3.55 (t, J=7.0 Hz, 2H), 3.23 (t, J=7.5 Hz, 2H), 2.93 (s, 6H), 2.11-2.05 (m, 2H). LC-MS: [M+H]+=405.0.

### Example 17: Preparation of 4-(3-(1H-indol-3-yl)piperidin-1-yl)-6-isopropylpyrimidin-2-amine (Compound 249)

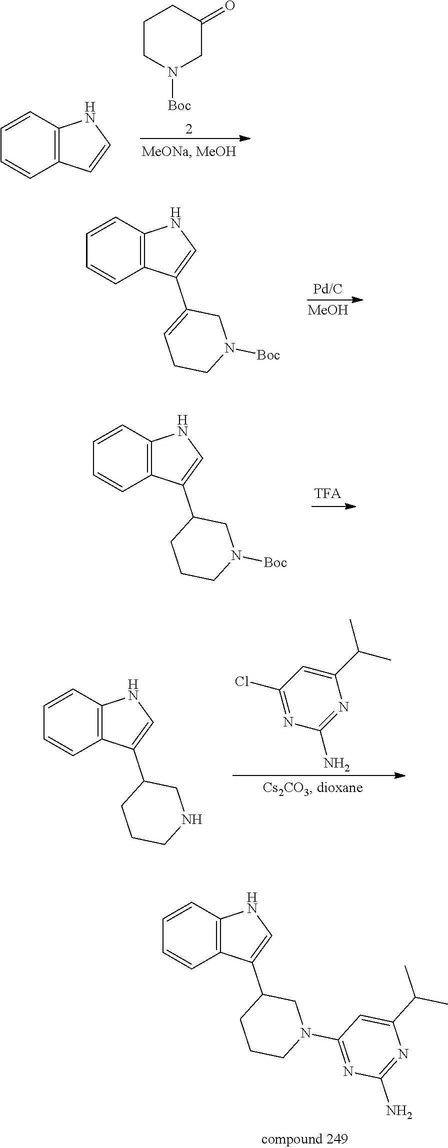

### Step 1: Preparation of Tert-Butyl 5-(1H-indol-3-yl)-3,6-dihydropyridine-1(2H)-carboxylate

A mixture of indole (1.0 g, 8.5 mmol, 1 eq), tert-butyl 3-oxopiperidine-1-carboxylate (3.4 g, 17.0 mmol, 2 eq) and MeONa (1.84 g, 34.1 mmol, 4 eq) in MeOH (50 ml) was stirred at 80° C. under N₂ for 40 h. TLC showed that the starting material remained and desired compound was detected. The mixture was cooled to room temperature and adjusted to pH 6˜7 with AcOH, concentrated and the residue was extracted with EtOAc and water. The combined organic layer was dried over anhydrous Na₂SO₄, filtered and concentrated, the residue was purified by column chromatography on silica gel to give tert-butyl 5-(1H-indol-3-yl)-3,6-dihydropyridine-1(2H)-carboxylate (400 mg, yield 15.70%) as a slight yellow solid. ¹H NMR: (CDCl₃, 500 MHz) δ 8.18-8.08 (m, 1H), 7.96-7.83 (m, 1H), 7.59-7.44 (m, 1H), 7.39-7.34 (m, 1H), 7.24-7.12 (m, 3H), 3.69-3.63 (m, 2H), 2.51-2.47 (m, 2H), 2.05-1.98 (m, 2H), 1.56-1.53 (m, 9H).

### Step 2: Preparation of Tert-Butyl 3-(1H-indol-3-yl)piperidine-1-carboxylate

Tert-butyl 5-(1H-indol-3-yl)-3,6-dihydropyridine-1 (2H)-carboxylate (400 mg, 1.341 mmol, 1 eq) and Pd/C (14.3 mg, 0.134 mmol, 0.1 eq) in MeOH (10 mL) was stirred at 15° C. under 15 psi of H₂ for 12 h. TLC showed that the starting material was consumed completely. The mixture was filtered and the filtrate was concentrated to give tert-butyl 3-(1H-indol-3-yl)piperidine-1-carboxylate (390 mg, yield 96.84%) as a slight yellow solid. ¹H NMR (CDCl₃, 500 MHz) δ 8.03 (s, 1H), 7.70 (d, J=8.0 Hz, 1H), 7.37 (d, J=8.0 Hz, 1H), 7.20 (t, J=7.5 Hz, 1H), 7.12 (t, J=7.5 Hz, 1H), 7.00 (d, J=2.5 Hz, 1H), 4.38 (br, 1H), 4.12 (br, 1H), 3.06-2.98 (m, 1H), 2.82 (dd, J=13.0, 11.0 Hz, 2H), 2.21-2.16 (m, 1H), 1.82-1.63 (m, 3H), 1.50 (s, 9H).

### Step 3: Preparation of 3-(piperidin-3-yl)-1H-indole

A mixture of tert-butyl 3-(1H-indol-3-yl)piperidine-1-carboxylate (200 mg, 0.666 mmol, 1 eq) and TFA (151.8 mg, 1.3 mmol, 2 eq) in DCM (2 mL) was stirred at room temperature for 1 h. LC-MS showed that the starting material was consumed and target molecule was detected. The mixture was concentrated to give 3-(piperidin-3-yl)-1H-indole (120 mg crude) as a black oil which was used directly for the next step without further purification.

### Step 4: Preparation of 4-(3-(1H-indol-3-yl)piperidin-1-yl)-6-isopropylpyrimidin-2-amine

A mixture of 3-(piperidin-3-yl)-1H-indole (60.000 mg, 0.300 mmol, 1 eq), 4-chloro-6-isopropylpyrimidin-2-amine (53.988 mg, 0.315 mmol, 1.05 eq), Cs₂CO₃ (292.837 mg, 0.899 mmol, 3 eq) in 1,4-dioxane (8 mL) was stirred at 110° C. for 20 h. The solvent was concentrated under vacuum and the residue was purified by preparative chromatography (MeCN/H₂O=3:2) to give compound 249 (29.000 mg, yield 28.86%) as a white solid. ¹H NMR (CD₃OD, 500 MHz) δ 7.64 (d, J=7.5 Hz, 1H), 7.34 (d, J=8.0 Hz, 1H), 7.11-7.07 (m, 2H), 7.04-7.00 (m, 1H), 5.98 (s, 1H), 4.63-4.59 (m, 1H), 4.41-4.38 (m, 1H), 3.07-2.97 (m, 3H), 2.68-2.62 (m, 1H), 2.23-2.18 (m, 1H), 1.94-1.84 (m, 2H), 1.74-1.68 (m, 1H), 1.22 (d, J=7.0 Hz, 6H). LC-MS: [M+H]+=336.0.

### Example 18: Preparation of 1-(2-amino-6-isopropylpyrimidin-4-yl)-3-(2-bromophenyl)piperidin-3-ol (Compound 236)

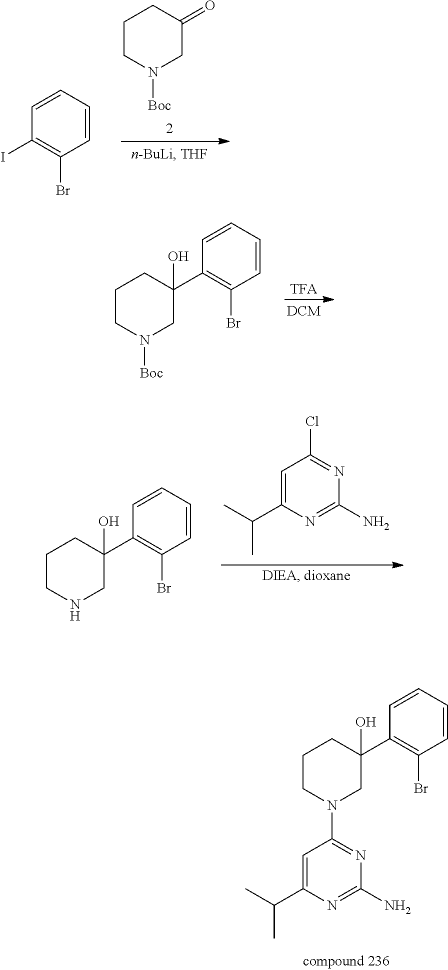

### Step1: Preparation of Tert-Butyl 3-(2-bromophenyl)-3-hydroxypiperidine-1-carboxylate

n-BuLi (339.6 mg, 5.3 mmol, 1.5 eq) was added to a mixture of 1-bromo-2-iodobenzene (1.0 g, 3.5 mmol, 1 eq) at −78° C. under N₂. After 10 mins, tert-butyl 3-oxopiperidine-1-carboxylate (774.744 mg, 3.888 mmol, 1.1 eq) was added and the mixture was stirred for another 1 h, then allowed to stir at room temperature for 2 h. TLC showed that the starting material was consumed completely. The mixture was quenched with NH₄Cl (saturated aqueous solution), extracted with EtOAc. The combined organic layer was dried over anhydrous Na₂SO₄, filtered and concentrated to give a residue which purified by column chromatography to give tert-butyl 3-(2-bromophenyl)-3-hydroxypiperidine-1-carboxylate (400 mg, purity 90%, yield 28.59%) as a yellow solid.

### Step 2: Preparation of 3-(2-bromophenyl)piperidin-3-ol

Tert-butyl 3-(2-bromophenyl)-3-hydroxypiperidine-1-carboxylate (200 mg, 0.561 mmol, 1 eq) in DCM (5 mL) and TFA (1 mL) was stirred at room temperature for 10 h. TLC showed that the starting material was consumed completely. The mixture was concentrated to give 3-(2-bromophenyl)piperidin-3-ol (120 mg crude) as a yellow oil which was used directly in next step without further purification.

### Step 3: Preparation of 1-(2-amino-6-isopropylpyrimidin-4-yl)-3-(2-bromophenyl)piperidin-3-ol

A mixture of 3-(2-bromophenyl)piperidin-3-ol (100.000 mg, 0.390 mmol, 1 eq), 4-chloro-6-isopropylpyrimidin-2-amine (201.019 mg, 1.171 mmol, 3 eq) and DIPEA (252.303 mg, 1.952 mmol, 5 eq) in dioxane (6 mL) was stirred at 110° C. under N₂ for 12 h. LC-MS showed that the starting material was consumed completely. The mixture was concentrated and the residue was purified by Perp-HPLC to give compound 236 (21.200 mg, 100% purity, yield 13.88%) as a white solid. ¹H NMR (CDCl₃, 500 MHz) δ 7.85 (dd, J=8.0, 1.5 Hz, 1H), 7.61 (d, J=8.0 Hz, 1H), 7.35 (t, J=7.5 Hz, 1H), 7.14 (td, J=7.5, 1.5 Hz, 1H), 5.95 (s, 1H), 4.66-4.60 (m, 3H), 4.14 (d, J=14.0 Hz, 1H), 3.89 (d, J=14.0 Hz, 1H), 3.71 (s, 1H), 3.14 (td, J=13.0, 3.0 Hz, 1H), 2.73-2.62 (m, 2H), 2.09-1.99 (m, 1H), 1.95 (d, J=13.5 Hz, 1H), 1.69-1.66 (m, 1H), 1.21 (d, J=7.0 Hz, 6H), LC-MS: [M+H]+=391.0

### Example 19: Preparation of 4-(3-(2-bromophenyl)-3-fluoropiperidin-1-yl)-6-isopropylpyrimidin-2-amine (Compound 233)

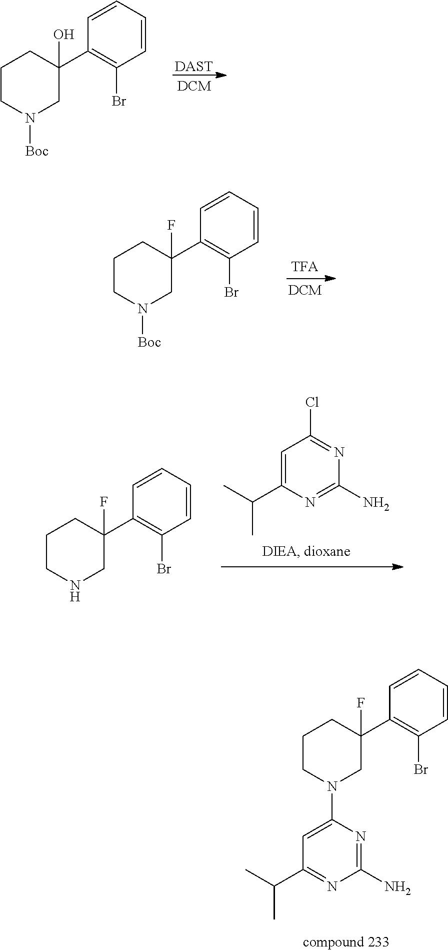

### Step 1: Preparation of Tert-Butyl 3-(2-bromophenyl)-3-fluoropiperidine-1-carboxylate

DAST (543 mg, 3.368 mmol, 1.2 eq) was added dropwise to a mixture of tert-butyl 3-(2-bromophenyl)-3-hydroxypiperidine-1-carboxylate (1.0 g, 2.81 mmol, 1 eq) in DCM (20 mL) at 0° C. under N₂, then the mixture was stirred at room temperature for 1 h. TLC showed the starting material was consumed completely. The mixture was quenched with NH₄Cl (sat), extracted with DCM, the combined organic layer was dried over anhydrous Na₂SO₄, filtered and concentrated to give a residue which was purified by column chromatography on silica gel to give compound 2 (530 mg, yield 52.71%) as a yellow solid.

### Step 2: Preparation of 3-(2-bromophenyl)-3-fluoropiperidine

To a solution of tert-butyl 3-(2-bromophenyl)-3-fluoropiperidine-1-carboxylate (530 mg, 1.479 mmol, 1 eq) in DCM (3 mL) was added TFA (505.9 mg, 4.438 mmol, 3 eq) at room temperature. The mixture was stirred for 2.5 h. TLC analysis indicated that the reactant was consumed completely. The reaction mixture was concentrated to give 3-(2-bromophenyl)-3-fluoropiperidine (360 mg, purity 95%, yield 89.56%) as a yellow solid.

### Step 3: Preparation of 4-(3-(2-bromophenyl)-3-fluoropiperidin-1-yl)-6-isopropylpyrimidin-2-amine

To a solution of 3-(2-bromophenyl)-3-fluoropiperidine (150.000 mg, 0.581 mmol, 1 eq) and 4-chloro-6-isopropylpyrimidin-2-amine (119.682 mg, 0.697 mmol, 1.2 eq) in dioxane (15 mL) was added DIEA (224.887 mg, 1.743 mmol, 3 eq) at room temperature. The mixture was stirred at 120° C. under N₂ atmosphere for 12 h. TLC analysis indicated that the reactant was consumed completely. The reaction mixture was concentrated, and the residue was purified by Prep-HPLC to give the desired product compound 233 (4.000 mg, yield 1.75%) as a white solid.

¹H NMR (CDCl₃, 500 MHz) δ 7.70 (dd, J=8.0, 2.0 Hz, 1H), 7.63 (d, J=8.0 Hz, 1H), 7.38 (td, J=8.0, 1.5 Hz, 1H), 7.20 (td, J=7.5, 2.0 Hz, 1H), 5.88 (s, 1H), 4.61 (s, 2H), 4.56-4.38 (m, 2H), 3.99 (dd, J=37.0, 14.0 Hz, 1H), 3.05 (t, J=12.0 Hz, 1H), 2.97-2.82 (m, 1H), 2.70-2.62 (m, 1H), 2.08-2.00 (m, 2H), 1.79-1.76 (m, 1H), 1.21 (d, J=7.0 Hz, 6H). LC-MS: [M+H]⁺=395.0

### Example 20: Preparation of 4-(5-(2-bromophenyl)-3,6-dihydropyridin-1(2H)-yl)-6-isopropylpyrimidin-2-amine (Compound 230)

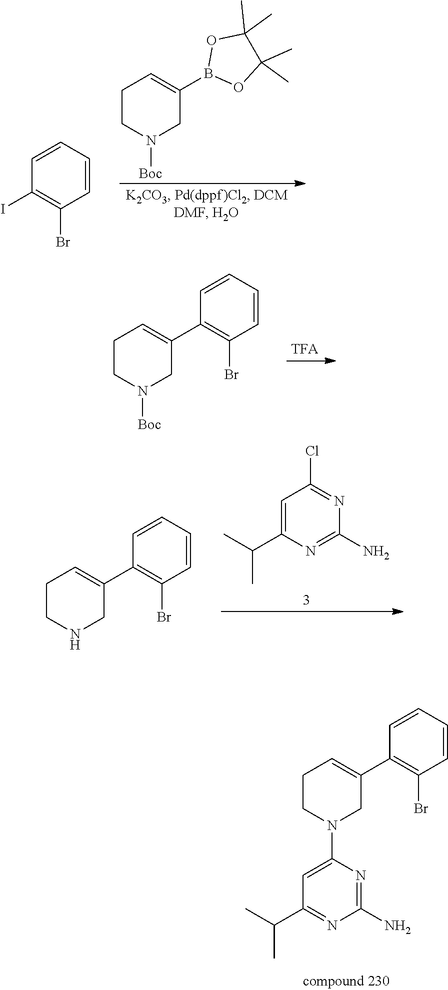

### Step 1: Preparation of Tert-Butyl 5-(2-bromophenyl)-3,6-dihydropyridine-1(2H)-carboxylate

A mixture of 1-bromo-2-iodobenzene (7.685 g, 27.166 mmol, 3 eq tert-butyl 5-(4,4,5,5-tetramethyl-1,3,2-dioxaborolan-2-yl)-3,6-dihydropyridine-1(2H)-carboxylate (2.800 g, 9.055 mmol, 1 eq), Pd(dppf)Cl₂·DCM (748.072 mg, 0.906 mmol, 0.1 eq, purity 98%), K₂CO₃ (3.792 g, 27.166 mmol, 3 eq, purity 99%) in DMF (10 mL) and H₂O (2 mL) was stirred at 60° C. for 20 h. TLC (EA/PE=1:40) showed the starting material was consumed completely and a newly generated spot was observed. The solvent was concentrated under reduced pressure and the residue was purified by flash column chromatography (EA/PE=3:97) to give tert-butyl 5-(2-bromophenyl)-3,6-dihydropyridine-1(2H)-carboxylate (2.300 g, yield 75.09%) as a white solid. ¹H NMR (CD₃OD, 500 MHz) δ 7.58 (dd, J=8.0, 1.5 Hz, 1H), 7.33 (td, J=7.5, 1.0 Hz, 1H), 7.24 (dd, J=7.5, 2.0 Hz, 1H), 7.20 (td, J=7.5, 2.0 Hz, 1H), 5.78-5.74 (m, 1H), 4.09 (s, 2H), 3.60-3.55 (m, 2H), 2.31-2.26 (m, 2H), 1.48 (s, 9H).

### Step 2: Preparation of 5-(2-bromophenyl)-1,2,3,6-tetrahydropyridine

A mixture of tert-butyl 5-(2-bromophenyl)-3,6-dihydropyridine-1(2H)-carboxylate (50 mg, 0.209 mmol, 1 eq) in DCM (5 mL) and TFA (1 mL) was stirred at room temperature for 2 h. LC-MS showed that the starting material was consumed completely and desired compound was detected. The mixture was concentrated to give compound 5-(2-bromophenyl)-1,2,3,6-tetrahydropyridine (35 mg crude) as a yellow oil which was used directly in next step without further purification.

### Step 3: Preparation of 4-(5-(2-bromophenyl)-3,6-dihydropyridin-1(2H)-yl)-6-isopropylpyrimidin-2-amine

A mixture of 5-(2-bromophenyl)-1,2,3,6-tetrahydropyridine (35.0 mg, 0.147 mmol, 1 eq), 4-chloro-6-isopropylpyrimidin-2-amine (27.7 mg, 0.162 mmol, 1.1 eq), Cs₂CO₃ (143.7 mg, 0.441 mmol, 3 eq) in dioxane (5 mL) was stirred at 110° C. for 20 h. LC-MS showed that the starting material was consumed completely and desired compound was detected. The reaction mixture was concentrated under reduced pressure and the residue was purified by preparative chromatography (MeCN/H₂O=3:2) to give compound 230 (4.9 mg, yield 8.93%) as a yellow solid after lyophilization. ¹H NMR (CD₃OD, 500 MHz) δ 7.60 (d, J=7.5 Hz, 1H), 7.35 (t, J=7.5 Hz, 1H), 7.30-7.27 (m, 1H), 7.21 (t, J=7.5 Hz, 1H), 5.96 (s, 1H), 5.84-5.81 (m, 1H), 4.23 (s, 2H), 3.82 (t, J=6.0 Hz, 2H), 2.68-2.62 (m, 1H), 2.38-2.35 (m, 2H), 1.21 (d, J=6.5 Hz, 6H). LC-MS: [M+H]+=373.0.

### Example 21: Preparation of 4-(3-(imidazo[1,5-a]pyridin-1-yl)piperidin-1-yl)-6-isopropylpyrimidin-2-amine (Compound 235)

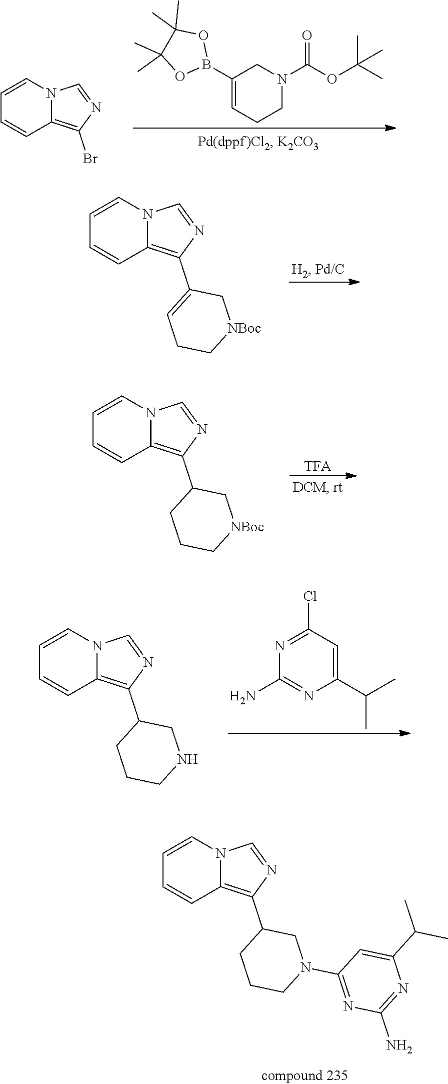

### Step 1: Preparation of Tert-Butyl 5-(imidazo[1,5-a]pyridin-1-yl)-3,6-dihydropyridine-1(2H)-carboxylate

A mixture of 1-bromoimidazo[1,5-a]pyridine (500.000 mg, 2.538 mmol, 1 eq), tert-butyl 5-(4,4,5,5-tetramethyl-1,3,2-dioxaborolan-2-yl)-3,6-dihydropyridine-1(2H)-carboxylate (1.177 g, 3.807 mmol, 1.5 eq), Pd(dppf)Cl₂·DCM (209.795 mg, 0.254 mmol, 0.1 eq) and K₂CO₃ (701.416 mg, 5.075 mmol, 2 eq) in dioxane (10 mL) and water (1 mL) was stirred at 80° C. under N₂ for 10 h. TLC showed that the starting material was consumed completely. The mixture was filtered and the filtrate was concentrated and purified by column chromatography to give tert-butyl 5-(imidazo[1,5-a]pyridin-1-yl)-3,6-dihydropyridine-1 (2H)-carboxylate (400.000 mg, purity 85%, yield 44.75%) as a yellow solid.

### Step 2: Preparation of Tert-Butyl 3-(imidazo[1,5-a]pyridin-1-yl)piperidine-1-carboxylate

Tert-butyl 5-(imidazo[1,5-a]pyridin-1-yl)-3,6-dihydropyridine-1(2H)-carboxylate (400.000 mg, 1.336 mmol, 1 eq) and Pd/C (142.192 mg, 0.134 mmol, 0.1 eq, purity 10%) in MeOH (10 mL) was stirred at room temperature under H₂ balloon for 2 h. LC-MS showed that the starting material was consumed completely and desired compound was detected. The mixture was filtered and the filtrate was concentrated to give tert-butyl 3-(imidazo[1,5-a]pyridin-1-yl)piperidine-1-carboxylate (360.000 mg, purity 90%, yield 80.46%) as a colorless oil which was used directly in next step without further purification.

### Step 3: Preparation of 1-(piperidin-3-yl)imidazo[1,5-a]pyridine

To a solution of tert-butyl 3-(imidazo[1,5-a]pyridin-1-yl)piperidine-1-carboxylate (360.000 mg, 1.194 mmol, 1 eq) in DCM (2.5 mL) was added TFA (408.5 mg, 3.583 mmol, 3 eq) at room temperature. The mixture was stirred for another 2.5 h. TLC analysis showed that the reactant was consumed completely. The reaction mixture was concentrated to give the desired crude product (240.000 mg, purity 90%, yield 89.85%) as a yellow solid.

### Step 4: Preparation of 4-(3-(imidazo[1,5-a]pyridin-1-yl)piperidin-1-yl)-6-isopropylpyrimidin-2-amine

To a solution of 1-(piperidin-3-yl)imidazo[1,5-a]pyridine (150.000 mg, 0.671 mmol, 1 eq, purity 90%) and 4-chloro-6-isopropylpyrimidin-2-amine 145.414 mg, 0.805 mmol, 1.2 eq, purity 95%) in dioxane (20 mL) was added DIEA (259.577 mg, 2.012 mmol, 3 eq) at rt. The reaction mixture was stirred at 120° C. under N₂ atmosphere for 12 h. TLC analysis showed that the reactant was completely consumed. The reaction mixture was concentrated and the residue was purified by Prep-HPLC to obtain compound 235 (8.900 mg, purity 97.5%, yield 3.94%) as a white solid. ¹H NMR (CDCl₃, 500 MHz) δ 8.05 (s, 1H), 7.86 (d, J=7.0 Hz, 1H), 7.44 (d, J=9.5 Hz, 1H), 6.66-6.62 (m, 1H), 6.54-6.50 (m, 1H), 5.87 (s, 1H), 4.69 (br, 2H), 4.51-4.45 (m, 2H), 3.19-3.08 (m, 2H), 2.95-2.88 (m, 1H), 2.69-2.63 (m, 1H), 2.11-2.05 (m, 2H), 1.89-1.86 (m, 1H), 1.72-1.66 (m, 1H), 1.21 (dd, J=7.0, 1.5 Hz, 6H). LC-MS: 97.52% purity, [M+H]+=337.0.

### Example 22: Preparation of R or S-4-(3-(2-bromo-5-(1,5-dimethyl-1H-pyrazol-4-yl)phenyl)piperazin-1-yl)-6-isopropylpyrimidin-2-amine (Compound 6/7)

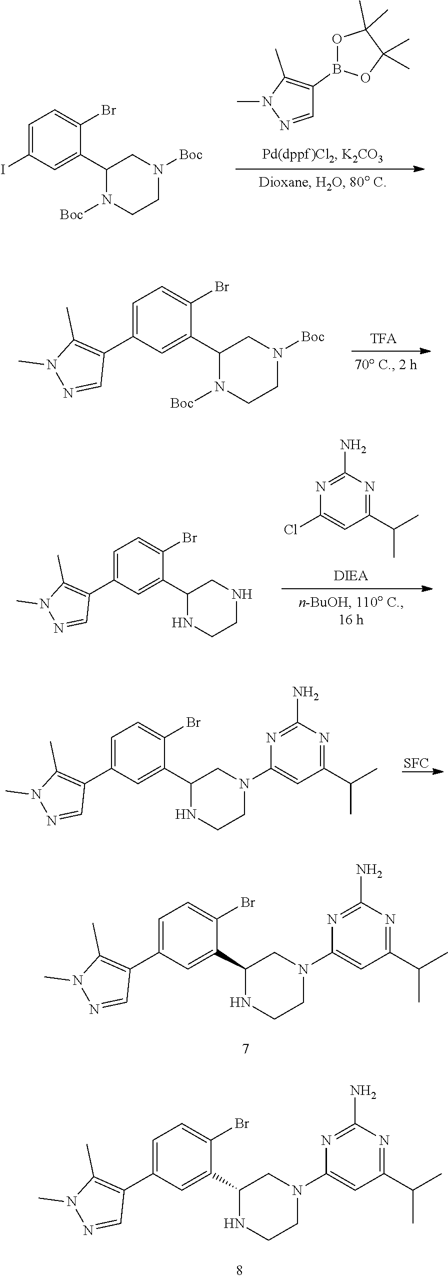

### Step 1: Preparation of di-tert-butyl 2-(2-bromo-5-(1,5-dimethyl-1H-pyrazol-4-yl)phenyl)piperazine-1,4-dicarboxylate

A mixture of di-tert-butyl 2-(2-bromo-5-iodophenyl)piperazine-1,4-dicarboxylate (500 mg, 881.44 umol), 1,5-dimethyl-4-(4,4,5,5-tetramethyl-1,3,2-dioxaborolan-2-yl)-1H-pyrazole (195.76 mg, 881.44 umol), K₂CO₃ (243.65 mg, 1.76 mmol) and Pd(dppf)Cl₂ (64.50 mg, 88.14 umol) in dioxane (5 mL) and H₂O (1 mL) was degassed and purged with N₂ for 3 times, and then the mixture was stirred at 60° C. for 3 h under N₂ atmosphere. LC-MS showed peak area of 70% with the desired mass. After cooling to room temperature, the reaction mixture was poured into water (100 mL), extracted with ethyl acetate (200 mL×2). The combined organic layer was washed with brine (100 mL×2), dried over anhydrous sodium sulfate, filtered and concentrated under reduced pressure to give a residue. The residue was purified by column chromatography (SiO₂, Petroleum ether/Ethyl acetate=8/1 to 4/1) to afford di-tert-butyl 2-(2-bromo-5-(1,5-dimethyl-1H-pyrazol-4-yl)phenyl)piperazine-1,4-dicarboxylate (230 mg) as a yellow solid. ¹H NMR (DMSO-d₆, 400 MHz) δ 7.64 (d, J=7.6 Hz, 1H), 7.46 (s, 1H), 7.23 (dd, J=2.0, 8.4 Hz, 2H), 5.09 (s, 1H), 3.99-3.80 (m, 2H), 3.77 (s, 3H), 3.74-3.58 (m, 2H), 3.47-3.33 (m, 1H), 3.29 (s, 1H), 2.31 (s, 3H), 1.31 (s, 18H).

### Step 2: Preparation of 2-(2-bromo-5-(1,5-dimethyl-1H-pyrazol-4-yl)phenyl)piperazine

A mixture of di-tert-butyl 2-(2-bromo-5-(1,5-dimethyl-1H-pyrazol-4-yl)phenyl)piperazine-1,4-dicarboxylate (230 mg, 429.53 umol, 1 eq) in TFA (2 mL) was stirred at 70° C. for 2 h. LC-MS showed peak area of 95% with the desired mass. After cooling to room temperature, the reaction mixture was concentrated under reduced pressure to afford 2-(2-bromo-5-(1,5-dimethyl-1H-pyrazol-4-yl)phenyl)piperazine (140 mg) as brown oil.

### Step 3: Preparation of 4-(3-(2-bromo-5-(1,5-dimethyl-1H-pyrazol-4-yl)phenyl)piperazin-1-yl)-6-isopropylpyrimidin-2-amine

To a mixture of 2-(2-bromo-5-(1,5-dimethyl-1H-pyrazol-4-yl)phenyl)piperazine (70 mg, 208.80 umol) and 4-chloro-6-isopropylpyrimidin-2-amine (35.84 mg, 208.80 umol) in n-BuOH (1 mL) was added DIEA (80.96 mg, 626.41 umol, 109.11 uL). The mixture was stirred at 110° C. for 16 h. LC-MS showed peak area of 82% with the desired mass. After cooling to room temperature, the reaction mixture was concentrated under reduced pressure to give a residue. The residue was purified by prep-HPLC (basic condition) to afford 4-(3-(2-bromo-5-(1,5-dimethyl-1H-pyrazol-4-yl)phenyl)piperazin-1-yl)-6-isopropylpyrimidin-2-amine (50 mg) as a red solid.

### Step 4: SFC Separation

4-(3-(2-bromo-5-(1,5-dimethyl-1H-pyrazol-4-yl)phenyl)piperazin-1-yl)-6-isopropylpyrimidin-2-amine (50 mg) was separated by SFC (column: DAICEL CHIRALPAK AD (250 mm*30 mm, 10 um); mobile phase: [0.1% NH3H2O ETOH]; B %: 40%-40%, A5.1; 30 min) to afford Peak 1 (Retention time 0.966) as compound 7 (10.6 mg) as a white solid. ¹H NMR (DMSO-d₆, 400 MHz) δ 7.70 (s, 1H), 7.62 (d, J=8.0 Hz, 1H), 7.57 (s, 1H), 7.25 (dd, J=2.4, 8.4 Hz, 1H), 5.93 (s, 1H), 5.89 (s, 2H), 4.43-4.18 (m, 2H), 3.98-3.93 (m, 1H), 3.78 (s, 3H), 3.10-2.96 (m, 2H), 2.85-2.75 (m, 2H), 2.59-2.58 (m, 1H), 2.36 (s, 3H), 1.12 (d, J=6.8 Hz, 6H), LC-MS: [M+H]+=470.2

Peak 2 (Retention time 1.466) to afford compound 6 (10.8 mg) as a white solid. ¹H NMR (DMSO-d₆, 400 MHz) δ 7.71 (d, J=2.4 Hz, 1H), 7.61 (d, J=8.0 Hz, 1H), 7.57 (s, 1H), 7.28-7.23 (m, 1H), 5.93 (s, 1H), 5.91-5.85 (m, 2H), 4.39-4.19 (m, 2H), 3.96 (dd, J=2.4, 10.0 Hz, 1H), 3.78 (s, 3H), 3.10-2.96 (m, 2H), 2.86-2.75 (m, 2H), 2.63-2.57 (m, 1H), 2.36 (s, 3H), 1.12 (d, J=7.0 Hz, 6H), LC-MS: [M+H]+=470.2.

### Example 23: Preparation of R or S-3-(4-(2-amino-6-isopropylpyrimidin-4-yl)piperazin-2-yl)-4-bromo-N-(2-(dimethylamino)ethyl)benzamide (Compound 20/21)

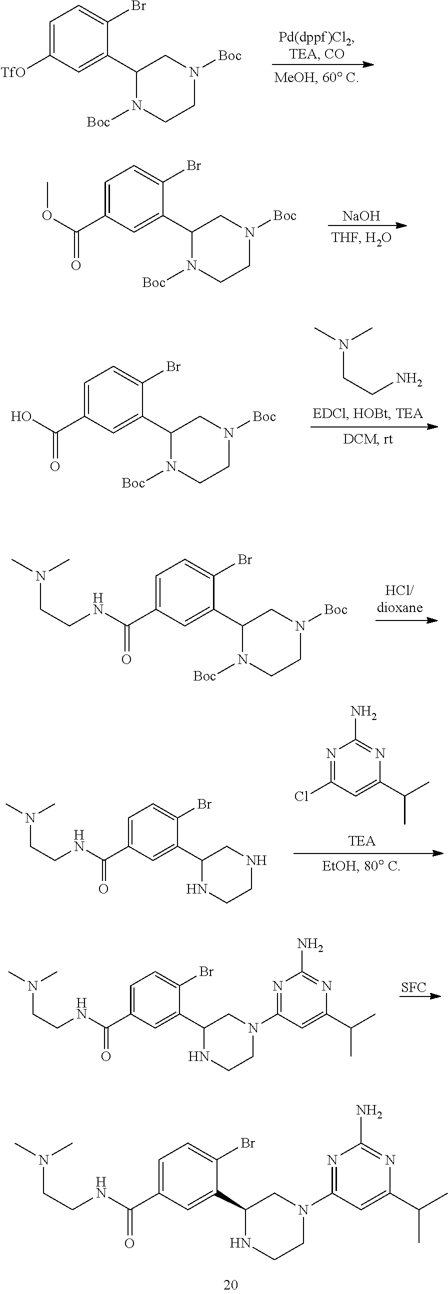

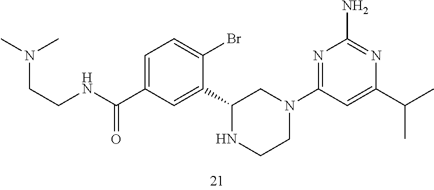 [SMILES: CC(C)c1cc(N2CCN[C@H](c3cc(C(=O)NCCN(C)C)ccc3Br)C2)nc(N)n1]

### Step 1: Preparation of di-tert-butyl 2-(2-bromo-5-(methoxycarbonyl)phenyl)piperazine-1,4-dicarboxylate

To a solution of di-tert-butyl 2-[2-bromo-5-(trifluoromethylsulfonyloxy)phenyl]piperazine-1,4-dicarboxylate (4 g, 6.79 mmol) in MeOH (40 mL) was added TEA (1.72 g, 16.97 mmol) and Pd(dppf)Cl₂ (496.56 mg, 678.63 umol). The mixture was stirred at 60° C. for 16 h under CO (50 Psi). LC-MS showed peak area of 75% with the desired mass. After cooling to room temperature, the mixture was concentrated under vacuum to give a residue. The residue was purified by column chromatography (SiO₂, Petroleum ether/Ethyl acetate=3/1) to afford crude di-tert-butyl 2-(2-bromo-5-(methoxycarbonyl)phenyl)piperazine-1,4-dicarboxylate (3.56 g) as a white solid.

### Step 2: Preparation of 3-(1,4-bis(tert-butoxycarbonyl)piperazin-2-yl)-4-bromobenzoic Acid

To a solution of di-tert-butyl 2-(2-bromo-5-methoxycarbonyl-phenyl)piperazine-1,4-dicarboxylate (3.56 g, 7.13 mmol) in THF (36 mL) and H₂O (7 mL) was added NaOH (570.25 mg, 14.26 mmol). The mixture was stirred at 70° C. for 16 h. LC-MS showed peak area of 83% with the desired mass. After cooling to room temperature, the mixture was adjusted to pH=7 with HCl (1 N). The mixture was concentrated under vacuum to give a residue. The residue was dried by azeotroping with toluene (30 mL×3) to afford crude 3-(1,4-bis(tert-butoxycarbonyl)piperazin-2-yl)-4-bromobenzoic acid (3.82 g) as a purple solid.

### Step 3: Preparation of Di-Tert-Butyl 2-(2-bromo-5-((2-(dimethylamino)ethyl)carbamoyl)phenyl)piperazine-1,4-dicarboxylate

To a solution of 3-[1,4-bis(tert-butoxycarbonyl)piperazin-2-yl]-4-bromo-benzoic acid (1.2 g, 2.47 mmol), N′,N′-dimethylethane-1,2-diamine (217.94 mg, 2.47 mmol) in DCM (12 mL) was added EDCI (710.93 mg, 3.71 mmol), HOBt (501.11 mg, 3.71 mmol) and TEA (625.44 mg, 6.18 mmol). The mixture was stirred at 20° C. for 16 h. LC-MS showed peak area of 45% with the desired mass. The mixture was concentrated under reduced pressure to give a residue. The residue was purified by column chromatography (SiO₂, Dichloromethane/Methanol=5/1) to afford di-tert-butyl 2-[2-bromo-5-[2-(dimethylamino)ethylcarbamoyl]phenyl]piperazine-1,4-dicarboxylate (900 mg) as a white solid.

### Step 4: Preparation of 4-bromo-N-(2-(dimethylamino)ethyl)-3-(piperazin-2-yl)benzamide

To a solution of di-tert-butyl 2-[2-bromo-5-[2-(dimethylamino)ethylcarbamoyl]phenyl]piperazine-1,4-dicarboxylate (344 mg, 619.26 umol) in DCM (4 mL) was added HCl/dioxane (4 N, 4 mL). The mixture was stirred at 20° C. for 2 h. LC-MS showed a peak with the desired mass. The mixture was concentrated under vacuum to give 4-bromo-N-[2-(dimethylamino)ethyl]-3-piperazin-2-yl-benzamide (325 mg) as a white solid.

### Step 5: Preparation of 3-(4-(2-amino-6-isopropylpyrimidin-4-yl)piperazin-2-yl)-4-bromo-N-(2-(dimethylamino)ethyl)benzamide

To a solution of 4-bromo-N-[2-(dimethylamino)ethyl]-3-piperazin-2-yl-benzamide (325 mg, 699.44 umol, 3HCl), 4-chloro-6-isopropyl-pyrimidin-2-amine (120.04 mg, 699.44 umol) in EtOH (3.25 mL) was added TEA (389.27 mg, 3.85 mmol). The mixture was stirred at 80° C. for 16 h. LC-MS showed peak area of 75% with the desired mass. After cooling to room temperature, the mixture was concentrated under reduced pressure to give a residue. The residue was purified by prep-HPLC (Base conditions) to afford 3-[4-(2-amino-6-isopropyl-pyrimidin-4-yl)piperazin-2-yl]-4-bromo-N-[2-(dimethylamino)ethyl]benzamide (94 mg) as a white solid.

### Step 6: SFC Separation

3-(4-(2-amino-6-isopropylpyrimidin-4-yl)piperazin-2-yl)-4-bromo-N-(2-(dimethylamino)ethyl)benzamide (94 mg) was separated by SFC (column: DAICEL CHIRALPAK IC (250 mm*30 mm, 10 um); mobile phase: [ACN/IPA (0.1% NH3H2O)]; B %: 58%-58%, A3.4; 23 min) to afford Peak 1 (Retention time 0.893) compound 21 (22.4 mg) as a white solid:¹H NMR (DMSO-d₆, 400 MHz) δ 8.57 (s, 1H), 8.19 (s, 1H), 7.75-7.70 (m, 2H), 5.96-5.95 (m, 3H), 4.35-4.33 (m, 2H), 4.00-3.98 (m, 1H), 3.41-3.38 (m, 2H), 3.08 (d, J=11.2 Hz, 1H), 2.87-2.80 (m, 1H), 2.78-2.75 (m, 1H), 2.63-2.58 (m, 4H), 2.29 (s, 6H), 1.13 (d, J=7.2 Hz, 6H), LC-MS: [M+H]+=490.2

Peak 2 (Retention time 1.436) to afford compound 20 (31.2 mg) as a white solid, ¹H NMR: (DMSO-d₆, 400 MHz) δ 8.62 (s, 1H), 8.19 (s, 1H), 7.76-7.69 (m, 2H), 6.02-5.97 (m, 3H), 4.36-4.34 (m, 2H), 4.01-3.98 (m, 1H), 3.44-3.41 (m, 2H), 3.08 (d, J=10.8 Hz, 1H), 2.88-2.78 (m, 1H), 2.78-2.75 (m, 1H), 2.68-2.63 (m, 4H), 2.38 (s, 6H), 1.13 (d, J=6.8 Hz, 6H), LC-MS: [M+H]+=490.3.

### Example 24: Preparation of R or S 6-(3-(2-bromo-5-(4-methylpiperazin-1-yl)phenyl)piperazin-1-yl)pyrimidine-2,4-diamine (Compound 50/51)

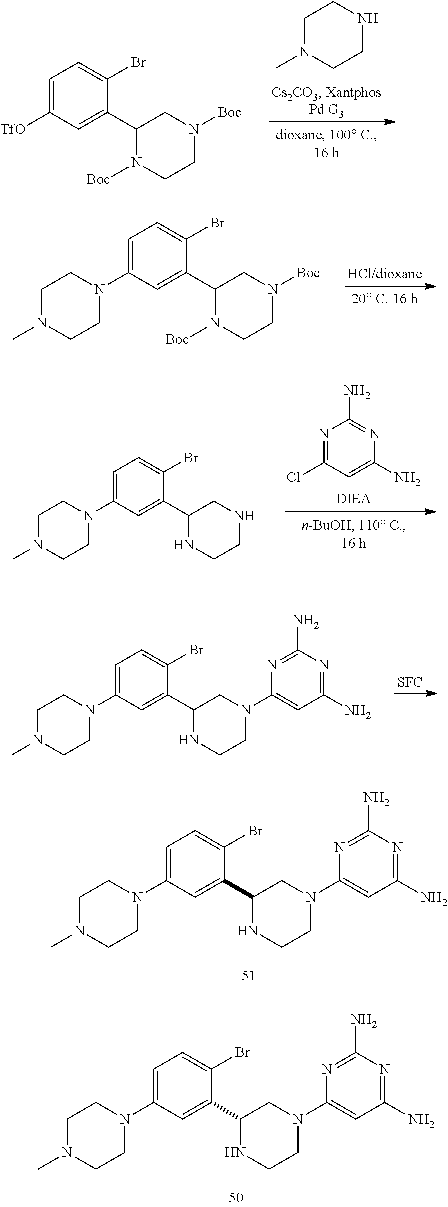

### Step 1: Preparation of Di-Tert-Butyl 2-(2-bromo-5-(4-methylpiperazin-1-yl)phenyl)piperazine-1,4-dicarboxylate

To a mixture of di-tert-butyl 2-(2-bromo-5-(((trifluoromethyl)sulfonyl)oxy)phenyl)piperazine-1,4-dicarboxylate (600 mg, 1.02 mmol) and 1-methylpiperazine (101.96 mg, 1.02 mmol) in dioxane (6 mL) was added XantPhos Pd G3 (96.54 mg, 101.80 umol) and Cs₂CO₃ (663.34 mg, 2.04 mmol). The mixture was degassed and purged with N₂ for 3 times and was stirred at 100° C. for 16 h under N₂ atmosphere. LC-MS showed peak area of 26% with the desired mass. After cooling to room temperature, the reaction mixture was poured into water (50 mL), extracted with ethyl acetate (30 mL×3). The combined organic layers were washed with brine (20 mL×2), dried over Na₂SO₄, filtered and concentrated under reduced pressure. The residue was purified by column chromatography (SiO₂, Dichloromethane/Methanol=10/1) to afford di-tert-butyl 2-(2-bromo-5-(4-methylpiperazin-1-yl)phenyl)piperazine-1,4-dicarboxylate (210 mg) as yellow oil.

### Step 2: Preparation of 1-(4-bromo-3-(piperazin-2-yl)phenyl)-4-methylpiperazine

Di-tert-butyl 2-(2-bromo-5-(4-methylpiperazin-1-yl)phenyl)piperazine-1,4-dicarboxylate (210 mg, 389.25 umol) was added to a solution of HCl in dioxane (1 M, 2 mL). The mixture was stirred at 20° C. for 16 h. LC-MS showed peak area of 36% with the desired mass. The reaction mixture was concentrated under reduced pressure to afford 1-(4-bromo-3-(piperazin-2-yl)phenyl)-4-methylpiperazine (210 mg, crude) as a yellow solid.

### Step 3: Preparation of 6-(3-(2-bromo-5-(4-methylpiperazin-1-yl)phenyl)piperazin-1-yl)pyrimidine-2,4-diamine

To a solution of 1-(4-bromo-3-(piperazin-2-yl)phenyl)-4-methylpiperazine (260 mg, 766.34 umol) and 6-chloropyrimidine-2,4-diamine (110.78 mg, 766.34 umol) in n-BuOH (3 mL) was added DIEA (396.18 mg, 3.07 mmol, 533.93 uL). The mixture was stirred at 110° C. for 16 h. LC-MS showed peak area of 27% with the desired mass. The reaction mixture was concentrated under reduced pressure to give a residue. The residue was purified by prep-HPLC (basic condition) to afford 6-(3-(2-bromo-5-(4-methylpiperazin-1-yl)phenyl)piperazin-1-yl)pyrimidine-2,4-diamine (60 mg) as a white solid.

### Step 4: SFC Separation

6-(3-(2-bromo-5-(4-methylpiperazin-1-yl)phenyl)piperazin-1-yl)pyrimidine-2,4-diamine (60 mg) was separated by SFC (column: DAICEL CHIRALCEL OD (250 mm*50 mm, 10 um); mobile phase: [0.1% NH3H2O ETOH]; B %: 50%-50%, A4; 20 min) to afford Peak 1 (Retention time 0.625) compound 51 (6.7 mg) as a white solid: ¹H NMR (DMSO-d₆, 400 MHz) δ 7.37 (d, J=8.8 Hz, 1H), 7.26-7.22 (m, 1H), 6.81 (dd, J=3.2, 9.2 Hz, 1H), 5.69 (s, 2H), 5.47-5.40 (m, 2H), 5.07 (s, 1H), 4.14-3.99 (m, 2H), 3.89-3.79 (m, 1H), 3.16-3.08 (m, 4H), 3.05-2.98 (m, 1H), 2.87-2.79 (m, 1H), 2.74-2.65 (m, 2H), 2.46-2.42 (m, 4H), 2.35-2.32 (m, 1H), 2.21 (s, 3H), LC-MS: [M+H]+=447.0

Peak 2 (Retention time 1.101) to afford compound 50 (6.3 mg) as a white solid, ¹H NMR (DMSO-d₆, 400 MHz) δ 7.37 (d, J=8.8 Hz, 1H), 7.24 (d, J=3.2 Hz, 1H), 6.81 (dd, J=3.2, 8.8 Hz, 1H), 5.69 (s, 2H), 5.44 (s, 2H), 5.07 (s, 1H), 4.16-4.01 (m, 2H), 3.90-3.83 (m, 1H), 3.15-3.09 (m, 4H), 3.05-2.98 (m, 1H), 2.88-2.80 (m, 1H), 2.74-2.66 (m, 2H), 2.46-2.41 (m, 4H), 2.38-2.32 (m, 1H), 2.21 (s, 3H), LC-MS: [M+H]+=447.1

### Assays

### GID4 Competitive Binding Assay

GID4 binding affinity of compounds disclosed herein was measured by monitoring the interaction of GID4 with its degron peptide (PGLW) in the presence of the test compounds. A truncated version of GID4 (Uniprot ID: Q81VV7, residues 124-289) with a 6*His at its C-terminus was used in this assay. Tetrapeptide, PGLW, was synthesized and tagged with an additional lysine labeled with biotin at its sidechain.

GID4 competitive binding assays were performed in a 384-well plate at 4° C. in a 20 μL reaction volume. Briefly, 24 nM GID4 (final concentration) proteins were pre-incubated with 48 nM biotinylated degron peptide (final concentration) in an assay buffer containing 20 mM HEPES, pH 7.5, 100 mM NaCl, 1 mM DTT, 0.1% Tween-20, and 0.05% BSA in the presence of the test compound in 2% DMSO (final concentration) at various concentrations for 2 h. 5 ug/ml Streptavidin ALphaScreen donor beads and 5 ug/ml nickel chelate (Ni-NTA) ALphaScreen acceptor beads (PerkinElmer, 6760619C) (final concentrations) were then added to the mixture. After an incubation of 1 h at 4° C., the fluorescent signals were obtained on the EnVision® 2105 Multilabel Plate Reader (PerkinElmer).

Raw ALphaScreen data were converted to a percentage of inhibition (relative to DMSO) using the following equations:

For a given test compound concentration X:

Signal (X)=Signal (GID4 & peptide & compound)−Signal (GID4 & buffer & compound)

Percentage of inhibition at concentration X=[1−Signal(X)/Signal (DMSO)]*100% b)

The IC50 values were determined by nonlinear regression of plots of [inhibitor] vs. percentage of inhibition with variable slope.

Table 3 summarizes the inhibition of binding between GID4 and degron peptide with compounds of the present disclosure. Compounds were ranked into bins as follows for IC₅₀: A indicates <500 nM; B indicates 500 nM-5000 nM; C indicates >5000 nM.

TABLE 18
Inhibition of GID4/Degron binding by test compounds

| Compound | IC |
|---|---|
| No. | 50 |
| 1 | B |
| 2 | B |
| 3 | A |
| 4 | B |
| 5 | A |
| 6 | A |
| 7 | B |
| 8 | C |
| 9 | A |
| 10 | A |
| 11 | B |
| 12 | A |
| 13 | A |
| 14 | B |
| 15 | A |
| 16 | B |
| 17 | A |
| 18 | B |
| 19 | A |
| 20 | B |
| 21 | A |
| 22 | A |
| 23 | B |
| 24 | A |
| 25 | A |
| 26 | B |
| 27 | C |
| 28 | A |
| 29 | A |
| 30 | A |
| 31 | B |
| 32 | A |
| 33 | A |
| 34 | C |
| 35 | B |
| 36 | C |
| 37 | A |
| 38 | A |
| 39 | C |
| 40 | B |
| 41 | A |
| 42 | C |
| 43 | B |
| 44 | B |
| 45 | A |
| 46 | B |
| 47 | B |
| 48 | C |
| 49 | C |
| 50 | A |
| 51 | B |
| 52 | B |
| 53 | C |
| 54 | A |
| 55 | B |
| 56 | B |
| 57 | B |
| 58 | B |
| 59 | C |
| 60 | C |
| 61 | B |
| 62 | A |
| 63 | B |
| 64 | C |
| 65 | B |
| 66 | C |
| 67 | B |
| 68 | C |
| 69 | C |
| 70 | B |
| 71 | A |
| 72 | A |
| 73 | B |
| 74 | B |
| 75 | B |
| 76 | A |
| 77 | C |
| 78 | A |
| 79 | C |
| 80 | B |
| 81 | B |
| 82 | B |
| 83 | B |
| 84 | B |
| 85 | B |
| 86 | B |
| 87 | A |
| 88 | C |
| 89 | C |
| 90 | C |
| 91 | A |
| 92 | B |
| 93 | C |
| 94 | C |
| 95 | C |
| 96 | C |
| 97 | C |
| 98 | A |
| 99 | A |
| 100 | C |
| 101 | A |
| 102 | B |
| 103 | B |
| 104 | C |
| 105 | C |
| 106 | A |
| 107 | B |
| 108 | C |
| 109 | B |
| 110 | B |
| 111 | B |
| 112 | A |
| 113 | B |
| 114 | A |
| 115 | C |
| 116 | B |
| 117 | B |
| 118 | B |
| 119 | C |
| 120 | B |
| 121 | C |
| 122 | A |
| 123 | B |
| 124 | B |
| 125 | B |
| 126 | A |
| 127 | C |
| 128 | C |
| 129 | B |
| 130 | C |
| 131 | A |
| 132 | B |
| 133 | B |
| 134 | A |
| 135 | B |
| 136 | C |
| 137 | C |
| 138 | B |
| 139 | A |
| 140 | B |
| 141 | A |
| 142 | A |
| 143 | A |
| 144 | C |
| 145 | B |
| 146 | A |
| 147 | B |
| 148 | C |
| 149 | C |
| 150 | C |
| 151 | C |
| 152 | B |
| 153 | B |
| 154 | B |
| 155 | A |
| 156 | C |
| 157 | C |
| 158 | C |
| 159 | B |
| 160 | B |
| 161 | B |
| 162 | B |
| 163 | A |
| 164 | A |
| 165 | C |
| 166 | C |
| 167 | A |
| 168 | B |
| 169 | A |
| 170 | B |
| 171 | B |
| 172 | A |
| 173 | A |
| 174 | B |
| 175 | B |
| 176 | A |
| 177 | C |
| 178 | A |
| 179 | B |
| 180 | C |
| 181 | C |
| 182 | C |
| 183 | A |
| 184 | B |
| 185 | B |
| 186 | C |
| 187 | B |
| 188 | C |
| 189 | C |
| 190 | C |
| 191 | C |
| 192 | B |
| 193 | C |
| 194 | B |
| 195 | C |
| 196 | C |
| 197 | C |
| 198 | C |
| 199 | C |
| 200 | B |
| 201 | C |
| 202 | C |
| 203 | C |
| 204 | C |
| 205 | C |
| 206 | C |
| 207 | C |
| 208 | C |
| 209 | C |
| 210 | C |
| 211 | C |
| 212 | C |
| 213 | C |
| 214 | C |
| 215 | C |
| 216 | C |
| 217 | C |
| 218 | C |
| 219 | C |
| 220 | B |
| 221 | A |
| 222 | B |
| 223 | B |
| 224 | C |
| 225 | B |
| 226 | C |
| 227 | B |
| 228 | B |
| 229 | C |
| 230 | C |
| 231 | C |
| 232 | B |
| 233 | B |
| 234 | B |
| 235 | A |
| 236 | B |
| 237 | B |
| 238 | B |
| 239 | B |
| 240 | A |
| 241 | C |
| 242 | B |
| 243 | C |
| 244 | B |
| 245 | A |
| 246 | B |
| 247 | B |
| 248 | B |
| 249 | B |
| 250 | B |
| 251 | B |
| 252 | B |
| 253 | B |
| 254 | C |
| 255 | B |
| 256 | A |
| 257 | C |
| 258 | B |
| 259 | C |
| 260 | C |
| 261 | A |
| 262 | A |
| 263 | A |
| 264 | A |
| 265 | B |
| 266 | C |
| 267 | A |
| 268 | A |
| 269 | A |
| 270 | A |
| 271 | A |
| 272 | A |
| 273 | B |
| 274 | A |
| 275 | A |
| 276 | A |
| 277 | A |
| 278 | B |
| 279 | A |
| 280 | A |
| 281 | A |
| 282 | A |
| 283 | A |
| 284 | C |
| 285 | A |
| 286 | A |
| 287 | A |
| 288 | A |
| 289 | A |
| 290 | A |
| 291 | A |
| 292 | A |
| 293 | B |
| 294 | A |
| 295 | A |
| 296 | A |
| 297 | A |
| 298 | A |
| 299 | A |
| 300 | A |
| 301 | B |
| 302 | B |
| 303 | A |
| 304 | B |
| 305 | B |
| 306 | B |
| 307 | A |
| 308 | A |
| 309 | B |
| 310 | B |
| 311 | B |
| 312 | A |
| 313 | B |
| 314 | B |
| 315 | A |
| 316 | A |
| 317 | B |
| 318 | A |
| 319 | B |
| 320 | B |
| 321 | A |
| 322 | A |
| 323 | B |
| 324 | B |
| 325 | B |
| 326 | A |
| 327 | B |
| 328 | A |
| 329 | B |
| 330 | B |
| 331 | A |
| 332 | A |
| 333 | A |
| 334 | A |
| 335 | A |
| 336 | A |
| 337 | A |
| 338 | A |
| 339 | A |
| 340 | C |
| 341 | A |
| 342 | B |
| 343 | C |
| 344 | C |
| 345 | B |
| 346 | B |
| 347 | C |
| 348 | C |
| 349 | C |
| 350 | C |
| 351 | C |
| 352 | B |
| 353 | B |
| 354 | C |
| 355 | B |
| 356 | A |
| 357 | B |
| 358 | B |
| 359 | B |
| 360 | C |
| 361 | A |
| 362 | B |
| 363 | C |
| 364 | C |
| 365 | B |
| 366 | B |
| 367 | B |
| 368 | B |
| 369 | A |
| 370 | A |
| 371 | A |
| 372 | A |
| 373 | A |
| 374 | A |
| 375 | A |
| 376 | A |

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described and accordingly, all suitable modifications and equivalents may be resorted to, falling within the scope of the present disclosure.

Moreover, those skilled in the art will appreciate that the conception upon which this disclosure is based may readily be used as a basis for designing other structures, methods, and systems for carrying out the several purposes of the present disclosure. Accordingly, the claims are not to be considered as limited by the foregoing description or examples.

What is claimed is:

1. A compound of Formula (1) or a tautomer, stereoisomer or a mixture of stereoisomers, or a pharmaceutically acceptable salt, or hydrate, or deuterated derivative thereof: 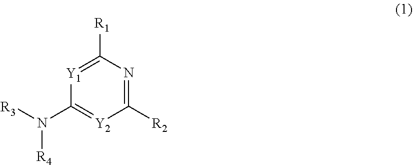
wherein:
Y₁ is either:
chosen from CH, N, and CX₁, wherein X₁ is a halogen, or
Y₁ and R₁ together form an optionally substituted 3 to 7 membered saturated or unsaturated ring G, wherein the ring has 0 to 3 heteroatoms;
Y₂ is chosen from CH, N, and CX₁, wherein X₁ is a halogen;
R₁ is either:
chosen from NHR₅, H, and C₁-C₄ alkyl, wherein R₅ is chosen from H and optionally substituted C₁-C₃ alkyl, or
Y₁ and R₁ together form a 3 to 7 membered saturated or unsaturated ring G, wherein the ring has 0 to 3 heteroatoms;
R₂ is chosen from NHR₆, H, and C₁-C₄ alkyl, wherein R₆ is chosen from H and optionally substituted C₁-C₃ alkyl; and
R₃ and R₄ are each independently chosen from hydrogen, an optionally substituted 5 to 7 membered ring, and optionally substituted C₁-C₃ alkyl; or
R₃ and R₄ together form a 5 to 9 membered ring system D, wherein the ring is chosen from monocyclic rings and bicyclic rings, and wherein the monocyclic rings and bicyclic rings are optionally substituted with a halogen, a hydroxyl, an optionally substituted phenyl, an acetyl group, a 5 membered heteroaryl, an optionally substituted 9 membered heterobicyclic aryl, an optionally substituted benzimidazolone, and/or an optionally substituted C₁-C₃ alkyl.

2. The compound according to claim 1, wherein the compound is a compound of Formula (1a): 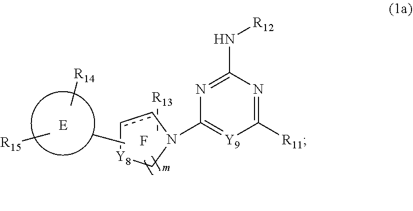
wherein:
m is 1, 2, or 3;
the dashed bond in ring F is either a single bond or a double bond;
R₁₁ is chosen from a C₁-C₃ alkyl and NHR₂₁, wherein R₂₁ is chosen from H and a C₁-C₃ alkyl;
R₁₂ is chosen from H and a C₁-C₄ alkyl optionally substituted with a hydroxyl;
R₁₃ is either:
absent,
chosen from a halogen, a hydroxyl, and a C₁-C₃ alkyl optionally substituted with a hydroxyl, or
R₁₃ and Y₈ together form a 5 membered heteroaryl ring;
Y₈ is either:
chosen from O, CH₂, an acetyl group, and NR₁₄, wherein R₁₄ is chosen from H and a C₁-C₃ alkyl optionally substituted with a hydroxyl, or
R₁₃ and Y₈ together form a 5 membered heteroaryl ring;
ring system E is chosen from 6 membered aryls and 9 membered bicyclic heteroaryls;
Y₉ is chosen from N and CX₂, wherein X₂ is chosen from H and a halogen;
R₁₄ is absent or a halogen; and
R₁₅ is absent or chosen from a halogen, a hydroxyl, a 5 membered heteroaryl having 1 to 3 heteroatoms and optionally substituted with up to two methyl groups, 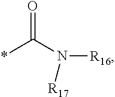
a trifluoromethyl group, a C₁-C₃ alkoxy group, 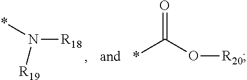
wherein:
R₁₆ is either:
chosen from H and a C₁-C₆ alkyl optionally substituted with a tertiary amine, or
R₁₆ and R₁₇ together form a 5 to 6 membered non-aromatic ring optionally substituted with up to two C₁-C₃ alkyls each independently optionally substituted with a hydroxyl;
R₁₇ is either:
chosen from H and a C₁-C₃ alkyl, or
R₁₆ and R₁₇ together form a 5 to 6 membered non-aromatic ring optionally substituted with up to two C₁-C₃ alkyls each independently optionally substituted with a hydroxyl;
R₁₈ is either:
chosen from H and a C₁-C₆ alkyl optionally substituted with a tertiary amine or a hydroxyl, or
R₁₈ and R₁₉ together form a 4 to 6 membered non-aromatic ring optionally substituted with a C₁-C₃ alkyl optionally substituted with a tertiary amine or a hydroxyl;
R₁₉ is either:
chosen from H and a C₁-C₃ alkyl, or
R₁₈ and R₁₉ together form a 4 to 6 membered non-aromatic ring optionally substituted with a C₁-C₃ alkyl optionally substituted with a tertiary amine or a hydroxyl; and
R₂₀ is chosen from C₁-C₃ alkyls;
and wherein Formula (1a) encompasses all tautomers, stereoisomers, deuterated derivatives, pharmaceutically acceptable salts, and mixtures thereof.

3. The compound according to claim 2, wherein ring system E is chosen from: 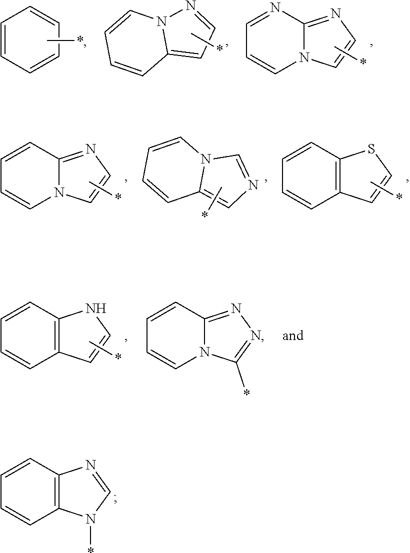
wherein each asterisk (*) denotes a point of attachment within Formula (1a).

4. The compound according to claim 3, wherein the compound is chosen from Formula (1aa) and Formula (1ab): 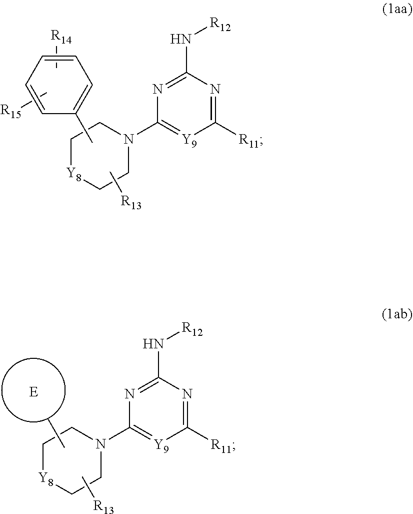
wherein R₁₄ and R₁₅ are not both absent, and wherein each of Formula (1aa) and Formula (1ab) encompass all tautomers, stereoisomers, pharmaceutically acceptable salts, and mixtures thereof.

5. The compound according to claim 1, wherein the compound is a compound of Formula (1 b): 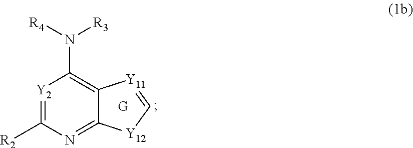
wherein:
each of Y₁₁ and Y₁₂ are chosen from N and CH;
at least one of Y₁₁ and Y₁₂ is N;
and wherein Formula (1 b) encompasses all tautomers, stereoisomers, deuterated derivatives, pharmaceutically acceptable salts, and mixtures thereof.

6. The compound according to claim 5, wherein the compound is a compound of Formula (1ba): 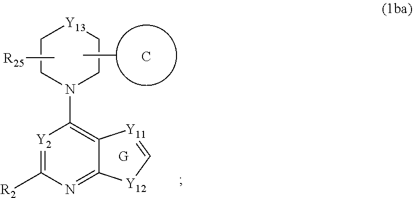
wherein:
ring C is chosen from 9 membered bicyclic heteroaryls and 6 membered aryls optionally substituted with a halogen and/or a hydroxyl;
Y₁₃ is chosen from CH₂, O, and NR₂₄ wherein R₂₄ is chosen from H and a C₁-C₃ alkyl optionally substituted with hydroxyl;
R₂₅ is absent or chosen from a halogen, a hydroxyl, and a C₁-C₃ alkyl optionally substituted with a tertiary amine;
and wherein Formula (1ba) encompasses all tautomers, stereoisomers, deuterated derivatives, pharmaceutically acceptable salts, and mixtures thereof.

7. The compound according to claim 1, wherein the compound is a compound of Formula (1c): 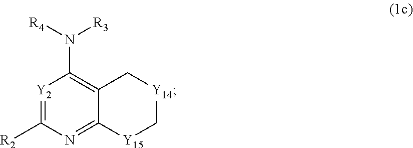
wherein:
Y₁₅ is chosen from NH and CH₂;
Y₁₄ is chosen from O, S, and CH₂;
at least one of Y₁₅ and Y₁₄ is not CH₂;
and wherein Formula (1c) encompasses all tautomers, stereoisomers, deuterated derivatives, pharmaceutically acceptable salts, and mixtures thereof.

8. The compound according to claim 7, wherein the compound is a compound of Formula (1ca): 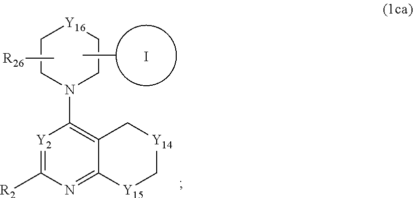
wherein:
ring I is chosen from a 9 membered bicyclic heteroaryl and a 5 or 6 membered aryl or heteroaryl ring optionally substituted with a halogen and/or a hydroxyl;
Y₁₆ is chosen from CH₂, O, and NR₂₇ wherein R₂₇ is chosen from H and a C₁-C₃ alkyl optionally substituted with hydroxyl;
R₂₆ is absent or chosen from a halogen, a hydroxyl, and a C₁-C₃ alkyl optionally substituted with a tertiary amine;
and wherein Formula (1 ca) encompasses all tautomers, stereoisomers, deuterated derivatives, pharmaceutically acceptable salts, and mixtures thereof.

9. The compound according to any one of claims 1, 5, and 7, wherein the compound is chosen from Formula (1), Formula (1b), and Formula (1c); and R₃ is a hydrogen or an optionally substituted C₁-C₃ alkyl.

10. The compound according to claim 9, wherein R₄ is chosen from:
C₁-C₃ alkyl optionally substituted with a 9 membered bicyclic heteroaryl, 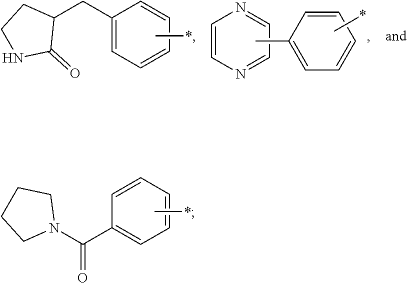
wherein the asterisks (*) denotes a point of attachment in Formula (1).

11. The compound according to claim 1, wherein R₃ and R₄ together form a 5 to 9 membered ring system D.

12. The compound according to claim 11, wherein the 5 to 9 membered ring system D is chosen from monocyclic rings optionally substituted with a halogen, a hydroxyl, an optionally substituted phenyl, an acetyl group, a 5 membered heteroaryl, an optionally substituted 9 membered heterobicyclic aryl, an optionally substituted benzimidazolone, and/or an optionally substituted C₁-C₃ alkyl.

13. The compound according to claim 1, wherein R₃ and R₄ together form a 5 to 9 membered monocyclic ring substituted with a substituted phenyl.

14. The compound according to claim 13, wherein the 5 to 9 membered monocyclic ring is a 6-membered heterocyclyl.

15. The compound according to claim 13, wherein the substituted phenyl is substituted with 1 or 2 groups chosen from halogen, haloalkyl, 4 to 10-membered heteroaryl, 4 to 10-membered heterocyclyl, —C(O)Rₐ, —C(O)NR_{b}R_{c}, —C(O)OR_{b}, C₁-C₄ alkyl, C₃-C₆ cycloalkyl, C₃-C₆ cycloalkoxy, and C₁-C₄ alkoxy, wherein Rₐ, R_{b}, and R_{c} are independently chosen from hydrogen, C₁-C₄ alkyl, C₃-C₆ cycloalkyl, C₁-C₄ alkylamino, 4 to 10-membered heteroaryl, and 4 to 10-membered heterocyclyl,
or R_{b} and R_{c} together form a 4 to 6-membered heterocyclyl optionally substituted with 1 or 2 groups chosen from cyano, halogen, C₁-C₄ alkyl, C₁-C₄ alkoxy, and C₁-C₄ hydroxyalkyl, and
wherein the 4 to 10-membered heteroaryl and 4 to 10-membered heterocyclyl are each optionally substituted with 1, 2, or 3 groups chosen from cyano, halogen, C₁-C₄ alkyl, C₁-C₄ alkoxy, C₁-C₄ haloalkyl, C₁-C₄ hydroxyalkyl, and C₃-C₆ cycloalkyl.

16. The compound according to claim 13, wherein the substituted phenyl is substituted with 1 or 2 substituents chose from halogen, C₁-C₄ haloalkyl, 5 to 10-membered heteroaryl, 4 to 6-membered heterocyclyl, —C(O)NR₉R₁ₒ, and C₁-C₂ alkoxy,
wherein the 5 to 10-membered heteroaryl is optionally substituted with 1 or 2 methyl; and
wherein R_{b} and R_{c} are independently chosen from hydrogen and C₄ alkylamino; or
R_{b} and R_{c} together form a 4 to 6-membered heterocyclyl which is optionally substituted with 1 or 2 groups chosen from cyano, halogen, methyl, —OMe, and —CH₂OH.

17. The compound according to claim 13, wherein the substituted phenyl is optionally substituted with 1 or 2 substituents chosen from bromo, —CF₃, —OEt,

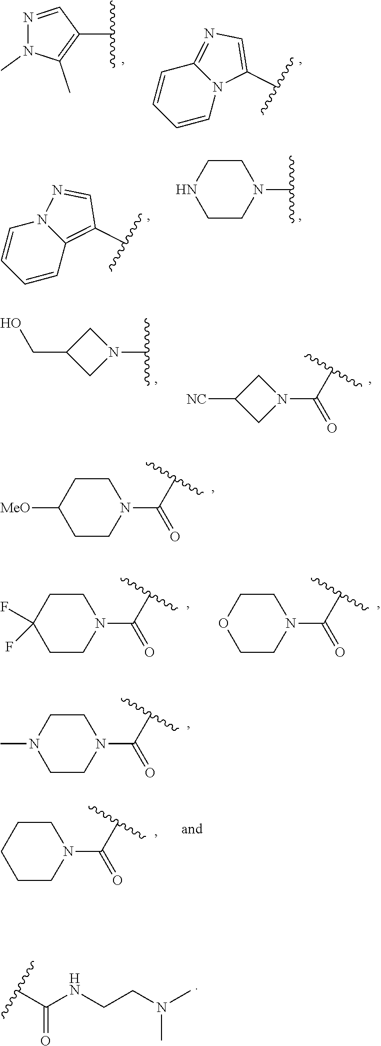

18. The compound according to claim 13, wherein the substituted phenyl is substituted with a substituent chosen from halogen and C₁-C₄ haloalkyl and a substituent chosen from —CF₃, —OEt,

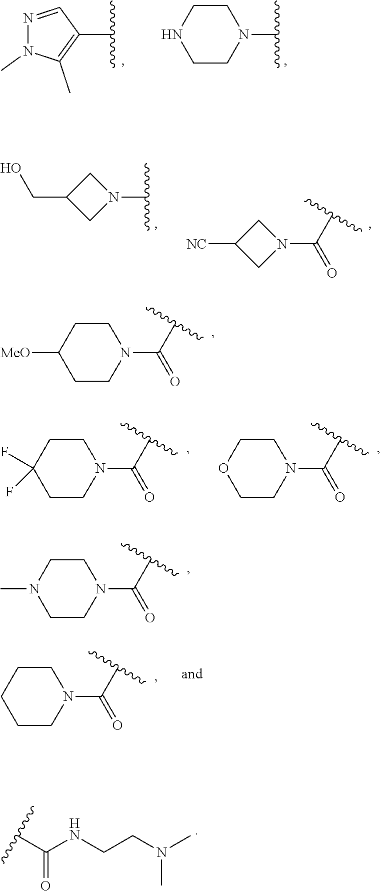

19. The compound according to claim 1, wherein the compound is chosen from:
4-(7-(2-bromophenyl)-6,7-dihydropyrazolo[1,5-a]pyrazin-5(4H)-yl)-6-isopropylpyrimidin-2-amine;
(R/S)-6-(3-(2-bromo-5-(1,5-dimethyl-1H-pyrazol-4-yl)phenyl)piperazin-1-yl)pyrimidine-2,4-diamine;
(S/R)-6-(3-(2-bromo-5-(1,5-dimethyl-1H-pyrazol-4-yl)phenyl)piperazin-1-yl)pyrimidine-2,4-diamine;
(R/S)-4-(3-(2-bromo-5-(1,5-dimethyl-1H-pyrazol-4-yl)phenyl)piperazin-1-yl)-6-isopropylpyrimidin-2-amine;
(S/R)-4-(3-(2-bromo-5-(1,5-dimethyl-1H-pyrazol-4-yl)phenyl)piperazin-1-yl)-6-isopropylpyrimidin-2-amine;
4-bromo-3-(4-(2,6-diaminopyrimidin-4-yl)piperazin-2-yl)-N-(2-(dimethylamino)ethyl)benzamide;
(R/S)-6-(3-(2-bromo-5-(trifluoromethyl)phenyl)piperazin-1-yl)pyrimidine-2,4-diamine;
(S/R)-6-(3-(2-bromo-5-(trifluoromethyl)phenyl)piperazin-1-yl)pyrimidine-2,4-diamine;
(R/S)-4-(3-(2-bromo-5-(trifluoromethyl)phenyl)piperazin-1-yl)-6-isopropylpyrimidin-2-amine;
(S/R)-4-(3-(2-bromo-5-(trifluoromethyl)phenyl)piperazin-1-yl)-6-isopropylpyrimidin-2-amine;
(R/S)-(4-bromo-3-(4-(2,6-diaminopyrimidin-4-yl)piperazin-2-yl)phenyl)(4-methylpiperazin-1-yl)methanone;
(S/R)-(4-bromo-3-(4-(2,6-diaminopyrimidin-4-yl)piperazin-2-yl)phenyl)(4-methylpiperazin-1-yl)methanone;
(R/S)-(3-(4-(2-amino-6-isopropylpyrimidin-4-yl)piperazin-2-yl)-4-bromophenyl)(4-methylpiperazin-1-yl)methanone;
(S/R)-(3-(4-(2-amino-6-isopropylpyrimidin-4-yl)piperazin-2-yl)-4-bromophenyl)(4-methylpiperazin-1-yl)methanone;
7-(3-(imidazo[1,2-a]pyridin-3-yl)piperidin-1-yl)-5-isopropyl-3H-imidazo[4,5-b]pyridine;
(R/S)-3-(4-(2-amino-6-isopropylpyrimidin-4-yl)piperazin-2-yl)-4-bromo-N-(2-(dimethylamino)ethyl)benzamide;
(S/R)-3-(4-(2-amino-6-isopropylpyrimidin-4-yl)piperazin-2-yl)-4-bromo-N-(2-(dimethylamino)ethyl)benzamide;
4-isopropyl-6-(3-(pyrazolo[1,5-a]pyridin-3-yl)piperazin-1-yl)pyrimidin-2-amine;
(R/S)-6-(3-(2-bromo-5-ethoxyphenyl)piperazin-1-yl)pyrimidine-2,4-diamine;
(S/R)-6-(3-(2-bromo-5-ethoxyphenyl)piperazin-1-yl)pyrimidine-2,4-diamine;
(R/S)-4-(3-(2-bromo-5-ethoxyphenyl)piperazin-1-yl)-6-isopropylpyrimidin-2-amine;
(S/R)-4-(3-(2-bromo-5-ethoxyphenyl)piperazin-1-yl)-6-isopropylpyrimidin-2-amine;
(R/S)-4-ethyl-6-(3-(imidazo[1,2-a]pyridin-3-yl)piperazin-1-yl)pyrimidin-2-amine;
(S/R)-4-ethyl-6-(3-(imidazo[1,2-a]pyridin-3-yl)piperazin-1-yl)pyrimidin-2-amine;
6-(2-(2-(trifluoromethyl)phenyl)morpholino)pyrimidine-2,4-diamine;
(R/S)-(1-(3-(4-(2-amino-6-isopropylpyrimidin-4-yl)piperazin-2-yl)-4-bromophenyl)azetidin-3-yl)methanol;
(S/R)-(1-(3-(4-(2-amino-6-isopropylpyrimidin-4-yl)piperazin-2-yl)-4-bromophenyl)azetidin-3-yl)methanol;
4-(3-(imidazo[1,2-a]pyridin-3-yl)piperazin-1-yl)-6-isopropylpyrimidin-2-amine;
(1-(4-bromo-3-(4-(2,6-diaminopyrimidin-4-yl)piperazin-2-yl)phenyl)piperidin-4-yl)methanol;
(S/R)-7-(3-(2-bromophenyl)piperazin-1-yl)-3H-imidazo[4,5-b]pyridine;
(R/S)-7-(3-(2-bromophenyl)piperazin-1-yl)-3H-imidazo[4,5-b]pyridine;
(R/S)-(2-(2-bromophenyl)-4-(2,6-diaminopyrimidin-4-yl)morpholin-2-yl)methanol;
(S/R)-(2-(2-bromophenyl)-4-(2,6-diaminopyrimidin-4-yl)morpholin-2-yl)methanol;
methyl 4-bromo-3-(4-(2,6-diaminopyrimidin-4-yl)piperazin-2-yl)benzoate;
(R/S)-4-(2-(2-bromophenyl)morpholino)-7H-pyrrolo[2,3-d]pyrimidin-2-amine;
(S/R)-4-(2-(2-bromophenyl)morpholino)-7H-pyrrolo[2,3-d]pyrimidin-2-amine;
4-(3-(2-bromo-5-(piperazin-1-yl)phenyl)piperazin-1-yl)-6-isopropylpyrimidin-2-amine;
(R/S)-4-(3-(2-bromophenyl)piperazin-1-yl)-5H-pyrrolo[3,2-d]pyrimidin-2-amine;
(S/R)-4-(3-(2-bromophenyl)piperazin-1-yl)-5H-pyrrolo[3,2-d]pyrimidin-2-amine;
(R/S)-2-(4-(2-amino-6-(cyclopropylamino)pyrimidin-4-yl)-2-(2-bromophenyl)piperazin-1-yl)ethan-1-ol;
(S/R)-2-(4-(2-amino-6-(cyclopropylamino)pyrimidin-4-yl)-2-(2-bromophenyl)piperazin-1-yl)ethan-1-ol;
(R/S)-4-(2-(2-bromophenyl)morpholino)-5H-pyrrolo[3,2-d]pyrimidin-2-amine;
(S/R)-4-(2-(2-bromophenyl)morpholino)-5H-pyrrolo[3,2-d]pyrimidin-2-amine;
(S/R)-6-(2-(2-bromophenyl)morpholino)-5-fluoropyrimidine-2,4-diamine;
(R/S)-6-(2-(2-bromophenyl)morpholino)-5-fluoropyrimidine-2,4-diamine;
(R/S)-6-(3-(2-bromo-5-(4-methylpiperazin-1-yl)phenyl)piperazin-1-yl)pyrimidine-2,4-diamine;
(S/R)-6-(3-(2-bromo-5-(4-methylpiperazin-1-yl)phenyl)piperazin-1-yl)pyrimidine-2,4-diamine;
1-(4-(6-amino-2-(cyclopropylamino)pyrimidin-4-yl)-2-(2-bromophenyl)piperazin-1-yl)ethan-1-one;
2-(4-(4-amino-6-(cyclopropylamino)-1,3,5-triazin-2-yl)-2-(2-bromophenyl)piperazin-1-yl)ethan-1-ol;
6-(3-(2-(trifluoromethyl)phenyl)piperazin-1-yl)pyrimidine-2,4-diamine;
4-(3-(imidazo[1,2-a]pyridin-3-yl)piperazin-1-yl)-5H-pyrrolo[3,2-d]pyrimidin-2-amine;
(R/S)-4-(3-(2-bromophenyl)piperazin-1-yl)-7H-pyrrolo[2,3-d]pyrimidin-2-amine;
(S/R)-4-(3-(2-bromophenyl)piperazin-1-yl)-7H-pyrrolo[2,3-d]pyrimidin-2-amine;
4-(3-(imidazo[1,2-a]pyridin-3-yl)piperidin-1-yl)-5,6,7,8-tetrahydropyrido[2,3-d]pyrimidine;
(S/R)-1-(2-(2-bromophenyl)-4-(2,6-diaminopyrimidin-4-yl)piperazin-1-yl)ethan-1-one;
(R/S)-1-(2-(2-bromophenyl)-4-(2,6-diaminopyrimidin-4-yl)piperazin-1-yl)ethan-1-one;
6-(2-(2-bromophenyl)morpholino)-N2-cyclopropyl-1,3,5-triazine-2,4-diamine;
7-(3-(imidazo[1,2-a]pyridin-3-yl)piperidin-1-yl)-3H-imidazo[4,5-b]pyridine;
6-(3-(2-bromophenyl)piperazin-1-yl)-5-chloro-N4-cyclopropylpyrimidine-2,4-diamine;
(R/S)-6-(3-(2-bromophenyl)piperazin-1-yl)-N2-cyclopropyl-1,3,5-triazine-2,4-diamine;
(S/R)-6-(3-(2-bromophenyl)piperazin-1-yl)-N2-cyclopropyl-1,3,5-triazine-2,4-diamine;
(S/R)-2-(2-(2-bromophenyl)-4-(2,6-diaminopyrimidin-4-yl)piperazin-1-yl)ethan-1-ol;
(R/S)-2-(2-(2-bromophenyl)-4-(2,6-diaminopyrimidin-4-yl)piperazin-1-yl)ethan-1-ol;
6-(3-(2-bromophenyl)piperazin-1-yl)-5-chloropyrimidine-2,4-diamine;
(S/R)-2-(4-(6-amino-2-(cyclopropylamino)pyrimidin-4-yl)-2-(2-bromophenyl)piperazin-1-yl)ethan-1-ol;
(R/S)-2-(4-(6-amino-2-(cyclopropylamino)pyrimidin-4-yl)-2-(2-bromophenyl)piperazin-1-yl)ethan-1-ol;
6-(3-(2-bromophenyl)-4-methylpiperazin-1-yl)-N2-cyclopropylpyrimidine-2,4-diamine;
(S/R)-6-(2-(2-bromophenyl)morpholino)-2-isopropylpyrimidin-4-amine;
(R/S)-6-(2-(2-bromophenyl)morpholino)-2-isopropylpyrimidin-4-amine;
6-(3-(2-bromophenyl)-4-methylpiperazin-1-yl)-2-isopropylpyrimidin-4-amine;
(S/R)-6-(3-(2-bromophenyl)piperazin-1-yl)-2-isopropylpyrimidin-4-amine;
(R/S)-6-(3-(2-bromophenyl)piperazin-1-yl)-2-isopropylpyrimidin-4-amine;
(R/S)-6-(2-(2-bromophenyl)morpholino)-N4-(cyclopropylmethyl)pyrimidine-2,4-diamine;
(S/R)-6-(2-(2-bromophenyl)morpholino)-N4-(cyclopropylmethyl)pyrimidine-2,4-diamine;
6-(3-(2-bromophenyl)piperazin-1-yl)-N-cyclopropylpyrimidin-4-amine;
(R/S)-6-(3-(2-bromophenyl)-4-methylpiperazin-1-yl)-N2,N4-dimethylpyrimidine-2,4-diamine;
(S/R)-6-(3-(2-bromophenyl)-4-methylpiperazin-1-yl)-N2,N4-dimethylpyrimidine-2,4-diamine;
6-(3-(benzo[b]thiophen-3-yl)piperidin-1-yl)-N4-methylpyrimidine-2,4-diamine;
(R/S)-6-(3-(2-bromophenyl)piperidin-1-yl)-N2,N4-dimethylpyrimidine-2,4-diamine;
(S/R)-6-(3-(2-bromophenyl)piperidin-1-yl)-N2,N4-dimethylpyrimidine-2,4-diamine;
6-(3-(2-bromophenyl)piperidin-1-yl)-N2-methylpyrimidine-2,4-diamine;
(R/S)-6-(3-(2-bromophenyl)piperidin-1-yl)-N4-methylpyrimidine-2,4-diamine;
(S/R)-6-(3-(2-bromophenyl)piperidin-1-yl)-N4-methylpyrimidine-2,4-diamine;
4-(3-(imidazo[1,2-a]pyridin-3-yl)piperidin-1-yl)-5,6,7,8-tetrahydropyrido[2,3-d]pyrimidin-2-amine;
6-(3-(1H-indazol-1-yl)piperidin-1-yl)pyrimidine-2,4-diamine;
4-(3-(imidazo[1,2-a]pyridin-3-yl)piperidin-1-yl)-7H-pyrrolo[2,3-d]pyrimidine;
3-(1-(1H-pyrrolo[2,3-b]pyridin-4-yl)piperidin-3-yl)imidazo[1,2-a]pyridine;
6-(3-(imidazo[1,2-a]pyridin-3-yl)piperazin-1-yl)-N4-methylpyrimidine-2,4-diamine;
6-(3-(1H-benzo[d][1,2,3]triazol-1-yl)piperidin-1-yl)-N4-methylpyrimidine-2,4-diamine;
6-(3-(imidazo[1,5-a]pyridin-3-yl)piperidin-1-yl)-N4-methylpyrimidine-2,4-diamine;
(S/R)-N4-cyclopropyl-6-(2-phenylmorpholino)pyrimidine-2,4-diamine;
(R/S)-N4-cyclopropyl-6-(2-phenylmorpholino)pyrimidine-2,4-diamine;
(R/S)-6-(2-(2-bromophenyl)morpholino)-N4-cyclopropylpyrimidine-2,4-diamine;
(S/R)-6-(2-(2-bromophenyl)morpholino)-N4-cyclopropylpyrimidine-2,4-diamine;
(R/S)-6-(3-(2-bromophenyl)-4-methylpiperazin-1-yl)-N4-(cyclopropylmethyl)pyrimidine-2,4-diamine;
(S/R)-6-(3-(2-bromophenyl)-4-methylpiperazin-1-yl)-N4-(cyclopropylmethyl)pyrimidine-2,4-diamine;
6-(3-(2-bromophenyl)-4-methylpiperazin-1-yl)-N4-cyclopropylpyrimidine-2,4-diamine;
6-(3-(benzo[b]thiophen-3-yl)piperazin-1-yl)-N4-methylpyrimidine-2,4-diamine;
(S/R)-4-cyclopropyl-6-(3-(imidazo[1,2-a]pyridin-3-yl)piperazin-1-yl)pyrimidin-2-amine;
(R/S)-4-cyclopropyl-6-(3-(imidazo[1,2-a]pyridin-3-yl)piperazin-1-yl)pyrimidin-2-amine;
(S/R)-4-(3-(imidazo[1,2-a]pyridin-3-yl)-4-methylpiperazin-1-yl)-6-isopropylpyrimidin-2-amine;
(R/S)-4-(3-(imidazo[1,2-a]pyridin-3-yl)-4-methylpiperazin-1-yl)-6-isopropylpyrimidin-2-amine;
N4-cyclopropyl-6-(3-(imidazo[1,2-a]pyridin-3-yl)piperidin-1-yl)pyrimidine-2,4-diamine;
(R/S)-N4-methyl-6-(2-phenylmorpholino)pyrimidine-2,4-diamine;
(S/R)-N4-methyl-6-(2-phenylmorpholino)pyrimidine-2,4-diamine;
N4-methyl-6-(4-methyl-3-phenylpiperazin-1-yl)pyrimidine-2,4-diamine;
(R/S)-6-(3-(2-bromophenyl)piperazin-1-yl)-N4-(cyclopropylmethyl)pyrimidine-2,4-diamine;
(S/R)-6-(3-(2-bromophenyl)piperazin-1-yl)-N4-(cyclopropylmethyl)pyrimidine-2,4-diamine;
(R/S)-6-(3-(2-bromophenyl)piperazin-1-yl)-N2-methylpyrimidine-2,4-diamine;
(S/R)-6-(3-(2-bromophenyl)piperazin-1-yl)-N2-methylpyrimidine-2,4-diamine;
(S/R)-6-(3-(imidazo[1,2-a]pyridin-3-yl)piperidin-1-yl)pyrimidine-2,4-diamine;
(R/S)-6-(3-(imidazo[1,2-a]pyridin-3-yl)piperidin-1-yl)pyrimidine-2,4-diamine;
(S/R)-6-(3-(imidazo[1,2-a]pyridin-3-yl)piperidin-1-yl)-N2-methylpyrimidine-2,4-diamine;
(R/S)-6-(3-(imidazo[1,2-a]pyridin-3-yl)piperidin-1-yl)-N2-methylpyrimidine-2,4-diamine;
(S/R)-6-(3-(imidazo[1,2-a]pyridin-3-yl)piperazin-1-yl)pyrimidine-2,4-diamine;
(R/S)-6-(3-(imidazo[1,2-a]pyridin-3-yl)piperazin-1-yl)pyrimidine-2,4-diamine;
(R/S)-6-(3-(2-bromophenyl)piperazin-1-yl)-N4-cyclopropylpyrimidine-2,4-diamine;
(S/R)-6-(3-(2-bromophenyl)piperazin-1-yl)-N4-cyclopropylpyrimidine-2,4-diamine;
(S/R)-6-(3-(imidazo[1,2-a]pyridin-3-yl)piperidin-1-yl)-N2,N4-dimethylpyrimidine-2,4-diamine;
(R/S)-6-(3-(imidazo[1,2-a]pyridin-3-yl)piperidin-1-yl)-N2,N4-dimethylpyrimidine-2,4-diamine;
(S/R)-6-(3-(imidazo[1,2-a]pyridin-3-yl)piperidin-1-yl)-2-isopropyl-N-methylpyrimidin-4-amine;
(R/S)-6-(3-(imidazo[1,2-a]pyridin-3-yl)piperidin-1-yl)-2-isopropyl-N-methylpyrimidin-4-amine;
2-((4-(3-(imidazo[1,2-a]pyridin-3-yl)piperidin-1-yl)-6-isopropylpyrimidin-2-yl)amino)ethan-1-ol;
(R/S)-N4-methyl-6-(3-phenylpiperazin-1-yl)pyrimidine-2,4-diamine;
(S/R)-N4-methyl-6-(3-phenylpiperazin-1-yl)pyrimidine-2,4-diamine;
(S/R)-6-(3-(2-bromophenyl)piperazin-1-yl)-N2,N4-dimethylpyrimidine-2,4-diamine;
(R/S)-6-(3-(2-bromophenyl)piperazin-1-yl)-N2,N4-dimethylpyrimidine-2,4-diamine;
(R/S)-4-(3-(2-bromophenyl)piperazin-1-yl)-6-cyclopropylpyrimidin-2-amine;
(S/R)-4-(3-(2-bromophenyl)piperazin-1-yl)-6-cyclopropylpyrimidin-2-amine;
4-(3-(imidazo[1,2-a]pyridin-3-yl)piperidin-1-yl)-6-isopropyl-N-methylpyrimidin-2-amine;
3-(1-(2-amino-5,6,7,8-tetrahydropyrido[2,3-d]pyrimidin-4-yl)piperidin-3-yl)-N,N-dimethylpropanamide;
(R/S)-6-(2-(imidazo[1,2-a]pyridin-3-yl)morpholino)pyrimidine-2,4-diamine;
(S/R)-6-(2-(imidazo[1,2-a]pyridin-3-yl)morpholino)pyrimidine-2,4-diamine;
4-(3-(1H-indol-3-yl)piperazin-1-yl)-6-isopropylpyrimidin-2-amine;
4-cyclopropyl-6-(3-(imidazo[1,2-a]pyridin-3-yl)piperidin-1-yl)pyrimidin-2-amine;
3-(1-(2-amino-5,6,7,8-tetrahydropyrido[2,3-d]pyrimidin-4-yl)piperidin-3-yl)-N-methylpropanamide;
(S/R)-6-(3-(imidazo[1,2-a]pyridin-3-yl)piperidin-1-yl)-2-isopropylpyrimidin-4-amine;
(R/S)-6-(3-(imidazo[1,2-a]pyridin-3-yl)piperidin-1-yl)-2-isopropylpyrimidin-4-amine;
(R/S)-6-(3-(2-bromophenyl)-4-methylpiperazin-1-yl)pyrimidine-2,4-diamine;
(S/R)-6-(3-(2-bromophenyl)-4-methylpiperazin-1-yl)pyrimidine-2,4-diamine;
(R/S)-6-(2-(2-bromophenyl)morpholino)pyrimidine-2,4-diamine;
(S/R)-6-(2-(2-bromophenyl)morpholino)pyrimidine-2,4-diamine;
4-(3-(imidazo[1,2-a]pyridin-3-yl)piperidin-1-yl)-6-methylpyrimidin-2-amine;
(R/S)-3-(1-(6-isopropylpyrimidin-4-yl)piperidin-3-yl)imidazo[1,2-a]pyridine;
(S/R)-3-(1-(6-isopropylpyrimidin-4-yl)piperidin-3-yl)imidazo[1,2-a]pyridine;
(S/R)-4-(3-(2-bromophenyl)piperazin-1-yl)-N,6-diisopropylpyrimidin-2-amine;
(R/S)-4-(3-(2-bromophenyl)piperazin-1-yl)-N,6-diisopropylpyrimidin-2-amine;
(R/S)-4-(3-(2-bromophenyl)piperazin-1-yl)-6-isopropyl-N-methylpyrimidin-2-amine;
(S/R)-4-(3-(2-bromophenyl)piperazin-1-yl)-6-isopropyl-N-methylpyrimidin-2-amine;
(R/S)-6-(3-(2-bromophenyl)-4-methylpiperazin-1-yl)-N4-methylpyrimidine-2,4-diamine;
(S/R)-6-(3-(2-bromophenyl)-4-methylpiperazin-1-yl)-N4-methylpyrimidine-2,4-diamine;
(R/S)-4-(2-(imidazo[1,2-a]pyridin-3-yl)morpholino)-6-isopropylpyrimidin-2-amine;
(S/R)-4-(2-(imidazo[1,2-a]pyridin-3-yl)morpholino)-6-isopropylpyrimidin-2-amine;
(R/S)-4-(2-(2-bromophenyl)morpholino)-6-isopropylpyrimidin-2-amine;
(S/R)-4-(2-(2-bromophenyl)morpholino)-6-isopropylpyrimidin-2-amine;
(R/S)-4-(3-(2-bromophenyl)-4-methylpiperazin-1-yl)-6-isopropylpyrimidin-2-amine;
(S/R)-4-(3-(2-bromophenyl)-4-methylpiperazin-1-yl)-6-isopropylpyrimidin-2-amine;
4-(3-(benzo[b]thiophen-3-yl)piperazin-1-yl)-6-isopropylpyrimidin-2-amine;
3-(4-(2-amino-6-isopropylpyrimidin-4-yl)piperazin-2-yl)-4-bromophenol;
4-(4-(2-amino-6-isopropylpyrimidin-4-yl)piperazin-2-yl)-3-bromophenol;
4-isopropyl-6-(3-phenylpiperazin-1-yl)pyrimidin-2-amine;
3-(1-(2-amino-6-isopropylpyrimidin-4-yl)piperidin-3-yl)-N-methyl-[1,2,4]triazolo[4,3-a]pyridine-6-carboxamide;
3-(1-(2-amino-6-isopropylpyrimidin-4-yl)piperidin-3-yl)-N-methyl-[1,2,4]triazolo[4,3-a]pyridine-6-carboxamide;
4-(3-(2-ethylphenyl)piperazin-1-yl)-6-isopropylpyrimidin-2-amine;
4-(3-(2-bromo-3-fluorophenyl)piperazin-1-yl)-6-isopropylpyrimidin-2-amine;
4-(3-(2-bromo-4,5-difluorophenyl)piperazin-1-yl)-6-isopropylpyrimidin-2-amine;
4-isopropyl-6-(3-(2-methoxyphenyl)piperazin-1-yl)pyrimidin-2-amine;
6-(3-(2-bromophenyl)piperazin-1-yl)pyrimidine-2,4-diamine;
4-(3-(2-bromo-4-fluorophenyl)piperazin-1-yl)-6-isopropylpyrimidin-2-amine;
4-(3-(2-bromo-5-fluorophenyl)piperazin-1-yl)-6-isopropylpyrimidin-2-amine;
4-(3-(2-chlorophenyl)piperazin-1-yl)-6-isopropylpyrimidin-2-amine;
4-(3-(2-iodophenyl)piperazin-1-yl)-6-isopropylpyrimidin-2-amine;
4-isopropyl-6-(3-(o-tolyl)piperazin-1-yl)pyrimidin-2-amine;
4-(3-(imidazo[1,2-a]pyridin-3-yl)piperidin-1-yl)-6-isopropylpyrimidin-2-amine;
N4-(2-(1H-indol-3-yl)ethyl)-6-isopropyl-N4-methylpyrimidine-2,4-diamine;
6-(3-([1,2,4]triazolo[4,3-a]pyridin-3-yl)piperidin-1-yl)-N4-methylpyrimidine-2,4-diamine;
4-(3-(1H-benzo[d]imidazol-2-yl)piperidin-1-yl)-6-isopropylpyrimidin-2-amine;
4-(5,6-dihydroimidazo[1,5-a]pyrazin-7(8H)-yl)-6-isopropylpyrimidin-2-amine;
4-(3-(2-bromophenyl)piperazin-1-yl)-6-isopropylpyrimidin-2-amine;
4-(3-(2-bromophenyl)piperazin-1-yl)-7,8-dihydro-5H-pyrano[4,3-d]pyrimidin-2-amine;
4-(3-(4H-1,2,4-triazol-3-yl)piperazin-1-yl)-5,7-dihydrothieno[3,4-d]pyrimidin-2-amine;
4-(3-(imidazo[1,2-a]pyridin-3-yl)piperidin-1-yl)-5,7-dihydrothieno[3,4-d]pyrimidin-2-amine;
4-(3-(1H-pyrazol-5-yl)piperidin-1-yl)-5,6,7,8-tetrahydropyrido[2,3-d]pyrimidin-2-amine;
4-(3-([1,2,4]triazolo[4,3-a]pyridin-3-yl)piperazin-1-yl)-6-isopropylpyrimidin-2-amine;
4-(3-(2-bromophenyl)piperazin-1-yl)-5,7-dihydrothieno[3,4-d]pyrimidin-2-amine;
N2,6-dimethyl-N4-(3-(pyrazin-2-yl)phenyl)pyrimidine-2,4-diamine;
4-(3-([1,2,4]triazolo[4,3-a]pyridin-3-yl)piperidin-1-yl)-6,7-dihydrothieno[3,2-d]pyrimidin-2-amine;
N4-(3-(pyrazin-2-yl)phenyl)pyrimidine-2,4,6-triamine;
6-(3-(1H-pyrazol-5-yl)piperidin-1-yl)pyrimidine-2,4-diamine;
4-(3-([1,2,4]triazolo[4,3-a]pyridin-3-yl)piperidin-1-yl)-6-isopropylpyrimidin-2-amine;
4-(3-(1H-pyrazol-5-yl)piperidin-1-yl)-6,7-dihydro-5H-cyclopenta[d]pyrimidin-2-amine;
4-(3-(1H-pyrazol-5-yl)piperidin-1-yl)-7,8-dihydro-5H-pyrano[4,3-d]pyrimidin-2-amine;
3-(3-((2-amino-6-isopropylpyrimidin-4-yl)amino)benzyl)pyrrolidin-2-one;
6-isopropyl-N4-(3-(pyrazin-2-yl)phenyl)pyrimidine-2,4-diamine;
4-(3-(1H-pyrazol-5-yl)piperidin-1-yl)-5,7-dihydrothieno[3,4-d]pyrimidin-2-amine;
4-(3-(2,5-dibromophenyl)piperazin-1-yl)-6-isopropylpyrimidin-2-amine;
3-(1-(2-amino-5,7-dihydrothieno[3,4-d]pyrimidin-4-yl)piperidin-3-yl)propanamide;
5-((1-(2-amino-6-methylpyrimidin-4-yl)piperidin-3-yl)amino)pyridin-2(1H)-one;
N4-(2-(1H-indol-3-yl)ethyl)-6-isopropylpyrimidine-2,4-diamine;
4-(3-(4H-1,2,4-triazol-3-yl)piperazin-1-yl)-6-isopropylpyrimidin-2-amine;
4-(3-(1H-pyrazol-5-yl)piperidin-1-yl)-7H-pyrrolo[2,3-d]pyrimidin-2-amine;
4-(3-(1H-pyrazol-5-yl)piperidin-1-yl)-5H-pyrrolo[3,2-d]pyrimidin-2-amine;
6-(3-(1H-pyrazol-5-yl)piperidin-1-yl)-7H-purin-2-amine;
4-(3-(1H-pyrazol-5-yl)piperidin-1-yl)-7,8-dihydro-5H-thiopyrano[4,3-d]pyrimidin-2-amine;
4-(3-(1H-pyrazol-5-yl)piperidin-1-yl)-6-methylpyrimidin-2-amine;
4-(3-(1H-pyrazol-5-yl)piperidin-1-yl)-6-isopropylpyrimidin-2-amine;
4-(3-(1H-pyrazol-5-yl)piperidin-1-yl)-6-cyclopropylpyrimidin-2-amine;
4-(3-(1H-pyrazol-5-yl)piperidin-1-yl)-6-cyclobutylpyrimidin-2-amine;
4-(3-(1H-pyrazol-5-yl)piperidin-1-yl)quinazolin-2-amine;
4-(3-(1H-pyrazol-5-yl)piperidin-1-yl)-5,6-dimethylpyrimidin-2-amine;
4-(3-(1-Methyl-1H-pyrazol-5-yl)piperidin-1-yl)-6,7-dihydro-5H-cyclopenta[d]pyrimidin-2-amine;
4-(3-(1H-pyrazol-5-yl)piperidin-1-yl)-5-bromo-6-methylpyrimidin-2-amine;
4-(3-(1H-pyrazol-5-yl)piperidin-1-yl)thieno[3,2-d]pyrimidin-2-amine;
4-(3-(1H-pyrazol-5-yl)piperidin-1-yl)pyrido[4,3-d]pyrimidin-2-amine;
4-(3-(1H-pyrazol-5-yl)piperidin-1-yl)-5,8-dihydro-6H-pyrano[3,4-d]pyrimidin-2-amine;
4-(3-(2-bromophenyl)-1,4-diazepan-1-yl)-6-isopropylpyrimidin-2-amine;
4-(3-(2-bromophenyl)-3-fluoropiperidin-1-yl)-6-isopropylpyrimidin-2-amine;
3-(2-bromophenyl)-1-(2,6-diaminopyrimidin-4-yl)piperidin-3-ol;
1-(2-amino-6-isopropylpyrimidin-4-yl)-3-(2-bromophenyl)piperidin-3-ol;
1-(2-amino-6-isopropylpyrimidin-4-yl)-3-(imidazo[1,5-a]pyridin-1-yl)piperidin-3-ol;
6-(3-(imidazo[1,5-a]pyridin-1-yl)piperidin-1-yl)pyrimidine-2,4-diamine;
1-(2-amino-6-isopropylpyrimidin-4-yl)-3-(2-bromophenyl)azepan-3-ol;
4-(5-(2-bromophenyl)-3,4-dihydropyridin-1(2H)-yl)-6-isopropylpyrimidin-2-amine;
6-(3-(2-bromophenyl)-3-fluoropiperidin-1-yl)pyrimidine-2,4-diamine;
rac-4-((3R,4R)-4-fluoro-3-(imidazo[1,5-a]pyridin-1-yl)piperidin-1-yl)-6-isopropylpyrimidin-2-amine;
4-(5-(2-bromophenyl)-3,6-dihydropyridin-1(2H)-yl)-6-isopropylpyrimidin-2-amine;
1-(2-amino-6-isopropylpyrimidin-4-yl)-3-(2-bromophenyl)pyrrolidin-3-ol;
4-isopropyl-6-(3-(1-methyl-1H-indazol-3-yl)piperidin-1-yl)pyrimidin-2-amine;
4-(3-(2-bromophenyl)-3-fluoropiperidin-1-yl)-6-isopropylpyrimidin-2-amine;
3-(2-bromophenyl)-1-(2,6-diaminopyrimidin-4-yl)piperidin-3-ol;
4-(3-(imidazo[1,5-a]pyridin-1-yl)piperidin-1-yl)-6-isopropylpyrimidin-2-amine;
1-(2-amino-6-isopropylpyrimidin-4-yl)-3-(2-bromophenyl)piperidin-3-ol;
6-(5-(imidazo[1,5-a]pyridin-1-yl)-3,6-dihydropyridin-1(2H)-yl)pyrimidine-2,4-diamine;
4-(5-(imidazo[1,5-a]pyridin-1-yl)-3,6-dihydropyridin-1(2H)-yl)-6-isopropylpyrimidin-2-amine;
4-(3-(imidazo[1,2-c]pyrimidin-3-yl)piperidin-1-yl)-6-isopropylpyrimidin-2-amine;
4-isopropyl-6-(3-(pyrazolo[1,5-a]pyridin-3-yl)piperidin-1-yl)pyrimidin-2-amine;
6-(3-(imidazo[1,2-a]pyrimidin-3-yl)piperidin-1-yl)pyrimidine-2,4-diamine;
4-(3-(6,8-difluoroimidazo[1,2-a]pyridin-3-yl)piperidin-1-yl)-6-isopropylpyrimidin-2-amine;
1-(2-amino-6-isopropylpyrimidin-4-yl)-3-(imidazo[1,2-a]pyridin-3-yl)piperidin-3-ol;
4-(3-(imidazo[1,5-a]pyridin-3-yl)piperidin-1-yl)-6-isopropylpyrimidin-2-amine;
4-(3-(imidazo[1,2-a]pyrimidin-3-yl)piperidin-1-yl)-6-isopropylpyrimidin-2-amine;
6-(3-(1H-indazol-3-yl)piperidin-1-yl)pyrimidine-2,4-diamine;
4-isopropyl-6-(3-(1-methyl-1H-indol-3-yl)piperidin-1-yl)pyrimidin-2-amine;
6-(3-(1-methyl-1H-indol-3-yl)piperidin-1-yl)pyrimidine-2,4-diamine;
4-(3-(1H-indol-3-yl)piperidin-1-yl)-6-isopropylpyrimidin-2-amine;
6-(3-(1H-indol-3-yl)piperidin-1-yl)pyrimidine-2,4-diamine;
4-(3-(1H-indazol-3-yl)piperidin-1-yl)-6-isopropylpyrimidin-2-amine;
1-(1-(2-amino-6-isopropylpyrimidin-4-yl)piperidin-3-yl)-1,3-dihydro-2H-benzo[d]imidazol-2-one;
4-(3-(imidazo[1,2-a]pyridin-3-yl)pyrrolidin-1-yl)-6-isopropylpyrimidin-2-amine;
6-(3-(1H-indazol-1-yl)piperidin-1-yl)-N4-methylpyrimidine-2,4-diamine;
6-(3-(imidazo[1,2-a]pyridin-3-yl)pyrrolidin-1-yl)pyrimidine-2,4-diamine;
4-(3-(1H-benzo[d]imidazol-1-yl)piperidin-1-yl)-6-isopropylpyrimidin-2-amine;
1-(1-(2,6-diaminopyrimidin-4-yl)piperidin-3-yl)-1,3-dihydro-2H-benzo[d]imidazol-2-one;
6-(3-(imidazo[1,2-c]pyrimidin-3-yl)piperidin-1-yl)pyrimidine-2,4-diamine;
6-(3-(2H-benzo[d][1,2,3]triazol-2-yl)piperidin-1-yl)-N4-methylpyrimidine-2,4-diamine;
6-(3-(1H-benzo[d]imidazol-1-yl)piperidin-1-yl)pyrimidine-2,4-diamine;
1-(3-(4-(2,6-diaminopyrimidin-4-yl)piperazin-2-yl)-4-(trifluoromethyl)benzoyl)azetidine-3-carbonitrile;
(3-(4-(2,6-diaminopyrimidin-4-yl)piperazin-2-yl)-4-(trifluoromethyl)phenyl)(4-methoxypiperidin-1-yl)methanone;
(S)-(3-(4-(2,6-diaminopyrimidin-4-yl)piperazin-2-yl)-4-(trifluoromethyl)phenyl)(piperidin-1-yl)methanone;
(R)-(3-(4-(2,6-diaminopyrimidin-4-yl)piperazin-2-yl)-4-(trifluoromethyl)phenyl)(piperidin-1-yl)methanone;
(S)-(3-(4-(2,6-diaminopyrimidin-4-yl)piperazin-2-yl)-4-(trifluoromethyl)phenyl)(4,4-difluoropiperidin-1-yl)methanone;
(R)-(3-(4-(2,6-diaminopyrimidin-4-yl)piperazin-2-yl)-4-(trifluoromethyl)phenyl)(4,4-difluoropiperidin-1-yl)methanone;
(S)-(3-(4-(2,6-diaminopyrimidin-4-yl)piperazin-2-yl)-4-(trifluoromethyl)phenyl)(morpholino)methanone; and
(R)-(3-(4-(2,6-diaminopyrimidin-4-yl)piperazin-2-yl)-4-(trifluoromethyl)phenyl)(morpholino)methanone.

20. The compound according to claim 1, wherein the compound is chosen from:
(R/S)-6-(3-(2-bromo-5-(1,5-dimethyl-1H-pyrazol-4-yl)phenyl)piperazin-1-yl)pyrimidine-2,4-diamine;
(S/R)-6-(3-(2-bromo-5-(1,5-dimethyl-1H-pyrazol-4-yl)phenyl)piperazin-1-yl)pyrimidine-2,4-diamine;
(R/S)-4-(3-(2-bromo-5-(1,5-dimethyl-1H-pyrazol-4-yl)phenyl)piperazin-1-yl)-6-isopropylpyrimidin-2-amine;
(S/R)-4-(3-(2-bromo-5-(1,5-dimethyl-1H-pyrazol-4-yl)phenyl)piperazin-1-yl)-6-isopropylpyrimidin-2-amine;
4-bromo-3-(4-(2,6-diaminopyrimidin-4-yl)piperazin-2-yl)-N-(2-(dimethylamino)ethyl)benzamide;
(R/S)-6-(3-(2-bromo-5-(trifluoromethyl)phenyl)piperazin-1-yl)pyrimidine-2,4-diamine;
(S/R)-6-(3-(2-bromo-5-(trifluoromethyl)phenyl)piperazin-1-yl)pyrimidine-2,4-diamine;
(R/S)-4-(3-(2-bromo-5-(trifluoromethyl)phenyl)piperazin-1-yl)-6-isopropylpyrimidin-2-amine;
(S/R)-4-(3-(2-bromo-5-(trifluoromethyl)phenyl)piperazin-1-yl)-6-isopropylpyrimidin-2-amine; (R/S)-(4-bromo-3-(4-(2,6-diaminopyrimidin-4-yl)piperazin-2-yl)phenyl)(4-methylpiperazin-1-yl)methanone;
(S/R)-(4-bromo-3-(4-(2,6-diaminopyrimidin-4-yl)piperazin-2-yl)phenyl)(4-methylpiperazin-1-yl)methanone;
(R/S)-(3-(4-(2-amino-6-isopropylpyrimidin-4-yl)piperazin-2-yl)-4-bromophenyl)(4-methylpiperazin-1-yl)methanone;
(S/R)-(3-(4-(2-amino-6-isopropylpyrimidin-4-yl)piperazin-2-yl)-4-bromophenyl)(4-methylpiperazin-1-yl)methanone;
7-(3-(imidazo[1,2-a]pyridin-3-yl)piperidin-1-yl)-5-isopropyl-3H-imidazo[4,5-b]pyridine;
(R/S)-3-(4-(2-amino-6-isopropylpyrimidin-4-yl)piperazin-2-yl)-4-bromo-N-(2-(dimethylamino)ethyl)benzamide;
(S/R)-3-(4-(2-amino-6-isopropylpyrimidin-4-yl)piperazin-2-yl)-4-bromo-N-(2-(dimethylamino)ethyl)benzamide;
4-isopropyl-6-(3-(pyrazolo[1,5-a]pyridin-3-yl)piperazin-1-yl)pyrimidin-2-amine;
(R/S)-6-(3-(2-bromo-5-ethoxyphenyl)piperazin-1-yl)pyrimidine-2,4-diamine;
(S/R)-6-(3-(2-bromo-5-ethoxyphenyl)piperazin-1-yl)pyrimidine-2,4-diamine;
(R/S)-4-(3-(2-bromo-5-ethoxyphenyl)piperazin-1-yl)-6-isopropylpyrimidin-2-amine;
(S/R)-4-(3-(2-bromo-5-ethoxyphenyl)piperazin-1-yl)-6-isopropylpyrimidin-2-amine;
(R/S)-4-ethyl-6-(3-(imidazo[1,2-a]pyridin-3-yl)piperazin-1-yl)pyrimidin-2-amine;
(S/R)-4-ethyl-6-(3-(imidazo[1,2-a]pyridin-3-yl)piperazin-1-yl)pyrimidin-2-amine;
(R/S)-(1-(3-(4-(2-amino-6-isopropylpyrimidin-4-yl)piperazin-2-yl)-4-bromophenyl)azetidin-3-yl)methanol;
(S/R)-(1-(3-(4-(2-amino-6-isopropylpyrimidin-4-yl)piperazin-2-yl)-4-bromophenyl)azetidin-3-yl)methanol;
4-(3-(imidazo[1,2-a]pyridin-3-yl)piperazin-1-yl)-6-isopropylpyrimidin-2-amine;
4-(3-(2-bromo-5-(piperazin-1-yl)phenyl)piperazin-1-yl)-6-isopropylpyrimidin-2-amine;
(R/S)-7-(3-(imidazo[1,2-a]pyridin-3-yl)piperazin-1-yl)-5-isopropyl-3H-imidazo[4,5-b]pyridine;
(S/R)-7-(3-(imidazo[1,2-a]pyridin-3-yl)piperazin-1-yl)-5-isopropyl-3H-imidazo[4,5-b]pyridine;
3-(1-(6-isopropyl-1H-pyrrolo[2,3-b]pyridin-4-yl)piperidin-3-yl)imidazo[1,2-a]pyridine;
(R/S)-3-(4-(6-isopropyl-1H-pyrrolo[2,3-b]pyridin-4-yl)piperazin-2-yl)imidazo[1,2-a]pyridine;
(S/R)-3-(4-(6-isopropyl-1H-pyrrolo[2,3-b]pyridin-4-yl)piperazin-2-yl)imidazo[1,2-a]pyridine;
6-(3-(imidazo[1,2-a]pyridin-3-yl)piperazin-1-yl)-2-isopropyl-9H-purine;
4-(3-(imidazo[1,2-a]pyridin-3-yl)piperidin-1-yl)-2-isopropyl-7H-pyrrolo[2,3-d]pyrimidine;
4-(3-(imidazo[1,2-a]pyridin-3-yl)piperazin-1-yl)-2-isopropyl-7H-pyrrolo[2,3-d]pyrimidine;
6-(3-(imidazo[1,2-a]pyridin-3-yl)piperidin-1-yl)-2-isopropyl-9H-purine;
1-(3-(4-(2,6-diaminopyrimidin-4-yl)piperazin-2-yl)-4-(trifluoromethyl)benzoyl)azetidine-3-carbonitrile;
(3-(4-(2,6-diaminopyrimidin-4-yl)piperazin-2-yl)-4-(trifluoromethyl)phenyl)(4-methoxypiperidin-1-yl)methanone;
(S)-(3-(4-(2,6-diaminopyrimidin-4-yl)piperazin-2-yl)-4-(trifluoromethyl)phenyl)(piperidin-1-yl)methanone;
(R)-(3-(4-(2,6-diaminopyrimidin-4-yl)piperazin-2-yl)-4-(trifluoromethyl)phenyl)(piperidin-1-yl)methanone;
(S)-(3-(4-(2,6-diaminopyrimidin-4-yl)piperazin-2-yl)-4-(trifluoromethyl)phenyl)(4,4-difluoropiperidin-1-yl)methanone;
(R)-(3-(4-(2,6-diaminopyrimidin-4-yl)piperazin-2-yl)-4-(trifluoromethyl)phenyl)(4,4-difluoropiperidin-1-yl)methanone;
(S)-(3-(4-(2,6-diaminopyrimidin-4-yl)piperazin-2-yl)-4-(trifluoromethyl)phenyl)(morpholino) methanone; and
(R)-(3-(4-(2,6-diaminopyrimidin-4-yl)piperazin-2-yl)-4-(trifluoromethyl)phenyl)(morpholino)methanone.

21. A compound chosen from Formula (2) or a tautomer, stereoisomer or a mixture of stereoisomers, or a pharmaceutically acceptable salt, or hydrate, or deuterated derivative thereof: 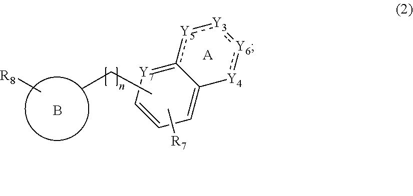
wherein:
Y₃ is chosen from CH, CH₂, N, CO, and NH;
Y₄ is chosen from CH, N, and CH₂;
Y₅ is chosen from CH, CH₂, CNHR₁₀, CO, N, 5 membered heteroaryl rings comprising two nitrogen atoms and three carbon atoms wherein the heteroaryl ring is optionally substituted with a methyl, and NH, wherein R₁₀ is chosen from H and C₁-C₄ alkyl;
Y₆ is chosen from CH and CH₂;
Y₇ is chosen from CH, N, and C—OH;
R₇ is absent or chosen from OH, halogen, 5 membered heteroaryl rings comprising two nitrogen atoms and three carbon atoms wherein the heteroaryl ring is optionally substituted with a methyl, and methyl;
ring A is an aromatic or non-aromatic 6-membered heterocyclic ring;
n is 0 or 1;
ring system B is chosen from:
5 to 6 membered rings optionally comprising up to three nitrogen atoms, a sulfur atom, and/or an oxygen atom,
bicyclic 9 or 10 membered rings comprising at least one aromatic ring, optionally comprising up to three nitrogen atoms, a sulfur atom, and/or a carbonyl, and
bicyclic 7 or 8 membered aliphatic rings comprising an amine, and wherein ring system B is optionally substituted with up to two halogens, a methyl, and/or NH₂;
R₈ is absent or chosen from:
aliphatic amines comprising up to two nitrogen atoms, up to 7 carbon atoms, and optionally an alkoxy,
N-substituted amides wherein at least one N-substituent comprises up to 7 carbon atoms and, optionally, an amine, a hydroxyl, and/or an alkoxy, and
C-substituted carbonyls wherein the C-substituent comprises up to 6 carbon atoms and a tertiary amine;
and wherein Formula (2) encompasses all tautomers, stereoisomers, isotopes, pharmaceutically acceptable salts, and mixtures thereof.

22. The compound according to claim 21, wherein the compound is a compound of Formula (2a): 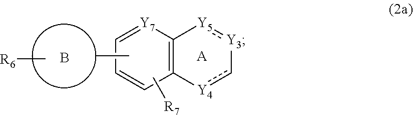
wherein:
Y₃ is chosen from N, NH, CH, CO, and C—NH₂;
Y₄ is chosen from N and CH;
Y₅ is chosen from N, NH, CH, CO, and C—NH₂;
at least one of Y₃, Y₄, and Y₅ is N;
each dashed bond in ring A independently represents a single or double bond, and R₇ is absent or chosen from OH, halogen, and methyl;
and wherein Formula (2a) encompasses all tautomers, stereoisomers, deuterated derivatives, pharmaceutically acceptable salts, and mixtures thereof.

23. The compound according to claim 22, wherein the compound is a compound of Formula (2aa): 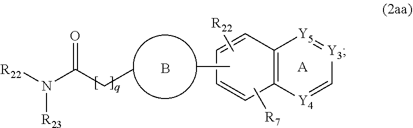
wherein:
q is 0 or 1;
R₂₂ is chosen from H and a C₁-C₃ alkyl;
R₂₃ consists of H, C, optionally N, and optionally O; and
R₂₃ comprises up to 7 carbon atoms and, optionally, an amine, a hydroxyl, and/or an alkoxy;
ring A is an aromatic 6-membered heterocyclic ring;
and wherein Formula (2aa) encompasses all tautomers, stereoisomers, deuterated derivatives, pharmaceutically acceptable salts, and mixtures thereof.

24. The compound according to any one of claims 21 to 23, wherein ring system B consists of C, H, optionally N, optionally S, optionally F, and optionally Cl.

25. The compound according to claim 24, wherein ring system B is chosen from: 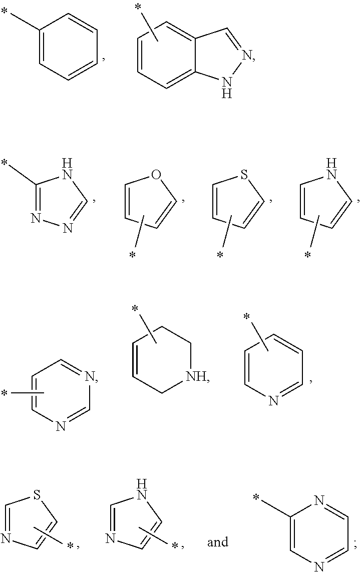
wherein each asterisk (*) denotes a point of attachment within Formula (2), Formula (2a), or Formula (2aa).

26. The compound according to claim 22, wherein the compound is chosen from:

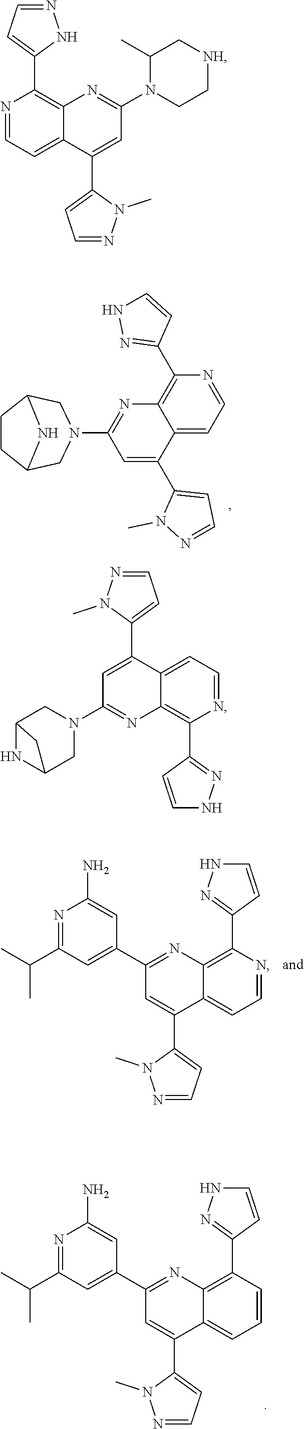

27. The compound according to claim 22, wherein the compound is chosen from:
5-(1-aminoisoquinolin-7-yl)-1,2-dihydro-3H-indazol-3-one;
N1-((5-(1-aminoisoquinolin-7-yl)-1H-indazol-3-yl)methyl)-N2,N2-dimethylethane-1,2-diamine;
5-(1-aminoisoquinolin-7-yl)-N-(2-(dimethylamino)ethyl)-1H-pyrrolo[2,3-b]pyridine-3-carboxamide;
5-(1-aminoisoquinolin-7-yl)-N-(1,4-dimethylpiperidin-4-yl)-1H-indazole-3-carboxamide;
4-(2-amino-8-hydroxyquinolin-6-yl)-N-(3-(dimethylamino)propyl)benzamide;
N-(3-(dimethylamino)propyl)-5-(8-hydroxyquinolin-6-yl)-4H-1,2,4-triazole-3-carboxamide;
N-(3-(dimethylamino)propyl)-5-(8-hydroxyquinolin-6-yl)thiophene-3-carboxamide;
N-((1,3-cis)-3-(dimethylamino)cyclobutyl)-5-(8-hydroxyquinolin-6-yl)thiophene-3-carboxamide;
4-(4-amino-8-hydroxyquinolin-6-yl)-N-(3-(dimethylamino)propyl)benzamide;
(3-(1-amino-5-hydroxyisoquinolin-7-yl)phenyl)(pyrrolidin-1-yl)methanone;
4-(4-amino-8-hydroxyquinazolin-6-yl)-N-(2-(dimethylamino)ethyl)benzamide;
N-(2-(dimethylamino)ethyl)-5-(8-hydroxyquinolin-6-yl)thiophene-3-carboxamide;
N-(3-(dimethylamino)propyl)-4-(8-hydroxyquinolin-6-yl)-1H-pyrrole-2-carboxamide;
N-(2-(dimethylamino)ethyl)-4-(8-hydroxyquinazolin-6-yl)benzamide;
N-(3-(dimethylamino)propyl)-5-(8-hydroxyquinolin-6-yl)pyrimidine-2-carboxamide;
N-(3-(dimethylamino)propyl)-5-(8-hydroxyquinolin-6-yl)thiazole-2-carboxamide;
N-(3-(dimethylamino)propyl)-4-(8-hydroxy-3-oxo-3,4-dihydroquinoxalin-6-yl)benzamide;
N-(3-(dimethylamino)propyl)-2-(8-hydroxyquinolin-6-yl)thiazole-5-carboxamide;
N-(3-(dimethylamino)propyl)-5-(8-hydroxyquinolin-6-yl)-1H-imidazole-2-carboxamide;
N-(3-(dimethylamino)propyl)-5-(8-hydroxyquinolin-6-yl)pyrazine-2-carboxamide;
N-(3-(dimethylamino)propyl)-2-(8-hydroxyquinolin-6-yl)-1H-imidazole-4-carboxamide;
6-(3-(pyrrolidine-1-carbonyl)phenyl)-3,4-dihydroisoquinolin-1 (2H)-one;
N-((1,3-cis)-3-(dimethylamino)cyclobutyl)-4-(8-hydroxyquinolin-6-yl)benzamide;
N-((1,3-trans)-3-(dimethylamino)cyclobutyl)-4-(8-hydroxyquinolin-6-yl)benzamide;
N-(3-(dimethylamino)propyl)-2-(8-hydroxyquinolin-6-yl)pyrimidine-5-carboxamide;
N-(3-(dimethylamino)propyl)-4-(8-hydroxyquinolin-6-yl)furan-2-carboxamide;
N-(3-(dimethylamino)propyl)-4-(8-hydroxy-1,2,3,4-tetrahydroquinolin-6-yl)benzamide;
N-(3-(dimethylamino)propyl)-4-(8-hydroxyquinoxalin-6-yl)benzamide;
N-((1 s,4s)-4-(dimethylamino)cyclohexyl)-4-(8-hydroxyquinolin-6-yl)benzamide;
N-((1,4-trans)-4-(dimethylamino)cyclohexyl)-4-(8-hydroxyquinolin-6-yl)benzamide;
N-(3-(dimethylamino)propyl)-2-(4-(8-hydroxyquinolin-6-yl)phenyl)acetamide;
N-(4-(dimethylamino)butyl)-4-(8-hydroxyquinolin-6-yl)benzamide;
4-(8-hydroxyquinolin-6-yl)-N-((1-methylpiperidin-3-yl)methyl)benzamide;
4-(8-hydroxyquinolin-6-yl)-N-(1-methylazetidin-3-yl)benzamide;
4-(8-hydroxyquinolin-6-yl)-N-((1-methylpyrrolidin-3-yl)methyl)benzamide;
(3-(8-hydroxyquinolin-6-yl)phenyl)(pyrrolidin-1-yl)methanone;
N-(3-(dimethylamino)propyl)-6-(8-hydroxyquinolin-6-yl)nicotinamide;
N-(3-(dimethylamino)propyl)-5-(8-hydroxyquinolin-6-yl)picolinamide;
N-(3-(dimethylamino)propyl)-5-(8-hydroxyquinolin-6-yl)thiophene-2-carboxamide;
4-(8-hydroxyquinolin-6-yl)-N-(1-methylpiperidin-4-yl)benzamide;
4-(8-hydroxyquinolin-6-yl)-N-((1-methylazetidin-3-yl)methyl)benzamide;
N-(2-(dimethylamino)ethyl)-4-(8-hydroxyquinolin-6-yl)benzamide;
4-(8-hydroxyquinolin-6-yl)-N-((1-methylpiperidin-4-yl)methyl)benzamide;
(4-(8-hydroxyquinolin-6-yl)phenyl)(pyrrolidin-1-yl)methanone;
4-(8-hydroxyquinolin-6-yl)-N-propylbenzamide;
4-(8-hydroxyquinolin-6-yl)-N-(3-(methylamino)propyl)benzamide;
N-(3-(dimethylamino)propyl)-4-(4-hydroxy-1,5-naphthyridin-2-yl)benzamide;
N-(3-(dimethylamino)propyl)-4-(8-hydroxyquinolin-6-yl)cyclohex-3-ene-1-carboxamide;
N-(3-(dimethylamino)propyl)-4-(8-hydroxyquinolin-6-yl)-3,6-dihydropyridine-1(2H)-carboxamide;
N-(3-(dimethylamino)propyl)-2-(4-(8-hydroxyquinolin-6-yl)-3,6-dihydropyridin-1(2H)-yl)acetamide;
N-(3-(dimethylamino)propyl)-4-(7-fluoro-8-hydroxyquinolin-6-yl)benzamide;
4-(dimethylamino)-1-(4-(8-hydroxyquinolin-6-yl)-3,6-dihydropyridin-1(2H)-yl)butan-1-one;
2-chloro-N-(3-(dimethylamino)propyl)-3-fluoro-4-(8-hydroxyquinolin-6-yl)benzamide;
N-(3-(dimethylamino)propyl)-4-(8-hydroxyquinolin-6-yl)-2-methylbenzamide;
2-amino-N-(3-(dimethylamino)propyl)-4-(8-hydroxyquinolin-6-yl)benzamide;
2-bromo-N-(3-(dimethylamino)propyl)-4-(8-hydroxyquinolin-6-yl)benzamide;
N-(3-(dimethylamino)propyl)-2,6-difluoro-4-(8-hydroxyquinolin-6-yl)benzamide;
2-chloro-N-(3-(dimethylamino)propyl)-5-fluoro-4-(8-hydroxyquinolin-6-yl)benzamide;
N-(3-(dimethylamino)propyl)-2,5-difluoro-4-(8-hydroxyquinolin-6-yl)benzamide;
3-chloro-N-(3-(dimethylamino)propyl)-4-(8-hydroxyquinolin-6-yl)benzamide;
2,6-dichloro-N-(3-(dimethylamino)propyl)-4-(8-hydroxyquinolin-6-yl)benzamide;
N-(3-(dimethylamino)propyl)-4-(8-hydroxy-5-methylquinolin-6-yl)benzamide;
N-(3-(dimethylamino)propyl)-4-(8-hydroxyquinolin-6-yl)-3-methylbenzamide;
4-(7-chloro-8-hydroxyquinolin-6-yl)-N-(3-(dimethylamino)propyl)benzamide;
2,5-dichloro-N-(3-(dimethylamino)propyl)-4-(8-hydroxyquinolin-6-yl)benzamide;
3-bromo-N-(3-(dimethylamino)propyl)-4-(8-hydroxyquinolin-6-yl)benzamide;
6-(1,2,3,6-tetrahydropyridin-4-yl)quinolin-8-ol;
N-(3-(dimethylamino)propyl)-3-fluoro-4-(8-hydroxyquinolin-6-yl)benzamide;
5-(1-aminoisoquinolin-7-yl)-N-((2-methoxypyridin-4-yl)methyl)-1H-indazole-3-carboxamide;
5-(1-aminoisoquinolin-7-yl)-N-(2-(dimethylamino)ethyl)-1H-pyrazolo[4,3-b]pyridine-3-carboxamide;
5-(1-aminoisoquinolin-7-yl)-N-((1-methyl-1H-pyrazol-4-yl)methyl)-1H-indazole-3-carboxamide;
5-(4-aminoquinazolin-6-yl)-N-(2-(dimethylamino)ethyl)-1H-indazole-3-carboxamide;
5-(4-aminoquinazolin-6-yl)-N-(3-(dimethylamino)propyl)-1H-indazole-3-carboxamide;
5-(1-aminoisoquinolin-7-yl)-N-(2-hydroxyethyl)-1H-indazole-3-carboxamide;
(5-(1-aminoisoquinolin-7-yl)-1H-indazol-3-yl)(4-methoxypiperidin-1-yl)methanone;
5-(1-aminoisoquinolin-7-yl)-N-(((1,4-cis)-4-hydroxycyclohexyl)methyl)-1H-indazole-3-carboxamide;
5-(1-aminoisoquinolin-7-yl)-N-((1-methylazetidin-3-yl)methyl)-1H-indazole-3-carboxamide;
(5-(1-aminoisoquinolin-7-yl)-1H-indazol-3-yl)(4-(dimethylamino)piperidin-1-yl)methanone;
(5-(1-aminoisoquinolin-7-yl)-1H-indazol-3-yl)(4-hydroxypiperidin-1-yl)methanone;
5-(1-aminoisoquinolin-7-yl)-N-((1-methylpiperidin-4-yl)methyl)-1H-indazole-3-carboxamide;
5-(1-aminoisoquinolin-7-yl)-N-(1-methylpiperidin-4-yl)-1H-indazole-3-carboxamide;
(5-(1-aminoisoquinolin-7-yl)-1H-indazol-3-yl)(4-aminopiperidin-1-yl)methanone;
3-(8-amino-1,7-naphthyridin-2-yl)-N-(3-(dimethylamino)propyl)benzamide;
5-(1-aminoisoquinolin-7-yl)-N-(2-(dimethylamino)ethyl)-1H-indazole-3-carboxamide;
(4-(1-aminoisoquinolin-7-yl)-5,6,7,8-tetrahydronaphthalen-2-yl)(pyrrolidin-1-yl)methanone;
(3-((1-aminoisoquinolin-7-yl)methyl)phenyl)(pyrrolidin-1-yl)methanone;
7-(3-(2-(4-methylpiperazin-1-yl)ethyl)phenyl)isoquinolin-1-amine;
3-(1-aminoisoquinolin-7-yl)-N-(1-methylpiperidin-4-yl)benzamide;
(3-(1-aminoisoquinolin-7-yl)phenyl)(4-methoxypiperidin-1-yl)methanone;
3-(1-aminoisoquinolin-7-yl)-N-((1-methylazetidin-3-yl)methyl)benzamide;
3-(1-aminoisoquinolin-7-yl)-N-(3-(dimethylamino)propyl)benzamide;
(4-(1-aminoisoquinolin-7-yl)thiophen-2-yl)(pyrrolidin-1-yl)methanone;
6-(3-(3-(dimethylamino)pyrrolidine-1-carbonyl)phenyl)isoquinolin-1 (2H)-one;
(3-(1-aminoisoquinolin-7-yl)phenyl)(4-methylpiperazin-1-yl)methanone;
6-(3-(pyrrolidine-1-carbonyl)phenyl)isoquinolin-1 (2H)-one;
(3-(1-aminoisoquinolin-7-yl)phenyl)(pyrrolidin-1-yl)methanone;
(1-(1-aminoisoquinolin-7-yl)pyrrolidin-3-yl)(pyrrolidin-1-yl)methanone;
5-(1-aminoisoquinolin-7-yl)-N-(2-(dimethylamino)ethyl)benzo[b]thiophene-3-carboxamide;
1′-amino-N-(2-(dimethylamino)ethyl)-[6,7′-biisoquinoline]-3-carboxamide;
3-(1-aminoisoquinolin-7-yl)-N-(3-(dimethylamino)propyl)benzo[b]thiophene-6-carboxamide;
7-(1-(4-(dimethylamino)butyl)-1H-benzo[d]imidazol-6-yl)isoquinolin-1-amine;
3-(1-aminoisoquinolin-7-yl)-N-(3-(dimethylamino)propyl)-1H-indole-6-carboxamide;
5-(1-aminoisoquinolin-7-yl)-N-(2-(dimethylamino)ethyl)-2-methyl-2H-indazole-3-carboxamide;
7-(1H-indazol-5-yl)isoquinolin-1-amine;
7-(1H-benzo[d]imidazol-5-yl)isoquinolin-1-amine;
4-(1-methyl-1H-pyrazol-5-yl)-2-(2-methylpiperazin-1-yl)-8-(1H-pyrazol-5-yl)-1,7-naphthyridine;
2-(3,8-diazabicyclo[3.2.1]octan-3-yl)-4-(1-methyl-1H-pyrazol-5-yl)-8-(1H-pyrazol-3-yl)-1,7-naphthyridine;
3-(4-(1-methyl-1H-pyrazol-5-yl)-8-(1H-pyrazol-3-yl)-1,7-naphthyridin-2-yl)-3,6-diazabicyclo[3.1.1]heptane;
6-isopropyl-4-(4-(1-methyl-1H-pyrazol-5-yl)-8-(1H-pyrazol-3-yl)-1,7-naphthyridin-2-yl)pyridin-2-amine; and
6-isopropyl-4-(4-(1-methyl-1H-pyrazol-5-yl)-8-(1H-pyrazol-3-yl)quinolin-2-yl)pyridin-2-amine.

28. A pharmaceutical composition comprising the compound according to any one of claims 1 to 27 and at least one additional component chosen from pharmaceutically acceptable carriers, pharmaceutically acceptable vehicles, and pharmaceutically acceptable excipients.

29. A method of treating cancer in a subject in need thereof, comprising administering to the subject an effective amount of the compound according to any one of claims 1 to 27 or of the pharmaceutical composition according to claim 28.

30. A method of inhibiting E3 ligase in cells, comprising treating cells with the compound of any one of claims 1 to 27 or of the pharmaceutical composition according to claim 28.
